SEQUENCE LISTING (379 sequences)

SEQ ID NO: 1 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-aminohexanoic acid
XWFGXXDXGXWXX

SEQ ID NO: 2 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
XWFGXXDXGXWXX

SEQ ID NO: 3 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-([1,1'-biphenyl]-3-yl)-2-aminoacetic acid
XWFGXXDXGXWXX

SEQ ID NO: 4 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1S,4R)-4-aminocyclopent-2-ene-1-carboxylic acid
  SITE 13: note = (S)-2-([1,1'-biphenyl]-3-yl)-2-aminoacetic acid
XWFGXXDXGXWXX

SEQ ID NO: 5 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-([1,1'-biphenyl]-3-yl)-2-aminoacetic acid
XWFGXXDXGXWXX

SEQ ID NO: 6 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-([1,1'-biphenyl]-3-yl)-2-aminoacetic acid
XWFGXXDXGXWXX

SEQ ID NO: 7 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-2,3,4,5-tetrahydro-1H-benzo[d]azepine-2-carboxy lic acid
XWFGXXDXGXWXX

SEQ ID NO: 8 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-([1,1'-biphenyl]-3-yl)-2-aminoacetic acid
FWFGXXDXGXWXX

SEQ ID NO: 9 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-([1,1'-biphenyl]-3-yl)-2-aminoacetic acid
FWFGXXDXGXWXX

SEQ ID NO: 10 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-([1,1'-biphenyl]-3-yl)-2-aminoacetic acid
FWFGXXDXGXWXX

SEQ ID NO: 11 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
XWFGXXDXGXWXX

SEQ ID NO: 12 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-methylhexanoic acid
XWFGXXDXGXWXX

SEQ ID NO: 13 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
FWFGXXDXGXWXX

SEQ ID NO: 14 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = 2-(3-(aminomethyl)phenyl)acetic acid
  SITE 13: note = (S)-2-([1,1'-biphenyl]-3-yl)-2-aminoacetic acid
XWFGXXDXGXWXX

SEQ ID NO: 15 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-phenylacetic acid
XWFGXXDXGXKXX

SEQ ID NO: 16 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
XWFGXXDXGXKXX

SEQ ID NO: 17 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-1-carboxylic acid
XWFGXXDXGXWXX

SEQ ID NO: 18 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(4-aminophenyl)propanoic acid
  SITE 12: note = 2-(3-(aminomethyl)phenyl)acetic acid
  SITE 13: note = (S)-2-aminohexanoic acid
XWFGXXDXGXFXX

SEQ ID NO: 19 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
XWFGXXDXGXQXX

SEQ ID NO: 20 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (2S)-piperidine-2-carboxylic acid
XWFGXXDXGXWXX

SEQ ID NO: 21 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXWXX

SEQ ID NO: 22 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-cyclohexylacetic acid
XWFGXXDXGXWXX

SEQ ID NO: 23 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
XWFGXXDXGXWXF

SEQ ID NO: 24 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-amino-2-cyclohexylacetic acid
XWFGXXDXGXWXX

SEQ ID NO: 25 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-amino-4-(3-chlorophenyl)butanoic acid
XWFGXXDXGXWXX

SEQ ID NO: 26 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-(4-(hydroxymethyl)phenyl)acetic acid
XWFGXXDXGXWXX

SEQ ID NO: 27 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = 2-(3-(aminomethyl)phenyl)acetic acid
  SITE 13: note = L-homotyrosine
XWFGXXDXGXWXX

SEQ ID NO: 28 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXWXX

SEQ ID NO: 29 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-1-carboxylic acid
XWFGXXDXGXKXX

SEQ ID NO: 30 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-aminophenyl)propanoic acid
  SITE 12: note = 2-(3-(aminomethyl)phenyl)acetic acid
  SITE 13: note = (S)-2-amino-2-phenylacetic acid
XWFGXXDXGXFXX

SEQ ID NO: 31 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-phenylacetic acid
XWFGXXDXGXQXX

SEQ ID NO: 32 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-aminohexanoic acid
XWFGXXDXGXHXX

SEQ ID NO: 33 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-(aminomethyl)phenyl)propanoic acid
  SITE 12: note = 2-(3-(aminomethyl)phenyl)acetic acid
  SITE 13: note = (S)-2-amino-2-phenylacetic acid
XWFGXXDXGXXXX

SEQ ID NO: 34 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-(aminomethyl)phenyl)propanoic acid
  SITE 12: note = 2-(3-(aminomethyl)phenyl)acetic acid
  SITE 13: note = (S)-2-aminohexanoic acid
XWFGXXDXGXXXX

SEQ ID NO: 35 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-[4-(aminomethyl)phenyl]propanoic acid
  SITE 12: note = 2-(3-(aminomethyl)phenyl)acetic acid
  SITE 13: note = (S)-2-amino-2-phenylacetic acid
XWFGXXDXGXFXX

SEQ ID NO: 36 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = [(5S)-5-amino-5-carboxy-pentyl]-trimethyl-ammonium
  SITE 12: note = 2-(3-(aminomethyl)phenyl)acetic acid
  SITE 13: note = (S)-2-aminohexanoic acid
XWFGXXDXGXXXX

SEQ ID NO: 37 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = 3-amino-2,2-dimethylpropanoic acid
  SITE 13: note = (S)-2-aminohexanoic acid
FWFGXXDXGXKXX

SEQ ID NO: 38 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-aminohexanoic acid
FWFGXXDXGXKXX

SEQ ID NO: 39 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-phenylacetic acid
XWFGXXDXGXHXX

SEQ ID NO: 40 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (2S)-2-amino-3-(4-carbamoylphenyl)propanoic acid
  SITE 12: note = 2-(3-(aminomethyl)phenyl)acetic acid
  SITE 13: note = (S)-2-amino-2-phenylacetic acid
XWFGXXDXGXFXX

SEQ ID NO: 41 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-(o-tolyl)acetic acid
XWFGXXDXGXWXX

SEQ ID NO: 42 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-3-(piperidin-4-yl)propanoic acid
XWFGXXDXGXWXX

SEQ ID NO: 43 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-2-amino-2-(3-(hydroxymethyl)phenyl)acetic acid
XWFGXXDXGXWXX

SEQ ID NO: 44 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(2-aminoethoxy)acetic acid
  SITE 13: note = (S)-2-amino-2-phenylacetic acid
XWFGXXDXGXWXX

SEQ ID NO: 45 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-phenylacetic acid
XWFGXXDXGXWXX

SEQ ID NO: 46 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-aminohexanoic acid
XWFGXXDXGXWXX

SEQ ID NO: 47 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-aminohexanoic acid
XWFGXXDXGXWXX

SEQ ID NO: 48 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)dimethylglycine
  SITE 13: note = (S)-2-aminohexanoic acid
XWFGXXDXGXWXX

SEQ ID NO: 49 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-phenylacetic acid
XWFGXXDXGXWXX

SEQ ID NO: 50 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2S,4S)-4-aminopyrrolidine-2-carboxylic acid
  SITE 13: note = (S)-2-aminohexanoic acid
FWFGXXDXGXWXX

SEQ ID NO: 51 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = D-N-methyl-Alanine
  SITE 10: note = l-Biphenylalanine-4-carboxylic acid
  SITE 12: note = (2S,4S)-4-aminopyrrolidine-2-carboxylic acid
  SITE 13: note = L-homotyrosine
XWFGXXDXGXWXX

SEQ ID NO: 52 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-amino-2-phenylacetic acid
XWFGXXDXGXHXX

SEQ ID NO: 53 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-aminohexanoic acid
XWFGXXDXGXHXX

SEQ ID NO: 54 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-aminohexanoic acid
XWFGXXDXGXQXX

SEQ ID NO: 55 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-aminohexanoic acid
XWFGXXDXGXQXX

SEQ ID NO: 56 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-aminohexanoic acid
FWFGXXDXGXHXX

SEQ ID NO: 57 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXHXX

SEQ ID NO: 58 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-aminohexanoic acid
FWFGXXDXGXKXX

SEQ ID NO: 59 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
FWFGXXDXGXWXX

SEQ ID NO: 60 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 61 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-cyclohexylacetic acid
XWFGXXDXGXWXX

SEQ ID NO: 62 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-(aminomethyl)phenyl)propanoic acid
  SITE 12: note = (1˜{R},2˜{S})-2-(aminomethyl)cyclopropane-1-carboxy lic acid
  SITE 13: note = (S)-2-amino-2-phenylacetic acid
XWFGXXDXGXXXX

SEQ ID NO: 63 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(pyridin-4-yl)acetamido)pyrrolidine-2- carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXKXX

SEQ ID NO: 64 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-aminophenyl)acetic acid
  SITE 13: note = (S)-2-aminohexanoic acid
FWFGXXDXGXKXX

SEQ ID NO: 65 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-amino-2-phenylacetic acid
XWFGXXDXGXKXX

SEQ ID NO: 66 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (1˜{R},2˜{S})-2-(aminomethyl)cyclopropane-1-carboxy lic acid
  SITE 13: note = (S)-2-amino-2-phenylacetic acid
XWFGXXDXGXXXX

SEQ ID NO: 67 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-aminohexanoic acid
FWFGXXHXGXWXX

SEQ ID NO: 68 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXKXX

SEQ ID NO: 69 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-1,2,3,4-tetrahydroisoquinoline-3-carboxylic acid
XWFGXXDXGXWXX

SEQ ID NO: 70 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-1,2,3,4-tetrahydroisoquinoline-3-carboxylic acid
XWFGXXDXGXWXX

SEQ ID NO: 71 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(2-aminoethoxy)acetic acid
  SITE 13: note = (S)-1,2,3,4-tetrahydroisoquinoline-3-carboxylic acid
XWFGXXDXGXWXX

SEQ ID NO: 72 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-6-hydroxy-1,2,3,4-tetrahydroisoquinoline-3-carb oxylic acid
XWFGXXDXGXWXX

SEQ ID NO: 73 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXWXX

SEQ ID NO: 74 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 75 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
XWFGXXDXGXWXX

SEQ ID NO: 76 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 3-amino-2,2-dimethylpropanoic acid
  SITE 13: note = (S)-2-amino-2-methylhexanoic acid
FWFGXXDXGXKXX

SEQ ID NO: 77 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 7: note = (2S)-2-amino-3-(3-methylimidazol-4-yl)propanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXXXGXWXX

SEQ ID NO: 78 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-phenylacetic acid
XWFGXXTXGXWXX

SEQ ID NO: 79 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 7: note = (S)-2-amino-2-(tetrahydro-2H-pyran-4-yl)acetic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-1,2,3,4-tetrahydroisoquinoline-3-carboxylic acid
XWFGXXXXGXKXX

SEQ ID NO: 80 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-phenylacetic acid
XWFGXXDXGXWXX

SEQ ID NO: 81 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-1,2,3,4-tetrahydroisoquinoline-1-carboxylic acid
XWFGXXDXGXWXX

SEQ ID NO: 82 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-1,2,3,4-tetrahydroisoquinoline-3-carboxylic acid
XWFGXXDXGXWXX

SEQ ID NO: 83 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-1-carboxylic acid
XWFGXXDXGXHXX

SEQ ID NO: 84 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (1˜{R},2˜{S})-2-(aminomethyl)cyclopropane-1-carboxy lic acid
  SITE 13: note = (S)-2-aminohexanoic acid
XWFGXXDXGXXXX

SEQ ID NO: 85 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(4-(aminomethyl)phenyl)acetic acid
  SITE 13: note = (S)-1,2,3,4-tetrahydroisoquinoline-3-carboxylic acid
XWFGXXDXGXXXX

SEQ ID NO: 86 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
FWFGXXDXGXWXX

SEQ ID NO: 87 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXWXX

SEQ ID NO: 88 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 7: note = l-homoserine
  SITE 8: note = (R)-piperidine-2-carboxylic acid
  SITE 10: note = l-Biphenylalanine-4-carboxylic acid
  SITE 12: note = (1˜{R},2˜{S})-2-(aminomethyl)cyclopropane-1-carboxy lic acid
  SITE 13: note = (S)-2-amino-2-phenylacetic acid
XWFGXXXXGXKXX

SEQ ID NO: 89 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXHXX

SEQ ID NO: 90 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXHXX

SEQ ID NO: 91 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 2-(2-aminoethylamino)acetic acid
  SITE 13: note = (S)-2-aminohexanoic acid
XWFGXXDXGXWXX

SEQ ID NO: 92 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-phenylacetic acid
XWFGXXDXGXWXX

SEQ ID NO: 93 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-phenylacetic acid
XWFGXXDXGXWXX

SEQ ID NO: 94 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXWXX

SEQ ID NO: 95 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (2S)-2,4-diaminobutanoic acid
  SITE 12: note = 2-(4-(aminomethyl)phenyl)acetic acid
  SITE 13: note = (S)-1,2,3,4-tetrahydroisoquinoline-3-carboxylic acid
XWFGXXDXGXXXX

SEQ ID NO: 96 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 4-(aminomethyl)piperidine-4-carboxylic acid
  SITE 13: note = (S)-2-aminohexanoic acid
FWFGXXDXGXWXX

SEQ ID NO: 97 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXWXX

SEQ ID NO: 98 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 99 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 100 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 101 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 102 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 103 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXWXX

SEQ ID NO: 104 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 105 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-phenylacetamido)pyrrolidine-2-carboxyl ic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXKXX

SEQ ID NO: 106 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(benzylamino)pyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXKXX

SEQ ID NO: 107 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-acetamidopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXKXX

SEQ ID NO: 108 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 4-(aminomethyl)tetrahydro-2H-pyran-4-carboxylic acid
  SITE 13: note = (S)-2-aminohexanoic acid
FWFGXXDXGXWXX

SEQ ID NO: 109 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (S)-1,2,3,4-tetrahydroisoquinoline-3-carboxylic acid
XWFGXXDXGXXXX

SEQ ID NO: 110 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-1,2,3,4-tetrahydroisoquinoline-3-carboxylic acid
XWFGXXDXGXXXX

SEQ ID NO: 111 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = 21-amino-21-carboxy-N,N,N-trimethyl-15-oxo-3,6,9,12 -tetraoxa-16-azahenicosan-1-aminium
  SITE 12: note = (2-aminoethyl)glycine
  SITE 13: note = (S)-2-aminohexanoic acid
XWFGXXDXGXXXX

SEQ ID NO: 112 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 4-(aminomethyl)-1,1-dimethylpiperidin-1-ium-4-carbo xylic acid
  SITE 13: note = (S)-2-aminohexanoic acid
FWFGXXDXGXWXX

SEQ ID NO: 113 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(2-aminoethoxy)acetic acid
  SITE 13: note = (S)-7-hydroxy-1,2,3,4-tetrahydroisoquinoline-3-carb oxylic acid
XWFGXXDXGXWXX

SEQ ID NO: 114 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-cyclohexylacetic acid
FWFGXXDXGXWXX

SEQ ID NO: 115 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(3-(aminomethyl)phenyl)acetic acid
  SITE 13: note = (S)-7-hydroxy-1,2,3,4-tetrahydroisoquinoline-3-carb oxylic acid
XWFGXXDXGXXXX

SEQ ID NO: 116 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 2-(3-(aminomethyl)phenyl)acetic acid
  SITE 13: note = (S)-7-hydroxy-1,2,3,4-tetrahydroisoquinoline-3-carb oxylic acid
XWFGXXDXGXKXX

SEQ ID NO: 117 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (2S)-2-amino-3-(7-fluoro-1H-indol-3-yl)propanoic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXWXX

SEQ ID NO: 118 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2S)-2,5-diaminopentanoic acid
  SITE 13: note = (S)-7-hydroxy-1,2,3,4-tetrahydroisoquinoline-3-carb oxylic acid
XWFGXXDXGXWXX

SEQ ID NO: 119 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(pyridin-4-yl)acetamido)pyrrolidine-2- carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-aminohexanoic acid
XWFGXXDXGXWXX

SEQ ID NO: 120 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = 4-(aminomethyl)-1-methylpiperidine-4-carboxylic acid
  SITE 13: note = (S)-2-aminohexanoic acid
FWFGXXDXGXWXX

SEQ ID NO: 121 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2S)-2,5-diaminopentanoic acid
  SITE 13: note = (S)-6-hydroxy-1,2,3,4-tetrahydroisoquinoline-3-carb oxylic acid
XWFGXXDXGXWXX

SEQ ID NO: 122 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2S)-2,5-diaminopentanoic acid
  SITE 13: note = (S)-6-hydroxy-1,2,3,4-tetrahydroisoquinoline-3-carb oxylic acid
XWFGXXDXGXWXX

SEQ ID NO: 123 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXWXX

SEQ ID NO: 124 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-6-(benzyloxy)-1,2,3,4-tetrahydroisoquinoline-3- carboxylic acid
XWFGXXDXGXWXX

SEQ ID NO: 125 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXWXX

SEQ ID NO: 126 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(4-(aminomethyl)phenyl)acetic acid
  SITE 13: note = (S)-6-hydroxy-1,2,3,4-tetrahydroisoquinoline-3-carb oxylic acid
XWFGXXDXGXWXX

SEQ ID NO: 127 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-(aminomethyl)phenyl)propanoic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXXXX

SEQ ID NO: 128 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-cyclohexylacetic acid
XWFGXXDXGXWXX

SEQ ID NO: 129 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-6-(benzyloxy)-1,2,3,4-tetrahydroisoquinoline-3- carboxylic acid
XWFGXXDXGXWXX

SEQ ID NO: 130 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(4-(aminomethyl)phenyl)acetic acid
  SITE 13: note = (S)-6-hydroxy-1,2,3,4-tetrahydroisoquinoline-3-carb oxylic acid
XWFGXXDXGXWXX

SEQ ID NO: 131 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-7-hydroxy-1,2,3,4-tetrahydroisoquinoline-3-carb oxylic acid
XWFGXXDXGXWXX

SEQ ID NO: 132 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-6-hydroxy-1,2,3,4-tetrahydroisoquinoline-3-carb oxylic acid
XWFGXXDXGXWXX

SEQ ID NO: 133 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 134 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 135 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXXXX

SEQ ID NO: 136 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 137 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 138 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-hydroxy-1,2,3,4-tetrahydroisoquinoline-3-carb oxylic acid
XWFGXXDXGXWXX

SEQ ID NO: 139 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(1H-indol-2-yl)acetamido)pyrrolidine-2 -carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 2-(2-aminoethylamino)acetic acid
  SITE 13: note = (S)-2-amino-2-methylhexanoic acid
XWFGXXDXGXWXX

SEQ ID NO: 140 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
XWFGXXDXGXHXX

SEQ ID NO: 141 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 142 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-1,2,3,4-tetrahydroisoquinoline-3-carboxylic acid
XWFGXXDXGXHXX

SEQ ID NO: 143 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(pyridin-3-yl)acetamido)pyrrolidine-2- carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 144 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(pyridin-2-yl)acetamido)pyrrolidine-2- carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 145 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-((R)-2-(pyridin-4-yl)propanamido)pyrrolid ine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 146 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-((S)-2-(pyridin-4-yl)propanamido)pyrrolid ine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 147 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(isonicotinamido)pyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 148 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(3-(pyridin-4-yl)propanamido)pyrrolidine- 2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 149 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-(4-fluorophenyl)acetamido)pyrrolidine- 2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 150 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(2-cyclohexylacetamido)pyrrolidine-2-carb oxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 151 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-[[2-(4-phenylphenyl)acetyl]amino]pyrrolid ine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 152 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(3-(aminomethyl)phenyl)propanoic acid
  SITE 12: note = 2-(2-aminoethylamino)acetic acid
  SITE 13: note = (S)-2-amino-2-methylhexanoic acid
XWFGXXDXGXXXX

SEQ ID NO: 153 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethylamino)acetic acid
  SITE 13: note = (S)-2-amino-2-methylhexanoic acid
XWFGXXDXGXWXX

SEQ ID NO: 154 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 155 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
XWFGXXDXGXWXX

SEQ ID NO: 156 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-cyclohexylacetic acid
XWFGXXDXGXWXX

SEQ ID NO: 157 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 158 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 159 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-3-cyclohexylpropanoic acid
FWFGXXDXGXWXX

SEQ ID NO: 160 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = Pi-Methyl-L-histidine
FWFGXXDXGXWXX

SEQ ID NO: 161 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = Pi-Methyl-L-histidine
XWFGXXDXGXWXX

SEQ ID NO: 162 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-fluoro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
XWFGXXDXGXXXX

SEQ ID NO: 163 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 164 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 165 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 166 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = 1-aminocyclohexane-1-carboxylic acid
FWFGXXDXGXWXX

SEQ ID NO: 167 - protein, 13 aa, synthetic construct
  SITE 1: note = (S)-3-(4-(4-acetylpiperazin-1-yl)phenyl)-2-aminopro panoic acid
  SITE 2: note = (2S)-2-amino-3-(6-methyl-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 168 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(4-(aminomethyl)phenyl)acetic acid
  SITE 13: note = (S)-7-hydroxy-1,2,3,4-tetrahydroisoquinoline-3-carb oxylic acid
FWFGXXDXGXWXX

SEQ ID NO: 169 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethoxy)acetic acid
  SITE 13: note = (S)-7-hydroxy-1,2,3,4-tetrahydroisoquinoline-3-carb oxylic acid
XWFGXXDXGXWXX

SEQ ID NO: 170 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXWXX

SEQ ID NO: 171 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-(benzyloxy)-1,2,3,4-tetrahydroisoquinoline-3- carboxylic acid
FWFGXXDXGXWXX

SEQ ID NO: 172 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXHXX

SEQ ID NO: 173 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (2S)-2-amino-3-(7-fluoro-1H-indol-3-yl)propanoic acid
  SITE 12: note = (2-aminoethyl)glycine
  SITE 13: note = (S)-7-(benzyloxy)-1,2,3,4-tetrahydroisoquinoline-3- carboxylic acid
FWFGXXDXGXWXX

SEQ ID NO: 174 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXTXGXWXX

SEQ ID NO: 175 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXTXGXWXX

SEQ ID NO: 176 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-cyclopentylacetic acid
FWFGXXDXGXWXX

SEQ ID NO: 177 - protein, 13 aa, synthetic construct
  SITE 1: note = (S)-3-(4-(4-acetylpiperazin-1-yl)phenyl)-2-aminopro panoic acid
  SITE 2: note = (2˜{S})-2-amino-3-(1-bicyclo[1.1.1]pentanyl)propano ic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FXFGXXDXGXWXX

SEQ ID NO: 178 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 179 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-cyclohexylacetic acid
FWFGXXDXGXWXX

SEQ ID NO: 180 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = N-(2-aminoethyl)-N-methylglycine
  SITE 13: note = (S)-2-amino-2-cyclohexylacetic acid
FWFGXXDXGXXXX

SEQ ID NO: 181 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-7-hydroxy-1,2,3,4-tetrahydroisoquinoline-3-carb oxylic acid
XWFGXXDXGXWXX

SEQ ID NO: 182 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 3-aminopropanoic acid
  SITE 13: note = (S)-2-amino-2-methylhexanoic acid
FWFGXXDXGXWXX

SEQ ID NO: 183 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = 3-aminopropanoic acid
  SITE 13: note = (S)-7-hydroxy-1,2,3,4-tetrahydroisoquinoline-3-carb oxylic acid
XWFGXXDXGXHXX

SEQ ID NO: 184 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
FWFGXXDXGXWXX

SEQ ID NO: 185 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXWXX

SEQ ID NO: 186 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 187 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 188 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 189 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 190 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
XWFGXXDXGXWXX

SEQ ID NO: 191 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
XWFGXXDXGXWXX

SEQ ID NO: 192 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2S)-2,6-diaminohexanoic acid
  SITE 13: note = (S)-7-hydroxy-1,2,3,4-tetrahydroisoquinoline-3-carb oxylic acid
XWFGXXDXGXWXX

SEQ ID NO: 193 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2R)-2,3-diaminopropanoic acid
  SITE 13: note = (S)-7-hydroxy-1,2,3,4-tetrahydroisoquinoline-3-carb oxylic acid
XWFGXXDXGXWXX

SEQ ID NO: 194 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
FWFGXXDXGXXXX

SEQ ID NO: 195 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-phenyl-1,2,3,4-tetrahydroisoquinoline-3-carbo xylic acid
XWFGXXDXGXWXX

SEQ ID NO: 196 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-(phenethylamino)pyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXWXX

SEQ ID NO: 197 - protein, 13 aa, synthetic construct
  SITE 1: note = (S)-2-(2-(3-(4-(4-(2-amino-2-carboxyethyl)phenyl)pi perazin-1-yl)-3-oxopropoxy)ethoxy)-N,N,N-trimethylethan-1- aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 198 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-((4-fluorobenzyl)oxy)-1,2,3,4-tetrahydroisoqu inoline-3-carboxylic acid
XWFGXXDXGXWXX

SEQ ID NO: 199 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXWXX

SEQ ID NO: 200 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 201 - protein, 13 aa, synthetic construct
  SITE 1: note = (S)-3-(4-(4-(2,5,8,11-tetraoxatetradecan-14-oyl)pip erazin-1-yl)phenyl)-2-aminopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 202 - protein, 13 aa, synthetic construct
  SITE 1: note = (S)-15-(4-(4-(2-amino-2-carboxyethyl)phenyl)piperaz in-1-yl)-N,N,N-trimethyl-15-oxo-3,6,9,12-tetraoxapentadeca n-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXWXX

SEQ ID NO: 203 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 204 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 205 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 206 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
XWFGXXDXGXWXX

SEQ ID NO: 207 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 208 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 209 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 210 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-3-cyclohexyl-2-methylpropanoic acid
FWFGXXDXGXWXX

SEQ ID NO: 211 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 212 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 213 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
XWFGXXDXGXWXX

SEQ ID NO: 214 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
XWFGXXDXGXWXX

SEQ ID NO: 215 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
XWFGXXDXGXWXX

SEQ ID NO: 216 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 217 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 218 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 219 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 220 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-hydroxy-1,2,3,4-tetrahydroisoquinoline-3-carb oxylic acid
FWFGXXDXGXWXX

SEQ ID NO: 221 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 222 - protein, 13 aa, synthetic construct
  SITE 1: note = (S)-2-amino-3-(4'-((2-morpholinoethyl)carbamoyl)-[1 ,1'-biphenyl]-4-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 223 - protein, 13 aa, synthetic construct
  SITE 1: note = (S)-2-amino-3-(4'-(morpholine-4-carbonyl)-[1,1'-bip henyl]-4-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 224 - protein, 13 aa, synthetic construct
  SITE 1: note = l-Biphenylalanine-4-carboxylic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 225 - protein, 13 aa, synthetic construct
  SITE 1: note = (S)-2-amino-3-(4-(4-isonicotinoylpiperazin-1-yl)phe nyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1-(2-morpholinoacetyl)piperidin-4-yl )propanoic acid
  SITE 12: note = (2-aminoethyl)glycine
  SITE 13: note = (S)-2-amino-2-methylhexanoic acid
FWFGXXDXGXXXX

SEQ ID NO: 226 - protein, 13 aa, synthetic construct
  SITE 1: note = (S)-2-amino-3-(4-(4-benzoylpiperazin-1-yl)phenyl)pr opanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1-(2-morpholinoacetyl)piperidin-4-yl )propanoic acid
  SITE 12: note = (2-aminoethyl)glycine
  SITE 13: note = (S)-2-amino-2-methylhexanoic acid
FWFGXXDXGXXXX

SEQ ID NO: 227 - protein, 13 aa, synthetic construct
  SITE 1: note = (S)-2-amino-3-(4-(4-isonicotinoylpiperazin-1-yl)phe nyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (2˜{S})-2-amino-3-(5-methyl-1,2,4-oxadiazol-3-yl)pr opanoic acid
  SITE 12: note = (2-aminoethyl)glycine
  SITE 13: note = (S)-2-amino-2-methylhexanoic acid
FWFGXXDXGXXXX

SEQ ID NO: 228 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (2˜{S})-2-amino-3-(5-methyl-1,2,4-oxadiazol-3-yl)pr opanoic acid
  SITE 12: note = (2-aminoethyl)glycine
  SITE 13: note = (S)-2-amino-2-methylhexanoic acid
FWFGXXDXGXXXX

SEQ ID NO: 229 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 9: note = (2S)-2,4-diaminobutanoic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXXXWXX

SEQ ID NO: 230 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = 5-[(2S)-2-amino-2-carboxy-ethyl]pyrimidine-2-carbox ylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWXGXXDXGXHXX

SEQ ID NO: 231 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = (2S)-2-amino-3-(2-carbamoylpyrimidin-5-yl)propanoic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWXGXXDXGXHXX

SEQ ID NO: 232 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(3-methylnaphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXHXX

SEQ ID NO: 233 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(3-fluoronaphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXHXX

SEQ ID NO: 234 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
XWFGXXDXGXHXX

SEQ ID NO: 235 - protein, 13 aa, synthetic construct
  SITE 1: note = (S)-2-amino-3-(6-(4-(2-morpholinoacetyl)piperazin-1 -yl)pyridin-3-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 236 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 237 - protein, 13 aa, synthetic construct
  SITE 1: note = (S)-2-amino-3-(6-(4-isonicotinoylpiperazin-1-yl)pyr idin-3-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-morpholin-4-ylpropanoic acid
  SITE 12: note = (2-aminoethyl)glycine
  SITE 13: note = (S)-2-amino-2-methylhexanoic acid
XWFGXXDXGXXXX

SEQ ID NO: 238 - protein, 13 aa, synthetic construct
  SITE 1: note = (S)-2-amino-3-(6-(4-isonicotinoylpiperazin-1-yl)pyr idin-3-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 239 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
FWFGXXDXGXHXX

SEQ ID NO: 240 - protein, 13 aa, synthetic construct
  SITE 1: note = (S)-2-amino-3-(4-(4-(2-amino-2-hydroxy-2-methyl-2l5 -propanoyl)piperazin-1-yl)phenyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(benzyloxy)-1,2,3,4-tetrahydroisoquinoline-3- carboxylic acid
FWFGXXDXGXHXX

SEQ ID NO: 241 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-morpholin-4-ylpropanoic acid
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 242 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-(4-R3-piperazin-1-yl)propanoic acid
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 243 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-(azetidin-3-yl)propanoic acid
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 244 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (3S)-7-(1,3-oxazol-2-ylmethoxy)-1,2,3,4-tetrahydroi soquinoline-3-carboxylic acid
FWFGXXDXGXXXX

SEQ ID NO: 245 - protein, 13 aa, synthetic construct
  SITE 1: note = (S)-2-amino-3-(6-(4-(2-morpholinoacetyl)piperazin-1 -yl)pyridin-3-yl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 246 - protein, 13 aa, synthetic construct
  SITE 1: note = (S)-2-amino-3-(4-(4-isonicotinoylpiperazin-1-yl)phe nyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 247 - protein, 13 aa, synthetic construct
  SITE 1: note = (S)-2-amino-3-(4-(4-nicotinoylpiperazin-1-yl)phenyl )propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 248 - protein, 13 aa, synthetic construct
  SITE 1: note = (S)-2-amino-3-(4-(4-picolinoylpiperazin-1-yl)phenyl )propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 249 - protein, 13 aa, synthetic construct
  SITE 1: note = (S)-2-amino-3-(4-(4-benzoylpiperazin-1-yl)phenyl)pr opanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-(4-R3-piperazin-1-yl)propanoic acid
  SITE 12: note = (2-aminoethyl)glycine
  SITE 13: note = (S)-2-amino-2-methylhexanoic acid
FWFGXXDXGXXXX

SEQ ID NO: 250 - protein, 13 aa, synthetic construct
  SITE 1: note = (S)-2-amino-3-(4-(4-benzoylpiperazin-1-yl)phenyl)pr opanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-morpholin-4-ylpropanoic acid
  SITE 12: note = (2-aminoethyl)glycine
  SITE 13: note = (S)-2-amino-2-methylhexanoic acid
FWFGXXDXGXXXX

SEQ ID NO: 251 - protein, 13 aa, synthetic construct
  SITE 1: note = (S)-2-amino-3-(4-(4-benzoylpiperazin-1-yl)phenyl)pr opanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-(azetidin-3-yl)propanoic acid
  SITE 12: note = (2-aminoethyl)glycine
  SITE 13: note = (S)-2-amino-2-methylhexanoic acid
FWFGXXDXGXXXX

SEQ ID NO: 252 - protein, 13 aa, synthetic construct
  SITE 1: note = (S)-2-amino-3-(4-(4-benzoylpiperazin-1-yl)phenyl)pr opanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 253 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2˜{S})-2-(2-aminoethylamino)propanoic acid
  SITE 13: note = (S)-2-amino-2-methylhexanoic acid
FWFGXXDXGXWXX

SEQ ID NO: 254 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = (2S)-2-amino-3-(4-carbamoylphenyl)propanoic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXHXX

SEQ ID NO: 255 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2˜{S})-2-(2-aminoethylamino)propanoic acid
  SITE 13: note = (S)-2-amino-2-methylhexanoic acid
FWFGXXDXGXWXX

SEQ ID NO: 256 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 257 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-(4-R3-piperazin-1-yl)propanoic acid
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 258 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (S)-7-hydroxy-1,2,3,4-tetrahydroisoquinoline-3-carb oxylic acid
XWFGXXDXGXXXX

SEQ ID NO: 259 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
FWFGXXDXGXXXX

SEQ ID NO: 260 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = 2˜{S})-2-amino-3-morpholin-4-ylpropanoic acid
  SITE 12: note = (2-aminoethyl)glycine
  SITE 13: note = (S)-7-phenyl-1,2,3,4-tetrahydroisoquinoline-3-carbo xylic acid
XWFGXXDXGXXXX

SEQ ID NO: 261 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1-methylpiperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 262 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 263 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXHXX

SEQ ID NO: 264 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
XWFGXXDXGXHXX

SEQ ID NO: 265 - protein, 13 aa, synthetic construct
  SITE 1: note = (S)-2-amino-3-(6-(4-(2-morpholinoacetyl)piperazin-1 -yl)pyridin-3-yl)propanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 266 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 267 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 268 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (S)-7-hydroxy-1,2,3,4-tetrahydroisoquinoline-3-carb oxylic acid
FWFGXXDXGXXXX

SEQ ID NO: 269 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (3S)-7-(1,3-oxazol-2-ylmethoxy)-1,2,3,4-tetrahydroi soquinoline-3-carboxylic acid
XWFGXXDXGXXXX

SEQ ID NO: 270 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (3S)-7-(1,3-oxazol-2-ylmethoxy)-1,2,3,4-tetrahydroi soquinoline-3-carboxylic acid
XWFGXXDXGXXXX

SEQ ID NO: 271 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = 2-(2-aminoethoxy)acetic acid
  SITE 13: note = (3S)-7-(1,3-oxazol-2-ylmethoxy)-1,2,3,4-tetrahydroi soquinoline-3-carboxylic acid
XWFGXXDXGXXXX

SEQ ID NO: 272 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXXXX

SEQ ID NO: 273 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = 2-(2-(((3R,5S)-5-carboxypyrrolidin-3-yl)amino)-2-ox oethoxy)-N,N,N-trimethylethan-1-aminium
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 274 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 275 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
XWFGXXDXGXXXX

SEQ ID NO: 276 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 277 - protein, 13 aa, synthetic construct
  SITE 1: note = (2S)-3-[6-[(1S,5R)-6-acetyl-3,6-diazabicyclo[3.1.1] heptan-3-yl]-3-pyridinyl]-2-aminopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 278 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 279 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 280 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 281 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(pyrimidin-5-yl)-1,2,3,4-tetrahydroisoquinoli ne-3-carboxylic acid
XWFGXXDXGXXXX

SEQ ID NO: 282 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(pyrimidin-5-yl)-1,2,3,4-tetrahydroisoquinoli ne-3-carboxylic acid
FWFGXXDXGXXXX

SEQ ID NO: 283 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(pyrimidin-5-yl)-1,2,3,4-tetrahydroisoquinoli ne-3-carboxylic acid
XWFGXXDXGXXXX

SEQ ID NO: 284 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
XWFGXXDXGXHXX

SEQ ID NO: 285 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(benzyloxy)-1,2,3,4-tetrahydroisoquinoline-3- carboxylic acid
FWFGXXDXGXXXX

SEQ ID NO: 286 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 287 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 288 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 289 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 290 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 291 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 292 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 293 - protein, 13 aa, synthetic construct
  SITE 1: note = (S)-2-amino-3-(4-(4-(2-morpholinoacetyl)piperazin-1 -yl)phenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 294 - protein, 13 aa, synthetic construct
  SITE 1: note = (S)-2-amino-3-(4-(4-isonicotinoylpiperazin-1-yl)phe nyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
FWFGXXDXGXXXX

SEQ ID NO: 295 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXXXX

SEQ ID NO: 296 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 297 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 298 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(benzyloxy)-1,2,3,4-tetrahydroisoquinoline-3- carboxylic acid
XWFGXXDXGXXXX

SEQ ID NO: 299 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 300 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(benzyloxy)-1,2,3,4-tetrahydroisoquinoline-3- carboxylic acid
FWFGXXDXGXXXX

SEQ ID NO: 301 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 302 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 303 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXXXX

SEQ ID NO: 304 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 305 - protein, 13 aa, synthetic construct
  SITE 1: note = (S)-2-amino-3-(4-(4-(2-morpholinoacetyl)piperazin-1 -yl)phenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 306 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-7-(benzyloxy)-1,2,3,4-tetrahydroisoquinoline-3- carboxylic acid
FWFGXXDXGXXXX

SEQ ID NO: 307 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 308 - protein, 13 aa, synthetic construct
  SITE 1: note = (S)-2-amino-3-(4-(4-isonicotinoylpiperazin-1-yl)phe nyl)propanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
FWFGXXDXGXXXX

SEQ ID NO: 309 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(pyrimidin-5-yl)-1,2,3,4-tetrahydroisoquinoli ne-3-carboxylic acid
FWFGXXDXGXXXX

SEQ ID NO: 310 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXWXX

SEQ ID NO: 311 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = 4-[4-[(2S)-2-amino-2-carboxyethyl]phenyl]cyclohexan e-1-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGFWXX

SEQ ID NO: 312 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 313 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXHXX

SEQ ID NO: 314 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2˜{S},4˜{S})-4-amino-1-methylpyrrolidine-2-carboxy lic acid
  SITE 13: note = (S)-2-amino-2-methylhexanoic acid
XWFGXXDXGXXXX

SEQ ID NO: 315 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
FWFGXXDXGXHXX

SEQ ID NO: 316 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-methylhexanoic acid
FWFGXXDXGXWXX

SEQ ID NO: 317 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = 4-[4-[(2S)-2-amino-2-carboxyethyl]phenyl]cyclohexan e-1-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGFWXX

SEQ ID NO: 318 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-methylhexanoic acid
FWFGXXDXGXWXX

SEQ ID NO: 319 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = N-(2-aminoethyl)-N-methylglycine
  SITE 13: note = (S)-2-amino-2-methylhexanoic acid
XWFGXXDXGXXXX

SEQ ID NO: 320 - protein, 13 aa, synthetic construct
  SITE 1: note = (2S)-2-amino-3-(4-phenylphenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-2-amino-2-methylhexanoic acid
XWFGXXDXGXXXX

SEQ ID NO: 321 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(benzyloxy)-1,2,3,4-tetrahydroisoquinoline-3- carboxylic acid
FWFGXXDXGXXXX

SEQ ID NO: 322 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-methylhexanoic acid
XWFGXXDXGXHXX

SEQ ID NO: 323 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXWXX

SEQ ID NO: 324 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 325 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = L-Ornithine
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXXXX

SEQ ID NO: 326 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = N-(2-aminoethyl)-N-methylglycine
  SITE 13: note = (S)-2-amino-2-methylhexanoic acid
FWFGXXDXGXWXX

SEQ ID NO: 327 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 328 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXXXX

SEQ ID NO: 329 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 330 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2˜{S},4˜{S})-4-amino-1-methylpyrrolidine-2-carboxy lic acid
  SITE 13: note = (S)-2-amino-2-methylhexanoic acid
FWFGXXDXGXHXX

SEQ ID NO: 331 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
FWFGXXDXGXHXX

SEQ ID NO: 332 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 333 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
XWFGXXDXGXHXX

SEQ ID NO: 334 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = N-(2-aminoethyl)-N-methylglycine
  SITE 13: note = (S)-2-amino-2-methylhexanoic acid
FWFGXXDXGXXXX

SEQ ID NO: 335 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 336 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXTXGXXXX

SEQ ID NO: 337 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 338 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = D-N-methyl-Alanine
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXTXGXXXX

SEQ ID NO: 339 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = 2-(2-(((3R,5S)-5-carboxypyrrolidin-3-yl)amino)-2-ox oethoxy)-N,N,N-trimethylethan-1-aminium
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 340 - protein, 13 aa, synthetic construct
  SITE 1: note = (S)-15-(4-(4-(2-amino-2-carboxyethyl)phenyl)piperaz in-1-yl)-N,N,N-trimethyl-15-oxo-3,6,9,12-tetraoxapentadeca n-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 341 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 342 - protein, 13 aa, synthetic construct
  SITE 1: note = (S)-3-(4-(4-acetylpiperazin-1-yl)phenyl)-2-aminopro panoic acid
  SITE 2: note = (2S)-2-amino-3-(7-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 343 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 344 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = (2-aminoethyl)glycine
  SITE 13: note = (S)-2-amino-2-methylhexanoic acid
FWFGXXDXGXHXX

SEQ ID NO: 345 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethoxy)acetic acid
  SITE 13: note = (S)-7-hydroxy-1,2,3,4-tetrahydroisoquinoline-3-carb oxylic acid
XWFGXXDXGXWXX

SEQ ID NO: 346 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 7: note = 21-amino-21-carboxy-N,N,N-trimethyl-15-oxo-3,6,9,12 -tetraoxa-16-azahenicosan-1-aminium
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (3˜{S})-7-(1,3-oxazol-2-ylmethoxy)-1,2,3,4-tetrahyd roisoquinoline-3-carboxylic acid
XWFGXXXXGXWXX

SEQ ID NO: 347 - protein, 13 aa, synthetic construct
  SITE 1: note = L-tyrosine O-ethyl acetamide
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
XWFGXXTXGXXXX

SEQ ID NO: 348 - protein, 13 aa, synthetic construct
  SITE 1: note = L-tyrosine O-ethyl acetamide
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-cyclopentylacetic acid
XWFGXXTXGXXXX

SEQ ID NO: 349 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-fluoro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(tetrahydro-2H-pyran-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 350 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-(2-(3-aminopyridin-4-yl)acetamido)pyrroli dine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 351 - protein, 13 aa, synthetic construct
  SITE 1: note = (2S)-2-amino-3-(4-(4-(tetrahydrofuran-3-carbonyl)pi perazin-1-yl)phenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethylamino)acetic acid
  SITE 13: note = (S)-2-amino-2-methylhexanoic acid
FWFGXXDXGXWXX

SEQ ID NO: 352 - protein, 13 aa, synthetic construct
  SITE 1: note = (S)-18-(4-(2-amino-2-carboxyethyl)phenoxy)-N,N,N-tr imethyl-15-oxo-3,6,9,12-tetraoxa-16-azaoctadecan-1-aminium
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-(oxazol-2-ylmethoxy)-1,2,3,4-tetrahydroisoqui noline-3-carboxylic acid
XWFGXXDXGXWXX

SEQ ID NO: 353 - protein, 13 aa, synthetic construct
  SITE 1: note = (S)-3-(4-(4-(2,5,8,11-tetraoxatetradecan-14-oyl)pip erazin-1-yl)phenyl)-2-aminopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 354 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-7-((tetrahydro-2H-pyran-4-yl)methoxy)-1,2,3,4-t etrahydroisoquinoline-3-carboxylic acid
XWFGXXDXGXWXX

SEQ ID NO: 355 - protein, 13 aa, synthetic construct
  SITE 1: note = (S)-2-amino-3-(4-(4-isonicotinoylpiperazin-1-yl)phe nyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethylamino)acetic acid
  SITE 13: note = (S)-2-amino-2-methylhexanoic acid
FWFGXXDXGXWXX

SEQ ID NO: 356 - protein, 13 aa, synthetic construct
  SITE 1: note = (S)-2-amino-3-(4-(4-(2-(5-methyl-1,3,4-oxadiazol-2- yl)acetyl)piperazin-1-yl)phenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethylamino)acetic acid
  SITE 13: note = (S)-2-amino-2-methylhexanoic acid
FWFGXXDXGXWXX

SEQ ID NO: 357 - protein, 13 aa, synthetic construct
  SITE 1: note = (S)-2-amino-3-(4-(4-(2-morpholinoacetyl)piperazin-1 -yl)phenyl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 358 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 359 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 360 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(naphthalen-1-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 6-((2-aminoethyl)(carboxymethyl)amino)-N,N,N-trimet hyl-6-oxohexan-1-aminium
  SITE 13: note = (S)-2-amino-2-methylhexanoic acid
FWFGXXDXGXWXX

SEQ ID NO: 361 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 12: note = N-(2-aminoethyl)-N-methylglycine
  SITE 13: note = (S)-2-amino-2-methylhexanoic acid
FWFGXXDXGXWXX

SEQ ID NO: 362 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
XWFGXXDXGXWXX

SEQ ID NO: 363 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
XWFGXXDXGXWXX

SEQ ID NO: 364 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 365 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 366 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = 2-(2-aminoethoxy)acetic acid
  SITE 13: note = (S)-7-hydroxy-1,2,3,4-tetrahydroisoquinoline-3-carb oxylic acid
FWFGXXDXGXWXX

SEQ ID NO: 367 - protein, 13 aa, synthetic construct
  SITE 1: note = L-tyrosine O-ethyl acetamide
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
XWFGXXDXGXWXX

SEQ ID NO: 368 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 369 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 370 - protein, 13 aa, synthetic construct
  SITE 1: note = S)-3-(6-(4-acetylpiperazin-1-yl)pyridin-3-yl)-2-ami nopropanoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 371 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = 1-aminocyclohexane-1-carboxylic acid
FWFGXXDXGXWXX

SEQ ID NO: 372 - protein, 13 aa, synthetic construct
  SITE 1: note = (S)-3-(4-(4-acetylpiperazin-1-yl)phenyl)-2-aminopro panoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 373 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-cyclopentylacetic acid
FWFGXXDXGXWXX

SEQ ID NO: 374 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = (2S)-2-amino-3-(4-carbamoylphenyl)propanoic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXDXGXXXX

SEQ ID NO: 375 - protein, 13 aa, synthetic construct
  SITE 1: note = S)-3-(6-(4-acetylpiperazin-1-yl)pyridin-3-yl)-2-ami nopropanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXNXGXWXX

SEQ ID NO: 376 - protein, 13 aa, synthetic construct
  SITE 1: note = S)-3-(6-(4-acetylpiperazin-1-yl)pyridin-3-yl)-2-ami nopropanoic acid
  SITE 3: note = (2S)-2-amino-3-(4-carbamoylphenyl)propanoic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = D-N-methyl-Alanine
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

SEQ ID NO: 377 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(piperidin-4-yl)propanoic acid
  SITE 12: note = (2-aminoethyl)glycine
  SITE 13: note = (S)-2-amino-2-methylhexanoic acid
FWFGXXNXGXXXX

SEQ ID NO: 378 - protein, 13 aa, synthetic construct
  SITE 1: note = (S)-3-(4-(4-acetylpiperazin-1-yl)phenyl)-2-aminopro panoic acid
  SITE 2: note = (S)-2-amino-3-(6-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXNXGXWXX

SEQ ID NO: 379 - protein, 13 aa, synthetic construct
  SITE 1: note = (S)-2-amino-3-(4-(4-(2-morpholinoacetyl)piperazin-1 -yl)phenyl)propanoic acid
  SITE 2: note = (S)-2-amino-3-(5-chloro-1H-indol-3-yl)propanoic acid
  SITE 3: note = L-phenylalanine-4-carboxylic acid
  SITE 5: note = (2S,4R)-4-aminopyrrolidine-2-carboxylic acid
  SITE 6: note = (2S,3S)-2-amino-3-(4-chloro-1H-indol-3-yl)butanoic acid
  SITE 8: note = (R)-piperidine-2-carboxylic acid
  SITE 10: note = l-Biphenylalanine-4-carboxylic acid
  SITE 11: note = (S)-2-amino-3-(1H-pyrrolo[2,3-b]pyridin-3-yl)propan oic acid
  SITE 12: note = (2-aminoethyl)glycine
  SITE 13: note = (S)-2-amino-2-methylhexanoic acid
FWFGXXDXGXWXX

(19) United States
(12) Patent Application Publication
(10) Pub. No.: US 2026/0116923 A1
(43) Pub. Date: Apr. 30, 2026

(54) CYCLIC PEPTIDE IL-1BETA TRAP FOR THE TREATMENT OF ATHEROSCLEROSIS AND INFLAMMATORY DISORDERS

(71) Applicant: Merck Sharp & Dohme LLC, Rahway, NJ (US)

(72) Inventors: Christopher W. Plummer, Westfield, NJ (US); Luigi Abate, Pomezia, Rome (IT); Elisabetta Bianchi, Rome (IT); Stefania Colarusso, Rome (IT); Emanuela Mandic, Ariccia, Rome (IT); Francesca Pavone, Rome (IT); Ali Mounaim Yousif, Pomezia, Rome (IT); Faben A. Cruz, Burlingame, CA (US); Jennifer Hanisak, Stewartsville, NJ (US); Jennifer L. Hickey, Fords, NJ (US); Ahmet Kekec, Redwood City, CA (US); Angela D. Kerekes, Marlborough, MA (US); Dmitri A. Pissarnitski, Scotch Plains, NJ (US)

(73) Assignee: Merck Sharp & Dohme LLC, Rahway, NJ (US)

(21) Appl. No.: 18/978,412

(22) Filed: Dec. 12, 2024

Related U.S. Application Data
(60) Provisional application No. 63/610,766, filed on Dec. 15, 2023.
(60) Provisional application No. 63/672,292, filed on Jul. 17, 2024.

(51) Int. Cl. C07K 7/64; A61K 38/00

(52) U.S. Cl. CPC C07K 7/64; A61K 38/00

(57) ABSTRACT

Provided are compounds of the Formula (I), or their pharmaceutically acceptable salts, wherein L¹, L², X¹-X³, C^{PC}, A¹, A², and R¹—R⁸ are as herein described.

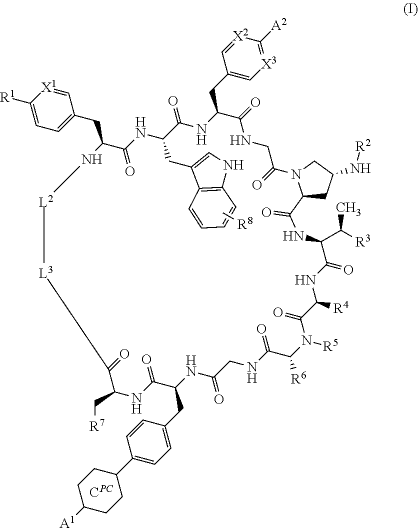

The compounds and their pharmaceutically acceptable salts can trap IL-1β and are expected to have utility as therapeutic agents, for example, for treating cardiovascular disease and inflammatory disorders. The disclosure also provides pharmaceutical compositions which comprise the compounds disclosed herein or pharmaceutically acceptable salts thereof. The disclosure also relates to methods for use of the compounds or their pharmaceutically acceptable salts in the therapy and prophylaxis of cardiovascular disease and inflammatory disorders and for preparing pharmaceuticals for this purpose.

### CROSS-REFERENCE TO RELATED APPLICATIONS

The application claims the benefit of priority to U.S. Provisional Application Nos. 63/672,292, filed Jul. 17, 2024, and 63/610,766, filed Dec. 15, 2023, the contents of each of which are incorporated herein by reference in their entireties.

### REFERENCE TO SEQUENCE LISTING SUBMITTED ELECTRONICALLY

The contents of the electronic sequence listing (25877-WO-PCT_SL.xml; Size: 1,659,851 bytes; and Date of Creation: Aug. 23, 2024) are herein incorporated by reference in their entirety.

### FIELD

The present disclosure relates to certain cyclic peptides that trap interleukin-1β (IL-1β), pharmaceutical compositions comprising such peptides, and methods for using the compounds for treating, inhibiting, or ameliorating one or more cardiovascular disease states that could benefit from trapping IL-1β, including atherosclerosis.

### BACKGROUND

Atherosclerosis is a disease of the arteries characterized by the accumulation of cholesterol plaques on the interior wall of the artery. Progression of atherosclerosis can result in hardening or narrowing of the arteries and increases the risk of plaque ruptures. These ruptures release cholesterol globules and other material into the bloodstream which may result in blockage of blood flow to the brain, heart, or other organ. Medically, these are known as Major Adverse Cardiac Events (MACE).

Risk factors for the development and progression of Atherosclerotic Cardiovascular Disease (ASCVD) include high cholesterol, high blood pressure, diet high in saturated fat, smoking, obesity, diabetes, lack of exercise, and elevated levels of C-reactive protein (CRP), a marker of inflammation.

The first line of treatment to prevent the progression of ASCVD is a healthy diet and exercise, however, compliance is generally poor. Pharmacological treatments for ASCVD have largely focused on cholesterol-lowering medications such as statins, cholesterol absorption inhibitors, and low-density lipoprotein (LDL) receptor inhibitors. These medications are highly effective at reducing the buildup of fatty acid deposits and improving arterial health. Other medications that are prescribed for ASCVD which do not ameliorate the disease state include blood thinners, such as aspirin, to prevent clumping of platelets in narrow arteries, and blood pressure medications to reduce the risk and severity of heart attacks. Surgical options for more aggressive intervention in advanced cases of ASCVD include angioplasty, stent placement, endarterectomy (surgical removal of plaques), and bypass surgery.

While cholesterol-lowering medications have served as an important standard of care for slowing the progression of atherosclerosis, clinical data support an additional critical role for inflammation in the progression of ASCVD that has remained untreated. Biomarkers of inflammation such as CRP are associated with increased risk of cardiovascular events, independent of cholesterol levels. The Canakinumab Anti-inflammatory Thrombosis Outcomes Study (CANTOS) was the first clinical trial to show that reducing vascular inflammation in the absence of concomitant lipid lowering reduces the rates of cardiovascular events. N Engl J Med 2017; 377:1119-1131. Canakinumab is an anti-interleukin-1 beta (IL-1β) human monoclonal antibody approved for clinical use in rheumatologic disorders. IL-1β is a proinflammatory cytokine that induces IL-6 and thereby elevates the downstream inflammatory biomarker high sensitivity CRP (hsCRP). Therefore, CANTOS provides proof of concept that therapies targeting IL-1β could reduce rates of MACE in certain patients in a manner that is complimentary and potentially additive to the LDL-lowering standard of care.

There is a need for additional, non-surgical therapeutic approaches beyond the standard of care cholesterol-lowering medications for slowing the progression of atherosclerosis and decreasing the risk of MACE. In addition, patients suffering from inflammatory disorders would benefit from orally administered agents which block the same cytokine, IL-1β, as canakinumab.

### SUMMARY

The present disclosure provides certain cyclic peptides that reduce inflammation by binding to the IL-1β cytokine and prevent engagement with the IL-1 receptor, resulting in inhibition of downstream pro-inflammatory signaling. These cyclic peptides can be valuable pharmaceutically active compounds for the treatment of cardiovascular diseases and inflammatory disorders. In one aspect, the present disclosure provides compounds of Formula (I)

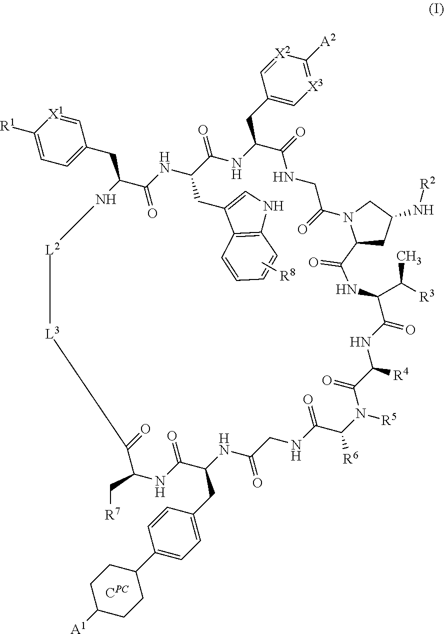

and their pharmaceutically acceptable salts.

The compounds can trap IL-1β and thereby affect the downstream pro-inflammatory signaling pathway which may be associated with cardiovascular disorders. Accordingly, in another aspect, the present disclosure provides a method for treating a cardiovascular disorder (e.g., atherosclerosis, vascular inflammation) comprising administering a therapeutically effective amount of the compound of the disclosure to a subject in need thereof. In some embodiments, the administration comprises an oral administration of the compound.

The disclosure furthermore provides processes for preparing compounds of the disclosure and pharmaceutical compositions which comprise compounds of the disclosure and a pharmaceutically acceptable carrier.

The summary of the technology described above is non-limiting and other features and advantages of the technology will be apparent from the following detailed description, and from the claims.

### BRIEF DESCRIPTION OF THE DRAWINGS

FIG. 1 describes the amino acid sequences of SEQ ID NOS: 1-379 and their associated properties.

### DETAILED DESCRIPTION

In one embodiment, the present disclosure provides a compound having structural Formula (I) as shown above wherein:
L³ is selected from the group consisting of:

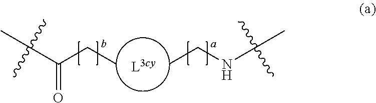

wherein L^{3cy} is C₃-C₆ cycloalkyl, C₅-C₆ cycloalkenyl, or phenyl;

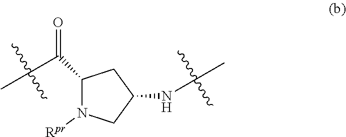

wherein R^{pr} is H or C₁-C₃ alkyl;

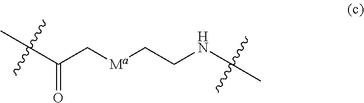

wherein R^{La} is —N(H)—, —N(CH₃)—, —N(CH₃)₂—, —N(C(O)[(CH₂)ᵣN⁺ (CH₃)₃]—, or —O—;

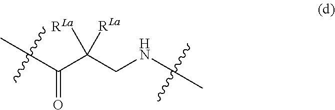

wherein each R^{La} is independently H or C₁-C₃ alkyl, or alternatively the two R^{La}, together with the carbon atom to which they are attached, form a 5- to 6-membered saturated heterocyclic ring containing 1 heteroatom group selected from the group consisting of N(H), N(CH₃), N(CH₃)₂, O and S; and

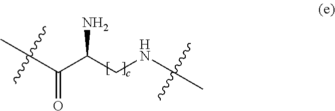

L² is Selected from the Group Consisting of:

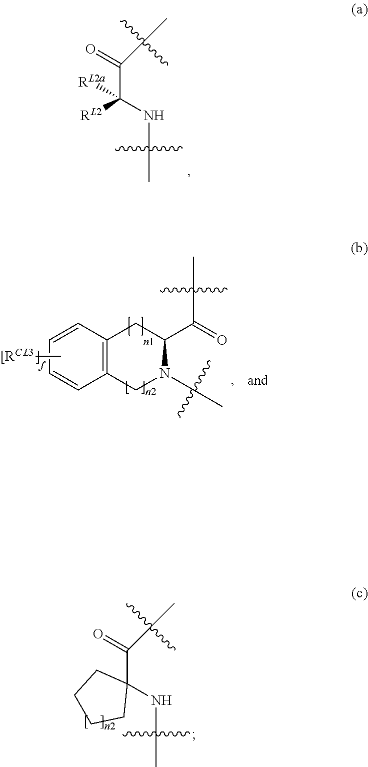

wherein:
   R^{L2a} is H or methyl;
   R^{L2} is:
      (i) C₁-C₅ alkyl; or
      (ii) —(CH₂)ₑ—C^{L2}, wherein
   C^{L2} is:
      (i) phenyl,
      (ii) C₃-C₆ cycloalkyl,
      (iii) a 5- to 6-membered, saturated heterocycle containing one heteroatom selected from the group consisting of N, O, and S; or
      (iv) a 5- to 6-membered heteroaryl containing one to two heteroatoms independently selected from the group consisting of N, O, and S wherein C^{L2} is unsubstituted or substituted by 1 to 3 R^{CL2} substituents independently selected from the group consisting of halo, C₁-C₃ alkyl, phenyl, hydroxy, and C₁-C₃ hydroxyalkyl;
   each R^{CL3} is independently selected from the group consisting of H, halo, C₁-C₃ alkyl, hydroxy, phenyl, pyrimidinyl, benzyloxy, and oxazolylmethoxy;
      wherein the phenyl, pyrimidinyl, benzyloxy and oxazolylmethoxy of R^{CL3} is unsubstituted or substituted by 1 to 3 substituents independently selected from the group consisting of halo, C₁-C₃ alkyl and C₁-C₃ alkoxy;
each of X¹, X², and X³ are independently C(H) or N;
R¹ is selected from the group consisting of:
(i) R^{1a}—C(O)N(H)—CH₂CH₂—O—, wherein
   R^{1a} is
(a) C₁-C₃ alkyl; or
(b)(CH₃)₃N⁺CH₂CH₂(OCH₂CH₂)ₘ—;
(ii) a group

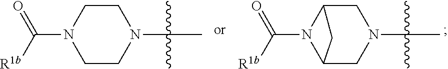

wherein
   R^{1b} is:
      (a) C₁-C₃ alkyl; or
      (b) C^{1b}—(CH₂)ₚ—, wherein C^{1b} is:
   (i) a 5- to 6-membered saturated heterocyclyl, wherein the 5- to 6-membered saturated heterocyclyl contains 1 to 2 heteroatoms independently selected from the group consisting of N, O, and S;
   (ii) a 5- to 6-membered heteroaryl, wherein the 5- to 6-membered heteroaryl contains 1 to 3 heteroatoms independently selected from the group consisting of N, O, and S; and
   (iii) phenyl;
      wherein C^{1b} is unsubstituted or substituted by 1 to 2 substituents
         independently selected from the group consisting of halo, C₁-C₃ alkyl and C₁-C₃ alkoxy;
      (c)(CH₃)₃N⁺CH₂CH₂(OCH₂CH₂)_{q}—;
      (d)(CH₃)₃N⁺CH₂CH₂OCH₂—; or
      (e)CH₃OCH₂CH₂(OCH₂CH₂)_{q}—;
(iii) a group

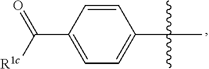

wherein
R^{1c} is hydroxy, amino, morpholinyl, morpholinyl(CH₂CH₂)—N(H)—;
A² is —C(O)OH or —C(O)NH₂;
R² is H, —C(O)—(C₁-C₃ alkyl), —C(O)—(CH₂)g-C², —C(O)—C(H)(CH₃)—C², or —(CH₂)ₕ—C², C² is
   (i) phenyl or
   (ii) a 5- to 6-membered mono- or a 9- to 10-membered bicyclic heteroaryl containing wherein said 5- to 6-membered or 9- to 10-membered bicyclic heteroaryl contains 1 to 2 heteroatoms independently selected from the group consisting of N, O, and S;
      wherein C² is unsubstituted or substituted by 1 to 3 R^{C2} substituents independently selected from the group consisting of halo, amino, hydroxy, C¹-C³ alkyl, C¹-C³ fluoroalkyl, C¹-C³ alkoxy, and phenyl;
R³ is
   (i) naphthyl; or
   (ii) a 9- to 10-membered heteroaryl, wherein said heteroaryl contains 1 to 2 heteroatoms independently selected from the group consisting of N, O, and S;
      wherein R³ is unsubstituted or substituted by 1 to 2 R^{3a} substituents independently selected from the group consisting of halo, C¹-C³ alkyl, and C¹-C³ alkoxy;
R⁴ is —(CH₂)ᵢCO₂H, —(CH₂)ᵢOH, —C(H)(OH)—CH₃, —C^{4a}, —C^{4b}, —CH₂—C^{4a}, or —CH₂—C^{4b};
   C^{4a} is tetrahydrofuryl, tetrahydropyranyl, 1,3-dioxolanyl, or 1,3-dioxanyl;
   C^{4b} is imidazolyl, wherein said imidazolyl is unsubstituted or substituted by 1 C₁-C₃ alkyl;
R⁵ is C₁-C₃ alkyl;
R⁶ is H or C₁-C₃ alkyl;
   or alternatively R⁵ and R⁶ together with the atoms to which they are attached form a pyrrolidinyl, piperidinyl or azepinyl ring;
Ring C^{PC} is phenyl or cyclohexyl;
A¹ is —CO₂H or —N(H)SO₂CH₃;
R⁷ is —(CH₂)ⱼNH₂; —(CH₂)ⱼN⁺(CH₃)₃;
   —(CH₂)ⱼN(H)C(O)CH₂CH₂O(CH₂CH₂—O)ₖ—CH₂CH₂N⁺(CH₃)₃; —(CH₂)ⱼC(O)NH₂;
   —(CH₂)ⱼOH; —(CH₂)ⱼOCH₃, or —C^{7a}
      C^{7a} is:
      (i) a 5- to 6-membered monocyclic aryl or heteroaryl, wherein said 5- to 6-membered heteroaryl contains 1 to 2 heteroatoms independently selected from the group consisting of N, O, and S;
      (ii) a 9- to 10-membered bicyclic aryl or heteroaryl, wherein said 9- to 10-membered bicyclic heteroaryl contains 1 to 4 heteroatoms independently selected from the group consisting of N, O, and S; or
      (iii) a 4- to 6-membered saturated monocyclic heterocycloalkyl, wherein said 4- to 6-membered saturated monocyclic heterocycloalkyl contains 1 to 2 heteroatoms independently selected from the group consisting of N, O, and S;
      wherein C^{7a} is unsubstituted or substituted by 1 to 3 R^{C7a} substituents independently selected from the group consisting of halo, amino, amino (C₁-C₃)alkyl, carbamoyl, hydroxy, carboxy, C₁-C₃ alkyl, C₁-C₃ fluoroalkyl, C₁-C₃ alkoxy, and —C(O)CH₂—C^{7b};
         C^{7b} is a 4- to 6-membered saturated monocyclic heterocycloalkyl, wherein said 4- to 6-membered saturated monocyclic heterocycloalkyl contains 1 to 2 heteroatoms independently selected from the group consisting of N and 0;
         wherein C^{7b} is unsubstituted or substituted by 1 to 2 R^{C7b} substituents independently selected from the group consisting of C₁-C₃ alkyl;
R⁸ is H, halo, or C₁-C₃ alkyl;
subscript a is 0 or 1;
subscript b is 0 or 1;
subscript c is 1, 2, 3, 4, or 5;
subscript d is 1 or 2;
subscript e is 0, 1, or 2;
subscript f is 0, 1, 2, or 3;
subscript g is 0, 1, or 2;
subscript h is 1 or 2;
subscript i is 1, 2, or 3;
subscript j is 1, 2, or 3; and
subscript k is 1, 2, 3, or 4;
subscript m is 0, 1, 2, 3, or 4;
subscript n1 n is 0, 1 or 2;
subscript n2 is 1 or 2;
subscript p is 0, 1, 2, or 3;
subscript q is 0, 1, 2, 3, or 4; and
subscript r is 1, 2, 3, 4, or 5; or
a pharmaceutically acceptable salt thereof.

In another embodiment, the present disclosure provides a compound of the Formula (I), wherein L³ is

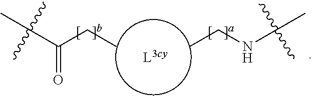

In certain embodiments, L^{3cy} is cyclopropyl, cyclopentenyl, or phenyl.

In another embodiment, the present disclosure provides a compound of the Formula (I), wherein L³ is

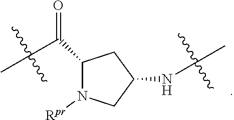

In particular embodiments, R^{pr} is H.

In another embodiment, the present disclosure provides a compound of the Formula (I), wherein L³ is

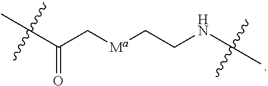

In another embodiment, the present disclosure provides a compound of the Formula (I), wherein L³ is

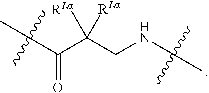

In certain embodiments,
each R^{La} is methyl; or
the two R^{La}, together with the carbon atom to which they are attached, form a piperidine ring.

In another embodiment, the present disclosure provides a compound of the Formula (I), wherein L³ is

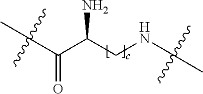

and subscript c is 1, 2, 3, or 4.

In another embodiment, the present disclosure provides a compound of the Formula (I), wherein L² is

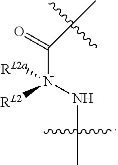

In specific embodiments, R^{L2} is C₁-C₄ alkyl.

In some embodiments, R^{L2a} is methyl.

In specific embodiments,
R^{L2a} is H and
R^{L2} is —(CH₂)ₑ—C_{L2}, wherein C_{L2} is unsubstituted or substituted phenyl, and subscript e is 0.

In another embodiment, the present disclosure provides a compound of the Formula (I), wherein L² is

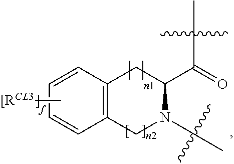

wherein subscript n1 is 0, 1, or 2 and subscript n2 is 1 or 2. In specific embodiments, R^{CL3} is selected from the group consisting of pyrimidin-5-yl, 4-fluorobenzyloxy, and 1,3-oxazolylmethoxy.

In some embodiments, L² is

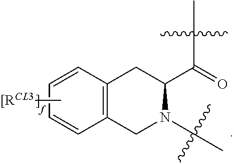

In specific embodiments, the present disclosure provides a compound of the Formula (I), wherein L² is

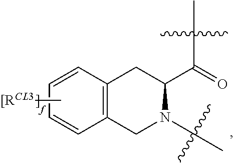

R^{CL3} is independently selected from the group consisting of benzyloxy and 1,3-oxazol-2-yl methoxy, and
subscript f is 0 or 1.

In another embodiment, the present disclosure provides a compound of the Formula (I), wherein L² is

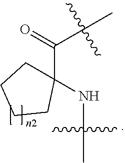

In another embodiment, the present disclosure provides a compound of the Formula (I) wherein R¹ is R^{1a}—C(O)N(H)—CH₂CH₂—O—. In certain embodiments, R^{1a} is methyl.

In another embodiment, the present disclosure provides a compound of the Formula (I) wherein:
R¹ is the group

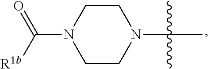

and
R^{1b} is methyl or morpholin-4-yl-CH₂—.

In a specific embodiment, the present disclosure provides a compound of the Formula (I) wherein:
R¹ is the group

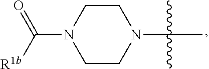

and
R^{1b} is morpholin-4-yl-CH₂—.

In another embodiment, the present disclosure provides a compound of the Formula (I) wherein R² is —H, —C(O)CH₂Ph, —CH₂Ph, —C(O)CH₃, —C(O)-pyridyl, —C(O)CH₂-indolyl, or —C(O)—C(H)(CH₃)-pyridyl.

In another embodiment, the present disclosure provides a compound of the Formula (I) wherein R³ is substituted or unsubstituted naphthyl or indolyl.

In another embodiment, the present disclosure provides a compound of the Formula (I) wherein R⁴ is —(CH₂)ᵢCO₂H. In a specific embodiment subscript i is 1.

In another embodiment, the present disclosure provides a compound of the Formula (I) wherein R⁵ and R⁶ together with the atoms to which they are attached form a piperidinyl ring.

In another embodiment, the present disclosure provides a compound of the Formula (I) wherein R⁵ and R⁶ are methyl.

In another embodiment, the present disclosure provides a compound of the Formula (I) wherein ring C^{PC} is phenyl.

In another embodiment, the present disclosure provides a compound of the Formula (I) wherein A¹ is —CO₂H.

In another embodiment, the present disclosure provides a compound of the Formula (I) wherein R⁷ is —C^{7a} In a particular embodiment, C^{7a} is indolyl.

In another embodiment, the present disclosure provides a compound of the Formula (I) wherein R⁸ is H, F or C₁. In one particular embodiment, R⁸ is substituted on the 6-position of the illustrated indole ring. In another particular embodiment, R⁸ is substituted on the 7-position of the illustrated indole ring.

In another embodiment, the present disclosure provides a compound of the Formula (I) wherein:
L³ is selected from the group consisting of:

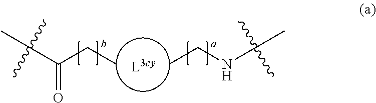

and L^{3cy} is cyclopropyl, cyclopentenyl, or phenyl;

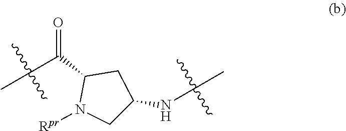

### and R^{pr} is H;

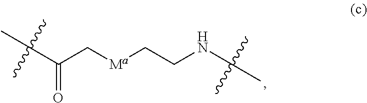

and M^{a} is —N(H)—, N(CH₃)—, or —O—;

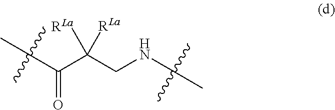

and
each R^{La} is methyl; or
the two R^{La}, together with the carbon atom to which they are attached, form a piperidine ring; and

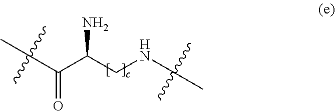

and subscript c is 1, 2, 3, or 4;
L² is selected from the group consisting of:

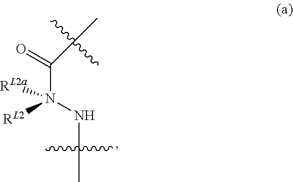

and
R^{L2a} is methyl and R^{L2} is C₁-C₄ alkyl (e.g., propyl);

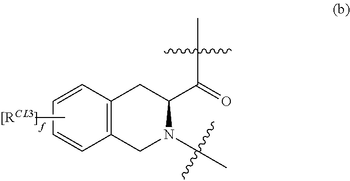

R^{CL3} is independently selected from benzyloxy and 1,3-oxazol-2-yl methoxy; and
subscript f is 0 or 1;
R¹ is:
   (i) R^{1a}—C(O)N(H)—CH₂CH₂—O— and R^{1a} is methyl; or
   (ii) the group

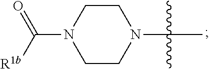

and
R^{1b} is methyl or morpholin-4-yl-CH₂—;
R² is —H;
R³ is substituted or unsubstituted naphthyl or indolyl (e.g., naphth-1-yl, 4-fluoroindol-3-yl, or 4-chloroindol-3-yl);
R⁴ is —(CH₂)ᵢCO₂H and subscript i is 1;
R⁵ and R⁶ are methyl, or alternatively, R⁵ and R⁶ together with the atoms to which they are attached form a piperidinyl ring;
Ring C^{PC} is phenyl;
A¹ is —CO₂H;
R⁷ is indolyl or 7-azaindolyl (e.g., 7-aza-3-indol-3-yl); and
R⁸ is H, F, or Cl.

In another embodiment, the present disclosure provides a compound of Formula (IA)

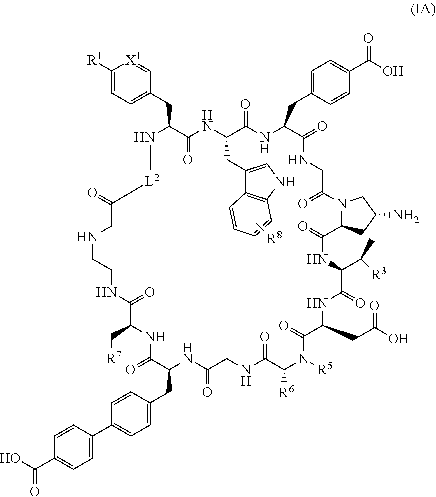

In some embodiments, the present disclosure provides the compound of the Formula (IA) or the pharmaceutically acceptable thereof, wherein:
L² is selected from the group consisting of:

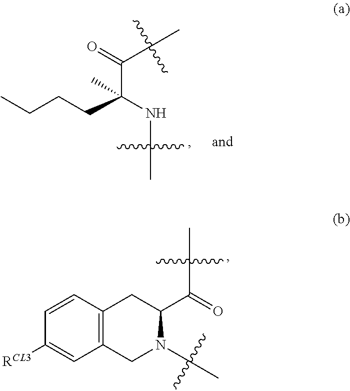

wherein
R^{CL3} is independently selected from benzyloxy and 1,3-oxazol-2-yl methoxy;
R¹ is:
   (i) R^{1a}—C(O)N(H)—CH₂CH₂—O— and R^{1a} is methyl; or
   (ii) the group

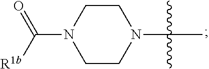

and
R^{1b} is methyl or morpholin-4-yl-CH₂—;
R³ is naphthyl or indolyl, wherein the naphthyl or indolyl is unsubstituted or substituted by 1 halo;
R⁵ and R⁶ are methyl, or alternatively, R⁵ and R⁶ together with the atoms to which they are attached form a piperidinyl ring;
R⁷ is indolyl or 7-azaindolyl; and
R⁸ is H, F, or Cl.

In another embodiment, the present disclosure provides a compound of Formula (I) wherein the compound is selected from the group consisting of SEQ ID NOS: 1-379.

In another embodiment, the present disclosure provides a compound of Formula (I) wherein the compound is selected from the group consisting of (SEQ ID NOS: 246, 292, 324, 189, 301, 302, 305, 265, 280, 199, 286, 188 and 379, respectively):

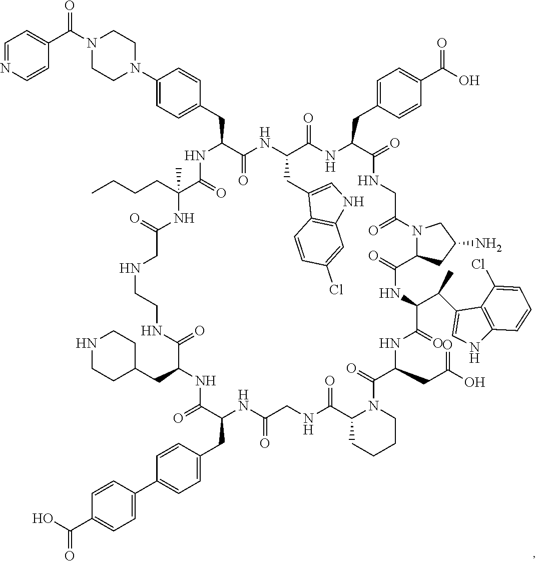 [SMILES: CCCC[C@]1(C)NC(=O)CNCCNC(=O)[C@H](CC2CCNCC2)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)c4ccncc4)CC3)cc2)NC1=O]

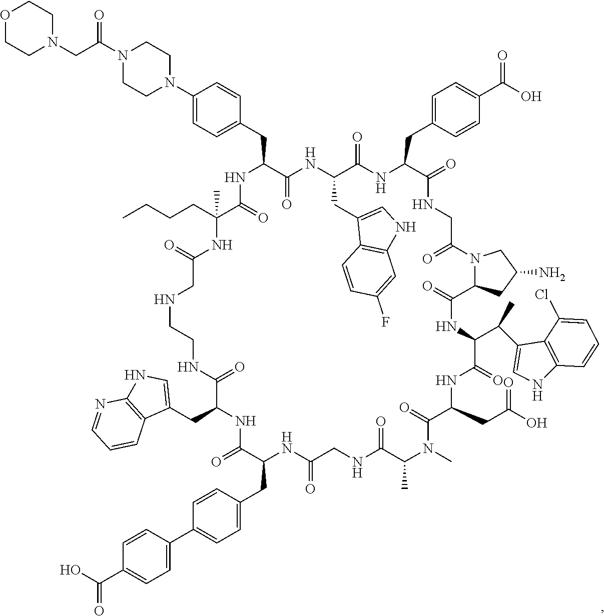 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(=O)CN4CCOCC4)CC3)cc2)NC1=O]

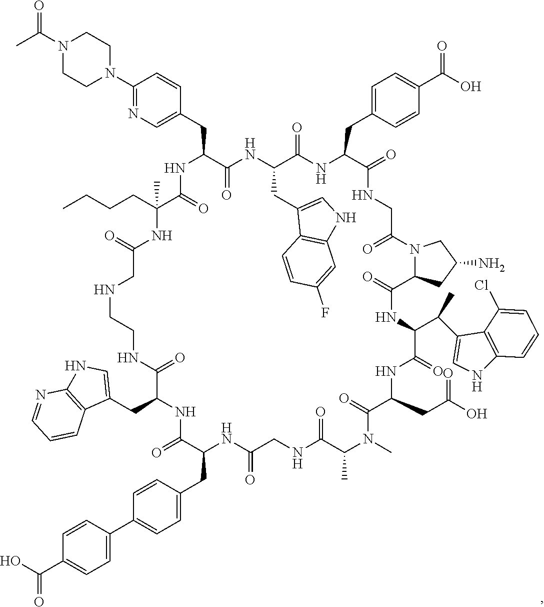 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(C)=O)CC3)nc2)NC1=O]

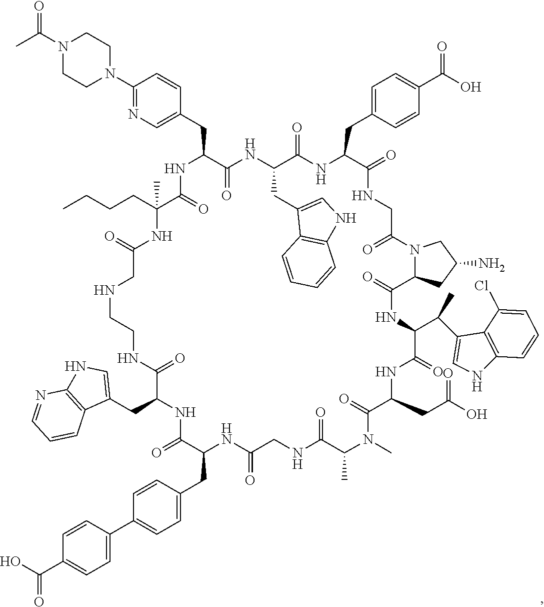 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3ccccc23)NC(=O)[C@H](Cc2ccc(N3CCN(C(C)=O)CC3)nc2)NC1=O]

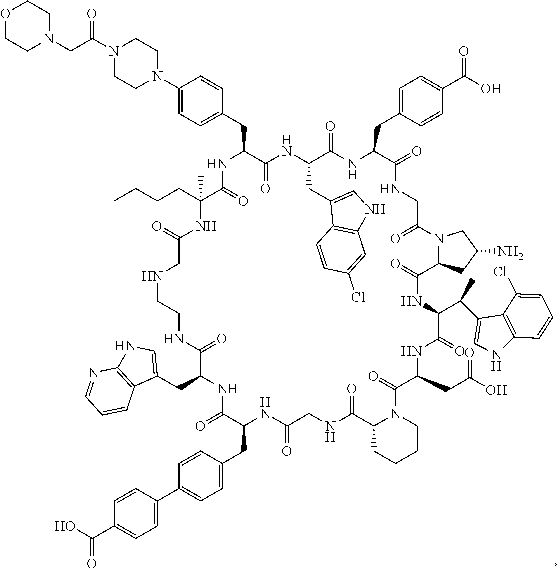 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)CN4CCOCC4)CC3)cc2)NC1=O]

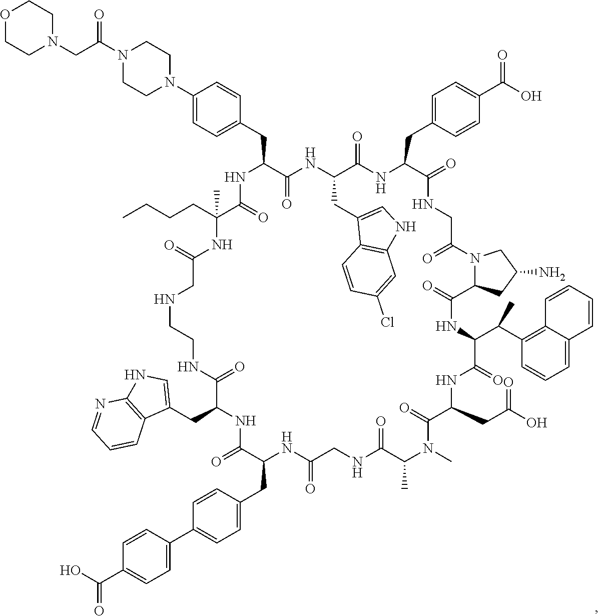 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2cccc3ccccc23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)CN4CCOCC4)CC3)cc2)NC1=O]

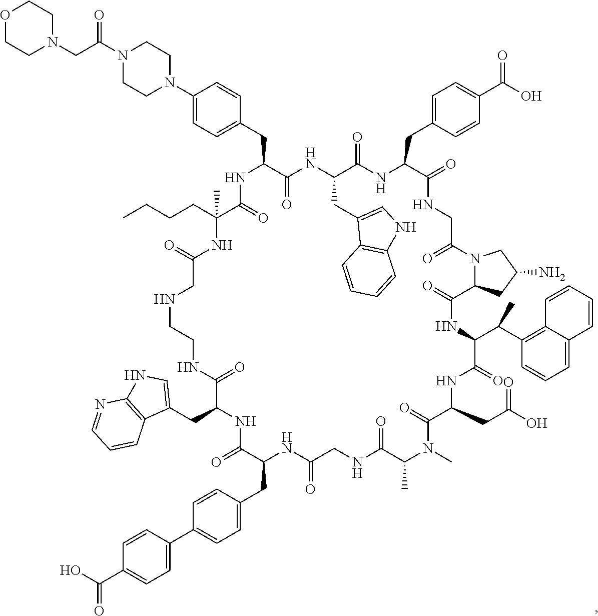 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2cccc3ccccc23)NC(=O)[C@@H]2C[C@@H](N)CN2C(=O)CNC(=O)[C@H](Cc2ccc(C(=O)O)cc2)NC(=O)[C@H](Cc2c[nH]c3ccccc23)NC(=O)[C@H](Cc2ccc(N3CCN(C(=O)CN4CCOCC4)CC3)cc2)NC1=O]

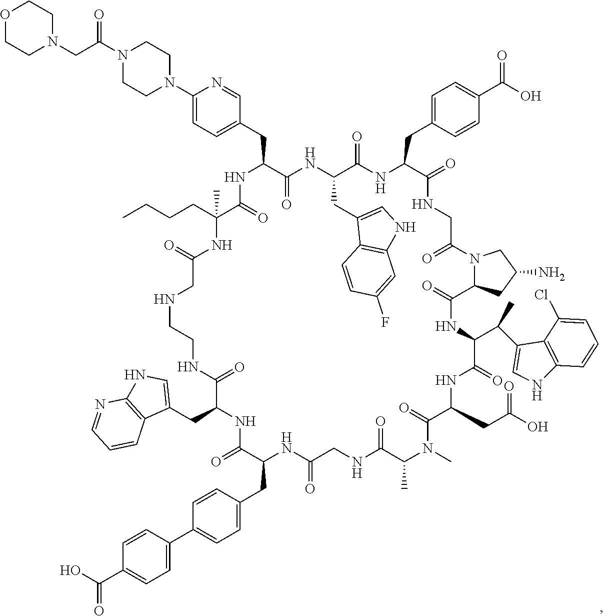 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(=O)CN4CCOCC4)CC3)nc2)NC1=O]

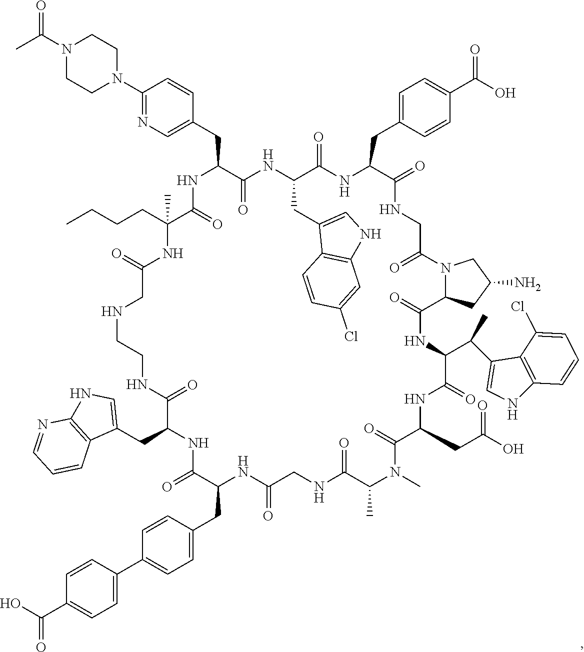 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(C)=O)CC3)nc2)NC1=O]

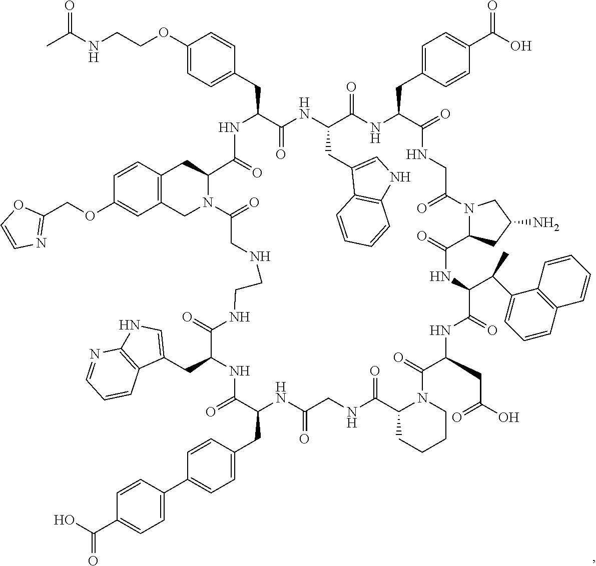 [SMILES: CC(=O)NCCOc1ccc(C[C@@H]2NC(=O)[C@@H]3Cc4ccc(OCc5ncco5)cc4CN3C(=O)CNCCNC(=O)[C@H](Cc3c[nH]c4ncccc34)NC(=O)[C@H](Cc3ccc(-c4ccc(C(=O)O)cc4)cc3)NC(=O)CNC(=O)[C@H]3CCCCN3C(=O)[C@H](CC(=O)O)NC(=O)[C@H]([C@@H](C)c3cccc4ccccc34)NC(=O)[C@@H]3C[C@@H](N)CN3C(=O)CNC(=O)[C@H](Cc3ccc(C(=O)O)cc3)NC(=O)[C@H](Cc3c[nH]c4ccccc34)NC2=O)cc1]

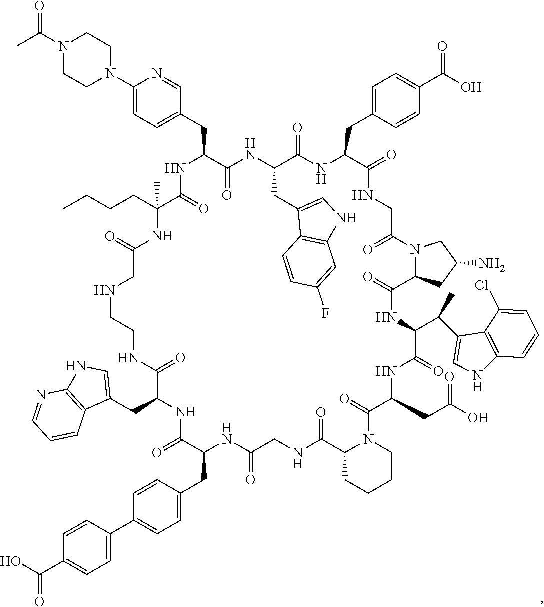 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(C)=O)CC3)nc2)NC1=O]

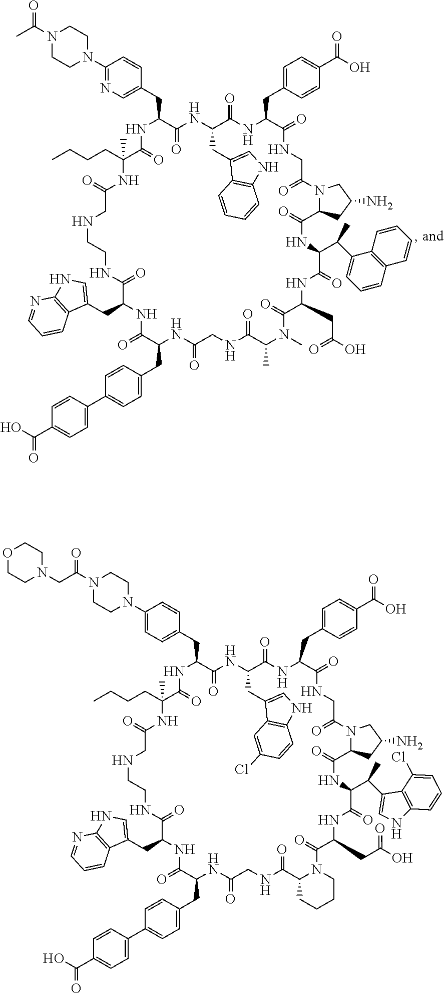

or a pharmaceutically acceptable salt thereof.

While not being bound by any specific theory, the Applicants believe that the compounds of the disclosure trap interleukin-1β, prevent signaling through the IL-1 receptor and hence reduce the downstream markers IL-6 and CRP. Hence the compounds can be useful to treat the inflammatory components of cardiovascular diseases such as ASCVD and heart failure with preserved ejection fraction (HFpEF). The compounds can also be useful to treat inflammatory disorders such as hidradenitis suppurativa (acne inversa), inflammatory bowel disease, and osteoarthritis.

### Definitions

Unless defined otherwise, all technical and scientific terms used herein have the same meaning as is commonly understood by one of skill in the art to which this invention belongs.

As used throughout this disclosure, “a compound of the disclosure”, “a compound of the present disclosure” and “a compound disclosed herein” are used interchangeably are to be understood to include the disclosed cyclic peptides and compounds of Formula (I). The compounds of Formula (I) can form salts which are also within the scope of the present disclosure. Reference to a compound of the disclosure (or compound of Formula (I)) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to an amino group, pyrrolidine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds of Formula (I) may be formed, for example, by reacting a compound of Formula (I) with an amount of acid or base, such as an equivalent amount, in a medium such as one in which the salt precipitates or in an aqueous medium followed by lyophilization.

“Acyl” means an alkyl-C(O)— group, wherein alkyl is as defined below. The bond to the parent group is through the carbon atom of the carbonyl group.

“Alkyl”, as well as other groups having the prefix “alk”, such as alkoxy, and the like, means carbon chains which may be linear or branched, or combinations thereof, containing the indicated number of carbon atoms. For instance, a C₁-C₆ alkyl means an alkyl group having one (i.e., methyl) up to 6 carbon atoms (i.e., hexyl). In particular embodiments, linear alkyl groups have 1-6 carbon atoms and branched alkyl groups have 3-7 carbon atoms. Examples of alkyl groups include methyl, ethyl, propyl, isopropyl, butyl, sec- and tert-butyl, pentyl, hexyl, heptyl, octyl, nonyl and the like.

“Alkoxy” and “alkyl-O—” are used interchangeably and refer to an alkyl group linked to oxygen.

“Amino” means a H₂N— group. The bond to the parent group is through the nitrogen atom.

“Amino acid” refers to naturally-occurring α-amino acids and their stereoisomers, as well as unnatural amino acids (such as β-amino acids and substituted amino acids) and their stereoisomers. In the sequences given for the peptides (compounds) according to the present disclosure, the amino acid residues have their conventional meaning. Thus, “G” is glycine, “W” is tryptophan, “A” is alanine, “S” is serine, and so on. It is to be understood that “D)” isomers are designated by a “d” before the one letter code or amino acid name, such that for example dA is the D isomer of L-alanine. Amino acid residues not encompassed by the foregoing have the definitions provided in the Table in the Examples section below.

“Aryl”, as used herein, represents a monocyclic 6-membered or bicyclic 10-membered ring system, wherein at least one ring is aromatic, and all the ring atoms are carbon.

“Bicyclic ring system” refers to two joined rings. The rings may be fused, i.e., share two adjacent atoms, or “spirocyclic”, i.e., share only a single atom.

“Carbamoyl” means a H₂N—C(O)— group, which is the univalent group formed by loss of —OH group of carbamic acid. The bond to the parent group is through the carbon atom of the carbonyl component.

“Carboxy” means a HO₂C— group. The bond to the parent group is through the carbon atom of the carbonyl component.

“Cycloalkenyl” means an unsaturated, non-aromatic hydrocarbon radical. In particular embodiments, the cycloalkenyl group has 4-6 carbon atoms, forming monocyclic carbocyclic rings. Examples of cycloalkyl include cyclobutenyl, cyclopentenyl, cyclohexenyl, and the like.

“Cycloalkyl” means a saturated cyclic hydrocarbon radical. In particular embodiments, the cycloalkyl group has 3-12 carbon atoms, forming 1-3 carbocyclic rings that. The rings may be fused, or “spirocyclic”, i.e., share only a single atom, or “bridged”, i.e., share three or more atoms with two bridgehead atoms being connected by a bridge containing at least one atom. Examples of cycloalkyl include cyclopropyl, cyclobutyl, cyclopentyl, cyclohexyl, cycloheptyl, adamantyl, bicyclo[1.1.1]pentanyl, and the like.

“Fluoroalkyl” includes mono-substituted as well as multiple fluoro-substituted alkyl groups, up to perfluoro substituted alkyl. For example, fluoromethyl, 1,1-difluoroethyl, trifluoromethyl or 1,1,1,2,2-pentafluorobutyl are included.

“Halogen” or “halo”, unless otherwise indicated, includes fluorine (fluoro), chlorine (chloro), bromine (bromo) and iodine (iodo). In one embodiment, halo is fluoro (—F) or chloro (—Cl).

“Heterocycloalkyl” or “heterocyclyl” means a non-aromatic monocyclic, bicyclic or tricyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. The rings of bi- and tricyclic ring may be fused, or “spirocyclic”, i.e., share only a single atom, or “bridged”, i.e., share three or more atoms with two bridgehead atoms being connected by a bridge containing at least one atom. There are no adjacent oxygen and/or sulfur atoms present in the ring system. In some embodiments, heterocycloalkyls contain about 5 to about 6 ring atoms. The prefix aza, oxa or thia before the heterocyclyl root name means that at least a nitrogen, oxygen or sulfur atom respectively is present as a ring atom. In some embodiments, the nitrogen or sulfur atom of the heterocycloalkyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydropyranyl, tetrahydrothiophenyl, and the like.

“Heteroaryl” refers to aromatic monocyclic, bicyclic and tricyclic ring structures in which one or more atoms in the ring, the heteroatom(s), is an element other than carbon. Heteroatoms are typically O, S, or N atoms. Examples of heteroaromatic groups include pyridinyl, pyrimidinyl, pyrrolyl, pyridazinyl, isoxazolyl, thiazolyl, oxazolyl, indolyl, benzoxazolyl, benzothiazolyl, and imidazolyl.

“Hydroxyalkyl” means a HO-alkyl— group in which alkyl is as previously defined. The bond to the parent moiety is through a carbon atom of the alkyl group. Preferred hydroxyalkyls contain from 1 to 3 carbon atoms. Non-limiting examples of suitable hydroxyalkyl groups include hydroxymethyl and 2-hydroxyethyl.

When any variable (e.g., R^{CL3}) occurs more than one time in any constituent or in Formula (I) or other generic formulas herein, its definition on each occurrence is independent of its definition at every other occurrence. Combinations of substituents and/or variables are permissible only if such combinations result in stable compounds. In choosing compounds of the present disclosure, one of ordinary skill in the art will recognize that the various substituents, e.g., R^{C9}, are to be chosen in conformity with well-known principles of chemical structure connectivity and stability. Unless expressly stated to the contrary, substitution by a named substituent is permitted on any atom in a ring (e.g., aryl, a heteroaryl ring, or a saturated heteroaryl ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Unless expressly depicted or described otherwise, variables depicted in a structural formula with a “floating” bond, are permitted on any available carbon atom in the ring to which the variable is attached. When a moiety is noted as being “optionally substituted” in Formula (I) or any embodiment thereof, it means that Formula (I) or the embodiment thereof encompasses compounds that contain the noted substituent (or substituents) on the moiety and also compounds that do not contain the noted substituent (or substituents) on the moiety.

The wavy line [symbol] , as used herein, indicates a point of attachment to the rest of the compound.

Some of the compounds described herein may exist as tautomers which have different points of attachment of hydrogen accompanied by one or more double bond shifts. For example, a ketone and its enol form are keto-enol tautomers. The individual tautomers as well as mixtures thereof are encompassed with compounds of the present disclosure.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure as described and claimed herein is meant to include all suitable isotopic variations of the compounds of the disclosure and embodiments thereof. For example, different isotopic forms of hydrogen (H) include protium (¹H) and deuterium (²H, also denoted herein as D). Protium is the predominant hydrogen isotope found in nature. Enriching for deuterium may afford certain therapeutic advantages, such as increasing in vivo half-life or reducing dosage requirements or may provide a compound useful as a standard for characterization of biological samples. Isotopically-enriched compounds of the disclosure, can be prepared without undue experimentation by conventional techniques well known to those skilled in the art or by processes analogous to those described in the Schemes and Examples herein using appropriate isotopically-enriched reagents and/or intermediates.

The term “pharmaceutically acceptable salts” refers to salts prepared from pharmaceutically acceptable non-toxic bases or acids. When the compound of the present disclosure is acidic (or has a functional group which may be anionic), its corresponding salt can be conveniently prepared from pharmaceutically acceptable non-toxic bases, including inorganic bases and organic bases. Examples of suitable inorganic cations include, but are not limited to, alkali metal ions such as Li⁺, Na⁺, and K⁺, alkaline earth metal cations such as Ca2⁺, and Mg2⁺, and other cations such as Al³⁺ and Zn⁺. Examples of suitable organic cations include, but are not limited to, ammonium ion (i.e., NH₄⁺) and substituted ammonium ions. Examples of suitable substituted ammonium ions are those derived from methylamine, ethylamine, diethylamine, triethylamine and ethylenediamine. When a compound of the present disclosure is basic, its corresponding salt can be conveniently prepared from pharmaceutically acceptable non-toxic acids, including inorganic acids and organic acids. Example of such acid addition salts include salts formed from hydrohalic acids (e.g., hydrochloric, hydrobromic, hydroiodic), formic acid, acetic acid, capric acid, and citric acids. Salts containing acetate, formate, caprate, chloride, or sodium salts are typical for use with the compounds of the present disclosure. In some embodiments, salts of compounds of the present disclosure can be formed by exchange well-known to those of ordinary skill in the art, such as by anion exchange, e.g., replacement of trifluoroacetate ions with chloride ions.

Furthermore, compounds of the present disclosure may exist in amorphous form and/or one or more crystalline forms, and as such all amorphous and crystalline forms and mixtures thereof of the compounds of Formula (I), including the Examples, are intended to be included within the scope of the present disclosure. In addition, some of the compounds of the instant disclosure may form solvates with water (i.e., a hydrate) or common organic solvents such as, but not limited to, acetic acid or acetonitrile. Such solvates and hydrates, particularly the pharmaceutically acceptable solvates and hydrates, of the instant compounds are likewise encompassed within the scope of this disclosure, along with un-solvated and anhydrous forms.

Any pharmaceutically acceptable pro-drug modification of a compound of this disclosure which results in conversion in vivo to a compound within the scope of this disclosure is also within the scope of this disclosure.

The present disclosure also relates to processes for the preparation of the compounds of Formula (I) which are described in the following Examples and by which the compounds of the disclosure are obtainable.

“Treatment” and “treating” refer to all processes in which there may be a slowing, interrupting, arresting, controlling, or stopping of the progression of a disease or disorder described herein. The terms do not necessarily indicate a total elimination of all disease or disorder symptoms.

“Preventing,” or “prophylaxis,” as used herein, refers to reducing the likelihood of contracting disease or disorder described herein, or reducing the severity of a disease or disorder described herein.

The terms “therapeutically effective (or efficacious) amount” and similar descriptions such as “an amount efficacious for treatment” or “an effective dose” are intended to mean that amount of a compound of the disclosure that will elicit the biological or medical response of a tissue, a system, animal or human that is being sought by a researcher, veterinarian, medical doctor or other clinician. In a preferred embodiment, the term “therapeutically effective amount” means an amount of a compound of the disclosure that alleviates at least one clinical symptom in a human patient. The terms “prophylactically effective (or efficacious) amount” and similar descriptions such as “an amount efficacious for prevention” are intended to mean that amount of a compound of the disclosure that will prevent or reduce the risk of occurrence of the biological or medical event that is sought to be prevented in a tissue, a system, animal or human by a researcher, veterinarian, medical doctor or other clinician.

### Dosages of the Compounds of the Present Disclosure

The dosage regimen utilizing a compound of the present disclosure is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an oncological condition, and a prophylactically effective amount, e.g., for prevention of an oncological condition.

While individual needs vary, determination of optimal ranges of effective amounts of the compound of the present disclosure is within the skill of the art. For administration to a human in the curative or prophylactic treatment of the conditions and disorders identified herein, for example, typical dosages of the compounds of the present disclosure can be about 0.05 mg/kg/day to about 50 mg/kg/day. In some embodiments, a patient is administered from about 5 mg/day to about 120 mg/day, such as from 10 mg/day, 20 mg/day, 30 mg/day, 40 mg/day, 50 mg/day, 60 mg/day, 70 mg/day, 80 mg/day, mg/day, 90 mg/day, or 100 mg/day of a compound of the present disclosure. In certain embodiments, a patient is administered from about 0.2 mg/kg to about 5 mg/kg, such as from 0.5 mg/kg, 0.75 mg/kg, 1.0 mg/kg, 1.25 mg/kg, or 1.5 mg/kg of a compound of the present disclosure. Such doses may be administered in a single dose or may be divided into multiple doses.

### Pharmaceutical Compositions

The compounds of the disclosure and their pharmaceutically acceptable salts can be administered to animals, preferably to mammals, and particularly to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition. Administering of the drug to the subject includes both self-administration and administration to the patient by another person. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

The present disclosure therefore also provides the compounds of the disclosure and their pharmaceutically acceptable salts for use as pharmaceuticals, their use for modulating the activity of the cytokine IL-1β, and in particular, their use in the therapy and prophylaxis of the below-mentioned diseases or disorders as well as their use for preparing medicaments for these purposes. In certain embodiments, the compounds of the disclosure and their pharmaceutically acceptable salts trap IL-1β.

Furthermore, the present disclosure provides pharmaceutical compositions which comprise as active component an effective dose of at least one compound of the disclosure and/or a pharmaceutically acceptable salt thereof and a customary pharmaceutically acceptable carrier, i.e., one or more pharmaceutically acceptable carrier substances and/or additives.

Thus, the present disclosure provides, for example, said compound and its pharmaceutically acceptable salts for use as pharmaceutical compositions which comprise as active component an effective dose of the compound of the disclosure and/or a pharmaceutically acceptable salt thereof and a customary pharmaceutically acceptable carrier, and the uses of said compound and/or a pharmaceutically acceptable salt thereof in the therapy or prophylaxis of the below-mentioned diseases or disorders, e.g., atherosclerosis, as well as their use for preparing medicaments for these purposes.

The pharmaceutical compositions according to the disclosure can be administered orally, for example, in the form of pills, tablets, lacquered tablets, sugar-coated tablets, granules, hard and soft gelatin capsules, aqueous, alcoholic or oily solutions, syrups, emulsions or suspensions, or rectally, for example, in the form of suppositories. Administration can also be carried out parenterally, for example, subcutaneously, intramuscularly or intravenously in the form of solutions for injection or infusion.

Other suitable administration forms are, for example, percutaneous or topical administration, for example, in the form of ointments, tinctures, sprays or transdermal therapeutic systems, or, for example, microcapsules, implants or rods. The preferred administration form depends, for example, on the disease to be treated and on its severity.

The present disclosure also provides pharmaceutical compositions comprising a compound of Formula (I). The compound of Formula (I) can be used in combination with any suitable pharmaceutical carrier or excipient. Such pharmaceutical compositions comprise a therapeutically effective amount of one or more compounds of Formula (I), and pharmaceutically acceptable excipient(s) and/or carrier(s). The specific pharmaceutic composition will suit the mode of administration. In particular aspects, the pharmaceutical acceptable carrier may be water or a buffered solution.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, absorption enhancers, administration aids, and combinations thereof.

Carriers are compounds and substances that improve and/or prolong the delivery of an active ingredient to a subject in the context of a pharmaceutical composition. Carriers may serve to prolong the in vivo activity of a drug or slow the release of the drug in a subject, using controlled-release technologies. Carriers may also decrease drug metabolism in a subject and/or reduce the toxicity of the drug. Carriers can also be used to target the delivery of the drug to particular cells or tissues in a subject. Common carriers (both hydrophilic and hydrophobic carriers) include fat emulsions, lipids, PEGylated phospholipids, PEGylated liposomes, PEGylated liposomes coated via a PEG spacer with a cyclic RGD peptide, liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the compounds of Formula (I). In addition to their use in the pharmaceutical compositions of the present disclosure, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatin capsules include lactose, maize starch or derivatives thereof, stearic acid or salts thereof. Suitable excipients for use with soft gelatin capsules include for example vegetable oils, waxes, fats, semi-solid, or liquid polyols etc. For the preparation of solutions and syrups, excipients which may be used include for example water, polyols and sugars. For the preparation of suspensions oils, e.g., vegetable oils, may be used to provide oil-in-water or water in oil suspensions. Excipients which promote absorption from the gastrointestinal tract, e.g., permeation enhancers, such as sodium caprate can be included. In certain situations, delayed release preparations may be advantageous and compositions which can deliver the compounds of the present disclosure in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerin and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilizing agents, stabilizing agents, wetting agents, emulsifiers, sweeteners, colorants, odorants, salts (substances of the present disclosure may themselves be provided in the form of a pharmaceutically acceptable salt), buffers, coating agents or antioxidants. They may also contain therapeutically-active agents in addition to the compounds of the present disclosure.

### Methods of Using the Compounds of the Disclosure

The present application provides a method of IL-1 mediated cell signaling comprising contacting a cell with a compound of the disclosure or a pharmaceutically acceptable salt thereof. Inhibition of IL-1 mediated cell signaling can be assessed by detecting decreases in the levels of downstream biomarker IL-6 and CRP (e.g., hsCRP).

The present application also provides methods of using the compounds of the disclosure (or their pharmaceutically acceptable salts) or pharmaceutical compositions containing such compounds to treat disease conditions, including but not limited to, conditions implicated by IL-1β.

In some embodiments, the present disclosure provides a method of treating cardiovascular disease, the method comprising administering a therapeutically effective amount a compound of the disclosure (or a pharmaceutically acceptable salt thereof) or any of the foregoing pharmaceutical compositions comprising such a compound to a subject in need of such treatment. In some embodiments, the cardiovascular disease is vascular inflammation. In some embodiments, the cardiovascular disease is atherosclerosis. In some embodiments, the cardiovascular disease is heart failure with preserved ejection fraction (HFpEF). In other embodiments the cardiovascular disease is heart failure with reduced ejection fraction (HFrEF).

In some embodiments, the present disclosure provides a method of treating a chronic kidney disease, the method comprising administering a therapeutically effective amount a compound of the disclosure (or a pharmaceutically acceptable salt thereof) or any of the foregoing pharmaceutical compositions comprising such a compound to a subject in need of such treatment.

In some embodiments, the present disclosure provides a method of treating inflammatory disorders, the method comprising administering a therapeutically effective amount of a compound of the disclosure (or a pharmaceutically acceptable salt thereof) or any of the foregoing pharmaceutical compositions comprising such a compound to a subject in need of such treatment. In certain embodiments, the inflammatory disorder is selected from the group consisting of hidradenitis suppurativa (acne inversa), inflammatory bowel disease, arthritis, and nonalcoholic steatohepatitis (NASH).

In some embodiments, the inflammatory disorder is hidradenitis suppurativa (acne inversa).

In certain embodiments, the inflammatory disorder is inflammatory bowel disease, such as Crohn's disease or ulcerative colitis.

In some embodiments, the inflammatory disorder is arthritis, e.g., osteoarthritis, rheumatoid arthritis, psoriatic arthritis, or gouty arthritis.

In other embodiments, the inflammatory disorder is nonalcoholic steatohepatitis (NASH).

### Combination Therapies

One or more additional pharmacologically active agents may be administered in combination with a compound of the disclosure. An additional active agent (or agents) is intended to mean a pharmaceutically active agent (or agents) that is active in the body, including pro-drugs that convert to pharmaceutically active form after administration, which are different from the compound of Formula I, and also includes free-acid, free-base and pharmaceutically acceptable salts of said additional active agents. Generally, any suitable additional active agent or agents, including but not limited to anti-hypertensive agents, anti-atherosclerotic agents such as a lipid modifying compound, anti-diabetic agents and/or anti-obesity agents, anti-inflammatory agents, may be used in any combination with the compound of the disclosure in a single dosage formulation (a fixed dose drug combination), or may be administered to the subject in one or more separate dosage formulations which allows for concurrent or sequential administration of the active agents (co-administration of the separate active agents).

Examples of additional active agents which may be employed in treating cardiovascular disorders include but are not limited to angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, i.e., COZAAR®, valsartan (including combinations with sacubitril), candesartan, olmesartan, telmesartan and any of these drugs used in combination with hydrochlorothiazide such as HYZAAR®); sGC activators (e.g., riociguat and vericiguat), PCSK9 inhibitors (e.g., evolocumab, alirocumab, MK-0616 and those disclosed in WO2019/246349), neutral endopeptidase inhibitors (e.g., thiorphan and phosphoramidon), aldosterone antagonists, aldosterone synthase inhibitors, renin inhibitors, endothelin receptor antagonists, phosphodiesterase-5 inhibitors (e.g., sildenafil, tadalafil and vardenafil), vasodilators, calcium channel blockers (e.g., amlodipine, nifedipine, verapamil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartrate), alpha adrenergic blocking drugs (e.g., doxazocin, prazocin or alpha methyldopa) central alpha adrenergic agonists, peripheral vasodilators (e.g., hydralazine); lipid lowering agents e.g., HMG-COA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-COA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), fluvastatin (particularly the sodium salt sold in LESCOL®), cerivastatin, and pitavastatin; a cholesterol absorption inhibitor such as ezetimibe (ZETIAR) in combination with any other lipid lowering agents such as the HMG-COA reductase inhibitors noted above and particularly with simvastatin (VYTORIN®) or with atorvastatin calcium; niacin in immediate-release or controlled release forms and/or with an HMG-CoA reductase inhibitor; niacin receptor agonists such as acipimox and acifran, as well as niacin receptor partial agonists; metabolic altering agents including insulin and insulin mimetics (e.g., insulin degludec, insulin glargine, insulin lispro), dipeptidyl peptidase-IV (DPP-4) inhibitors (e.g., sitagliptin, alogliptin, omarigliptin, linagliptin, vildagliptin); insulin sensitizers, including (i) PPARy agonists, such as the glitazones (e.g., pioglitazone, mitoglitazone, lobeglitazone, rosiglitazone, and balaglitazone), and other PPAR ligands, including (1) PPARα/γ dual agonists (e.g., chiglitazar, muraglitazar, aleglitazar, sodelglitazar, and naveglitazar); (2) PPARα agonists such as fenofibric acid derivatives (e.g., gemfibrozil, clofibrate, ciprofibrate, fenofibrate, bezafibrate), (3) selective PPAR γ modulators (SPPAR γ M's), (e.g., such as those disclosed in WO 02/060388, WO 02/08188, WO 2004/019869, WO 2004/020409, WO 2004/020408, and WO 2004/066963); and (4) PPAR γ partial agonists; (ii) biguanides, such as metformin and its pharmaceutically acceptable salts, in particular, metformin hydrochloride, and extended-release formulations thereof, such as Glumetza™, Fortamet™, and GlucophageXR™; and (iii) protein tyrosine phosphatase-1 B (PTP-1 B) inhibitors; insulin or insulin analogs (e.g., insulin detemir, insulin glulisine, insulin degludec, insulin glargine, insulin lispro and inhalable formulations of each); leptin and leptin derivatives and agonists; amylin and amylin analogs (e.g., pramlintide); sulfonylurea and non-sulfonylurea insulin secretagogues (e.g., tolbutamide, glyburide, glipizide, glimepiride, mitiglinide, meglitinides, nateglinide and repaglinide); α-glucosidase inhibitors (e.g., acarbose, voglibose and miglitol); glucagon receptor antagonists; incretin mimetics, such as GLP-1, GLP-1 analogs, derivatives, and mimetics; and GLP-1 receptor agonists (e.g., dulaglutide, semaglutide, albiglutide, exenatide, liraglutide, lixisenatide, taspoglutide, including intranasal, transdermal, and once-weekly formulations thereof); bile acid sequestering agents (e.g., colestilan, colestimide, colesevalam hydrochloride, colestipol, cholestyramine, and dialkylaminoalkyl derivatives of a cross-linked dextran), acyl CoA:cholesterol acyltransferase inhibitors, (e.g., avasimibe); antiobesity compounds; agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs); inhibitors of 11 β-hydroxysteroid dehydrogenase type 1, (e.g., such as those disclosed in U.S. Pat. No. 6,730,690); inhibitors of fructose 1,6-bisphosphatase, (e.g., such as those disclosed in U.S. Pat. Nos. 6,054,587; 6,110,903; 6,284,748; 6,399,782; and 6,489,476); inhibitors of acetyl CoA carboxylase-1 or 2 (ACC1 or ACC2); AMP-activated Protein Kinase (AMPK) activators; other agonists of the G-protein-coupled receptors: (i) GPR-109, (ii) GPR-119, and (iii) GPR-40; SSTR3 antagonists (e.g., such as those disclosed in WO 2009/001836); neuromedin U receptor agonists (e.g., such as those disclosed in WO 2009/042053, including, but not limited to, neuromedin S (NMS)); SCD modulators; GPR-105 antagonists (e.g., such as those disclosed in WO 2009/000087); SGLT inhibitors (e.g., empagliflozin, dapagliflozin, canagliflozin, ertugliflozin, remogliflozin, tofogliflozin, and ipragliflozin); inhibitors of acyl coenzyme A: diacylglycerol acyltransferase 1 and 2 (DGAT-1 and DGAT-2); inhibitors of fatty acid synthase; inhibitors of acyl coenzyme A: monoacylglycerol acyltransferase 1 and 2 (MGAT-1 and MGAT-2); agonists of the TGR5 receptor (also known as GPBAR1, BG37, GPCR19, GPR131, and M-BAR); ileal bile acid transporter inhibitors; PACAP, PACAP mimetics, and PACAP receptor 3 agonists; PPAR agonists; protein tyrosine phosphatase-1 B (PTP-1 B) inhibitors; IL-1β antibodies, (e.g., gevokizumab and canakinumab); and bromocriptine mesylate and rapid-release formulations thereof; or with other drugs beneficial for the treatment of the above-mentioned conditions or disorders including the free-acid, free-base, and pharmaceutically acceptable salt forms of the above active agents where chemically possible.

Examples of additional active agents which may be employed in treating inflammatory disorders include but are not limited to steroidal and non-steroidal anti-inflammatory agents, glucocorticoids, and therapeutic hormones. In particular embodiments, in treating hidradenitis suppurativa (acne inversa), the additional active agent can be an antibiotic, an injectable steroid, a therapeutic hormone, a TNF inhibitor (e.g., infliximab, adalimumab, etanercept, golimumab, certolizumab), a pain medication (e.g., codeine, hydrocodone, morphine, pregabalin, gabapentin, Intralesional triamcinolone, a corticosteroid, naproxen, ketoprofen, diclofenac, ibuprofen, acetaminophen). In other embodiments, in treating an inflammatory bowel disease, the additional active agent can be methotrexate, a TNF inhibitor, an oral sphingosine 1-phosphate receptor modulator (e.g., fingolimod, siponimod, ozanimod, ponesimod) or a selective JAK inhibitor (e.g., tofacitinib, baricitinib, upadacitinib). In some embodiments, in treating osteoarthritis, the additional active agent can be a pain medication (examples listed above). In other embodiments, in treating gouty arthritis, the additional active agent can be colchicine, a non-steroidal anti-inflammatory agent, or a glucocorticoid.

### EXAMPLES

The compounds described herein can be prepared according to the procedures of the following schemes and examples, using appropriate materials and are further exemplified by the following specific examples. The Examples also include methods for testing such compounds in cellular assays. The compounds illustrated in the examples are not, however, to be construed as forming the only genus that is considered as the disclosure.

The examples further illustrate details for the preparation of the compounds of the present disclosure. Those skilled in the art will readily understand that known variations of the conditions and processes of the following preparative procedures can be used to prepare these compounds. For instance, in some cases, the order of carrying out the steps of reaction schemes may be varied to facilitate the reaction or to avoid unwanted reaction products. Starting materials and intermediates for the final compounds are purchased, made from known procedures, or as otherwise illustrated. The examples are provided for the purpose of further illustration only and are not intended to be limitations on the disclosure.

NMR data were obtained on a 300 MHz or 400 MHz instrument in CDCl₃, DMSO-d₆, or Methanol-d₄ with the chemical shifts referenced to solvent signals relative to tetramethylsilane. Multiplets are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

The following examples are meant to be illustrative and should not be construed as further limiting. The contents of the figures and all references, patents, and published patent applications cited throughout this application are expressly incorporated herein by reference.

Throughout the synthetic schemes and examples, abbreviations and acronyms may be used with the following meanings unless otherwise indicated:

Abbreviations

| Abbreviation | Definition |
|---|---|
| AA | Amino acid |
| AcOH | Acetic acid |
| 3AlTyrEtNAc | (2~{S})-3-[4-(2-acetamidoethoxy)-3-prop-2- |
| | enylphenyl]-2-aminopropanoic acid |
| 3AMPA | 2-(3-(aminomethyl)phenyl)acetic acid |
| 3AzaPhe4AcPip | S)-3-(6-(4-acetylpiperazin-1-yl)pyridin-3-yl)-2- |
| | aminopropanoic acid |
| 3Pal4Pipz\|MorphNAcOH | (S)-2-amino-3-(6-(4-(2-morpholinoacetyl)piperazin-1- |
| | yl)pyridin-3-yl)propanoic acid |
| A | 1-Alanine |
| Acc6 | 1-aminocyclohexane-1-carboxylic acid |
| ACN | Acetonitrile |
| 1S4RAcpc | (1S,4R)-4-aminocyclopent-2-ene-1-carboxylic acid |
| AEF | (2~{S})-2-amino-3-[4-(2-aminoethoxy)phenyl]propanoic |
| | acid |
| AEF\|3CH3NPEG4 | (S)-18-(4-(2-amino-2-carboxyethyl)phenoxy)-N,N,N- |
| | trimethyl-15-oxo-3,6,9,12-tetraoxa-16-azaoctadecan-1- |
| | aminium |
| AEF\|PEG4AcOMe | (S)-2-amino-3-(4-((14-oxo-2,5,8,11-tetraoxa-15- |
| | azaheptadecan-17-yl)oxy)phenyl)propanoic acid |
| AlaAze3 | (2~{S})-2-amino-3-(azetidin-3-yl)propanoic acid |
| AlaMph | (2~{S})-2-amino-3-morpholin-4-ylpropanoic acid |
| AlaPent | (2~{S})-2-amino-3-(1-bicyclo[1.1.1]pentanyl)propanoic |
| | acid |
| AlaPip4 | (S)-2-amino-3-(piperidin-4-yl)propanoic acid |
| AlaPip4\|MorphNAcOH | (S)-2-amino-3-(1-(2-morpholinoacetyl)piperidin-4- |
| | yl)propanoic acid |
| AlaPip4Me | (S)-2-amino-3-(1-methylpiperidin-4-yl)propanoic acid |
| AlaPiperaz | 2~{S})-2-amino-3-(4-R3-piperazin-1-yl)propanoic acid |
| AlaPyrim4CONH2 | (2S)-2-amino-3-(2-carbamoylpyrimidin-5-yl)propanoic |
| | acid |
| AlaPyrim4COOH | 5-[(2S)-2-amino-2-carboxy-ethyl]pyrimidine-2- |
| | carboxylic acid |
| AlaTHP4 | (S)-2-amino-3-(tetrahydro-2H-pyran-4-yl)propanoic acid |
| Alloc | allyloxycarbonyl |
| aMeCha | (S)-2-amino-3-cyclohexy1-2-methylpropanoic acid |
| aMeNle | (S)-2-amino-2-methylhexanoic acid |
| 3APA | 2-(3-aminophenyl)acetic acid |
| ASCVD | Atherosclerotic cardiovascular disease |
| 3AzaPhe4AcBCPip | (2S)-3-(6-(6-acetyl-3,6-diazabicyclo[3.1.1]heptan-3- |
| | yl)pyridin-3-yl)-2-aminopropanoic acid |
| Azep | (S)-2,3,4,5-tetrahydro-1H-benzo[d]azepine-2-carboxylic |
| | acid |
| AzG | hydrazinecarboxylic acid |
| bA | 3-aminopropanoic acid |
| b2Aib | 3-amino-2,2-dimethylpropanoic acid |
| b2Dap | (R)-2,3-diaminopropanoic acid |
| b2GlyTHP4 | 4-(aminomethyl)tetrahydro-2H-pyran-4-carboxylic acid |
| b2hPipH | 4-(aminomethyl)piperidine-4-carboxylic acid |
| b2hPip1Me | 4-(aminomethyl)-1-methylpiperidine-4-carboxylic acid |
| b2hPip1Me2 | 4-(aminomethyl)-1,1-dimethylpiperidin-1-ium-4- |
| | carboxylic acid |
| Bip | (2S)-2-amino-3-(4-phenylphenyl)propanoic acid |
| Bip4CO2H | 1-Biphenylalanine-4-carboxylic acid or (S)-4′-(2-amino- |
| | 2-carboxyethyl)-[1,1′-biphenyl]-4-carboxylic acid |
| Bip4COAEMph | (S)-2-amino-3-(4′-((2-morpholinoethyl)carbamoyl)-[1,1′- |
| | biphenyl]-4-yl)propanoic acid |
| Bip4COMph | (S)-2-amino-3-(4′-(morpholine-4-carbonyl)-[1,1′- |
| | biphenyl]-4-yl)propanoic acid |
| Bip4COPipz | (S)-2-amino-3-(4′-(piperazine-1-carbonyl)-[1,1′- |
| | biphenyl]-4-yl)propanoic acid |
| Boc | tert-Butoxy-carbonyl |
| Boc₂O | Di-tert-butyl dicarbonate |
| bProc4NH2 | (2S,4S)-4-aminopyrrolidine-2-carboxylic acid |
| CANTOS | Canakinumab Anti-inflammatory Thrombosis Outcomes |
| | Study |
| Cha | (S)-2-amino-3-cyclohexylpropanoic acid |
| Chg | (S)-2-amino-2-cyclohexylacetic acid |
| CpeG | (S)-2-amino-2-cyclopentylacetic acid |
| CRP | C-reactive protein |
| cSRamcPr | (1~{R},2~{S})-2-(aminomethyl)cyclopropane-1- |
| | carboxylic acid |
| CV | Column volume |
| D | 1-Aspartic acid |
| Dab | (2S)-2,4-diaminobutanoic acid |
| DCM | dichloromethane |
| Dde | 1-(4,4-dimethyl-2,6-dioxocyclohexylidene) ethyl |
| deNC2NH2Gly | (2-aminoethyl)glycine |
| deNC2NH2Gly\|C6acidMe3 | 6-((2-aminoethyl)(carboxymethyl)amino)-N,N,N- |
| | trimethyl-6-oxohexan-1-aminium |
| deNC2NHMeGly | N-(2-aminoethyl)-N-methylglycine |
| deOrn | (2S)-2,5-diaminopentanoic acid |
| DIC | N,N′-Diisopropylcarbodiimide |
| DIPEA | N,N-Diisopropylethylamine or Hunig′s base |
| DMA | N,N-Dimethylacetamide |
| DMAP | 4-Dimethylaminopyridine |
| DMF | N,N-Dimethylformamide |
| DMSO | Dimethylsulfoxide |
| dNMeA | D-N-methyl-Alanine |
| dPip | (R)-piperidine-2-carboxylic acid |
| E | L-Glutamic acid |
| eK | (2S)-2,6-diaminohexanoic acid |
| EtOAc | Ethyl acetate |
| ES | Electrospray |
| F | L-Phenylalanine |
| Fmoc | 9H-fluoren-9-ylmethoxycarbonyl |
| G | Glycine |
| H | L-histidine |
| h | hour |
| GlyTHP4 | (S)-2-amino-2-(tetrahydro-2H-pyran-4-yl)acetic acid |
| HATU | 1-[Bis(dimethylamino)methylene]-1H-1,2,3-triazolo[4,5- |
| | b]pyridinium 3-oxid hexafluorophosphate or N- |
| | [(Dimethylamino)-1H-1,2,3-triazolo-[4,5-b]pyridin-1- |
| | ylmethylene]-N-methylmethanaminium |
| | hexafluorophosphate N-oxide |
| HEPES | N-2-hydroxyethylpiperazine-N-2-ethane sulfonic acid |
| hF3Cl | (S)-2-amino-4-(3-chlorophenyl)butanoic acid |
| HFIP | hexafluoroisopropanol |
| His1Me | π-Methyl-L-histidine |
| His3Me | (2S)-2-amino-3-(3-methylimidazol-4-yl)propanoic acid |
| HisAll | Nt-allyl-L-histidine |
| HOAT | 3-hydroxytriazolo[4,5-b]pyridine |
| hS | L-homoserine |
| hY | L-homotyrosine |
| K | L-Lysine |
| K\|3CH3NPEG4 | 21-amino-21-carboxy-N,N,N-trimethyl-15-oxo-3,6,9,12- |
| | tetraoxa-16-azahenicosan-1-aminium |
| L | L-Leucine |
| LDL | low-density lipoprotein |
| LysMe3 | [(5S)-5-amino-5-carboxy-pentyl]-trimethyl-ammonium |
| mAF | (S)-2-amino-3-(3-(aminomethyl)phenyl)propanoic acid |
| MeCN | acetonitrile |
| N | L-Asparagine |
| NC2NH2Ala | (2~{S})-2-(2-aminoethylamino)propanoic acid |
| NC2NH2Gly | 2-(2-aminoethylamino)acetic acid |
| NC2NHMeGly | N-(2-aminoethyl)-N-methylglycine |
| NC2NMe2Gly | N-(2-aminoethyl)dimethylglycine |
| Nle | (S)-2-aminohexanoic acid |
| NMeProc4NH2 | (2~{S},4~{S})-4-amino-1-methylpyrrolidine-2- |
| | carboxylic acid |
| NMP | N-methylpyrrolidone |
| NMR | Nuclear magnetic resonance |
| Orn | L-Ornithine |
| OSu | O-succinimide |
| 3Pal4Pipz\|MorphNAcOH | (S)-2-amino-3-(6-(4-(2-morpholinoacetyl)piperazin-1- |
| | yl)pyridin-3-yl)propanoic acid |
| 3Pal4Pipz\|4Pyr | (S)-2-amino-3-(6-(4-isonicotinoylpiperazin-1-yl)pyridin- |
| | 3-yl)propanoic acid |
| pBA | 2-(4-(aminomethyl)phenyl)acetic acid |
| PE | Petroleum ether |
| PEG2acidNMe3 | 2-[2-(2-carboxyethoxy)ethoxy]ethyl-trimethylazanium |
| PEG4acidNMe3 | 14-carboxy-N,N,N-trimethyl-3,6,9,12- |
| | tetraoxatetradecan-1-aminium |
| PEG4AcOMe | 3-[2-[2-(2-methoxyethoxy)ethoxy]ethoxy]propanoic acid |
| PEGAcOHNH2 | 2-(2-aminoethoxy)acetic acid |
| PfTrpB-0B2 | Engineered tryptophan synthase from Pyrococcus |
| | furiosus |
| PfTrpB-4D11 | Engineered tryptophan synthase from Pyrococcus |
| | furiosus |
| PfTrpB-7E6 | Engineered tryptophan synthase from Pyrococcus |
| | furiosus |
| Phe3NH2 | (S)-2-amino-3-(3-aminophenyl)propanoic acid |
| Phe4AcPip | (S)-3-(4-(4-acetylpiperazin-1-yl)phenyl)-2- |
| | aminopropanoic acid |
| Phe4CH2NH2 | (S)-2-amino-3-[4-(aminomethyl)phenyl]propanoic acid |
| Phe4CONH2 | (2S)-2-amino-3-(4-carbamoylphenyl)propanoic acid |
| Phe4COOH | L-phenylalanine-4-carboxylic acid or (S)-4-(2-amino-2- |
| | carboxyethyl)benzoic acid |
| Phe4NH2 | (S)-2-amino-3-(4-aminophenyl)propanoic acid |
| Phe4Piperaz | (2~{S})-2-amino-3-(4-piperazin-1-ylphenyl)propanoic |
| | acid |
| Phe4piperaz\|2OH2MePA | (S)-2-amino-3-(4-(4-(2-amino-2-hydroxy-2-methyl-2l5- |
| | propanoyl)piperazin-1-yl)phenyl)propanoic acid |
| Phe4Piperaz\|3CH3NPEG4 | (S)-15-(4-(4-(2-amino-2-carboxyethyl)phenyl)piperazin- |
| | 1-yl)-N,N,N-trimethyl-15-oxo-3,6,9,12- |
| | tetraoxapentadecan-1-aminium |
| Phe4Piperaz\|3Oxad2AcOH5Me | (S)-2-amino-3-(4-(4-(2-(5-methyl-1,3,4-oxadiazol-2- |
| | yl)acetyl)piperazin-1-yl)phenyl)propanoic acid |
| Phe4Piperaz\|3Pyr | (S)-2-amino-3-(4-(4-nicotinoylpiperazin-1- |
| | yl)phenyl)propanoic acid |
| Phe4Piperaz\|4Pyr | (S)-2-amino-3-(4-(4-isonicotinoylpiperazin-1- |
| | yl)phenyl)propanoic acid |
| Phe4Piperaz\|2Pyr | (S)-2-amino-3-(4-(4-picolinoylpiperazin-1- |
| | yl)phenyl)propanoic acid |
| Phe4Piperaz\|5Me134Oxad2AcOH | (S)-2-amino-3-(4-(4-(2-(5-methyl-1,3,4-oxadiazol-2- |
| | yl)acetyl)piperazin-1-yl)phenyl)propanoic acid |
| Phe4Piperaz\|Bn | (S)-2-amino-3-(4-(4-benzoylpiperazin-1- |
| | yl)phenyl)propanoic acid |
| Phe4Piperaz\|MorphNAcOH | (S)-2-amino-3-(4-(4-(2-morpholinoacetyl)piperazin-1- |
| | yl)phenyl)propanoic acid |
| Phe4Piperaz\|PEG2acidNMe3 | (S)-2-(2-(3-(4-(4-(2-amino-2- |
| | carboxyethyl)phenyl)piperazin-1-yl)-3- |
| | oxopropoxy)ethoxy)-N,N,N-trimethylethan-1-aminium |
| Phe4Piperaz\|PEG4AcOMe | (S)-3-(4-(4-(2,5,8,11-tetraoxatetradecan-14- |
| | oyl)piperazin-1-yl)phenyl)-2-aminopropanoic acid |
| Phe4Piperaz\|THF3OH | (2S)-2-amino-3-(4-(4-(tetrahydrofuran-3- |
| | carbonyl)piperazin-1-yl)phenyl)propanoic acid |
| Phe4ptCCA | 4-[4-[(2S)-2-amino-2-carboxyethyl]phenyl]cyclohexane- |
| | 1-carboxylic acid |
| PhG | (S)-2-amino-2-phenylacetic acid |
| PhG2Me | (S)-2-amino-2-(o-tolyl)acetic acid |
| Phg3OH | (S)-2-amino-2-(3-hydroxyphenyl)acetic acid |
| Phg3CH2OH | (S)-2-amino-2-(3-(hydroxymethyl)phenyl)acetic acid |
| Phg4CH2OH | (S)-2-amino-2-(4-(hydroxymethyl)phenyl)acetic acid |
| Phg4OH | (S)-2-amino-2-(4-hydroxyphenyl)acetic acid |
| PhGmPh (=mBIP) | (S)-2-([1,1′-biphenyl]-3-yl)-2-aminoacetic acid |
| Pip | (2S)-piperidine-2-carboxylic acid |
| PpG | (S)-2-amino-2-(piperidin-4-yl)acetic acid |
| Prot4NH2 | (2S,4R)-4-aminopyrrolidine-2-carboxylic acid |
| Prot4NHAc | (2S,4R)-4-acetamidopyrrolidine-2-carboxylic acid |
| Prot4NHBn | (2S,4R)-4-(benzylamino)pyrrolidine-2-carboxylic acid |
| Prot4NHCOBn | (2S,4R)-4-(2-phenylacetamido)pyrrolidine-2-carboxylic |
| | acid |
| Prot4NHCO2Py | (2S,4R)-4-(2-(pyridin-2-yl)acetamido)pyrrolidine-2- |
| | carboxylic acid |
| Prot4NHCO3Py | (2S,4R)-4-(2-(pyridin-3-yl)acetamido)pyrrolidine-2- |
| | carboxylic acid |
| Prot4NHCO4Py | (2S,4R)-4-(2-(pyridin-4-yl)acetamido)pyrrolidine-2- |
| | carboxylic acid |
| Prot4NHCO4PyaRMe | (2S,4R)-4-((R)-2-(pyridin-4-yl)propanamido)pyrrolidine- |
| | 2-carboxylic acid |
| Prot4NHCO4PyaSMe | (2S,4R)-4-((S)-2-(pyridin-4-yl)propanamido)pyrrolidine- |
| | 2-carboxylic acid |
| Prot4NHCOBn4F | (2S,4R)-4-(2-(4-fluorophenyl)acetamido)pyrrolidine-2- |
| | carboxylic acid |
| Prot4NHCOEt4Py | (2S,4R)-4-(3-(pyridin-4-yl)propanamido)pyrrolidine-2- |
| | carboxylic acid |
| Prot4NHCOisoNic | (2S,4R)-4-(isonicotinamido)pyrrolidine-2-carboxylic |
| | acid |
| Prot4NHCOMe4Bip | (2S,4R)-4-[[2-(4-phenylphenyl)acetyl]amino]pyrrolidine- |
| | 2-carboxylic acid |
| Prot4NHCOMeChx | (2S,4R)-4-(2-cyclohexylacetamido)pyrrolidine-2- |
| | carboxylic acid |
| Prot4NHCOMeInd2yl | (2S,4R)-4-(2-(1H-indol-2-yl)acetamido)pyrrolidine-2- |
| | carboxylic acid |
| Prot4NHEtPh | (2S,4R)-4-(phenethylamino)pyrrolidine-2-carboxylic |
| | acid |
| Prot4NH2\|3A4PyA | (2S,4R)-4-(2-(3-aminopyridin-4- |
| | yl)acetamido)pyrrolidine-2-carboxylic acid |
| Prot4NH2\|PEG1NMe3 | 2-(2-(((3R,5S)-5-carboxypyrrolidin-3-yl)amino)-2- |
| | oxoethoxy)-N,N,N-trimethylethan-1-aminium |
| PyOAp | (7-Azabenzotriazol-1-yloxy)tripyrrolidinophosphonium |
| | hexafluorophosphate |
| Q | L-Glutamine |
| rt | Room temperature |
| S | L-Serine |
| sbMe1Nal | (2S,3S)-2-amino-3-(naphthalen-1-yl)butanoic acid |
| SbMe1Nal3F | (2S,3S)-2-amino-3-(3-fluoronaphthalen-1-yl)butanoic |
| | acid |
| SbMe1Nal3Me | (2S,3S)-2-amino-3-(3-methylnaphthalen-1-yl)butanoic |
| | acid |
| sbMeW4Cl | (2S,3S)-2-amino-3-(4-chloro-1H-indol-3-yl)butanoic |
| | acid |
| sbMeW4F | (2S,3S)-2-amino-3-(4-fluoro-1H-indol-3-yl)butanoic acid |
| SFC | Supercritical fluid chromatography |
| SerOAll | (2~{S})-2-amino-3-prop-2-enoxypropanoic acid |
| T | L-threonine |
| TCEP | tris(2-carboxyethyl)phosphine |
| THF | tetrahydrofuran |
| Tic | (S)-1,2,3,4-tetrahydroisoquinoline-3-carboxylic acid |
| Tic6OBn | (S)-6-(benzyloxy)-1,2,3,4-tetrahydroisoquinoline-3- |
| | carboxylic acid |
| Tic6OH | (S)-6-hydroxy-1,2,3,4-tetrahydroisoquinoline-3- |
| | carboxylic acid |
| Tic7OBn | (S)-7-(benzyloxy)-1,2,3,4-tetrahydroisoquinoline-3- |
| | carboxylic acid |
| Tic7OBn4F | (S)-7-((4-fluorobenzyl)oxy)-1,2,3,4- |
| | tetrahydroisoquinoline-3-carboxylic acid |
| Tic7OCH2THP | (S)-7-((tetrahydro-2H-pyran-4-yl)methoxy)-1,2,3,4- |
| | tetrahydroisoquinoline-3-carboxylic acid |
| Tic7OH | (S)-7-hydroxy-1,2,3,4-tetrahydroisoquinoline-3- |
| | carboxylic acid |
| Tic7OMe2Oxa | (3S)-7-(1,3-oxazol-2-ylmethoxy)-1,2,3,4- |
| | tetrahydroisoquinoline-3-carboxylic acid |
| Tic7OMeOxazole | (S)-7-(oxazol-2-ylmethoxy)-1,2,3,4- |
| | tetrahydroisoquinoline-3-carboxylic acid |
| Tic7Pyrim | (S)-7-(pyrimidin-5-yl)-1,2,3,4-tetrahydroisoquinoline-3- |
| | carboxylic acid |
| Tic7Ph | (S)-7-phenyl-1,2,3,4-tetrahydroisoquinoline-3-carboxylic |
| | acid |
| Tiq | (S)-1,2,3,4-tetrahydroisoquinoline-1-carboxylic acid |
| TIS | Triisopropylsilane |
| TFA | Trifluoroacetic acid |
| Trp6Cl | (S)-2-amino-3-(6-chloro-1H-indol-3-yl)propanoic acid |
| Trp5Cl | (S)-2-amino-3-(5-chloro-1H-indol-3-yl)propanoic acid |
| Trp6F | (S)-2-amino-3-(6-fluoro-1H-indol-3-yl)propanoic acid |
| Trp7F | (2S)-2-amino-3-(7-fluoro-1H-indol-3-yl)propanoic acid |
| Trp7az | (S)-2-amino-3-(1H-pyrrolo[2,3-b]pyridin-3-yl)propanoic |
| | acid |
| Trp6Me | (2S)-2-amino-3-(6-methyl-1H-indol-3-yl)propanoic acid |
| TyrEtNAc | L-tyrosine O-ethyl acetamide or (S)-3-(4-(2- |
| | acetamidoethoxy)phenyl)-2-aminopropanoic acid |
| UPLC | Ultra Performance Liquid Chromatography or Ultrahigh |
| | Pressure Liquid Chromatography |
| W | L-Tryptophan |

### Intermediate Syntheses

The following schemes and descriptions disclose method of preparing suitably protected amino acid intermediates and other intermediates for preparing the compounds of the disclosure. The following examples are meant to be illustrative and should not be construed as further limiting. The contents of the figures and all references, patents, and published patent applications cited throughout this application are expressly incorporated herein by reference.

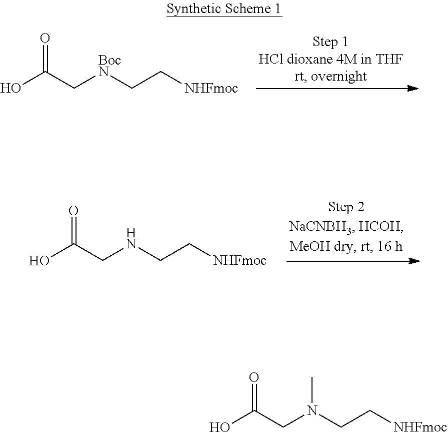

### Precursor to NC2NHMeGly

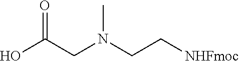

### N-(2-((((9H-fluoren-9-yl)methoxy)carbonyl)amino)ethyl)-N-(tert-butoxycarbonyl)glycine

Step 1: N-(2-((((9H-fluoren-9-yl)methoxy)carbonyl)amino)ethyl)-N-(tert-butoxycarbonyl)glycine (2 g, 4.5 mmol) was dissolved in a mixture of THF (8.84 mL) and 4 M HCl in dioxane (8.84 mL) and the rection was stirred overnight at rt. After 20 h the reaction was complete. The solvent was removed in vacuo to afford the product (2-((((9H-fluoren-9-yl)methoxy)carbonyl)amino)ethyl)glycine, HCl as a white solid. UPLC-MS: (ES, m/z): 341 [M+H]+.

Step 2: (2-((((9H-fluoren-9-yl)methoxy)carbonyl)amino)ethyl)glycine (1 g, 2.94 mmol) was dissolved in dry methanol (10 mL), then formaldehyde (0.441 g, 14.69 mmol) and NaCNBH₃ (0.277 g, 4.41 mmol) were added and the mixture was stirred at rt overnight. After 20 h the reaction was complete. The solvent was removed in vacuo. The crude product was purified by reverse phase chromatography (C18 150 g column: elution A=H2O+0.1% TFA; B=ACN+0.1% TFA from 5% B to 80% B in 8 CV) to give N-(2-((((9H-fluoren-9-yl)methoxy)carbonyl)amino)ethyl)-N-methylglycine. UPLC-MS: (ES, m/z): 355 [M+H]+.

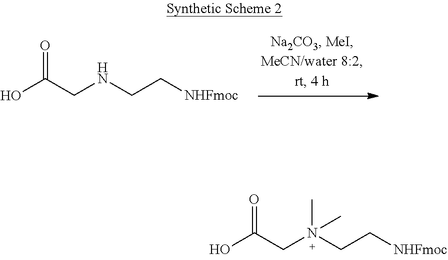

### Precursor to NC2NMe2Gly

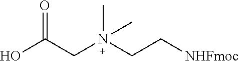

### 2-((((9H-fluoren-9-yl)methoxy)carbonyl)amino)-N-(carboxymethyl)-N,N-dimethylethan-1-aminium

To a solution of (2-((((9H-fluoren-9-yl)methoxy)carbonyl)amino)ethyl)glycine (0.8 g, 2.350 mmol) in MeCN/H2O (8:2), was added Na₂CO₃ (0.747 g, 7.05 mmol), followed by iodomethane (2.93 mL, 47.0 mmol). The reaction mixture was left under stirring for 4 h. The solvent was removed by rotary evaporator. The residue was dissolved in EtOAc, acidified with 6N HCl and extracted several times with EtOAc, dried on Na₂SO₄, filtered, concentrated to dryness and lyophilized to afford 2-((((9H-fluoren-9-yl)methoxy)carbonyl)amino)-N-(carboxymethyl)-N,N-dimethylethan-1-aminium, HCl as a yellow solid. UPLC-MS: (ES, m/z): 369 [M+H]+.

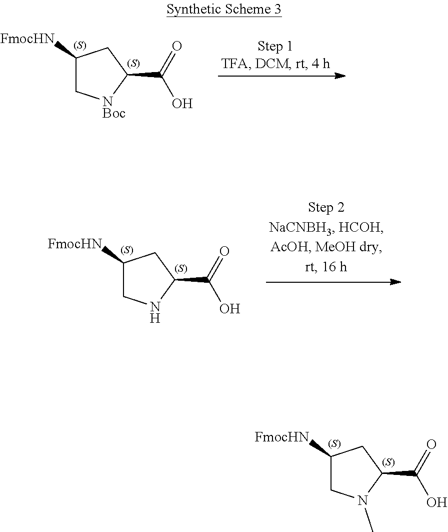

### Precursor to NMeProc4NH2

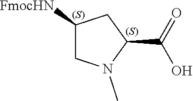

### (2S,4S)-4-((((9H-fluoren-9-yl)methoxy)carbonyl)amino)-1-methylpyrrolidine-2-carboxylic Acid, HCl Salt

Step 1: (2S,4S)-4-((((9H-fluoren-9-yl)methoxy)carbonyl)amino)-1-(tert-butoxycarbonyl)pyrrolidine-2-carboxylic acid (2.1 g, 4.64 mmol) was suspended in DCM and treated with TFA (10 mL, 130 mmol) at rt for 4 h. Then the volatiles were removed under reduced pressure to give the crude product (2S,4S)-4-((((9H-fluoren-9-yl)methoxy)carbonyl)amino)pyrrolidine-2-carboxylic acid. UPLC-MS: (ES, m/z): 353 [M+H]+.

Step 2: (2S,4S)-4-((((9H-fluoren-9-yl)methoxy)carbonyl)amino)pyrrolidine-2-carboxylic acid, HCl (1.789 g, 4.6 mmol) was dissolved in dry MeOH (46.0 mL), then formaldehyde (3.42 mL, 46.0 mmol), acetic acid (0.527 mL, 9.20 mmol) and NaCNBH₃ (1.445 g, 23.00 mmol) were added. The mixture was stirred for at room temperature for 2 h under nitrogen. The solvent was removed in vacuo and the residue purified by reverse phase chromatography (C18 300 g column: elution A=H₂O+0.1% TFA; B=ACN+0.1% TFA initial 10% B for 1 CV then from 10% B to 90% B in 6 CV) to give after lyophilization and salt exchange to HCl the title compound. UPLC-MS: (ES, m/z): 367 [M+H]+.

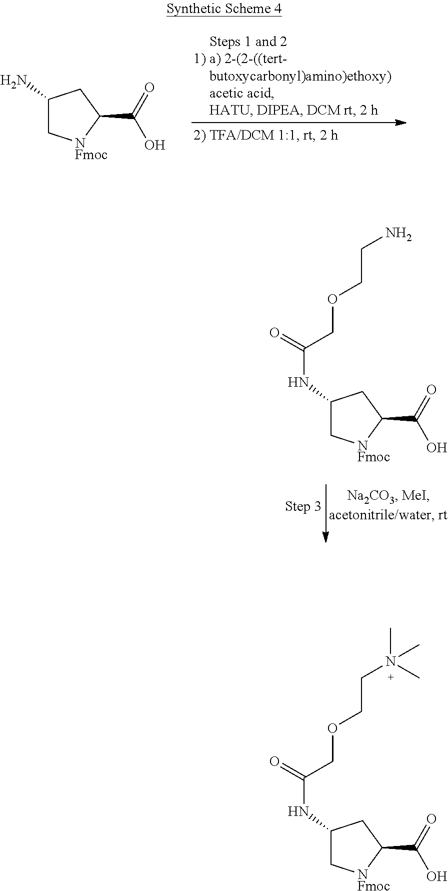

### Precursor to Prot4NH2|PEG1NMe3

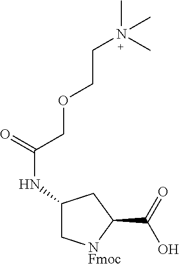

### 2-(2-(((3R,5S)-1-(((9H-fluoren-9-yl)methoxy)carbonyl)-5-carboxypyrrolidin-3-yl)amino)-2-oxoethoxy)-N,N,N-trimethylethan-1-aminium

Step 1:2-(2-((tert-butoxycarbonyl)amino) ethoxy) acetic acid (0.266 g, 1.216 mmol) in 20 mL DMF was treated with HATU (0.420 g, 1.105 mmol) and DIPEA (0.386 ml, 2.210 mmol) for 5 minutes and then added to a solution of (9H-fluoren-9-yl)methyl (2S,4R)-2-acetyl-4-aminopyrrolidine-1-carboxylate (0.389 g, 1.105 mmol) in 15 mL DMF. The pH was adjusted to 8-9 with the addition of DIPEA and the mixture was stirred at rt for 2 h. 1 mL of TFA was added to the reaction mixture, then the volatiles were removed in vacuo to give crude (2S,4R)-1-(((9H-fluoren-9-yl)methoxy)carbonyl)-4-(2-(2-((tert-butoxycarbonyl)amino) ethoxy) acetamido)pyrrolidine-2-carboxylic acid (orange-yellow oil). UPLC-MS: (ES, m/z): 554 [M+H]+.

Step 2: (2S,4R)-1-(((9H-fluoren-9-yl)methoxy)carbonyl)-4-(2-(2-((tert-butoxycarbonyl)amino) ethoxy) acetamido)pyrrolidine-2-carboxylic acid (0.612 g, 1.105 mmol) was dissolved in DCM/TFA 1:1 (40 ml) and the reaction was stirred 1 h at rt. The solvent was removed in vacuo to afford the crude product (2S,4R)-1-(((9H-fluoren-9-yl)methoxy)carbonyl)-4-(2-(2-aminoethoxy) acetamido)pyrrolidine-2-carboxylic acid as an orange-yellow oil. UPLC-MS: (ES, m/z): 454 [M+H]+.

Step 3: To a solution of (2S,4R)-1-(((9H-fluoren-9-yl)methoxy)carbonyl)-4-(2-(2-aminoethoxy) acetamido)pyrrolidine-2-carboxylic acid (0.501 g, 1.105 mmol) in acetonitrile (4.42 ml) were added iodomethane (1.382 ml, 22.10 mmol) and a solution of Na₂CO₃ (0.351 g, 3.32 mmol) in water (1.105 ml) and the mixture was stirred at rt. Then the volatiles were removed under reduced pressure. Purification on Biotage: Luknova C18 110 g flow 85 ml/min; method: 10% B 3 CV, 10 to 70% B in 7 CV (A=water+0.1% TFA; B=acetonitrile+0.1% TFA) afforded the title compound. UPLC-MS: (ES, m/z): 496 [M]+.

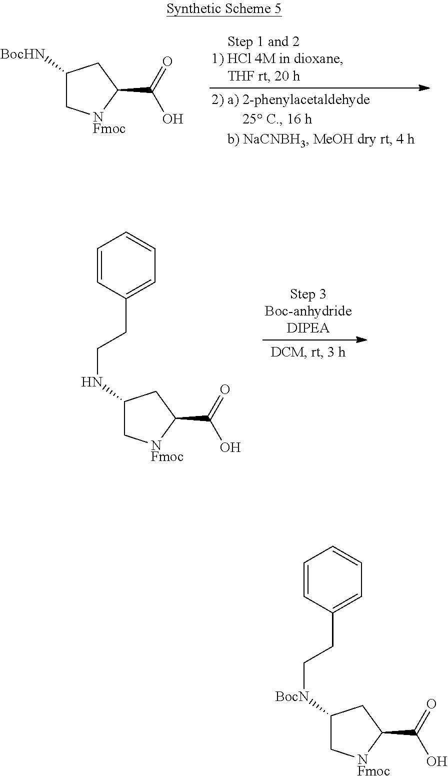

### Precursor to Prot4NHEtPh

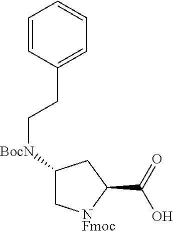

### (2S,4R)-1-(((9H-fluoren-9-yl)methoxy)carbonyl)-4-((tert-butoxycarbonyl)(phenethyl)amino)pyrrolidine-2-carboxylic Acid

Step 1: (2S,4R)-1-(((9H-fluoren-9-yl)methoxy)carbonyl)-4-((tert-butoxycarbonyl)amino)pyrrolidine-2-carboxylic acid (3.2 g, 7.07 mmol) was dissolved in THF 30 mL and treated with HCl in dioxane (15 mL, 60.0 mmol) 4 M for 20 h. The volatiles were removed under reduced pressure to give the crude product (2S,4R)-1-(((9H-fluoren-9-yl)methoxy)carbonyl)-4-aminopyrrolidine-2-carboxylic acid, HCl salt as white solid which was used as such without further purification. UPLC-MS: (ES, m/z): 353 [M+H]+.

Step 2: (2S,4R)-1-(((9H-fluoren-9-yl)methoxy)carbonyl)-4-aminopyrrolidine-2-carboxylic acid, HCl salt (600 mg, 1.543 mmol) was dissolved in MeOH dry (25 mL), 2-phenylacetaldehyde (185 mg, 1.543 mmol) was added and the mixture was stirred at rt under nitrogen flux 16 h, then NaCNBH₃ (107 mg, 1.697 mmol) was added and the mixture was stirred at room temperature for 4 h. The reaction was quenched with water then the solvent was removed in vacuo. The residue was lyophilized to afford the crude product (2S,4R)-1-(((9H-fluoren-9-yl)methoxy)carbonyl)-4-(phenethylamino)pyrrolidine-2-carboxylic acid as a white solid that was used in the next step without further purification. UPLC-MS: (ES, m/z): 457 [M+H]+.

Step 3: A solution of (2S,4R)-1-(((9H-fluoren-9-yl)methoxy)carbonyl)-4-(phenethylamino)pyrrolidine-2-carboxylic acid (704 mg, 1.543 mmol) in DCM (30 mL) was treated with BOC-Anhydride (1.791 mL, 7.72 mmol) and DIPEA (1.347 mL, 7.72 mmol) for 3 h. The reaction monitored by UPLC MS. The solvent was removed under reduced pressure to give the crude product which was purified by reverse phase chromatography on a Biotage C18 column 110 g elution from water 0.1% TFA/acetonitrile 0.1% TFA 90:10 to 10:90 in 10 CV) to give the title compound after lyophilization. UPLC-MS: (ES, m/z): 557 [M+H]+.

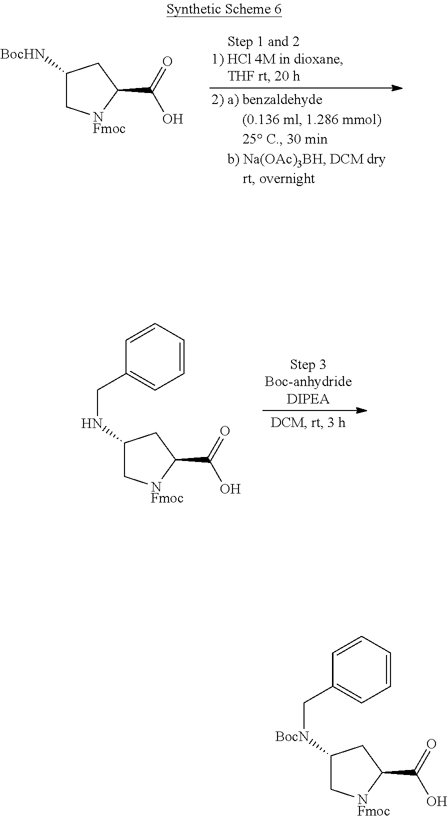

### Precursor to Prot4NHBn

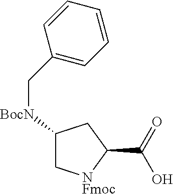

### (2S,4R)-1-(((9H-fluoren-9-yl)methoxy)carbonyl)-4-(benzyl(tert-butoxycarbonyl)amino)pyrrolidine-2-carboxylic Acid

Step 1: (2S,4R)-1-(((9H-fluoren-9-yl)methoxy)carbonyl)-4-((tert-butoxycarbonyl)amino)pyrrolidine-2-carboxylic acid (3.2 g, 7.07 mmol) was dissolved in THF 30 mL and treated with HCl in dioxane (15 mL, 60.0 mmol) 4 M for 20 h. The volatiles were removed under reduced pressure to give the crude product (2S,4R)-1-(((9H-fluoren-9-yl)methoxy)carbonyl)-4-aminopyrrolidine-2-carboxylic acid, HCl salt as white solid which used without further purification in the next step. UPLC-MS: (ES, m/z): 353 [M+H]+.

Step 2: (2S,4R)-1-(((9H-fluoren-9-yl)methoxy)carbonyl)-4-aminopyrrolidine-2-carboxylic acid, HCl (500 mg, 1.286 mmol) was dissolved in dry DCM (50 mL). Benzaldehyde (0.136 mL, 1.286 mmol) was added and the mixture was stirred at rt for 30 minutes. Sodium triacetoxyhydroborate (409 mg, 1.929 mmol) was added and the mixture was stirred at room temperature overnight. The solvent was removed in vacuo. The residue was dissolved in ethyl acetate and the organic phase was washed with HCl. The organic layer was dried over Na₂SO₄, filtered and then the solvent was removed in vacuo to afford (2S,4R)-1-(((9H-fluoren-9-yl)methoxy)carbonyl)-4-(benzylamino)pyrrolidine-2-carboxylic acid as a white solid which was used in the next step without further purification. UPLC-MS: (ES, m/z): 443 [M+H]+.

Step 3: A solution of (2S,4R)-1-(((9H-fluoren-9-yl)methoxy)carbonyl)-4-(benzylamino)pyrrolidine-2-carboxylic acid (569 mg, 1.286 mmol) in DCM (30 mL) was treated with BOC-anhydride (1.493 mL, 6.43 mmol) and DIPEA (1.123 mL, 6.43 mmol) for 3 h. The reaction monitored by UPLC MS. The solvent was removed under reduced pressure to give the crude product which was purified by reverse phase chromatography on Biotage (C18 column 120 g elution from water 0.1% TFA/acetonitrile 0.1% TFA 100:0 to 20:80 in 10 CV) to give the title compound after lyophilization. UPLC-MS: (ES, m/z): 543 [M+H]+.

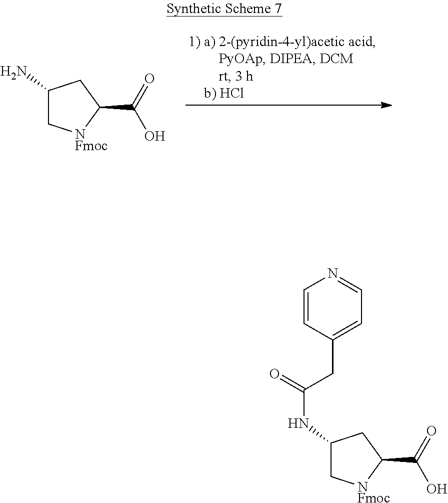

### Precursor to Prot4NHCO4Py

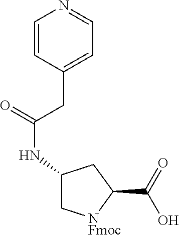

### (2S,4R)-1-(((9H-fluoren-9-yl)methoxy)carbonyl)-4-(2-(pyridin-4-yl) acetamido)pyrrolidine-2-carboxylic Acid

2-(Pyridin-4-yl) acetic acid, HCl (192 mg, 1.105 mmol) in DCM 10 mL was treated with PyOAP (576 mg, 1.105 mmol) and DIPEA (0.772 mL, 4.42 mmol) for 10 minutes. The mixture was then added to a solution of (2S,4R)-1-(((9H-fluoren-9-yl)methoxy)carbonyl)-4-aminopyrrolidine-2-carboxylic acid, HCl salt (430 mg, 1.105 mmol) in DCM 10 mL and the mixture was stirred at rt for 3 h. Then the volatiles were removed and the crude product was purified by flash chromatography on silica (80 g HP column, DCM/MeOH each eluent containing 0.5% of AcOH, elution from 100:0 2 CV then to 90:10 in 10 CV and 90:10 for 3 CV) to give after salt exchange with HCl and removal of excess of AcOH with toluene (2S,4R)-1-(((9H-fluoren-9-yl)methoxy)carbonyl)-4-(2-(pyridin-4-yl) acetamido)pyrrolidine-2-carboxylic acid, HCl. UPLC-MS: (ES, m/z): 472 [M+H]+. ¹H NMR (300 MHz, DMSO-d₆) δ 9.16 (s, 1H), 8.81 (s, 3H), 7.89 (m, 4H), 7.66 (d, 2H), 7.43-7.19 (m, 4H), 4.30 (m, 4H), 4.16 (m, 1H), 3.82 (s, 2H), 3.61 (m, 2H), 3.37 (m, 1H), 3.17 (m, 1H), 2.21 (m, 1H).

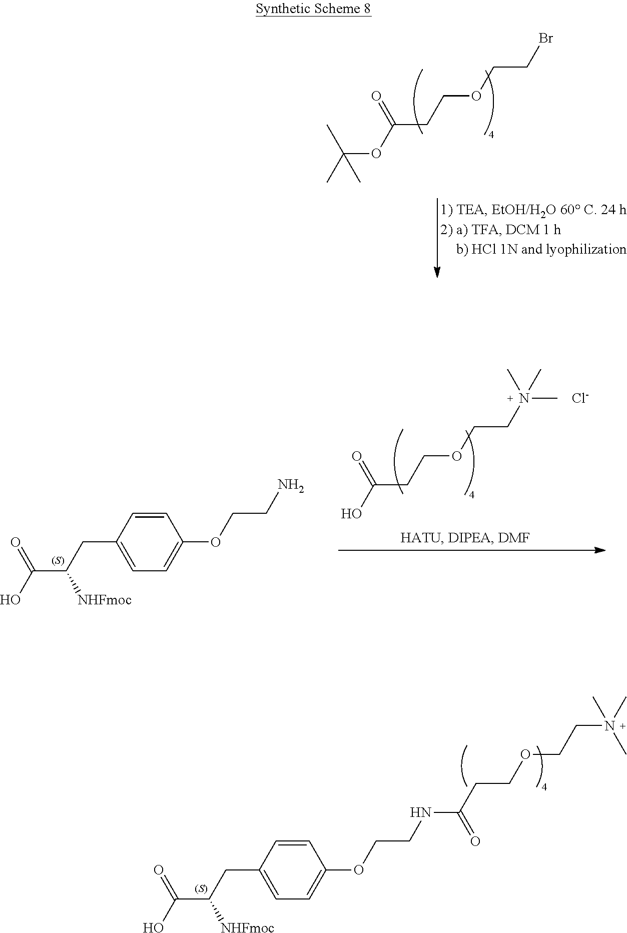

### Precursor to AEF|3CH3NPEG₄

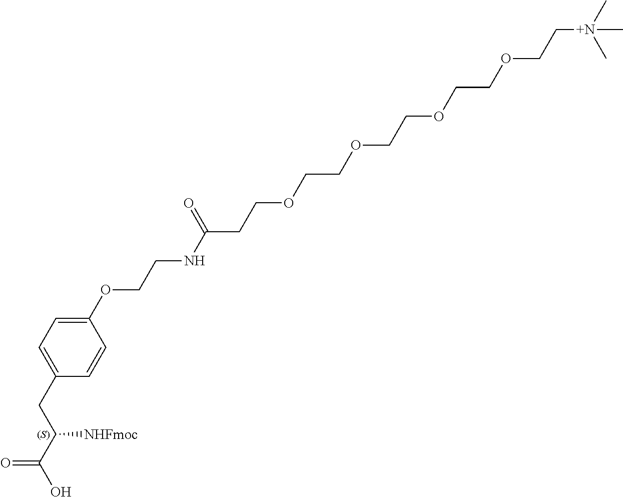

### (S)-18-(4-(2-((((9H-fluoren-9-yl)methoxy)carbonyl)amino)-2-carboxyethyl) phenoxy)-N,N,N-trimethyl-15-oxo-3,6,9,12-tetraoxa-16-azaoctadecan-1-aminium

Step 1: Tert-butyl 1-bromo-3,6,9,12-tetraoxapentadecan-15-oate (3.74 g, 9.71 mmol) was dissolved in trimethylamine (15.36 mL, 97 mmol) and ethanol (8 mL) was added. The reaction mixture was stirred at 60° C. for 24 h in an ACE tube. The solvent was removed in vacuo. The starting material and the product were detected only by mass. The product was lyophilized to give N,N,N, 17,17-pentamethyl-15-oxo-3,6,9,12,16-pentaoxaoctadecan-1-aminium. UPLC-MS: (ES, m/z): 364 [M+H]+.

Step 2: N,N,N, 17,17-pentamethyl-15-oxo-3,6,9,12,16-pentaoxaoctadecan-1-aminium was treated with 30 mL of TFA and 30 mL of DCM and stirred at rt. After 1 h, the reaction was completed, and the solvent was removed in vacuo. 0.5 mL of HCl 1N was added and the product was lyophilized to give 14-carboxy-N,N,N-trimethyl-3,6,9,12-tetraoxatetradecan-1-aminium chloride salt. UPLC-MS: (ES, m/z): 308 [M+H]+.

Step 3:14-carboxy-N,N,N-trimethyl-3,6,9,12-tetraoxatetradecan-1-aminium chloride (1.013 g, 1.971 mmol) in 20 mL DMF was treated with HATU (0.681 g, 1.792 mmol) and DIPEA (0.626 mL, 3.58 mmol) for 5 minutes and then added to a solution of 2-((((9H-fluoren-9-yl)methoxy)carbonyl)amino)-3-(4-(2-aminoethoxy)phenyl)propanoic acid (0.8 g, 1.792 mmol) in 15 mL DMF and the mixture was stirred at rt for 1 h. The conversion of the starting material was almost complete, 1 mL of 14-carboxy-N,N,N-trimethyl-3,6,9,12-tetraoxatetradecan-1-aminium activated solution was added. After 5 minutes, the reaction was complete. 1 mL of TFA was added to the reaction mixture, then the volatiles were removed in vacuo. The crude product was purified by flash chromatography on silica (Luknova C18 110 g column; water/acetonitrile, each eluent containing 0.1% of TFA, elution from 20:80 to 80:20 in 15 CV) to give 18-(4-(2-((((9H-fluoren-9-yl)methoxy)carbonyl)amino)-2-carboxyethyl) phenoxy)-N,N,N-trimethyl-15-oxo-3,6,9,12-tetraoxa-16-azaoctadecan-1-aminium. UPLC-MS: (ES, m/z): 736 [M]+.

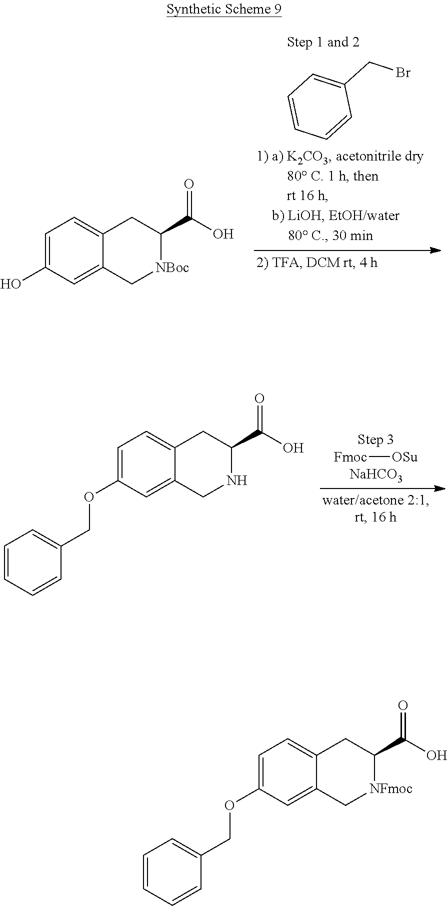

### Precursor to Tic7OBn

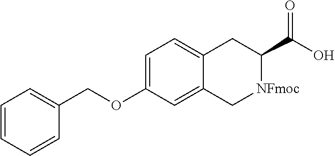

### ((S)-2-(((9H-fluoren-9-yl)methoxy)carbonyl)-7-(benzyloxy)-1,2,3,4-tetrahydroisoquinoline-3-carboxylic Acid

Step 1:2 A solution of(S)-2-(tert-butoxycarbonyl)-7-hydroxy-1,2,3,4-tetrahydroisoquinoline-3-carboxylic acid (1 g, 3.41 mmol) was dissolved in dry acetonitrile (38 mL) and treated with K₂CO₃ (1.414 g, 10.23 mmol) followed by dropwise addition of (bromomethyl)benzene (0.892 mL, 7.50 mmol) under stirring. The reaction mixture was heated at 80° C. for 1 h and at rt for 16 h overnight. Once the reaction was done the mixture was filtered to remove the solids (KBr and K₂CO₃) and acetonitrile was evaporated under reduced pressure. The residue was taken up with EtOAc and washed with water (2 times) and brine. The combined organic was dried (Na₂SO₄) and the solvent removed under reduced pressure. The product was purified by flash chromatography on silica (8 0 g column, EtOAc/Petroleum ether from 0:100 to 30:70 in 10 CV) to give the product as benzyl ester which was dissolved in EtOH and water treated with lithium hydroxide (0.408 g, 17.05 mmol) at 80° C. for 30 minutes. Then the mixture was acidified with HCl 1N aqueous solution and extracted with EtOAc. The organic phase was dried (Na₂SO₄) filtered and the solvent removed under reduced pressure to give crude product(S)-7-(benzyloxy)-2-(tert-butoxycarbonyl)-1,2,3,4-tetrahydroisoquinoline-3-carboxylic acid (1.8 g). UPLC-MS: (ES, m/z): 384 [M+H]+.

Step 2: (S)-7-(benzyloxy)-2-(tert-butoxycarbonyl)-1,2,3,4-tetrahydroisoquinoline-3-carboxylic acid (1.304 g, 3.4 mmol) was dissolved in DCM (10 mL) and treated at rt with TFA (5 mL, 64.9 mmol) for 2 h. The volatiles were removed, and the crude residue lyophilized to give(S)-7-(benzyloxy)-1,2,3,4-tetrahydroisoquinoline-3-carboxylic acid, TFA salt (1.19 g) which used in the next step. UPLC-MS: (ES, m/z): 284 [M+H]+.

Step 3: (S)-7-(benzyloxy)-1,2,3,4-tetrahydroisoquinoline-3-carboxylic acid (482 mg, 1.7 mmol) was dissolved in was dissolved in acetone (22 mL) and water (44 mL) then treated with sodium bicarbonate (857 mg, 10.20 mmol) and Fmoc-Osu (516 mg, 1.530 mmol) for 16 h. The reaction mixture turned limpid. EtOAc (15 mL) was added, and the mixture was acidified with HCl 1N 10 mL. The aqueous and organic phases were separated and the aqueous phase was extracted with another 15 mL of EtOAc. The combined organic layers were dried (Na₂SO₄), filtered and the solvent removed under reduced pressure. The residue was lyophilized to give the crude product which was purified by reverse phase chromatography (C18 column 55 g, elution A=H2O+0.1% TFA; B=MeCN+0.1% TFA 10% B 2 CV then from 10% B to 90% B in 15 CV) to give(S)-2-(((9H-fluoren-9-yl)methoxy)carbonyl)-7-(benzyloxy)-1,2,3,4-tetrahydroisoquinoline-3-carboxylic acid (580 mg). UPLC-MS: (ES, m/z): 506 [M+H]+. 1H NMR (300 MHz, DMSO-d₆) δ 12.81 (s, 1H), 7.91 (m, 2H), 7.72-7.63 (m, 2H), 7.43-7.48-7.29 (m, 9H), 7.14 (d, 1H), 6.94-6.85 (m, 2H), 5.10 (d, 2H), 4.85 (m, 1H), 4.63-4.56 (m, 1H), 4.50-4.25 (m, 4H), 3.12-3.01 (m, 2H).

### Precursor to Tic6OBn

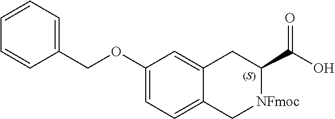

### ((S)-2-(((9H-fluoren-9-yl)methoxy)carbonyl)-6-(benzyloxy)-1,2,3,4-tetrahydroisoquinoline-3-carboxylic Acid

((S)-2-(((9H-fluoren-9-yl)methoxy)carbonyl)-6-(benzyloxy)-1,2,3,4-tetrahydroisoquinoline-3-carboxylic acid was prepared according to same procedure as for ((S)-2-(((9H-fluoren-9-yl)methoxy)carbonyl)-7-(benzyloxy)-1,2,3,4-tetrahydroisoquinoline-3-carboxylic acid. UPLC-MS: (ES, m/z): 506 [M+H]+

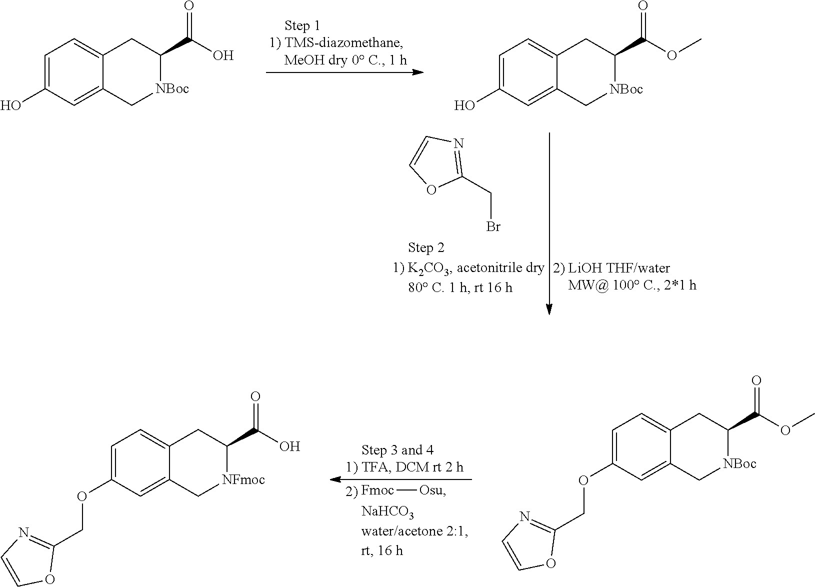

### Precursor to Tic7OMeOxazole or Tic70 Me2Oxa

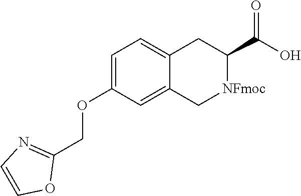

### (S)-2-(((9H-fluoren-9-yl)methoxy)carbonyl)-7-(oxazol-2-ylmethoxy)-1,2,3,4-tetrahydroisoquinoline-3-carboxylic Acid

Step 1: (S)-2-(tert-butoxycarbonyl)-7-hydroxy-1,2,3,4-tetrahydroisoquinoline-3-carboxylic acid (500 mg, 1.705 mmol) was dissolved in toluene (10.00 mL) and MeOH (10 mL) then treated with TMS-Diazomethane (1.705 mL, 3.41 mmol) at 0° C. for 1 h. Then the volatiles were removed under reduced pressure to give the crude product 2-(tert-butyl) 3-methyl(S)-7-hydroxy-3,4-dihydroisoquinoline-2,3 (1H)-dicarboxylate. UPLC-MS: (ES, m/z): 284 [M+H]+.

Step 2:2-(tert-butyl) 3-methyl(S)-7-hydroxy-3,4-dihydroisoquinoline-2,3 (1H)-dicarboxylate (0.522 g, 1.7 mmol) was dissolved in dry ACN (15 mL), K₂CO₃ (0.775 g, 5.61 mmol) and 2-(bromomethyl) oxazole (0.289 g, 1.785 mmol) at 50° C. for 16 h. The mixture was left to reach rt and filtered to remove the solids (KBr and K₂CO₃). Acetonitrile was evaporated under reduced pressure, the residue was taken up with EtOAc and washed with water (2 times) and brine. The combined organic was dried (Na₂SO₄) and the solvent removed under reduced pressure. The product was dissolved in THF and water treated with lithium hydroxide (0.102 g, 4.25 mmol) at 100° C. for 2×1 h under MW irradiation. Then the mixture was acidified with citric acid (5% aqueous solution) and extracted with EtOAc. The organic phase was dried (Na₂SO₄) and the solvent removed under reduced pressure to give(S)-2-(tert-butoxycarbonyl)-7-(oxazol-2-ylmethoxy)-1,2,3,4-tetrahydroisoquinoline-3-carboxylic acid. UPLC-MS: (ES, m/z): 375 [M+H]+.

Step 3: (S)-2-(tert-butoxycarbonyl)-7-(oxazol-2-ylmethoxy)-1,2,3,4-tetrahydroisoquinoline-3-carboxylic acid (0.636 g, 1.7 mmol) was dissolved in DCM (8 mL) and treated at rt with TFA (2.62 mL, 34.0 mmol) for 2 h. UPLC MS monitor showed that the reaction was complete. The volatiles were removed to give the crude product(S)-7-(oxazol-2-ylmethoxy)-1,2,3,4-tetrahydroisoquinoline-3-carboxylic acid which was used in the next step.

Step 4: (S)-7-(oxazol-2-ylmethoxy)-1,2,3,4-tetrahydroisoquinoline-3-carboxylic acid (466 mg, 1.7 mmol) was dissolved in was dissolved in acetone (22 mL) and water (44 mL). The mixture treated with sodium bicarbonate (857 mg, 10.20 mmol) and Fmoc-Osu (516 mg, 1.530 mmol) for 16 h. The reaction mixture turns limpid. EtOAc 30 mL was added, and the mixture acidified with HCl 1N. The phases were separated and the aqueous phase was extracted with another 30 mL of EtOAc. The combined organic layers were dried (Na₂SO₄) filtered and the solvent removed under reduced pressure to give the crude product which was lyophilized to give the crude product which was purified by reverse phase chromatography (column 55 g, elution A=H2O+0.1% TFA; B=ACN+0.1% TFA, 10% B 2 CV then from 10% B to 90% B in 15 CV) to give(S)-2-(((9H-fluoren-9-yl)methoxy)carbonyl)-7-(oxazol-2-ylmethoxy)-1,2,3,4-tetrahydroisoquinoline-3-carboxylic acid. UPLC-MS: (ES, m/z): 330 [M+Na]+. ¹H NMR (300 MHz, DMSO-d₆) δ 12.8 (bs, 1H), 8.18 (d, 1H), 7.93-7.89 (m, 2H), 7.71-7.69 (m, 1H), 7.65-7.62 (m, 1H), 7.46-7.27 (m, 6H), 7.15-7.13 (m, 1H), 6.95-6.86 (m, 3H), 5.22 (d, 2H), 4.86-4.84 (m, 1H), 4.63-4.56 (m, 1H), 4.5-4.26 (m, 4H), 3.13-3.01 (m, 2H).

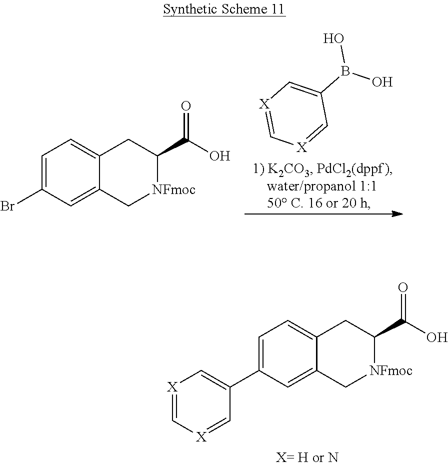

### Precursor to Tic7Ph

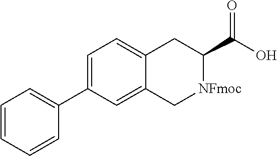

### (S)-2-(((9H-fluoren-9-yl)methoxy)carbonyl)-7-phenyl-1,2,3,4-tetrahydroisoquinoline-3-carboxylic Acid

(S)-2-(((9H-fluoren-9-yl)methoxy)carbonyl)-7-bromo-1,2,3,4-tetrahydroisoquinoline-3-carboxylic acid (500 mg, 1.045 mmol) and phenylboronic acid (382 mg, 3.14 mmol) were dissolved in water (20 mL)/2-propanol (20 mL) and the solution degassed under N₂ for 30 minutes then the solution heated at 50° C. and potassium carbonate (722 mg, 5.23 mmol) and PdCl₂dppf (38.2 mg, 0.052 mmol) were added under N₂ flux. The reaction mixture was heated for 20 h. The reaction mixture was cooled EtOAc was added, and the mixture washed with HCl 1N the phases separated and the organic dried (Na₂SO₄), filtered and the solvent removed under reduced pressure to give the crude product which was purified by reverse phase chromatography (column C18 110 g, water/acetonitrile each eluent with 0.1% TFA, gradient: 90:10 to 10:90 in 10 CV) to give title compound, as beige solid. UPLC-MS: (ES, m/z): 476 [M+H]+.

### Tic7Pyrim

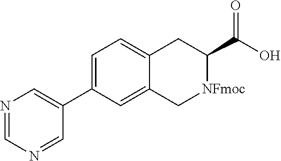

### (S)-2-(((9H-fluoren-9-yl)methoxy)carbonyl)-7-(pyrimidin-5-yl)-1,2,3,4-tetrahydroisoquinoline-3-carboxylic Acid

Pyrimidin-5-ylboronic acid (777 mg, 6.27 mmol), (S)-2-(((9H-fluoren-9-yl)methoxy)carbonyl)-7-bromo-1,2,3,4-tetrahydroisoquinoline-3-carboxylic acid (500 mg, 1.045 mmol) and K₂CO₃ (433 mg, 3.14 mmol) were dissolved in 2-propanol (20 mL) and water (20 mL). The mixture was degassed with N₂ through bubbling of the solution then the mixture heated at 50° C. PdCl₂ (dppf)(50 mg, 0.068 mmol) was added, and the mixture heated at 50° C. for 16 h under nitrogen. Then the mixture was cooled to rt. The mixture was acidified with aqueous HCl (1 M) and extracted with DCM. The collected organic phase was evaporated to dryness under reduced pressure to give the crude product which was purified by reverse phase chromatography (C18 column 110 g Luknova, water/acetonitrile 0.1% TFA in both eluents, gradient from 100:0 to 90:10 in 12 CV) to give after lyophilization the title compound. UPLC-MS: (ES, m/z): 478 [M+H]+. ¹H NMR (300 MHz, DMSO-d₆) δ 12.8 (bs, 1H), 9.2 (d, 1H), 9.17 (d, 2H), 7.92 (m, 2H), 7.75-7.62 (m, 4H), 7.46-7.20 (m, 5H), 5.1-4.9 (m, 1H), 4.82-4.15 (m, 6H), 3.25 (m, 2H).

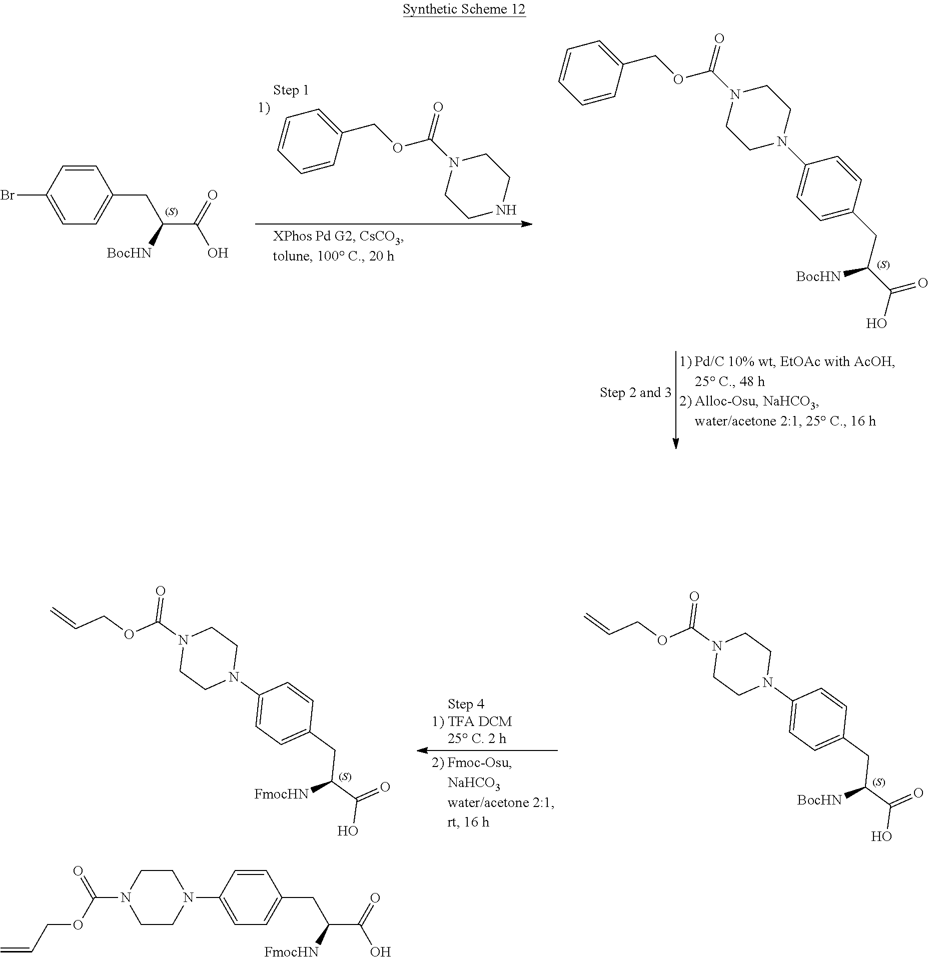

### (S)-2-((((9H-fluoren-9-yl)methoxy)carbonyl)amino)-3-(4-(4-((allyloxy)carbonyl)piperazin-1-yl)phenyl)propanoic Acid

Step 1: (S)-3-(4-bromophenyl)-2-((tert-butoxycarbonyl)amino)propanoic acid (10 g, 29.1 mmol) and benzyl piperazine-1-carboxylate (5.58 mL, 29.1 mmol), were dissolved/suspended in toluene (120 mL). Bubbling with N₂ was performed for 5 minutes and the mixture heated at 90-100° C. X Phos Pd G2 (3.43 g, 4.36 mmol) and cesium carbonate (14.20 g, 43.6 mmol) were added under nitrogen flux and the mixture heated at 110° C. for 20 h under nitrogen. Then the mixture was cooled to rt and acidified with citric acid, the aqueous phase was extracted with EtOAc. The organic phase was washed with brine. The collected organic phase was dried (Na₂SO₄) and filtered. The solvent was removed under reduced pressure to give the crude product which was purified by reverse phase flash chromatography (column 330 g Lucknova, eluent petroleum ether/EtOAc 0.5% AcOH in both eluents, gradient from 100:0 to 40:60 in 10 CV then to 20:80 in 1 CV and 20:80 2 CV) to give(S)-3-(4-(4-((benzyloxy)carbonyl)piperazin-1-yl)phenyl)-2-((tert-butoxycarbonyl)amino)propanoic acid, AcOH salt (residual excess of AcOH present). UPLC-MS: (ES, m/z): 484 [M+H]+.

Step 2: (S)-3-(4-(4-((benzyloxy)carbonyl)piperazin-1-yl)phenyl)-2-((tert-butoxycarbonyl)amino)propanoic acid (12 g, 24.82 mmol) was dissolved in EtOAc (310 mL) and the solution was degassed under vacuum and fluxed with N2, Pd on carbon 0.2 eq. was added under N₂, and the mixture treated with H2 for 48 h. The reaction was complete. The solid catalyst was filtered and the solvent removed under reduced pressure to give the crude product(S)-2-((tert-butoxycarbonyl)amino)-3-(4-(piperazin-1-yl)phenyl)propanoic acid as the AcOH salt. UPLC-MS: (ES, m/z): 450 [M+H]+.

Step 3: A solution of(S)-2-((tert-butoxycarbonyl)amino)-3-(4-(piperazin-1-yl)phenyl)propanoic acid, AcOH salt (6 g, 14.65 mmol) in acetone (16.5 mL) was treated with sodium bicarbonate (4.92 g, 58.6 mmol) dissolved in water (33 mL) and with Alloc-OSu (2.043 g, 10.26 mmol) at rt for 16 hours. Then Alloc-OSu 0.1 eq was added and the reaction mixture stirred for 16 h. The reaction mixture was washed with Et₂O, acidified with HCl 1N and extracted with EtOAc two times. The collected organic layer was dried (Na₂SO₄), filtered and the solvent was removed under reduced pressure. The crude product was lyophilized (water:acetonitrile 1:1) to give(S)-3-(4-(4-((allyloxy)carbonyl)piperazin-1-yl)phenyl)-2-((tert-butoxycarbonyl)amino)propanoic acid. UPLC-MS: (ES, m/z): 434 [M+H]+.

Step 4: (S)-3-(4-(4-((allyloxy)carbonyl)piperazin-1-yl)phenyl)-2-((tert-butoxycarbonyl)amino)propanoic acid (6.2 g, 14.30 mmol) was dissolved in DCM (70 mL) and treated with TFA 22 mL at rt for 4 h. The volatiles were removed, and the residue suspended in acetone (33 mL). The suspension was brought to pH 8 with sodium bicarbonate (6.01 g, 71.5 mmol) dissolved in water (66 mL) and Fmoc-OSu (3.22 g, 10.01 mmol) was added and the mixture stirred at rt for 16 h. The mixture was acidified with citric acid 5% aqueous solution and the aqueous extracted two times with EtOAc. The combined organic phases were dried (Na₂SO₄) filtered and the solvent removed under reduced pressure to give the crude product which was purified by flash chromatography on silica (240 g column Lucknova, eluent petroleum ether/EtOAc 0.5% AcOH in both eluents, gradient from 100:0 to 40:60 in 10 CV) to give after lyophilization (water/acetonitrile 1:1) and salt exchange with HCl the title compound, HCl salt. UPLC-MS: (ES, m/z): 556 [M+H]+. ¹H NMR (300 MHz, DMSO-d₆) δ7.98 (d, 2H), 7.71-7.63 (m, 2H), 7.43-7.37 (m, 2H), 7.35-6.5 (m, 6H), 6.00-5.95 (m, 1H), 5.27 (d, 1H), 5.2 (d, 1H), 4.57 (s, 2H), 4.19 (m, 4H), 3.62 (m, 4H), 3.18 (m, 4H), 3.02 (d, 1H), 2.8 (d, 1H).

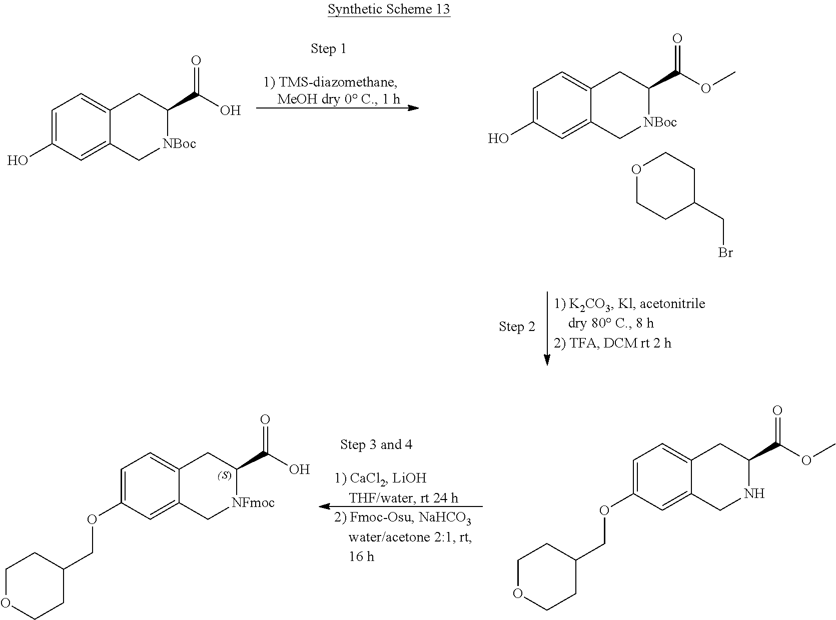

### Precursor to Tic7OCH2THP

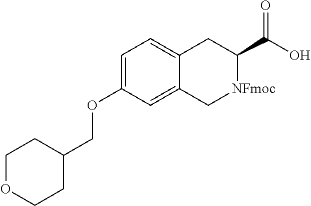

### (S)-2-(((9H-fluoren-9-yl)methoxy)carbonyl)-7-((tetrahydro-2H-pyran-4-yl)methoxy)-1,2,3,4-tetrahydroisoquinoline-3-carboxylic Acid

Step 1: (S)-2-(tert-butoxycarbonyl)-7-hydroxy-1,2,3,4-tetrahydroisoquinoline-3-carboxylic acid (500 mg, 1.705 mmol) was dissolved in dry toluene (10.00 mL) and dry MeOH (10 mL) then treated with TMS-Diazomethane (1.705 mL, 3.41 mmol) at 0° C. for 1 h under N₂. Then the volatiles were removed under reduced pressure to give the crude product 2-(tert-butyl) 3-methyl(S)-7-hydroxy-3,4-dihydroisoquinoline-2,3 (1H)-dicarboxylate. UPLC-MS: (ES, m/z): 284 [M+H]+.

Step: 2-(tert-butyl) 3-methyl(S)-7-hydroxy-3,4-dihydroisoquinoline-2,3 (1H)-dicarboxylate (600 mg, 1.952 mmol) was dissolved in acetonitrile dry (30 mL) and treated with K₂CO₃ (890 mg, 6.44 mmol) and 4-(bromomethyl)tetrahydro-2H-pyran (419 mg, 2.343 mmol) at rt for 16 h. Then K₂CO₃ (1 eq) and KI (162 mg, 0.976 mmol) and 4-(bromomethyl)tetrahydro-2H-pyran (419 mg, 2.343 mmol) were added and the mixture stirred at 80° C. for 8 h. 4-(bromomethyl)tetrahydro-2H-pyran (419 mg, 2.343 mmol) and KI (162 mg, 0.976 mmol) were added and the mixture stirred for another 20 h. The reaction was not complete. About 60% conversion was observed by UPLC MS. Then the reaction mixture was left to cool to rt and water was added and the aqueous extracted with EtOAc (2×100 mL). The combined organic phases were dried (Na₂SO₄), filtered and the solvent was evaporated under reduced pressure. The residue was purified by column chromatography on silica gel (column Lucknova 40 g column, eluting with petroleum ether/EtOAc 100:0 to 40:60 in 10 CV) to give 2-(tert-butyl) 3-methyl(S)-7-((tetrahydro-2H-pyran-4-yl)methoxy)-3,4-dihydroisoquinoline-2,3 (1H)-dicarboxylate mixture 70:30 with starting material as a colorless gum. UPLC-MS: (ES, m/z): 428 [M+Na]+.

Step 3:2-(tert-butyl) 3-methyl(S)-7-((tetrahydro-2H-pyran-4-yl)methoxy)-3,4-dihydroisoquinoline-2,3 (1H)-dicarboxylate (0.373 g, 0.920 mmol) was dissolved in DCM (5 mL) and treated at rt with TFA (1.417 mL, 18.40 mmol) for 2 h. The volatiles were removed to give the crude product methyl(S)-7-((tetrahydro-2H-pyran-4-yl)methoxy)-1,2,3,4-tetrahydroisoquinoline-3-carboxylate which used in the next step.

UPLC-MS: (ES, m/z): 306 [M+H]+.

Step 4: Methyl(S)-7-((tetrahydro-2H-pyran-4-yl)methoxy)-1,2,3,4-tetrahydroisoquinoline-3-carboxylate (291 mg, 0.953 mmol) was dissolved in acetone (22 mL) and water (44 mL) then treated with NaHCO₃ (480 mg, 5.72 mmol) and Fmoc-Osu (289 mg, 0.858 mmol) for 16 h. The reaction mixture turns limpid. EtOAc (15 mL) was added, and the mixture acidified with HCl 1N 10 mL. The aqueous phase was extracted, the phases were separated and the aqueous extracted with another 15 mL of EtOAc. The combined organic layers were dried (Na₂SO₄), filtered and the solvent removed under reduced pressure to give the crude product which was dissolved in IPA (6.00 mL) and water (12 mL) and treated with CaCl₂ (1904 mg, 17.15 mmol) and LiOH (91 mg, 3.81 mmol) and the mixture was left stirring for 24 h. Then the mixture was acidified with citric acid (5% aqueous solution) and the aqueous extracted with EtOAc. The combined organic layers were dried (Na₂SO₄) filtered and the solvent removed under reduced pressure to give the crude product which was purified by reverse phase chromatography (column C18 55 g, water/ACN each eluent with 0.1% TFA, gradient: 90:10 to 10:90 in 10 CV) to give the title compound as white solid. UPLC-MS: (ES, m/z): 514 [M+H]+. ¹H NMR (300 MHz, DMSO-d₆) δ 12.8 (bs, 1H), 7.93-7.89 (m, 2H), 7.71-7.69 (m, 1H), 7.65-7.62 (m, 1H), 7.46-7.27 (m, 4H), 7.11-7.09 (m, 1H), 6.6-6.8 (m, 2H), 4.84 (d, 1H), 4.65-4.2 (m, 5H), 3.9-3.6 (m, 4H), 3.08 (m, 2H), 1.99 (m, 1H), 1.67 (m, 2H), 1.4-1.0 (m, 4H).

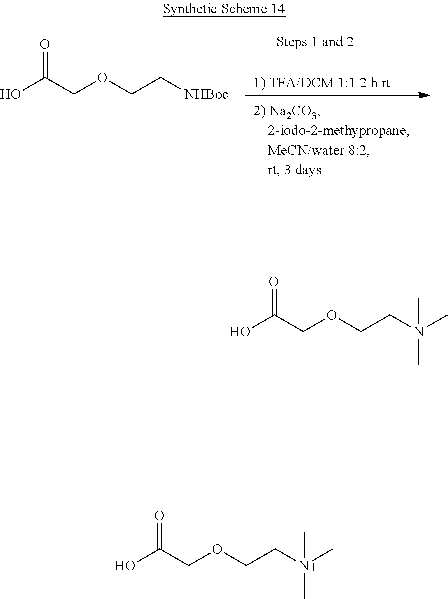

### 2-(carboxymethoxy)-N,N,N-trimethylethan-1-aminium

Step 1: 2-(2-((tert-butoxycarbonyl)amino) ethoxy) acetic acid (1 g, 4.56 mmol) was dissolved in a mixture of DCM (9.12 mL) and TFA (9.12 mL) and the rection was stirred for 2 h at rt. The solvent was removed in vacuo to afford the product 2-(2-aminoethoxy) acetic acid as a colorless oil. ¹H NMR (300 MHz, DMSO-d₆) δ 7.87 (bs, 2H), 4.09 (s, 2H), 3.67 (t, 2H), 3.01-2.97 (m, 2H).

Step 2: To a solution of 2-(2-aminoethoxy) acetic acid (0.543 g, 4.56 mmol) in MeCN/H₂O (8:2), was added sodium bicarbonate (1.450 g, 13.68 mmol), followed by 2-iodo-2-methylpropane (7.36 mL, 91 mmol). The reaction mixture was left stirring at rt 3 days. The solvent was removed by rotary evaporator. The crude product was dissolved in methanol, filtered and the solvent was removed with a rotary evaporator. The residue was dissolved in water/acetonitrile and freeze-dried to afford the crude product as a white solid 2-(carboxymethoxy)-N,N,N-trimethylethan-1-aminium. UPLC-MS: (ES, m/z): 162 [M]+.

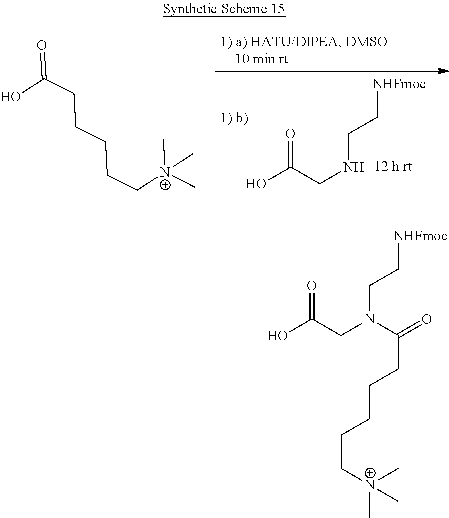

### Precursor to deNC2NH2Gly|C6acidMe3

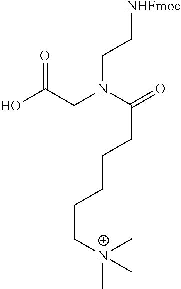

### 6-((2-((((9H-fluoren-9-yl)methoxy)carbonyl)amino)ethyl)(carboxymethyl)amino)-N,N,N-trimethyl-6-oxohexan-1-aminium

To a solution of 6-(trimethyl-14-azaneyl) hexanoic acid (600 mg, 3.44 mmol) in DMSO were added HATU (1571 mg, 4.13 mmol) and DIPEA (1443 μl, 8.26 mmol). The resulting reaction mixture was stirred for 10 min. Later, the amine (2-((((9H-fluoren-9-yl)methoxy)carbonyl)amino)ethyl)glycine (1172 mg, 3.44 mmol) was added and the reaction was left stirring at room temperature for 12 h. The crude product was purified by Biotage Flash Chromatography by using a Biotage Sfar Bio C4 duo 300 Å 20 μm column to afford the title compound. UPLC-MS: (ES, m/z): 496 [M]+.

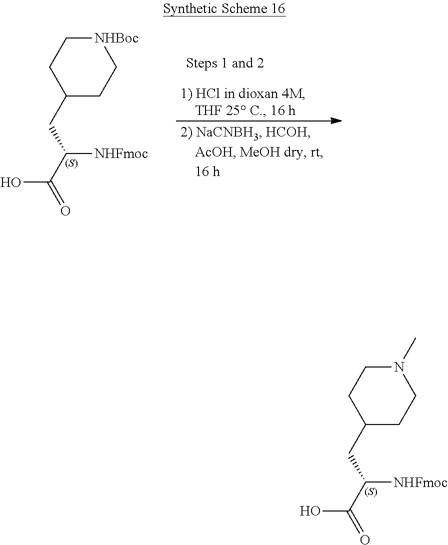

### Precursor to AlaPip4 Me

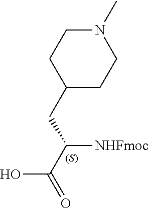

### (S)-2-((((9H-fluoren-9-yl)methoxy)carbonyl)amino)-3-(1-methylpiperidin-4-yl)propanoic Acid

Step 1: A solution of(S)-2-((((9H-fluoren-9-yl)methoxy)carbonyl)amino)-3-(1-(tert-butoxycarbonyl)piperidin-4-yl)propanoic acid (1.5 g, 3.03 mmol) in THF (10 mL) was treated with HCl 4 M in dioxane (10 mL, 40.0 mmol) for 16 h. The volatiles were removed under reduced pressure to give the crude product(S)-2-((((9H-fluoren-9-yl)methoxy)carbonyl)amino)-3-(piperidin-4-yl)propanoic acid, HCl salt. UPLC-MS: (ES, m/z): 395 [M+H]+.

Step 2: A solution of(S)-2-((((9H-fluoren-9-yl)methoxy)carbonyl)amino)-3-(piperidin-4-yl)propanoic acid, HCl salt (500 mg, 1.160 mmol) in dry MeOH (12 mL) was treated with acetic acid (0.133 mL, 2.321 mmol), formaldehyde (0.864 mL, 11.60 mmol) and NaCNBH₃ (365 mg, 5.80 mmol) for 16 h under nitrogen flux. The solvents were removed, and the crude product purified by reverse phase chromatography (C18 150 g column: elution A=H2O+0.1% TFA; B=ACN+0.1% TFA initial 5% B for 1 CV then from 5% B to 40% B in 12 CV) to give after salt exchange the title compound, HCl salt. UPLC-MS: (ES, m/z): 409 [M+H]+.

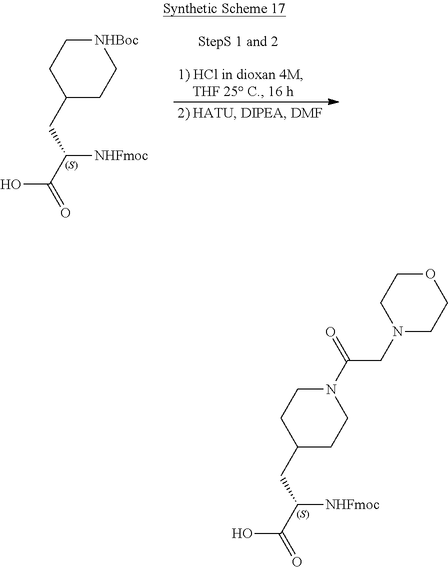

### Precursor to AlaPip4|MorphNAcOH

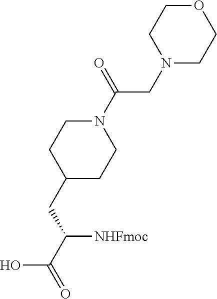

### (S)-2-((((9H-fluoren-9-yl)methoxy)carbonyl)amino)-3-(1-(2-morpholinoacetyl)piperidin-4-yl)propanoic Acid

Step 1: A solution of(S)-2-((((9H-fluoren-9-yl)methoxy)carbonyl)amino)-3-(1-(tert-butoxycarbonyl)piperidin-4-yl)propanoic acid (1.5 g, 3.03 mmol) in THF (10 mL) was treated with HCl 4 M in dioxane (10 mL, 40.0 mmol) for 16 h. The volatiles were removed under reduced pressure to give the crude product(S)-2-((((9H-fluoren-9-yl)methoxy)carbonyl)amino)-3-(piperidin-4-yl)propanoic acid, HCl salt. UPLC-MS: (ES, m/z): 395 [M+H]+.

Step 2: A solution of 2-morpholinoacetic acid (440 mg, 3.03 mmol), HATU (1267 mg, 3.33 mmol) and DIPEA (1.588 mL, 9.09 mmol) in DMF 25 mL was added to a solution of(S)-2-((((9H-fluoren-9-yl)methoxy)carbonyl)amino)-3-(piperidin-4-yl)propanoic acid (1195 mg, 3.03 mmol) in DMF (25 mL). An aggregate gel was observed after few minutes from addition. The gel was filtered and the DMF was removed under reduced pressure to give the crude desired product which was purified by reverse phase flash chromatography (C18 column 150 g Luknova elution A=H2O+0.1% TFA; B=ACN+0.1% TFA Flow: 80 mL/min, 10% B 2 CV, from 10% B to 70% B in 10 CV). The product obtained was not pure enough due to presence of 2-morpholinoacetic acid so half of it (500 mg) was further purified by RP-HPLC; deltapak C18 mm 15 μm: eluted with (% B): 20 (5 min); 20 to 40 (20 min) then 45 (5 min) then 80 (5 min); A=H2O+0.1% TFA; B=ACN+0.1% TFA Flow: 80 mL/min, λ: 214 nm. Sample load: DMSO (2.5 mL), to give the title compound. UPLC-MS: (ES, m/z): 522 [M+H]+.

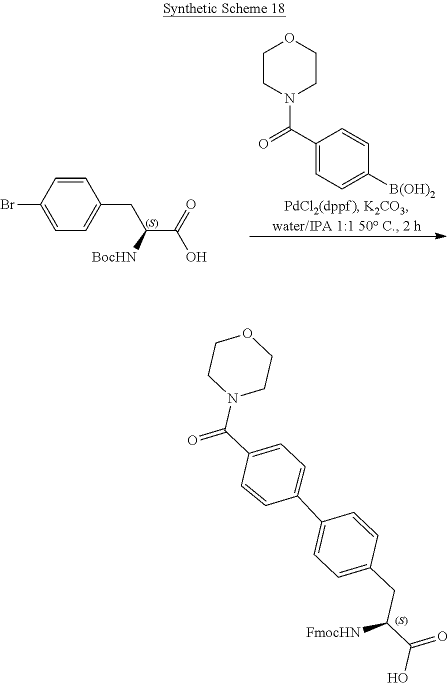

### Precursor to Bip4COMph

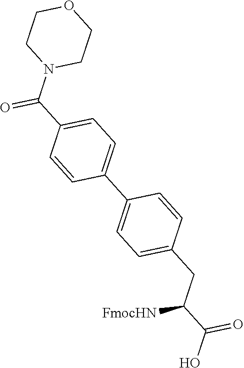

### (S)-2-((((9H-fluoren-9-yl)methoxy)carbonyl)amino)-3-(4′-(morpholine-4-carbonyl)-[1,1′-biphenyl]-4-yl)propanoic Acid

(S)-2-((((9H-fluoren-9-yl)methoxy)carbonyl)amino)-3-(4-bromophenyl)propanoic acid (500 mg, 1.072 mmol), morpholino (4-(4,4,5,5-tetramethyl-1,3,2-dioxaborolan-2-yl)phenyl)methanone (408 mg, 1.287 mmol), K₂CO₃ (741 mg, 5.36 mmol) and PdCl₂ (dppf)(39.2 mg, 0.054 mmol) were dissolved in a mixture 1:1 of IPA (10 mL) and water (10 mL) and heated at 45° C. for 1 h under nitrogen. Then the mixture was cooled to rt and IPA removed under reduced pressure. The aqueous layer was acidified to pH 3 with HCl (1N) and extracted with EtOAc, the organic washed with brine. The collected organic phase was dried (Na₂SO₄) and filtered on CELITE and the solvent removed under reduced pressure to give the crude solid title compound. UPLC-MS: (ES, m/z): 577 [M+H]+.

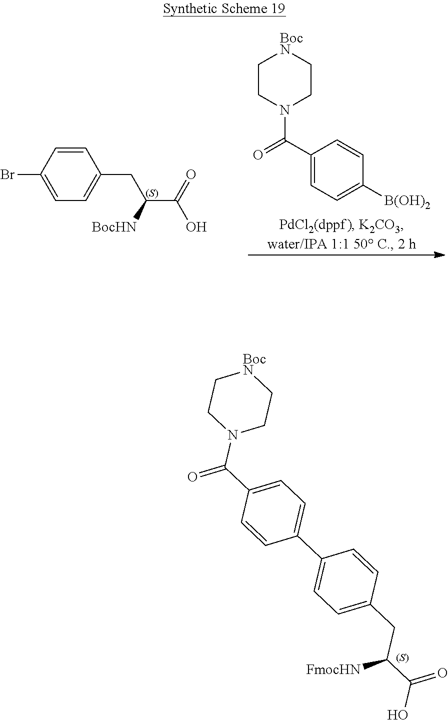

### Precursor to Bip4COPipz

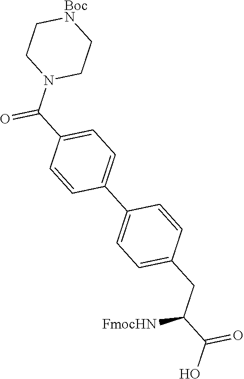

### (S)-2-((((9H-fluoren-9-yl)methoxy)carbonyl)amino)-3-(4′-(4-(tert-butoxycarbonyl)piperazine-1-carbonyl)-[1,1′-biphenyl]-4-yl)propanoic Acid

(S)-2-((((9H-fluoren-9-yl)methoxy)carbonyl)amino)-3-(4-bromophenyl)propanoic acid (500 mg, 1.072 mmol), tert-butyl 4-(4-(4,4,5,5-tetramethyl-1,3,2-dioxaborolan-2-yl)benzoyl)piperazine-1-carboxylate (580 mg, 1.394 mmol), K₂CO₃ (741 mg, 5.36 mmol) and PdCl₂ (dppf)(39.2 mg, 0.054 mmol) were dissolved in a mixture 1:1 of IPA (10 mL) and water (10 mL) and heated at 45° C. for 16 h under nitrogen. Then the mixture was cooled to rt and yjr IPA removed under reduced pressure. The aqueous layer was acidified to pH 3 with HCl 1N and extracted with EtOAc. The organic layer was washed with brine. The collected organic phase was dried (Na₂SO₄) and filtered. The solvent was removed under reduced pressure to give the crude product which was purified by flash chromatography on silica (column 80 g HP Luknova, eluent petroleum ether/EtOAc 0.5% AcOH in both eluents, gradient from 90:10 to 70:30 in 13 CV) to give the title compound as a white solid. UPLC-MS: (ES, m/z): 676 [M+H]+.

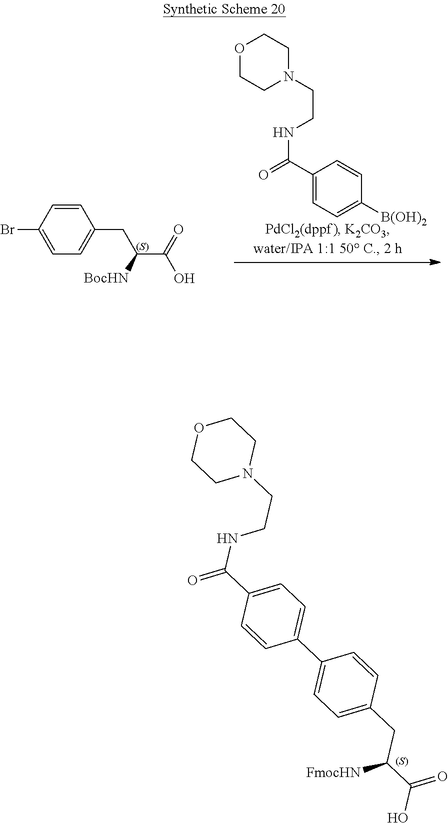

### Precursor to Bip4COAEMph

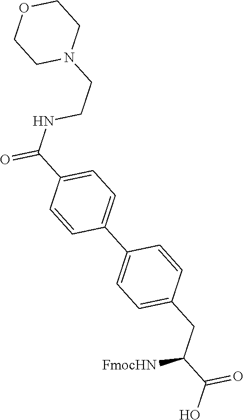

### (S)-2-((((9H-fluoren-9-yl)methoxy)carbonyl)amino)-3-(4′-((2-morpholinoethyl) carbamoyl)-[1,1′-biphenyl]-4-yl)propanoic Acid

(S)-2-((((9H-fluoren-9-yl)methoxy)carbonyl)amino)-3-(4-bromophenyl)propanoic acid (300 mg, 0.643 mmol), N-(2-morpholinoethyl)-4-(4,4,5,5-tetramethyl-1,3,2-dioxaborolan-2-yl)benzamide (278 mg, 0.772 mmol), K₂CO₃ (445 mg, 3.22 mmol) were dissolved in a 1:1 mixture of IPA (10 mL) and water (10 mL). Bubbling of the mixture with nitrogen was performed. The mixture was heated at 45° C. and PdCl₂ (dppf)(23.54 mg, 0.032 mmol) was added. The reaction mixture heated at the same temperature for 1 h under nitrogen. Then the mixture was cooled to rt and the IPA was removed under reduced pressure. The aqueous phase was acidified to pH 3 with HCl 1N and extracted with EtOAc. The organic phase washed with brine. The collected organic phase was dried (Na₂SO₄) and filtered. The solvent was removed under reduced pressure to give the crude product which was purified by flash chromatography on silica C18 (column 50 g, eluent acetonitrile/water 1% TFA in both eluents, gradient from 10:10 (10 CV) to 10:60 in (3 CV)) to give the title compound TFA salt, white solid. UPLC-MS: (ES, m/z): 620 [M+H]+.

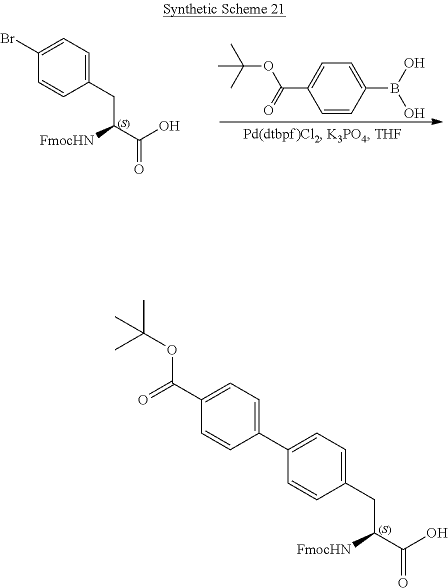

### Precursor to Bip4CO2H

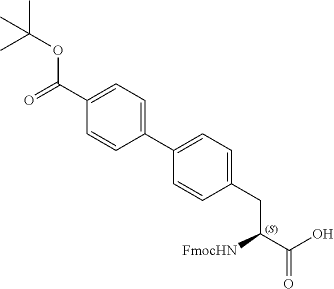

### (S)-2-((((9H-fluoren-9-yl)methoxy)carbonyl)amino)-3-(4′-(tert-butoxycarbonyl)-[1,1′-biphenyl]-4-yl)propanoic Acid

Argon gas was bubbled through a mixture of(S)-2-((((9H-fluoren-9-yl)methoxy)carbonyl)amino)-3-(4-bromophenyl)propanoic acid (6 g, 12.87 mmol), (4-(tert-butoxycarbonyl)phenyl) boronic acid (4.29 g, 19.30 mmol) and K₃PO₄ (8.19 g, 38.6 mmol) in THF (40 mL) for 10 min, then [1,1′-bis(di-tert-butylphosphino) ferrocene]dichloropalladium (II) (0.839 g, 1.287 mmol) was added. After the resulting mixture was stirred at 50° C. for 16 h, it was diluted with EtOAc (300 mL) and washed with aqueous saturated NaHCO₃ (3×80 mL), brine (2×40 mL), dried over Na₂SO₄ and filtered. The filtrate was concentrated under reduced pressure. The residue was purified by silica gel column chromatography, eluted with gradient 0%-50% EtOAc in PE. The product-containing fractions were collected and roto-evaporated in vacuo. The residue was re-purified by combi-Flash with the following conditions: Column: Column: C18 gel column (330 g), 20-35 μm; Mobile Phase A: 0.5% aq. TFA; Mobile Phase B: MeCN; (Gradient: 0% B hold 10 min, up to 62.3% B within 25 min, 62.3% B hold 6.2 min; up to 95% B within 2 min, 95% B hold 10 min); Flow rate: 90 mL/min; Detector: UV 254 & 210 nm; RT: 32.32 min. The product-containing fractions were collected and concentrated under reduced pressure to afford(S)-2-((((9H-fluoren-9-yl)methoxy)carbonyl)amino)-3-(4′-(tert-butoxycarbonyl)-[1,1′-biphenyl]-4-yl)propanoic acid. MS ESI calculated for C₃₅H₃₄NO₆ [M+1]⁺564.23, found 564.15. ¹H NMR (300 MHz, Methanol-d₄) δ 7.97-7.95 (m, 2H), 7.78-7.76 (m, 2H), 7.61-7.53 (m, 6H), 7.38-7.21 (m, 2H), 4.51-4.11 (m, 4H), 3.32-3.25 (m, 1H), 3.03-2.95 (m, 1H), 1.61 (s, 9H).

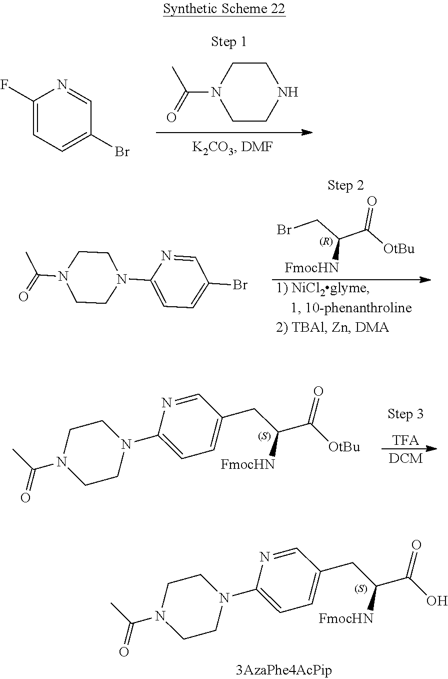

### Precursor to 3AzaPhe4AcPip

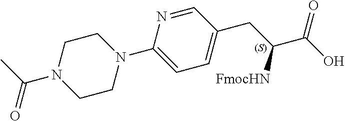

### (S)-2-((((9H-fluoren-9-yl)methoxy)carbonyl)amino)-3-(6-(4-acetylpiperazin-1-yl) pyridin-3-yl)propanoic Acid

Step 1: To a mixture of 1-(piperazin-1-yl) ethan-1-one (21.85 g, 170 mmol) in DMF (150 mL) was added 5-bromo-2-fluoropyridine (15 g, 85 mmol) under argon at rt. The reaction was stirred at 100° C. for 2 h, then diluted with 300 mL EtOAc and washed with H2O (3×80 mL), aqueous saturated NaCl (80 mL), dried over Na₂SO₄ and filtered. The filtrate was concentrated under reduced pressure. The residue was purified by silica gel column chromatography, eluting with a gradient of 0%-100% EtOAc in PE. The fractions containing desired product were combined and concentrated under reduced pressure to afford 1-(4-(5-bromopyridin-2-yl)piperazin-1-yl) ethan-1-one. MS ESI calculated for C₁₁H₁₅BrN₃O [M+H]⁺ 284.03 and 286.03, found 283.90 and 285.90.

Step 2: The mixture of nickel (II) chloride ethylene glycol dimethyl ether complex (0.696 g, 3.17 mmol) and 1,10-phenanthroline (0.571 g, 3.17 mmol) in DMA (2 mL) was heated at 50° C. for 0.5 hours. The mixture of 1-(4-(5-bromopyridin-2-yl)piperazin-1-yl) ethan-1-one (4.5 g, 15.84 mmol), tert-butyl (R)-2-((((9H-fluoren-9-yl)methoxy)carbonyl)amino)-3-bromopropanoate (7.78 g, 17.42 mmol) and tetrabutylammonium iodide (5.85 g, 15.84 mmol) in DMA (2 mL) were added at 25° C. Then zinc powder (2.071 g, 31.7 mmol) was added. The resulting mixture was stirred for 1 h at 50° C. The resulting mixture was poured into water (300 mL) and extracted with EtOAc (3×300 mL). The organic layer was washed with water (100 mL) and brine (2×80 mL), dried over anhydrous sodium sulfate and concentrated under reduced pressure. The residue was purified by column chromatography on silica gel eluting with DCM-MeOH (10:1) to afford tert-butyl(S)-2-((((9H-fluoren-9-yl)methoxy)carbonyl)amino)-3-(6-(4-acetylpiperazin-1-yl) pyridin-3-yl)propanoate. MS ESI calculated for C₃₃H₃₉N₄O₅ [M+H]⁺ 571.28, found 571.40.

Step 3: To a mixture of tert-butyl(S)-2-((((9H-fluoren-9-yl)methoxy)carbonyl)amino)-3-(6-(4-acetylpiperazin-1-yl) pyridin-3-yl)propanoate (7.3 g, 12.79 mmol) in DCM (70 mL) was added TFA (70 mL, 909 mmol) under argon at rt. The reaction was stirred at rt for 1 h then concentrated under reduced pressure. The residue was dissolved in THF (20 mL) and the resultant mixture was purified by reverse phase Combi-Flash with the following conditions: Column C18 silica gel column (330 g), 20-35 μm; Mobile Phase A: 5 mM aq. TFA; Mobile Phase B: MeCN; (Gradient: 0% B hold 10 min, up to 42.3% B within 35 min, 42.3% B hold 3.2 min; up to 95% B within 2 min, 95% B hold 10 min); Flow rate: 60 mL/min; Detector: UV 254 & 210 nm; RT: 35.32 min. The product-containing fractions were collected and concentrated under vacuum to afford(S)-2-((((9H-fluoren-9-yl)methoxy)carbonyl)amino)-3-(6-(4-acetylpiperazin-1-yl) pyridin-3-yl)propanoic acid. MS ESI calculated for C₂₉H₃₁N₄O₅ [M+H]⁺515.22, found 515.15. ¹H NMR (300 MHz, Methanol-d₄) δ 7.87-7.79 (m, 4H), 7.62-7.55 (m, 2H), 7.42-7.27 (m, 4H), 7.11-7.09 (m, 1H), 4.51-4.46 (m, 1H), 4.32-4.09 (m, 3H), 3.65-3.54 (m, 8H), 3.29-3.20 (m, 1H), 2.94-2.89 (m, 1H), 2.12 (s, 3H).

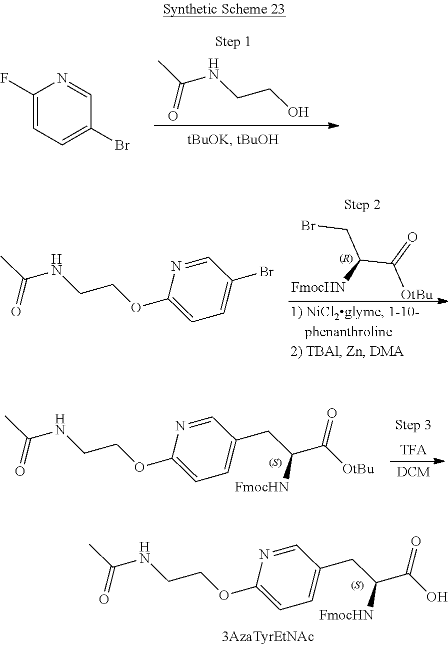

### Precursor to 3AzaTyrEtNAc

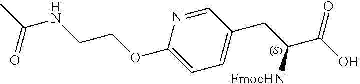

### (S)-2-((((9H-fluoren-9-yl)methoxy)carbonyl)amino)-3-(6-(2-acetamidoethoxy)pyridin-3-yl)propanoic Acid

Step 1: To a mixture of N-(2-hydroxyethyl) acetamide (17.58 g, 170 mmol) in t-BuOH (150 mL) was added under argon at rt 5-bromo-2-fluoropyridine (15 g, 85 mmol) followed by potassium tert-butoxide (19.13 g, 170 mmol). The reaction was stirred at rt for 1 h then concentrated under reduced pressure. The residue was diluted with 500 mL EtOAc and washed with aqueous saturated NaHCO₃ (3×250 mL), aqueous saturated NaCl (250 mL), dried over Na₂SO₄ and filtered. The filtrate was concentrate under reduced pressure to afford crude N-(2-((5-bromopyridin-2-yl)oxy)ethyl) acetamide. MS ESI calculated for C9H12BrN2O2 [M+H]⁺ 259.00 and 261.00, found 258.90 and 261.90.

Step 2: The mixture of nickel (II) chloride ethylene glycol dimethyl ether complex (1.187 g, 5.40 mmol) and 1,10-phenanthroline (0.974 g, 5.40 mmol) in DMA (70 mL) was heated at 50° C. for 0.5 hours. The mixture of N-(2-((5-bromopyridin-2-yl)oxy)ethyl) acetamide (7 g, 27.0 mmol), tert-butyl (R)-2-((((9H-fluoren-9-yl)methoxy)carbonyl)amino)-3-bromopropanoate (13.26 g, 29.7 mmol) and tetrabutylammonium iodide (9.98 g, 27.0 mmol) in DMA (70 mL) were added at 25° C. Then zinc powder (3.53 g, 54.0 mmol) was added and the resulting mixture was stirred for 1 h at 50° C. The reaction mixture was diluted with 300 mL EtOAc and washed with aqueous saturated NaHCO₃ (3×80 mL), brine (80 mL), dried over Na₂SO₄ and filtered. The filtrate was concentrated under reduced pressure. The residue was purified by silica gel column chromatography, eluting with gradient 0%-20% DCM in MeOH. The fractions containing the desired product were combined and concentrated under reduced pressure to afford tert-butyl(S)-2-((((9H-fluoren-9-yl)methoxy)carbonyl)amino)-3-(6-(2-acetamidoethoxy)pyridin-3-yl)propanoate. MS ESI calculated for C31H36N3O6 [M+H]⁺ 546.25, found 546.40.

Step 3: To a mixture of tert-butyl(S)-2-((((9H-fluoren-9-yl)methoxy)carbonyl)amino)-3-(6-(2-acetamidoethoxy)pyridin-3-yl)propanoate (8 g, 14.66 mmol) in DCM (80 mL) was added TFA (80 mL, 1038 mmol) under argon at rt. The reaction was stirred at rt for 1 h then concentrated under reduced pressure. The residue was purified by RP flash column chromatography with the following conditions: Column: C18 silica gel column (330 g), 20-35 μm; Mobile Phase A: 5 mM aq. NH₄HCO₃; Mobile Phase B: MeCN; (Gradient: 0% B hold 5 min, up to 45% B within 20 min, 45% B hold 10 min; up to 95% B within 15 min, 95% B hold 10 min); Flow rate: 60 mL/min; Detector: UV 254 & 210 nm; RT: 35.32 min. The product-containing fractions were collected and concentrated under reduced pressure to give(S)-2-((((9H-fluoren-9-yl)methoxy)carbonyl)amino)-3-(6-(2-acetamidoethoxy)pyridin-3-yl)propanoic acid. MS ESI calculated for C27H28N3O6 [M+H]⁺ 490.19, found 490.10. ¹H NMR (400 MHz, Methanol-d₄) δ 8.02-8.00 (m, 1H), 7.79-7.77 (m, 2H), 7.67-7.64 (m, 1H), 7.61-7.57 (m, 2H), 7.40-7.36 (m, 2H), 7.31-7.27 (m, 2H), 6.82-6.80 (m, 1H), 4.42-4.39 (m, 1H), 4.33-4.22 (m, 4H), 4.16-4.13 (m, 1H), 3.53-3.51 (m, 2H), 3.20-3.15 (m, 1H), 2.93-2.87 (m, 1H), 1.92 (s, 3H).

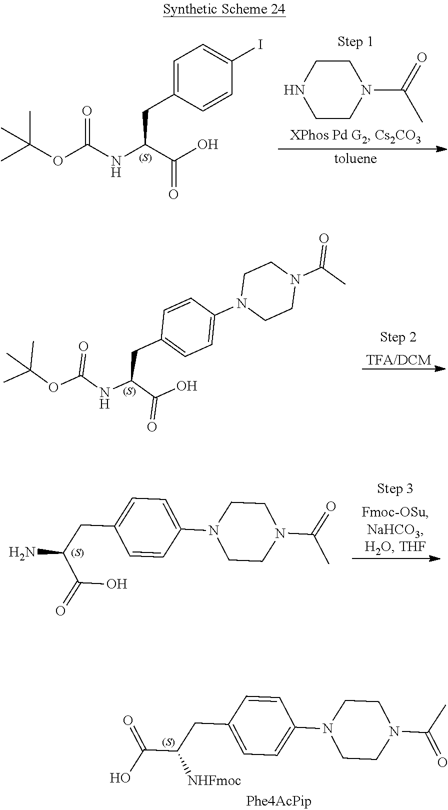

### Precursor to Phe4AcPip

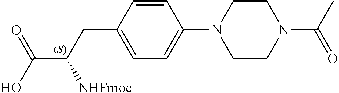

### (S)-2-((((9H-fluoren-9-yl)methoxy)carbonyl)amino)-3-(4-(4-acetylpiperazin-1-yl)phenyl)propanoic Acid

Step 1: To a stirred solution of(S)-3-(4-bromophenyl)-2-((tert-butoxycarbonyl)amino)propanoic acid (6 g, 17.43 mmol) in toluene (180 mL) was added XPhos Pd G₂ (2.057 g, 2.61 mmol) at 25° C. under nitrogen. The resulting solution was stirred at 100° C. for 10 min. 1-(Piperazin-1-yl) ethan-1-one (2.234 g, 17.43 mmol) and Cs₂CO₃ (5.04 g, 26.1 mmol) were added and the resulting solution was stirred at 110° C. for 2 h. The reaction was cooled to rt and quenched with H₂O (500 mL), extracted with EtOAc (2×500 mL). The combined organic layer was washed with brine (3×200 mL), dried over anhydrous Na₂SO₄ and filtered. The filtrate was concentrated under reduced pressure and the residue was purified by silica gel column chromatography, eluted with 0-60% EtOAc in PE to give(S)-3-(4-(4-acetylpiperazin-1-yl)phenyl)-2-((tert-butoxycarbonyl)amino)propanoic acid. MS ESI calculated for C₂₀H₃₀N₃O₅ [M+H]⁺392.21, found 392.25.1H NMR (400 MHz, Methanol-d₄) δ 7.12 (d, J=8 Hz, 2H), 6.89 (d, J=8 Hz, 2H), 4.16-4.13 (m, 1H), 3.72-3.65 (m, 4H), 3.14-3.04 (m, 4H), 2.93-2.81 (m, 2H), 2.13 (s, 3H), 1.38-1.29 (m, 9H).

Step 2: To a stirred solution of(S)-3-(4-(4-acetylpiperazin-1-yl)phenyl)-2-((tert-butoxycarbonyl)amino)propanoic acid (10 g, 25.5 mmol) in DCM (30 mL) was added TFA (30 mL) at rt. The solution was stirred at 25° C. for 1 h then concentrated under reduced pressure. The crude(S)-3-(4-(4-acetylpiperazin-1-yl)phenyl)-2-aminopropanoic acid was used to the next step directly without any further purification. MS ESI calculated for C₁₅H₂₂N₃O₃ [M+H]⁺292.16, found 292.20.

Step 3: To a stirred solution of(S)-3-(4-(4-acetylpiperazin-1-yl)phenyl)-2-(carboxyamino)propanoic acid (7 g, 20.87 mmol) in THF (25 mL) and water (25 mL) was added Fmoc-OSu (6.34 g, 18.79 mmol) at 25° C. under nitrogen. And then NaHCO₃ (8.77 g, 104 mmol) was added. The resulting mixture was stirred at 25° C. for 2 h. The pH was adjusted to 5 with 1N HCl and extracted with EtOAc (2×200 mL). The combined organic layer was washed with brine (3×100 mL), dried over anhydrous Na₂SO₄ and filtered. The filtrate was concentrated under reduced pressure and the residue was purified by reverse phase flash chromatography with the following conditions: Column: C18 silica gel (330 g); Mobile Phase A: water (0.1% TFA), Mobile Phase B: MeCN; (Gradient: 5% B hold 5 min, up to 55% B within 15 min, 55% B hold 5 min; up to 95% B within 20 min, 95% B hold 5 min); Flow rate: 90 mL/min; Detector: UV 210 nm; RT=45 min. The product-containing fractions were collected and concentrated in vacuo to give(S)-2-((((9H-fluoren-9-yl)methoxy)carbonyl)amino)-3-(4-(4-acetylpiperazin-1-yl)phenyl)propanoic acid. MS ESI calculated for C₃₀H₃₂N₃O₅ [M+H]⁺514.23, found 514.30. ¹H NMR (400 MHz, Methanol-d₄) δ 7.78-7.76 (m, 2H), 7.61-7.52 (m, 2H), 7.40-7.37 (m, 2H), 7.32-7.22 (m, 4H), 7.08-6.98 (m, 2H), 4.47-4.43 (m, 1H), 4.33-4.31 (m, 1H), 4.14-4.02 (m, 2H), 3.68-3.63 (m, 4H), 3.23-3.08 (m, 5H), 2.91-2.85 (m, 1H), 2.11 (s, 3H).

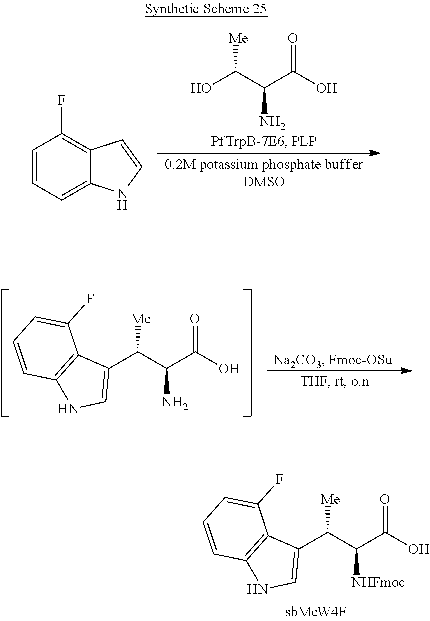

### Precursor to sbMeW4F

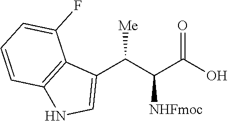

### (2S,3S)-2-amino-3-(4-fluoro-1H-indol-3-yl)butanoic Acid

Into a 1-L 3-necked round-bottom flask purged and maintained with an inert atmosphere of nitrogen, was placed 4-fluoro-1H-indole (10 g, 1.00 eq), L-threonine (10.6 g, 1.20 eq), DMSO (100 mL), potassium phosphate buffer (0.2 M, 300 mL, pH=7.4). The reaction mixture was heated to 65° C., then PfTrpB-7 E6 (2.5 g, 25 wt %) and 3-hydroxy-2-methyl-5-([phosphonooxy]methyl)-4-pyridinecarboxaldehyde (0.078 g, 0.004 eq) were added. The resulting solution was stirred overnight at 65° C. The mixture was then cooled to rt and used directly in the next step.

Into the above reaction mixture, THF (100 mL), sodium carbonate (23.56 g, 3.0 eq.) and 2,5-dioxopyrrolidin-1-yl 9H-fluoren-9-ylmethyl carbonate (29.96 g, 1.20 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. The mixture was applied onto a silica gel column with MeOH:DCM=1:5. HPLC-MS: (ES, m/z): [M+1]: 459. ¹H NMR (300 MHz, DMSO-d₆) δ 12.60 (s, 1H), 11.15 (s, 1H), 7.87 (d, J=7.6 Hz, 2H), 7.76-7.49 (m, 3H), 7.47-7.34 (m, 2H), 7.34-7.16 (m, 4H), 7.03 (td, J=7.9, 5.0 Hz, 1H), 6.73 (dd, J=11.8, 7.7 Hz, 1H), 4.36 (t, J=8.5 Hz, 1H), 4.31-4.02 (m, 3H), 3.51 (q, J=7.4 Hz, 1H), 1.31 (d, J=7.0 Hz, 4H), 0.78 (s, 1H).

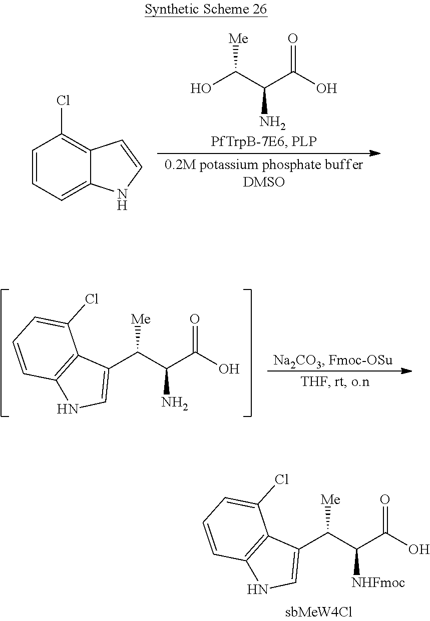

### Precursor to sbMeW4C₁

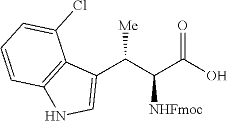

### (2S,3S)-2-amino-3-(4-chloro-1H-indol-3-yl)butanoic Acid

Into a 1-L 3-necked round-bottom flask purged and maintained with an inert atmosphere of nitrogen, was placed 4-chloro-1H-indole (10 g, 1.00 eq.), L-threonine (14.09 g, 1.8 eq.), DMSO (100 mL), potassium phosphate buffer (0.2 M, 300 mL, pH=7.4), the reaction mixture was heated to 65° C., then PfTrpB-7 E6 (7.5 g, 25 wt %) and 3-hydroxy-2-methyl-5-([phosphonooxy]methyl)-4-pyridinecarboxaldehyde (174 mg, 0.01 eq.) were added. The resulting solution was stirred for 36 h at 65° C. The mixture was then cooled to rt and used directly in the next step.

Into the above reaction mixture, THF (100 mL), sodium carbonate (20.9 g, 3.0 eq.) and 2,5-dioxopyrrolidin-1-yl 9H-fluoren-9-ylmethyl carbonate (31.0 g, 1.40 eq.) were added at 0° C. The resulting solution was stirred overnight at rt. The pH was adjusted to 4 by 3 M HCl and the solid precipitate was filtered away. The resulting solution was extracted with EtOAc (3×500 mL). The organic fractions were combined, and washed with brine (1 L), dried over anhydrous sodium sulfate and concentrated under vacuum. HPLC-MS: (ES, m/z): [M+1]: 475

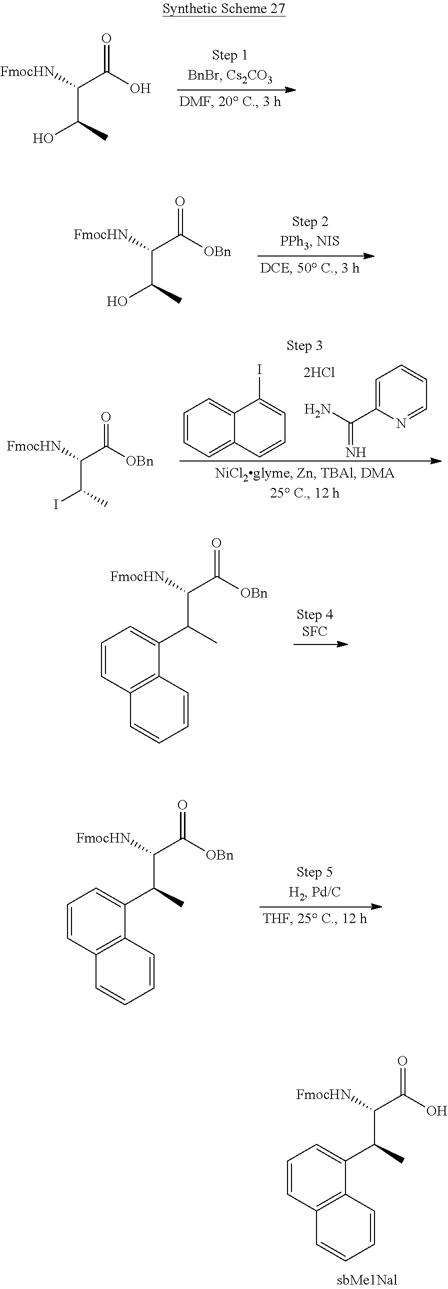

### Precursor to sbMe1Nal

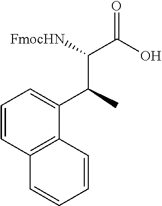

### (2S,3S)-2-amino-3-(naphthalen-1-yl)butanoic Acid

Step 1: To a solution of (2S,3R)-2-((((9H-fluoren-9-yl)methoxy)carbonyl)amino)-3-hydroxybutanoic acid (250 g, 1.00 eq) in DMF (1.5 L) was added benzyl bromide (250 g, 2.00 eq) dropwise at 20° C. Then, cesium carbonate (477 g, 2.00 eq) was added and the solution was stirred at 20° C. for 3 h. The reaction was poured into ice H₂O (3 L) and extracted with EtOAc (500 mL×3). The organic phase was washed with 3% LiCl solution (500 mL×2) and brine (500 mL), dried over sodium sulfate and concentrated under vacuum at 40° C. The crude product was triturated with methyl tert-butyl ether:PE=6:1. ¹H NMR (400 MHz, CDCl₃): δ 7.77 (d, J=7.6 Hz, 1H), 7.40 (d, J=8.0 Hz, 1H), 7.31-7.36 (m, 10H), 5.65-5.71 m, 1H), 5.13-5.31 (m, 3H), 4.39-4.43 (m, 3H), 4.22-4.25 (m, 1H), 1.25 (d, J=6.4 Hz, 3H)

Step 2: To a 3-neck round-bottom flask was added (2S,3R)-benzyl 2-((((9H-fluoren-9-yl)methoxy)carbonyl)amino)-3-hydroxybutanoate (125 g, 1.00 eq) and DCE (750 mL) with an inert atmosphere of nitrogen. The reaction was cooled to 0° C. followed by the addition of NIS (195 g, 3.00 eq) and PPh₃ (228 g, 3.00 eq). The temperature was raised to 50° C. and the reaction mixture was stirred for 3 h. The reaction was poured into ice H₂O (500 mL) and extracted with DCM (500 mL×2). The organic phase was dried over sodium sulfate and concentrated under vacuum at 40° C. The residue was purified by silica gel column chromatography (PE/EtOAc=I/O to 0/1). ¹H NMR (400 MHz, CDCl₃): δ 7.78 (d, J=7.6 Hz, 2H), 7.68 (d, J=7.2 Hz, 2H), 7.33-7.43 (m, 9H), 5.27-5.68 (m, 1H), 5.21-5.23 (m, 2H), 4.39-4.52 (m, 3H), 4.25-4.38 (m, 1H), 1.91-1.95 (m, 3H).

Step 3: To a 3-neck round-bottom flask was placed 2-((((9H-fluoren-9-yl)methoxy) carbonyl)amino)-3-iodobutanoate, 1-iodonaphthalene (42.2 g, 1.20 eq), TBAI (76.7 g, 1.50 eq), Zn (19.0 g, 2.10 eq) and DMA (750 mL). To a second 3-neck round-bottom flask was placed picolinimidamide·2HCl (42.2 g, 1.20 eq), NiCl₂·glyme (7.61 g, 0.25 eq) and DMA (750 mL) at 25° C. Under argon, the contents of the second flask were added to the first flask. The resulting mixture was then stirred for 12 h at 25° C. The reaction was poured into ice H₂O (3 L) and extracted with EtOAc (1 L×2). The organic phase was dried over sodium sulfate and concentrated under vacuum at 40° C. The crude product was purified by reversed-phase HPLC (MeCN:H₂O). HPLC-MS: [M+23]: 564. ¹H NMR (400 MHz, CDCl₃) δ: 8.17-8.24 (m, 1H), 8.15-8.17 (m, 1H), 7.77-7.87 (m, 2H), 7.76-7.77 (m, 4H), 7.30-7.41 (m, 10H), 5.30-5.38 (m, 1H), 4.96-5.04 (m, 3H), 4.85-4.87 (m, 1H), 4.30-4.34 (m, 1H), 4.18-4.26 (m, 4H), 1.43-1.45 (m, 3H).

Step 4:143 g of (2S)-benzyl 2-((((9H-fluoren-9-yl)methoxy)carbonyl)amino)-3-(naphthalen-1-yl)butanoate was separated by SFC. The organic phase was concentrated under vacuum at 35° C.

Peak 1: (2S,3R)-benzyl 2-((((9H-fluoren-9-yl)methoxy)carbonyl)amino)-3-(naphthalene-1-yl)butanoate. ¹H NMR (400 MHz, DMSO-d₆): δ 8.11-8.12 (m, 2H), 8.10-8.11 (m, 1H), 7.88-7.90 (m, 2H), 7.54-7.88 (m, 1H), 7.44-7.53 (m, 2H), 7.42-7.44 (m, 4H), 7.33-7.42 (m, 3H), 7.27-7.33 (m, 6H), 7.08-7.09 (m, 2H), 4.91-4.94 (m, 1H), 4.79-4.82 (m, 1H), 4.58 (t, J=8.0 Hz), 4.17-4.25 (m, 4H), 1.39 (d, J=6.8 Hz, 3H). Peak 2: (2S,3S)-benzyl 2-((((9H-fluoren-9-yl)methoxy)carbonyl)amino)-3-(naphthalen-1-yl)butanoate. ¹H NMR (400 MHz, DMSO-d₆): δ 7.92-8.15 (m, 1H), 7.86-7.92 (m, 1H), 7.84-7.86 (m, 1H), 7.57-7.84 (m, 2H), 7.56-7.57 (m, 1H), 7.41-7.54 (m, 4H), 7.30-7.38 (m, 4H), 7.27-7.30 (m, 7H), 5.08-5.14 (m, 2H), 4.65 (t, J=8.0 Hz), 4.23-4.26 (m, 1H), 4.05-4.18 (m, 3H), 1.30 (d, J=6.8 Hz, 3H).

Step 5: To a 3-neck round-bottom flask was added (2S,3S)-benzyl 2-((((9H-fluoren-9-yl)methoxy)carbonyl)amino)-3-(naphthalen-1-yl)butanoate (40.0 g, 1.00 eq) and THF (200 mL). 10% wet Pd/C (7.00 g) was added and the reaction was purged 3 times with H₂ and stirred at 25° C. for 12 h under H₂ (15 psi). The reaction was filtered through a celite pad and concentrated under vacuum at 35° C. The crude product was triturated with PE at 25° C. for 1 h. After filtration, the filter cake was dissolved in MeCN (100 mL) and concentrated under vacuum at 35° C. to remove residual solvent. HPLC-MS: [M+23]: 474. ¹H NMR (400 MHz, DMSO-d₆) δ 12.78 (s, 1H), 8.23 (d, J=7.6 Hz, 1H), 7.86-7.88 (m, 1H), 7.80-7.86 (m, 2H), 7.61-7.80 (m, 2H), 7.55-7.59 (m, 4H), 7.481-7.55 (m, 1H), 7.40-7.48 (m, 3H), 7.27-7.29 (m, 2H), 4.28-4.60 (m, 1H), 4.24-4.28 (m, 1H), 4.17-4.24 (m, 2H), 4.04-4.15 (m, 1H), 1.36 (d, J=6.8 Hz, 3H).

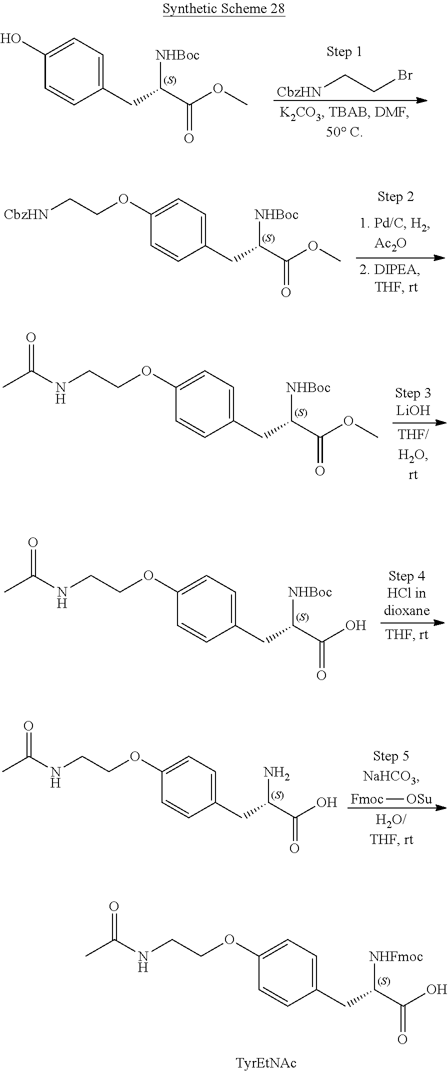

### Precursor to TyrEtNAc

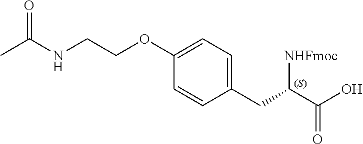

### L-Tyrosine O-ethyl acetamide or(S)-3-(4-(2-acetamidoethoxy)phenyl)-2-aminopropanoic Acid

Step 1: To a stirred solution of methyl (tert-butoxycarbonyl)-L-tyrosinate (10.0 g, 33.9 mmol), benzyl (2-bromoethyl) carbamate (26.2 g, 102 mmol) and TBAB (5.46 g, 16.93 mmol) in DMF (150 mL) was added potassium carbonate (14.04 g, 102 mmol) at rt. The mixture was then stirred at 50° C. for 24 h. The mixture was cooled to rt, quenched with water (250 mL) and extracted with EtOAc (2×500 mL). The combined organic layers were washed with brine (3×150 mL), dried over anhydrous sodium sulfate and filtered. The filtrate was concentrated under reduced pressure, and the residue was purified by silica gel column chromatography, eluted with 0-30% EtOAc in PE. MS ESI calculated for C₂₅H₃₂N₂O₇ [M+Na]⁺ 495.22, found 495.10; 1H NMR (300 MHz, CDCl₃) δ 7.38-7.32 (m, 5H), 7.04 (d, J=8.4 Hz, 2H), 6.81 (d, J=8.4 Hz, 2H), 5.31 (br, 1H), 5.21 (s, 2H), 4.97 (br, 1H), 4.56-4.53 (m, 1H), 4.03 (t, J=5.0 Hz, 2H), 3.72 (s, 3H), 3.64-3.58 (m, 2H), 3.06-3.01 (m, 2H), 1.43 (s, 9H).

Step 2: To a stirred solution of methyl(S)-3-(4-(2-(((benzyloxy)carbonyl)amino) ethoxy)phenyl)-2-((tert-butoxycarbonyl)amino)propanoate (16.0 g, 33.9 mmol) and acetic anhydride (6.39 mL, 67.7 mmol) in THF (200 mL) was added Pd/C (3.60 g, 33.9 mmol, dry, 10% wt) at rt under nitrogen atmosphere. The mixture was degassed with hydrogen 3 times and stirred at 20° C. for 4 h. DIPEA (17.74 mL, 102 mmol) was added to the mixture and stirred at 20° C. for 1 h. The mixture was filtered, and the filtrate was concentrated under reduced pressure. The residue was purified by silica gel column chromatography, eluted with 0-3% MeOH in DCM. MS ESI calculated for C₁₉H₂₈N₂O₆ [M+Na]⁺ 403.19, found 403.10; ¹H NMR (300 MHz, CDCl₃) δ 7.05 (d, J=8.4 Hz, 2H), 6.85-6.80 (m, 2H), 5.99 (br, 1H), 5.32 (br, 1H), 4.99-4.97 (m, 1H), 4.02 (t, J=5.0 Hz, 2H), 3.72 (s, 3H), 3.69-3.63 (m, 2H), 3.10-2.89 (m, 2H), 2.02 (s, 3H), 1.42 (s, 9H).

Step 3: To a stirred solution of methyl(S)-3-(4-(2-acetamidoethoxy)phenyl)-2-((tert-butoxycarbonyl)amino)propanoate (12.5 g, 32.9 mmol) THF (100 mL) was added lithium hydroxide (65.7 mL, 65.7 mmol, 1N in water) at rt. The solution was stirred at 20° C. for 2 h. The pH of the solution was adjusted to 3 with 1N HCl. The aqueous layer was extracted with EtOAc (2×250 mL). The combined organic layers were washed with brine (150 mL), dried over anhydrous sodium sulfate, filtered, and concentrated under reduced pressure.

MS ESI [M+H]⁺: 367.10.

Step 4: To a stirred solution of(S)-3-(4-(2-acetamidoethoxy)phenyl)-2-((tert-butoxycarbonyl)amino)propanoic acid (12.5 g, 30.7 mmol) in THF (20 mL) was added 4N HCl in dioxane (200 mL) at rt. The solution was stirred at 20° C. for 1 h. The solvent was concentrated under reduced pressure. MS ESI [M+H]⁺: 267.05.

Step 5: To a stirred mixture of(S)-3-(4-(2-acetamidoethoxy)phenyl)-2-aminopropanoic acid hydrochloride (9.50 g, 25.1 mmol) and NaHCO₃ (10.54 g, 126 mmol) in THF (100 mL) and water (100 mL) was added Fmoc-OSu (7.62 g, 22.59 mmol) at rt. The mixture was stirred at 20° C. for 1 h. The pH value of the solution was adjusted to 3 with 1N HCl. The aqueous phase was extracted with EtOAc (2×500 mL). The combined organic layers were washed with brine (150 mL), dried over anhydrous sodium bicarbonate and filtered. The filtrate was concentrated under reduced pressure, and the residue was recrystallized from EtOAc (200 mL). The solid was collected by filtration and dried under vacuum. MS ESI [M+H]+: 489.05; ¹H NMR (300 MHz, Methanol-d₄) δ 7.79 (d, J=7.6 Hz, 2H), 7.62-7.57 (m, 2H), 7.42-7.26 (m, 4H), 7.17-7.14 (m, 2H), 6.83 (d, J=8.4 Hz, 2H), 4.41-4.31 (m, 2H), 4.29-4.10 (m, 2H), 3.96 (t, J=4.8 Hz, 2H), 3.51 (t, J=5.4 Hz, 2H), 3.19-3.13 (m, 1H), 2.92-2.84 (m, 1H), 1.94 (s, 3H).

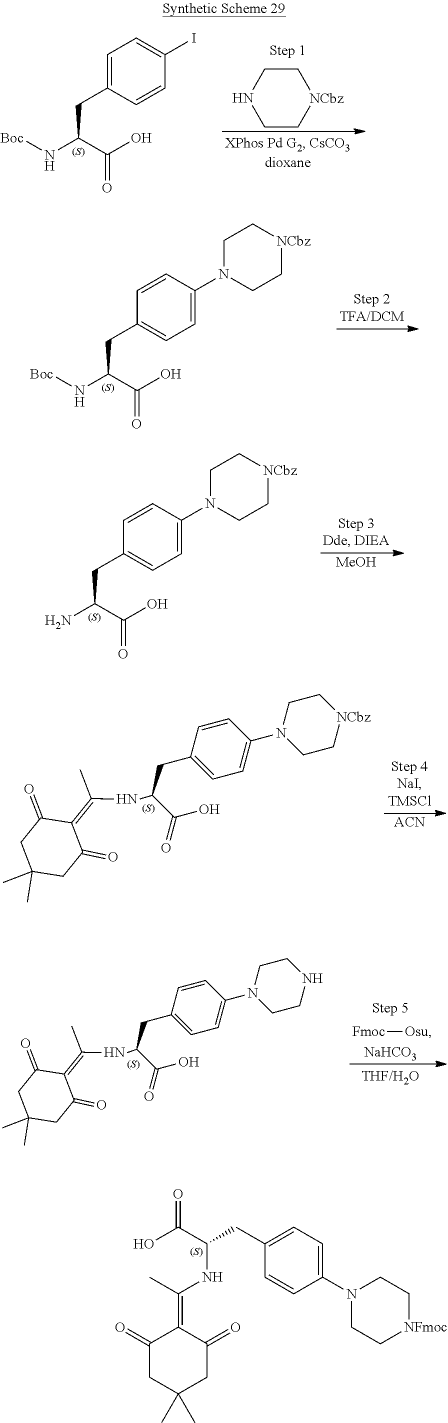

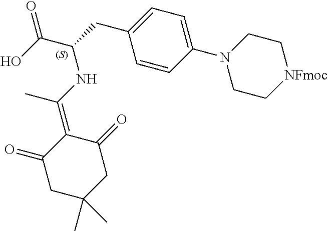

### (S)-3-(4-(4-(((9H-fluoren-9-yl)methoxy)carbonyl)piperazin-1-yl)phenyl)-2-((1-(4,4-dimethyl-2,6-dioxocyclohexylidene)ethyl)amino)propanoic Acid

Step 1: To a solution of benzyl piperazine-1-carboxylate (16.89 g, 77 mmol) and(S)-2-((tert-butoxycarbonyl)amino)-3-(4-iodophenyl)propanoic acid (10 g, 25.6 mmol) in dioxane (300 mL) were added chloro(2-dicyclohexylphosphino-2′,4′,6′-triisopropyl-1,1′-biphenyl) [2-(2′-amino-1,1′-biphenyl)]palladium (II)(2.011 g, 2.56 mmol), Cs₂CO₃ (24.99 g, 77 mmol) at room temperature. The resulted mixture was stirred for 18 h at 100° C. then cooled to ambient temperature, diluted with water (200 mL) and the pH value was adjusted to 5 with hydrochloric acid (1 M). After filtration, the filtrate was concentrated under vacuum to afford red solid which was purified by Rp-flash with the following conditions: Column: C18 gel column (330 g); Mobile Phase A: water (0.1% TFA); Mobile Phase B: MeCN; (Gradient: 0% B hold 5 min, up to 45% B within 20 min, 50% B hold 10 min; up to 95% B within 5 min, 95% B hold 10 min). The product-containing fractions were collected and roto-evaporated in vacuo to give(S)-3-(4-(4-((benzyloxy)carbonyl)piperazin-1-yl)phenyl)-2-((tert-butoxycarbonyl)amino)propanoic acid (6.8 g, 12.94 mmol, 50.6% yield) as a yellow oil. MS ESI calculated for C₂₆H₃₄N₃O₆ [M+H]⁺ 484.24, found 484.50. ¹H NMR (300 MHz, CDCl₃) δ 7.42-7.30 (m, 5H), 7.19-7.18 (m, 2H), 7.16-7.11 (m, 2H), 5.17 (s, 2H), 5.02-5.00 (m, 1H), 4.55-4.49 (m, 1H), 3.79 (s, 4H), 3.26 (s, 4H), 3.25-3.12 (m, 2H), 1.41 (s, 9H).

Step 2: To a stirred mixture of(S)-3-(4-(4-((benzyloxy)carbonyl)piperazin-1-yl)phenyl)-2-((tert-butoxycarbonyl)amino)propanoic acid (5.8 g, 11.99 mmol) in DCM (40 mL) was added TFA (30 mL, 389 mmol) at 25° C. The resulting mixture was stirred for 2.5 h at 25° C. The solvent was evaporated under reduced pressure to give crude(S)-2-amino-3-(4-(4-((benzyloxy)carbonyl)piperazin-1-yl)phenyl)propanoic acid (4.6 g, 10.56 mmol, 88% yield) as a colorless oil. MS ESI calculated for C₂₁H₂₆N₃O₄ [M+H]⁺ 384.18, found 384.10.

Step 3: To a mixture of(S)-2-amino-3-(4-(4-((benzyloxy)carbonyl)piperazin-1-yl)phenyl)propanoic acid (4.6 g, 12.00 mmol) in MeOH (70 mL) were added DIEA (4.19 mL, 23.99 mmol) and 2-acetyldimedone (3.28 g, 17.99 mmol) at room temperature. The reaction was stirred at room temperature for 16 h then concentrated in vacuum. The residue was acidified with aqueous HCl to pH=3 and extracted with EtOAc (2×500 mL). The organic layers were combined, washed with brine (3×350 mL), dried over anhydrous sodium sulfate and filtered. The residue was purified by silica gel chromatography, eluting with a gradient of EtOAc in PE from 0% to 100% to afford(S)-3-(4-(4-((benzyloxy)carbonyl)piperazin-1-yl)phenyl)-2-((1-(4,4-dimethyl-2,6-dioxocyclohexylidene)ethyl)amino)propanoic acid (5.9 g, 10.45 mmol, 87% yield) as a white solid. MS ESI calculated for C₃₁H₃₈N₃O₆ [M+H]⁺ 548.27, found 548.15.

Step 4: To a solution of(S)-3-(4-(4-((benzyloxy)carbonyl)piperazin-1-yl)phenyl)-2-((1-(4,4-dimethyl-2,6-dioxocyclohexylidene)ethyl)amino)propanoic acid (5.9 g, 10.77 mmol) in acetonitrile (120 mL) were added sodium iodide (16.15 g, 108 mmol) and chlorotrimethylsilane (13.61 mL, 108 mmol) at 0° C. The resulted mixture was stirred for 16 h at room temperature. The solvent was concentrated under reduced pressure and diluted with water (100 mL). The aqueous was extracted with ethyl ether (2×40 mL). The aqueous phase was used to the next step directly. MS ESI calculated for C₂₃H₃₂N₃O₄ [M+H]⁺ 414.23, found 414.15.

Step 5: To a stirred solution of(S)-2-((1-(4,4-dimethyl-2,6-dioxocyclohexylidene)ethyl)amino)-3-(4-(piperazin-1-yl)phenyl)propanoic acid (4.45 g, 10.76 mmol) in THF (50 mL) and water (50 mL) were added NaHCO₃ (4.52 g, 53.8 mmol) and Fmoc-OSu (3.63 g, 10.76 mmol) at room temperature. The resulting mixture was stirred at 25° C. for 2 h. The pH was adjusted to 3 with 1N HCl then extracted with EtOAc (2×400 mL), washed with brine (3×250 mL), dried over anhydrous (Na₂SO₄), filtered. The filtrate was concentrated under reduced pressure and the residue was purified by Rp-flash with the following conditions: 330 g C18 column, 2% 46% in 25 min, 46%-55% in 30 min, MeCN in water (0.05% TFA) to give(S)-3-(4-(4-(((9H-fluoren-9-yl)methoxy)carbonyl)piperazin-1-yl)phenyl)-2-((1-(4,4-dimethyl-2,6-dioxocyclohexylidene)ethyl)amino)propanoic acid (2.3107 g, 3.61 mmol, 33.5% yield) as an off-white solid. MS ESI calculated for C₃₈H₄₂N₃O₆ [M+H]⁺636.30, found 636.60. ¹H NMR (400 MHz, DMSO-d₆) δ 13.49-13.47 (m, 1H), 7.90-7.88 (m, 2H), 7.65-7.63 (m, 2H), 7.45-7.38 (m, 2H), 7.34-7.32 (m, 2H), 7.10-7.03 (m, 2H), 6.90-6.82 (m, 2H), 4.82-4.80 (m, 1H), 4.43-4.41 (m, 2H), 4.29-4.27 (m, 1H), 3.43 (s, 4H), 3.15-3.14 (m, 1H), 3.10-2.97 (m, 4H), 3.95-2.93 (m, 1H), 2.31-2.27 (m, 7H), 0.94 (s, 6H).

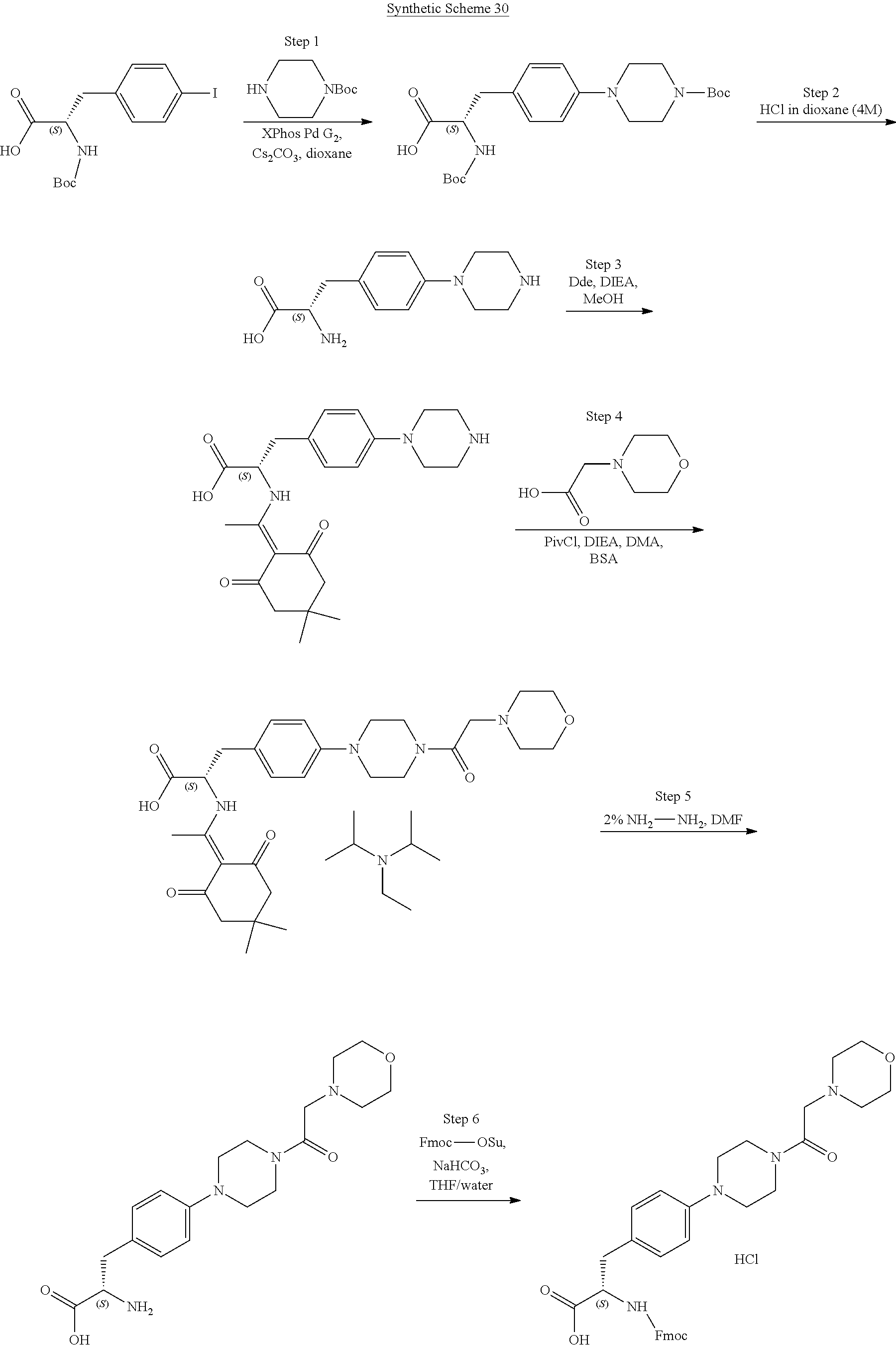

### Precursor to Phe4Piperaz|MorphNAcOH

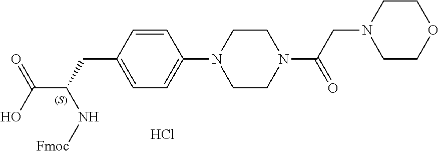

### (S)-2-((((9H-fluoren-9-yl)methoxy)carbonyl)amino)-3-(4-(4-(2-morpholinoacetyl)piperazin-1-yl)phenyl)propanoic Acid Hydrochloride

Step 1: To a solution of tert-butyl piperazine-1-carboxylate (47.6 g, 256 mmol) and(S)-2-((tert-butoxycarbonyl)amino)-3-(4-iodophenyl)propanoic acid (20 g, 51.1 mmol) in dioxane (1000 mL) were added chloro(2-dicyclohexylphosphino-2′,4′,6′-triisopropyl-1,1′-biphenyl) [2-(2′-amino-1,1′-biphenyl)]palladium (II)(8.05 g, 10.22 mmol), Cs₂CO₃ (50.0 g, 153 mmol) at room temperature. The resulted mixture was stirred for 18 h at 100° C. The reaction was cooled down to ambient temperature, diluted with water (200 mL). The pH value of the reaction solution was adjusted to 5 with hydrochloric acid (1 M). The mixture was filtered. The filtrate was concentrated under vacuum to afford red solid. The residue was diluted with water (300 mL) and ethyl acetate (1000 mL). The organic layer was washed with brine (2×300 mL), dried over anhydrous Na₂SO₄ and filtered. The filtrate was concentrated in vacuum and the residue was purified by silica gel chromatography, eluted with a gradient of MeOH in DCM from 0 to 10% to afford(S)-2-((tert-butoxycarbonyl)amino)-3-(4-(4-(tert-butoxycarbonyl)piperazin-1-yl)phenyl)propanoic acid (15.5 g, 31.0 mmol, 60.7% yield) as an orange oil. MS ESI calculated for C₂₃H₃₆N₃O₆ [M+H]⁺ 450.25, found 450.55.

Step 2: A mixture of(S)-2-((tert-butoxycarbonyl)amino)-3-(4-(4-(tert-butoxycarbonyl)piperazin-1-yl)phenyl)propanoic acid (15.5 g, 34.5 mmol) in 4N HCl in 1,4-dioxane (300 mL) was stirred for 2.5 h at 25° C. The solvent was evaporated under reduced pressure to afford(S)-2-amino-3-(4-(piperazin-1-yl)phenyl)propanoic acid (11.2 g, 31.4 mmol, 91% yield) as a brown oil. MS ESI calculated for C₁₃H₂₀N₃O₂ [M+H]⁺ 250.15, found 250.05.

Step 3: To a mixture of(S)-2-amino-3-(4-(piperazin-1-yl)phenyl)propanoic acid (11.2 g, 44.9 mmol) in MeOH (220 mL) were added DIEA (15.69 mL, 90 mmol) and Dde (12.28 g, 67.4 mmol) at room temperature. The reaction was stirred at room temperature for 16 h. The residue was acidified with 1 M HCl to pH=4. The resulting solution was concentrated in vacuum to afford crude product. The residue was purified by RP-flash with the following conditions: 330 g C¹⁸ column, 2%-2% in 5 min, 2%-30% in 30 min, ACN in water (0.05% TFA) to afford(S)-2-((1-(4,4-dimethyl-2,6-dioxocyclohexylidene)ethyl)amino)-3-(4-(piperazin-1-yl)phenyl)propanoic acid (7.9 g, 16.81 mmol, 37.4% yield) as a yellow oil. MS ESI calculated for C₂₃H₃₂N₃O₄ [M+H]⁺ 414.23, found 414.45.

Step 4: The mixture of(S)-2-((1-(4,4-dimethyl-2,6-dioxocyclohexylidene)ethyl)amino)-3-(4-(piperazin-1-yl)phenyl)propanoic acid (10.87 g, 26.3 mmol) and BSA (8.91 g, 43.8 mmol) in DMA (130 mL) was heated at 50° C. for 1 hours. The solution was cooled to room temperature and marked as solution A. The mixture of 2-morpholinoacetic acid (3.18 g, 21.91 mmol) and DIEA (4.59 mL, 26.3 mmol) in DMA (36 mL) was added PivCl (3.23 mL, 26.3 mmol) at 0° C. Then stirred for 1 h at 0° C. This mixture was added to solution A and stirred at 0° C. for 2 h. The solution was quenched with water (20 mL). The solution was concentrated under reduced pressure. The residue was purified by RP-flash with the following conditions: 330 g C18 column, 2%-2% in 5 min, 2%-35% in 30 min, ACN in water to give N-ethyl-N-isopropylpropan-2-amine(S)-2-((1-(4,4-dimethyl-2,6-dioxocyclohexylidene)ethyl)amino)-3-(4-(4-(2-morpholinoacetyl)piperazin-1-yl)phenyl)propanoate (3.1584 g, 4.55 mmol, 20.79% yield) as an off-white solid. MS ESI calculated for C₂₉H₄₁N₄O₆ [M+H]⁺ 541.29, found 541.30. ¹H NMR (400 MHz, CD₃OD) δ 7.17-7.08 (m, 2H), 6.96-6.84 (m, 2H), 4.49-4.47 (m, 1H), 3.80-3.65 (m, 8H), 3.52 (s, 2H), 3.29-3.18 (m, 1H), 3.21-3.05 (m, 4H), 2.93-2.91 (m, 1H), 2.72-2.70 (m, 4H), 2.32 (s, 4H), 2.16 (s, 3H), 1.01 (s, 6H).

Step 5: To a stirred mixture of N-ethyl-N-isopropylpropan-2-amine(S)-2-((1-(4,4-dimethyl-2,6-dioxocyclohexylidene)ethyl)amino)-3-(4-(4-(2-morpholinoacetyl)piperazin-1-yl)phenyl) propanoate (30 mg, 0.045 mmol) in DMF (1 mL) was added 2% NH₂—NH₂ (1 mL, 0.045 mmol) at 25° C. The resulting mixture was stirred for 1 h at 25° C. The solvent was evaporated under reduced pressure. The crude product was used to the next step directly. MS ESI calculated for C₁₉H₂₉N₄O₄ [M+H]⁺ 377.21, found 377.45.

Step 6: To the solution of(S)-2-amino-3-(4-(4-(2-morpholinoacetyl)piperazin-1-yl)phenyl)propanoic acid (18 mg, 0.048 mmol) in THF (2 mL) and water (2 mL) were added NaHCO₃ (20.08 mg, 0.239 mmol) and Fmoc-OSu (32.3 mg, 0.096 mmol) at room temperature. The resulting mixture was stirred at 25° C. for 2.5 h. The pH was adjusted to 3 with 1N HCl. The reaction mixture was extracted with EtOAc (100 mL×3). The organic layers were washed with brine (80 mL×3), dried over anhydrous sodium sulfate. The solvent was concentrated under reduced pressure and the residue was purified by RP-flash with the following conditions: 40 g C¹⁸ column, 2%-2% in 5 min, 2%-25% in 25 min, ACN in water to give(S)-2-((((9H-fluoren-9-yl)methoxy)carbonyl)amino)-3-(4-(4-(2-morpholinoacetyl)piperazin-1-yl)phenyl)propanoic acid hydrochloride (20.9 mg, 0.031 mmol, 65.6% yield) as a white solid. MS ESI calculated for C₃₄H₃₉N₄O₆ [M−Cl]⁺ 599.28, found 599.15. ¹H NMR (400 MHz, DMSO-d₆) δ 7.88 (d, J=7.5 Hz, 2H), 7.63-7.61 (m, 2H), 7.42-7.40 (m, 2H), 7.33-7.31 (m, 2H), 6.98 (d, J=8.1 Hz, 2H), 6.75 (d, J=8.3 Hz, 2H), 6.36 (d, J=6.6 Hz, 1H), 4.31-4.30 (m, 1H), 4.18-4.16 (m, 1H), 4.10-4.08 (m, 1H), 3.76-3.75 (m, 1H), 3.65-3.55 (m, 2H), 3.56-3.56 (m, 6H), 3.17 (s, 2H), 3.05-2.97 (m, 2H), 2.97-2.95 (m, 3H), 2.84-2.82 (m, 1H), 2.50-2.40 (m, 4H).

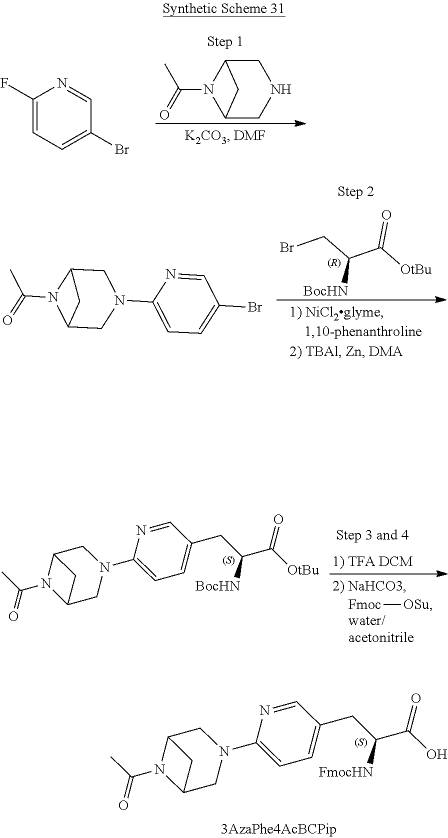

### Precursor to 3AzaPhe4AcBCPip

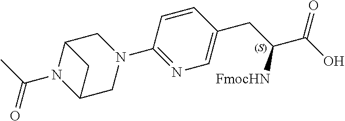

### (2S)-2-((((9H-fluoren-9-yl)methoxy)carbonyl)amino)-3-(6-(6-acetyl-3,6-diazabicyclo[3.1.1]heptan-3-yl) pyridin-3-yl)propanoic Acid

Step 1: To a mixture of 1-(3,6-diazabicyclo[3.1.1]heptan-6-yl) ethan-1-one (308 mg, 2.2 mmol) and potassium carbonate (235 mg, 1.7 mmol) in DMF (5 mL) was added 5-bromo-2-fluoropyridine (211 mg, 1.2 mmol) under argon at rt. The reaction was stirred at 100° C. for 2 h, then diluted with 30 mL EtOAc and washed with H₂O (3×80 mL), aqueous saturated NaCl (80 mL), dried over Na₂SO₄ and filtered. The filtrate was concentrated under reduced pressure. The residue was purified by BIOTAGE reversed-phase chromatography, eluting with a gradient of 10%-90% MeCN in water with 0.1% of TFA affording the desired product 1-(3-(5-bromopyridin-2-yl)-3,6-diazabicyclo[3.1.1]heptan-6-yl) ethan-1-one (300 mg). MS ESI calculated for C₁₂H₁₄BrN₃O [M+H]⁺296.16 and 298.16, found 296.2 and 298.2.

Step 2: A mixture of nickel (II) chloride (26.3 mg, 0.203 mmol) and 1,10-phenanthroline (36.5 mg, 0.203 mmol) in DMA (2 mL) was heated at 50° C. for 0.5 hours. A mixture of tert-butyl (R)-3-bromo-2-((tert-butoxycarbonyl)amino)propanoate (361 mg, 1.114 mmol), 1-(3-(5-bromopyridin-2-yl)-3,6-diazabicyclo[3.1.1]heptan-6-yl) ethan-1-one (300 mg, 1.013 mmol) and tetrabutylammonium iodide (374 mg, 1.013 mmol) in DMA (2 mL) was added at 25° C. Then zinc (132 mg, 2.026 mmol) was added. The resulting mixture was stirred for 4 h at 50° C. The reaction mixture was filtered through CELITE and reduced to a residue by rotary evaporator using high vacuum pump.

The residue was purified by reversed-phase flash chromatography on Biotage (34 g C₁₈ column, eluting with a gradient of 10%-90% MeCN in water with 0.1% of TFA) affording the desired product tert-butyl (2S)-3-(6-(6-acetyl-3,6-diazabicyclo[3.1.1]heptan-3-yl) pyridin-3-yl)-2-((tert-butoxycarbonyl)amino)propanoate (280 mg). MS ESI calculated for C₂₄H₃₆N₄O₅ [M+H]⁺461.57, found 461.5.

Step 3: The residue was treated with a solution of HCl 4N in 1,4-dioxane (10 mL) for 2 hour. After this time, the reaction mixture was checked by UPLC. The reaction mixture was concentrated to a residue and dissolved in water:acetonitrile. The solution was lyophilized to afford (2S)-3-(6-(6-acetyl-3,6-diazabicyclo[3.1.1]heptan-3-yl) pyridin-3-yl)-2-aminopropanoic acid.

Step 4: A solution of NaHCO₃ (3 eq) in H₂O (50 mL) was prepared. The resulting solution was diluted with CH₃CN (50 mL) and (2S)-3-(6-(6-acetyl-3,6-diazabicyclo[3.1.1]heptan-3-yl) pyridin-3-yl)-2-aminopropanoic acid (1 veq) and Fmoc-OSu (0.95 eq.) were added to the stirred solution. The reaction mixture was stirred at room temperature for 4 h. HCl (1N) was added and then the mixture was lyophilized. The residue was purified by reversed-phase flash chromatography on Biotage (eluting with a gradient of 10%-90% MeCN in water with 0.1% of TFA) affording the title compound (130 mg). MS ESI calculated for C₃₀H₃₀N₄O₅ [M+H]⁺527.59, found 527.4.

### Precursor to sbMe1Nal3 Me

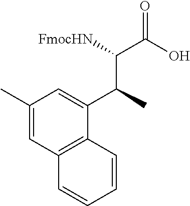

### (2S,3S)-2-((((9H-fluoren-9-yl)methoxy)carbonyl)amino)-3-(3-methylnaphthalen-1-yl)butanoic Acid

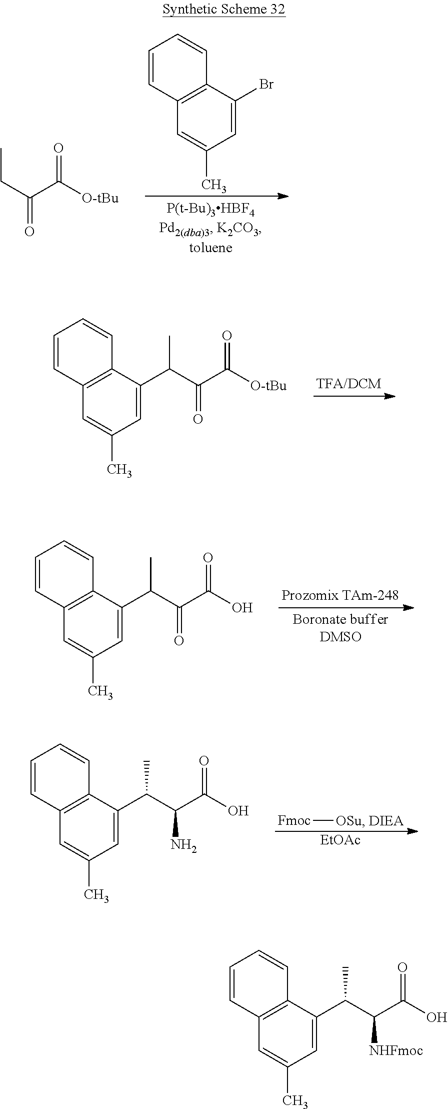

Step 1: Tert-butyl 2-oxobutanoate (50.0 g, 316 mmol, 1.00 eq) and K₂CO₃ (131 g, 948 mmol, 3.00 eq) were dissolved in in toluene (1.50 L) at 25° C. 1-bromo-3-methylnaphthalene (55.9 g, 253 mmol, 0.80 eq) and P (t-Bu)₃·HBF₄ (7.34 g, 25.3 mmol, 0.08 eq) were added to the reaction mixture at 25° C., and the reaction was purged with N₂ for three times. Pd₂(dba)₃ (5.79 g, 6.32 mmol, 0.02 eq) was added to the reaction mixture at 25° C. under N₂, and the reaction was purged with N₂ for three times. The reaction mixture was stirred at 110° C. for 3 h under N₂. TLC (petroleum ether/ethyl acetate=8/1, product R_{f}=0.38) showed the reaction was completed. The reaction mixture was poured into aq. NH₄Cl (1.00 L) and extracted with EtOAc (500 mL×3). The combined organic layers were washed with brine (500 mL×2), dried over with Na₂SO₄, filtered and concentrated under reduced pressure to give the crude product. Purification by silica chromatography (SiO₂, petroleum ether/ethyl acetate=80/1 to 1/1) gave tert-butyl 3-(3-methylnaphthalen-1-yl)-2-oxobutanoate.

Step 2: To tert-butyl 3-(3-methylnaphthalen-1-yl)-2-oxobutanoate (62.0 g, 208 mmol, 1.00 eq) in DCM (620 mL) was added TFA (620 mL) dropwise to the reaction mixture at 0° C. The reaction mixture was stirred at 20° C. for 2 h under N₂·TLC (petroleum ether/ethyl acetate=3/1, product R_{f}=0.01) showed the reaction was completed. The reaction mixture was concentrated under reduced pressure to give the crude product. The crude product was further purification by prep-HPLC (column: Phenomenex luna c18 250 mm*100 mm*10 μm; mobile phase: [H₂O (0.1% TFA)-ACN]; gradient: 30%-60% B over 20.0 min) to obtain 3-(3-methylnaphthalen-1-yl)-2-oxobutanoic acid.

Step 3: DL-Lysine (94.5 g, 646 mmol, 3.10 eq) was dissolved in DMSO (295 mL) and Na₂B₄O₇ (0.1 M, 458 mL) at 20° C. Na₂B₄O₇·10H₂O (23.9 g, 62.5 mmol, 0.30 eq) was added to the reaction mixture at 20° C. 3-(3-Methylnaphthalen-1-yl)-2-oxobutanoic acid (50.5 g, 208 mmol, 1.00 eq) was added to the reaction mixture at 20° C. Add Prozomix TAm-248 (29.5 g) to the reaction mixture at 20° C. Pyridoxal-5-phosphate hydrate (2.58 g, 10.4 mmol, 0.05 eq) was added to the reaction mixture at 20° C. The reaction mixture was stirred at 45° C. for 12 hrs. TLC (ethyl acetate/methanol=10/1, product R_{f}=0.01) showed the reaction was completed. The reaction mixture was cooled to 20° C. The pH was adjusted to 5.0 with 5N HCl (200 mL) at 20° C. The reaction mixture was stirred at 20° C. for 30 min. The mixture was filtered and the filter cake was washed with H₂O (200 mL×3). The filter cake was collected and concentrated under reduced pressure to obtain (2S,3S)-2-amino-3-(3-methylnaphthalen-1-yl)butanoic acid, which was used to next step without purification.

Step 4: (2S,3S)-2-amino-3-(3-methylnaphthalen-1-yl)butanoic acid (50.7 g, 208 mmol, 1.00 eq) was dissolved in EtOAc (507 mL) at 20° C. DIEA (32.3 g, 250 mmol, 43.6 mL, 1.20 eq) was added to reaction mixture at 20° C. Fmoc-OSu (70.3 g, 208 mmol, 1.00 eq) was added to the reaction mixture at 20° C. The reaction mixture was stirred at 20° C. for 12 h under N₂. TLC (petroleum ether/ethyl acetate=0/1, product R_{f}=0.25) showed the reaction was completed. The reaction was filtered and the filter cake was washed with EtOAc (100 mL×5). The organic layers were combined and washed with brine (300 mL×2), dried over with Na₂SO₄, filtered and concentrated under reduced pressure to give the crude product. Purification by pre-HPLC (column: WePure Biotech XP t C18 250*70*10 μm; mobile phase: [H₂O (10 mM NH₄HCO₃)-ACN]; gradient: 25%-60% B over 20.0 min) gave (2S,3S)-2-((((9H-fluoren-9-yl)methoxy)carbonyl)amino)-3-(3-methylnaphthalen-1-yl)butanoic acid. MS ESI calculated for C₃₀H₂₇NO₄ [M+H]⁺ 465.19, found 489.1 ¹H NMR (400 MHz, DMSO-d₆) δ 8.24 (d, J=6.8 Hz, 1H), 7.83-7.90 (m, 2H), 7.77-7.82 (m, 1H), 7.57-7.64 (m, 1H), 7.50-7.56 (m, 2H), 7.34-7.49 (m, 5H), 7.18-7.30 (m, 3H), 4.43 (t, J=7.6 Hz, 1H), 4.22-4.31 (m, 1H), 4.08-4.17 (m, 2H), 3.95-4.04 (m, 1H), 2.40 (s, 3H), 1.32 (d, J=6.8 Hz, 3H).

### Precursor to sbMe1Nal3F

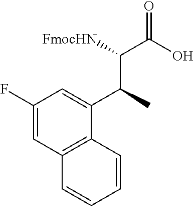

### (2S,3S)-2-((((9H-fluoren-9-yl)methoxy)carbonyl)amino)-3-(3-fluoronaphthalen-1-yl)butanoic Acid

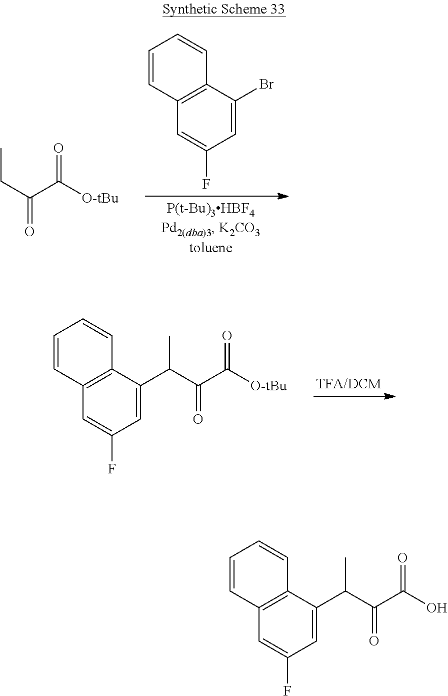

Step 1: Tert-butyl 2-oxobutanoate (1582 mg, 10 mmol, 1.00 eq) and K₂CO₃ (4146 mg, 30 mmol, 3.00 eq) were dissolved in in toluene (4.75 mL) at 25° C. 1-Bromo-3-fluoronaphthalene (4501 mg, 20 mmol, 2.0 eq) and P (t-Bu)₃·HBF₄ (290 mg, 1 mmol, 0.1 eq) were added to the reaction mixture at 25° C., and the reaction was purged with N₂ for three times. Pd₂(dba)₃ (229 mg, 0.25 mmol, 0.025 eq) was added to the reaction mixture at 25° C. under N₂, and the reaction was purged with N₂ for three times. The reaction mixture was stirred at 110° C. for 12 h under N₂. The reaction mixture was concentrated and redissolved in 1:1 MTBE:hexanes and purified by silica chromatography (SiO₂, 5-40% MTBE in hexanes) gave tert-butyl 3-(3-fluoronaphthalen-1-yl)-2-oxobutanoate.

Step 2: To tert-butyl 3-(3-fluoronaphthalen-1-yl)-2-oxobutanoate (1.21 g, 4 mmol, 1.00 eq) in DCM (3.16 mL) was added TFA (1.58 mL) dropwise to the reaction mixture. The reaction mixture was stirred at 20° C. for 2 h under N₂. The reaction mixture was concentrated under reduced pressure to give the crude product. The crude product was purified by silica chromatography (SiO₂, 10-60% % MTBE in hexanes) to 3-(3-fluoronaphthalen-1-yl)-2-oxobutanoic acid.

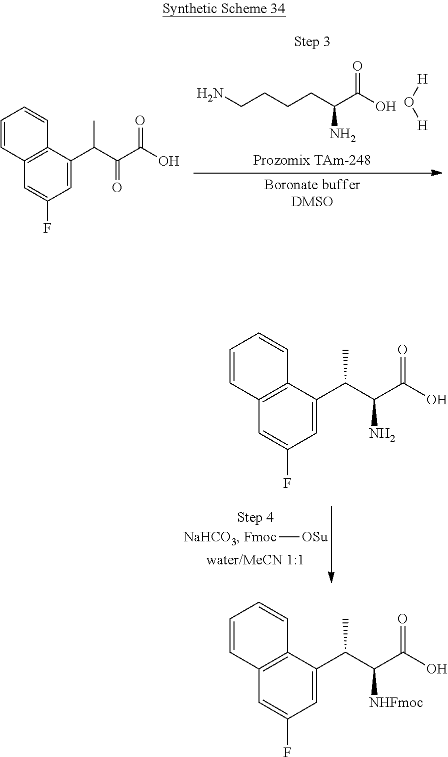

Step 3: To a solution of 3-(3-fluoronaphthalen-1-yl)-2-oxobutanoic acid (246 mg, 1.0 mmol) in DMSO (1231 μl) and borate buffer (pH 10, 0.1 M)(1.11 E+04 μl) was added L-lysine monohydrate (246 mg, 1.500 mmol). Pyridoxal Phosphate (12.36 mg, 0.050 mmol) was added, followed by Prozomix TAm-248 (172 mg, 1.000 mmol). The mixture was stirred for 48 h at which time complete disappearance of starting material was observed. The mixture was quenched with 1N HCl to adjust to pH 3-4 and directly purified by reverse phase chromatography (5-90% MeCN in water with no modifier). The compound eluted at 15-20% MeCN/water. Product-containing fractions were concentrated in vacuo to provide the product. HPLC-MS: (ES, m/z): [M+1]: 248.

Step 4: NaHCO₃ (102 mg, 1.213 mmol) was added to a solution of Fmoc-OSu (130 mg, 0.384 mmol) and (2S,3S)-2-amino-3-(3-fluoronaphthalen-1-yl)butanoic acid (100 mg, 0.404 mmol) in water/MeCN (1:1, 20 mL). The reaction mixture was stirred at room temperature overnight. The reaction mixture was cooled to room temperature and dried to residue by rotary evaporator to afford the desired product (2S,3S)-2-((((9H-fluoren-9-yl)methoxy)carbonyl)amino)-3-(3-fluoronaphthalen-1-yl)butanoic acid (150 mg, 0.319 mmol) which was used in the next reaction without further purification. UPLC-MS: (ES, m/z): [M+1]: 270.5.

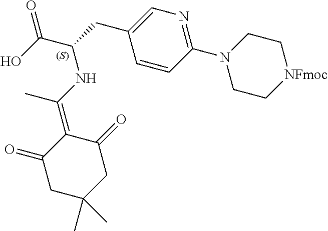

### (S)-3-(6-(4-(((9H-fluoren-9-yl)methoxy)carbonyl)piperazin-1-yl) pyridin-3-yl)-2-((1-(4,4-dimethyl-2,6-dioxocyclohexylidene)ethyl)amino)propanoic Acid

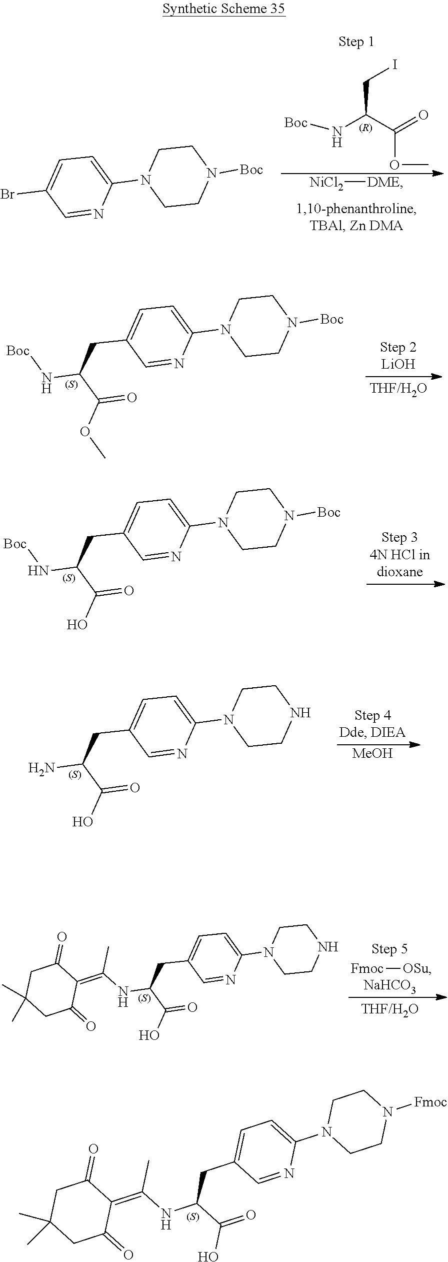

Step 1: A solution of Nickel (II) chloride ethylene glycol dimethyl ether complex (0.642 g, 2.92 mmol) and 1,10-phenanthroline (0.527 g, 2.92 mmol) in DMA (100 mL) at room temperature was prepared. The solution was stirred at 50° C. for 2 h. The solution was cooled to room temperature and tert-butyl 4-(5-bromopyridin-2-yl)piperazine-1-carboxylate (5 g, 14.61 mmol), methyl (R)-2-((tert-butoxycarbonyl)amino)-3-iodopropanoate (5.77 g, 17.53 mmol), TBAI (5.40 g, 14.61 mmol) and zinc (1.910 g, 29.2 mmol) were added. The reaction was stirred 16 h at room temperature. The resulting solution was quenched with water (100 mL) and extracted with EtOAc (3×250 mL). The organic layers were combined, washed with brine (4×200 mL), dried over anhydrous Na₂SO₄ and filtered. The filtrate was concentrated under reduced pressure and the residue was purified by silica gel column chromatography, eluted with 0-20% ethyl acetate in PE to give tert-butyl(S)-4-(5-(2-((tert-butoxycarbonyl)amino)-3-methoxy-3-oxopropyl)pyridin-2-yl)piperazine-1-carboxylate. MS ESI calculated for C₂₃H₃₇N₄O₆ [M+H]⁺ 465.26, found 465.35.

Step 2: To a mixture of tert-butyl(S)-4-(5-(2-((tert-butoxycarbonyl)amino)-3-methoxy-3-oxopropyl)pyridin-2-yl)piperazine-1-carboxylate (6.5 g, 13.99 mmol) in THF (70 mL) was added LiOH (28 mL, 28.0 mmol, 1N in water) at room temperature. The solution was stirred at 25° C. for 2 h. The pH was adjusted to 7 with 1N HCl and concentrated under reduced pressure to afford(S)-2-((tert-butoxycarbonyl)amino)-3-(6-(4-(tert-butoxycarbonyl)piperazin-1-yl) pyridin-3-yl)propanoic acid (6.3 g, 11.19 mmol, 80% yield) as an off-white solid. MS ESI calculated for C₂₂H₃₅N₄O₆ [M+H]⁺ 451.25, found 451.20.

Step 3: To a stirred solution of(S)-2-((tert-butoxycarbonyl)amino)-3-(6-(4-(tert-butoxycarbonyl)piperazin-1-yl) pyridin-3-yl)propanoic acid (6.3 g, 13.98 mmol) in THF (30 mL) was added HCl (35 mL, 140 mmol, 4N in dioxane) at ambient temperature. The reaction was stirred at ambient temperature for 2 h. The resulting solution was concentrated in vacuum to afford(S)-2-amino-3-(6-(piperazin-1-yl) pyridin-3-yl)propanoic acid (3.5 g, 11.19 mmol, 80% yield)(crude product). MS ESI calculated for C₁₂H₁₉N₄O₂ [M+H]⁺ 251.14, found 251.15.

Step 4: To a stirred solution of(S)-2-amino-3-(6-(piperazin-1-yl) pyridin-3-yl)propanoic acid (3.5 g, 13.98 mmol) in MeOH (60 mL) were added DIEA (12.21 mL, 69.9 mmol) and 2-acetyldimedone (3.82 g, 20.97 mmol) at room temperature. The solution was stirred at 25° C. for 2 h. The resulting solution was concentrates in vacuum to afford crude product. The crude product was purified by RP flash with the following conditions: Flash C18 (330 g); Mobile Phase A: water (0.05% TFA), Mobile Phase B: ACN; (Gradient: 2% B hold 5 min, up to 75% B within 15 min, 49.5% B hold 6 min; up to 60% B within 5 min, 95% B hold 5 min); The product-containing fractions were collected to give(S)-2-((1-(4,4-dimethyl-2,6-dioxocyclohexylidene)ethyl)amino)-3-(6-(piperazin-1-yl) pyridin-3-yl)propanoic acid (5.8 g, 13.29 mmol, 95% yield) as a yellow solid. MS ESI calculated for C₂₂H₃₁N₄O₄ [M+H]⁺ 415.23, found 415.25.

Step 5: To a mixture of(S)-2-((1-(4,4-dimethyl-2,6-dioxocyclohexylidene)ethyl)amino)-3-(6-(piperazin-1-yl) pyridin-3-yl)propanoic acid (4.5 g, 10.86 mmol) in THF (50 mL) and water (50 mL) were added NaHCO₃ (2.74 g, 32.6 mmol) and Fmoc-OSu (3.30 g, 9.77 mmol) at ambient temperature. The reaction was stirred at ambient temperature for 2 h. The resulting solution was acidified with HCl (1 M) and extracted with EA (4×100 mL). The combined organic layers were dried over anhydrous Na₂SO₄ and filtered. The residue was purified by silica gel chromatography, eluting with 0%-60% EA in PE to afford(S)-3-(6-(4-(((9H-fluoren-9-yl)methoxy)carbonyl)piperazin-1-yl) pyridin-3-yl)-2-((1-(4,4-dimethyl-2,6-dioxocyclohexylidene)ethyl)amino)propanoic acid (6.5655 g, 9.83 mmol, 91% yield) as an off-white solid. MS ESI calculated for C₃₇H₄₁N₄O₆ [M+H]⁺ 637.29, found 637.55. ¹H NMR (400 MHz, CDCl₃) δ 13.66 (s, 1H), 10.84 (s, 1H), 8.14 (s, 1H), 7.76-7.75 (m, 2H), 7.57-7.52 (m, 2H), 7.46-7.24 (m, 5H), 6.67-6.65 (m, 1H), 4.64-4.63 (m, 1H), 4.46 (d, J=6.8 Hz, 2H), 4.25 (t, J=6.4 Hz, 1H), 3.64-3.45 (m, 8H), 3.25 (s, 1H), 3.07-3.05 (m, 1H), 2.50 (s, 3H), 2.33 (s, 4H), 0.98 (s, 6H).

### Preparation of Final Compounds

### A. Generalized Procedure for Synthesizing Linear Peptide Precursors

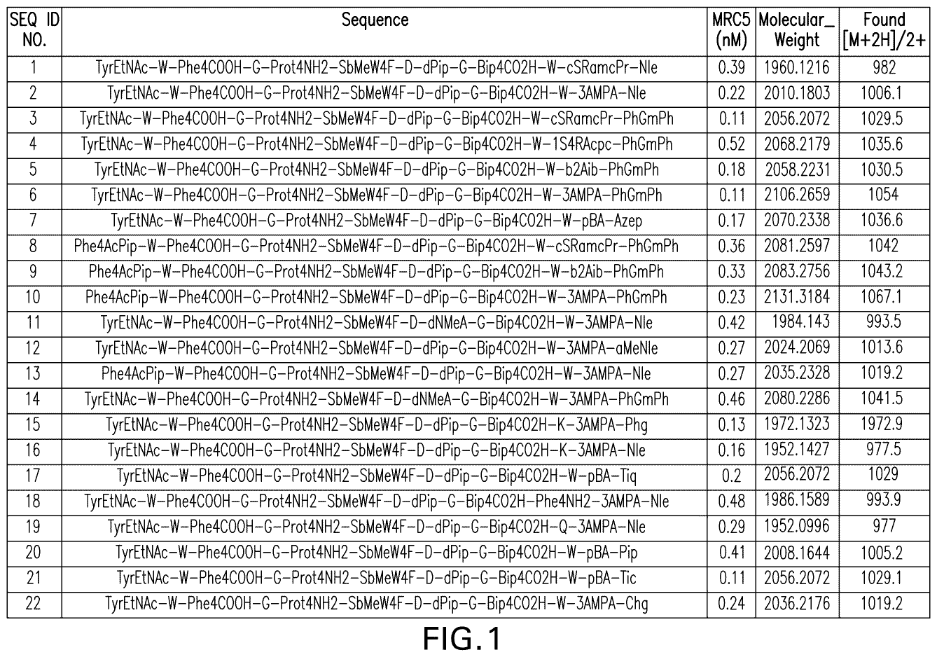

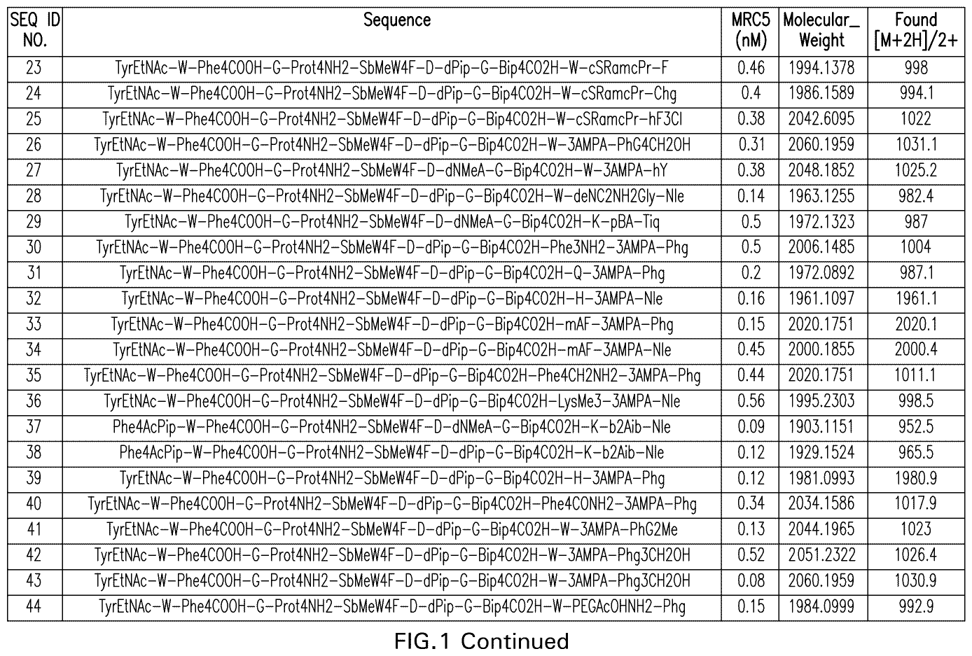

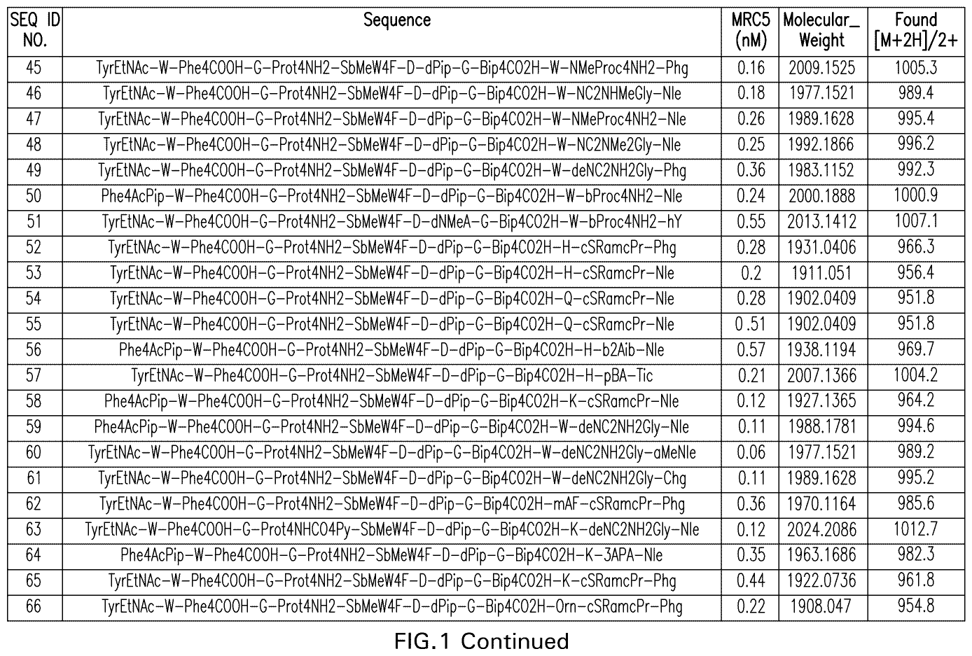

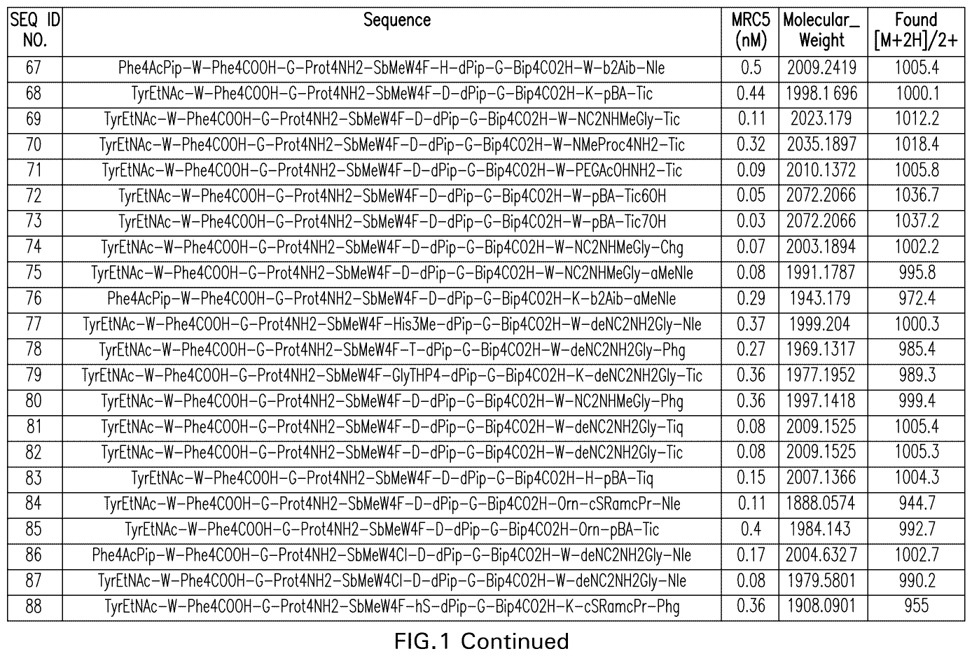

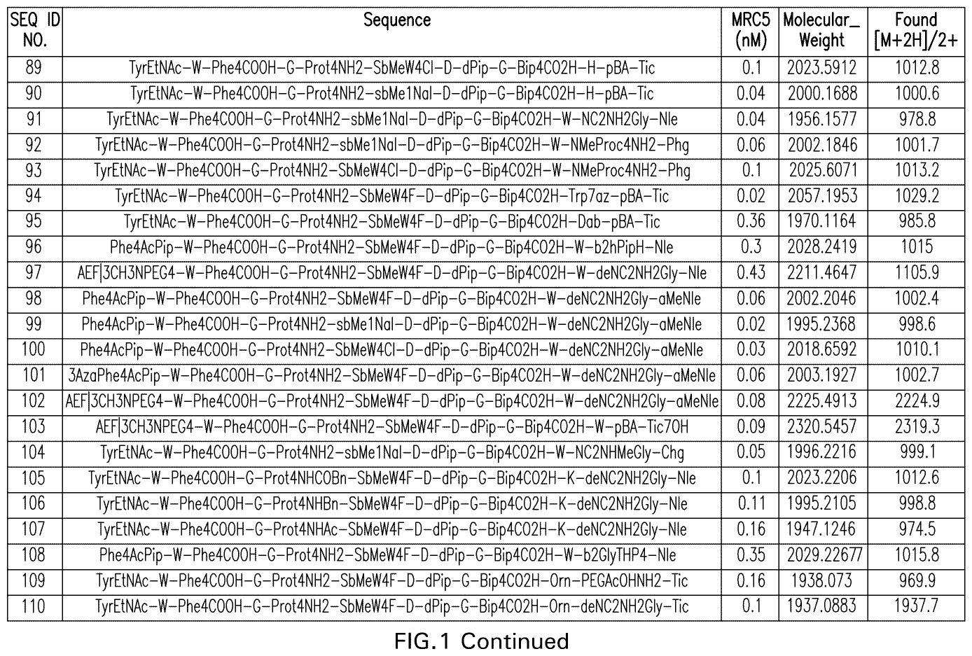

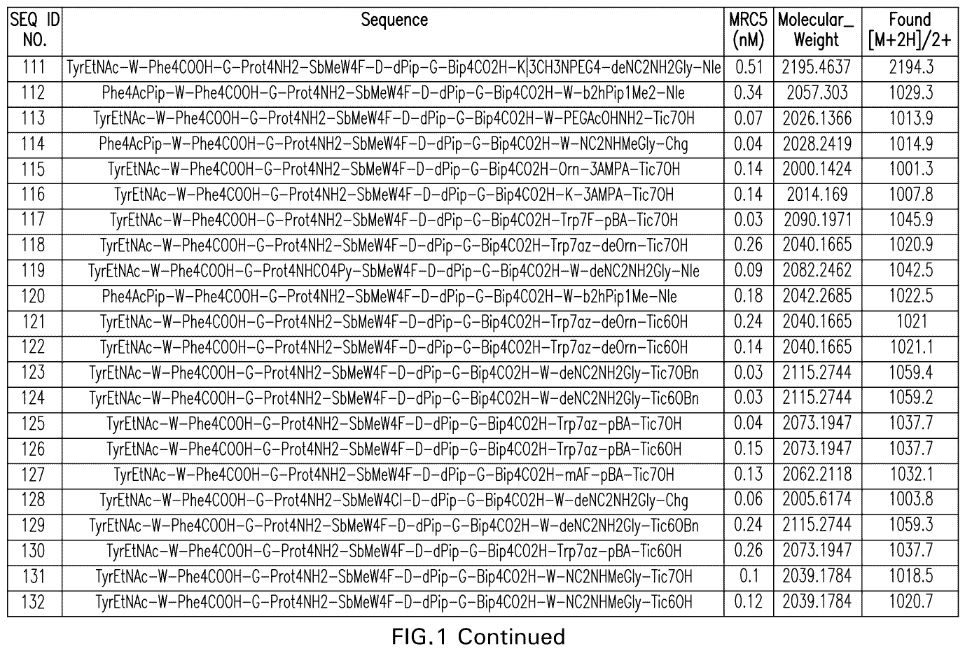

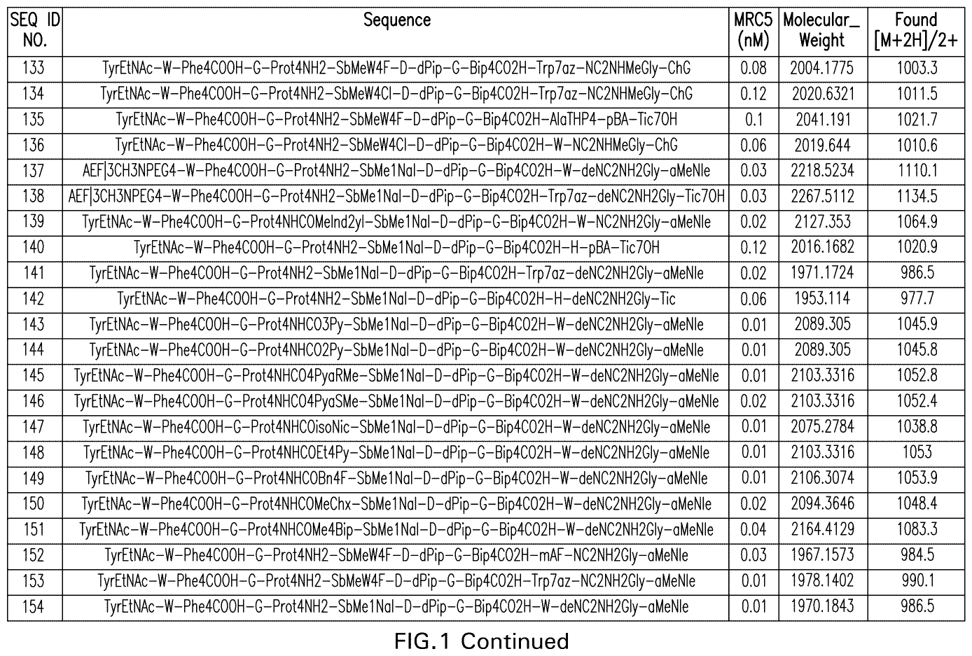

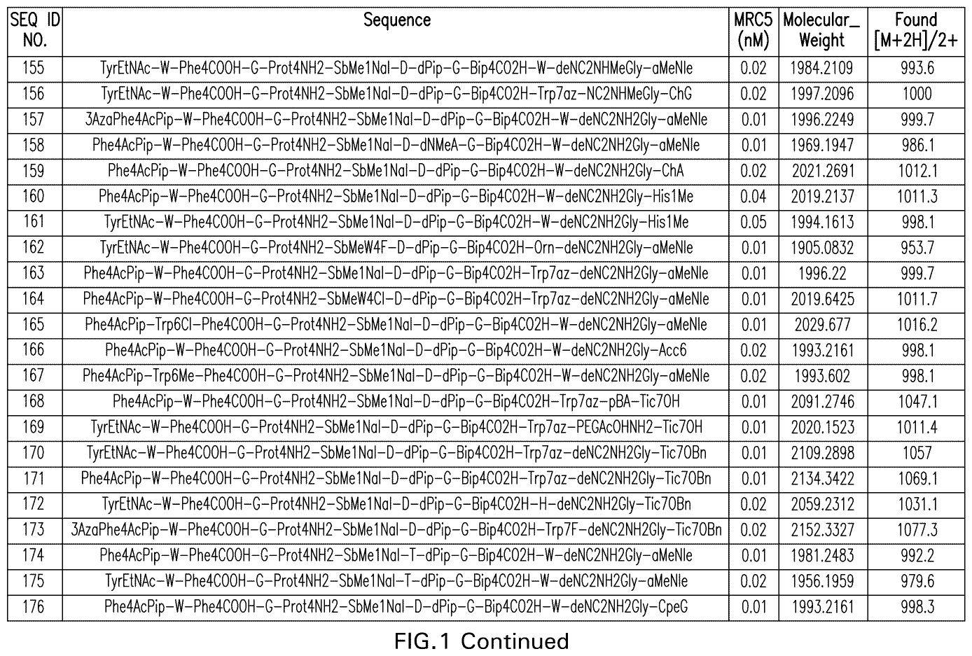

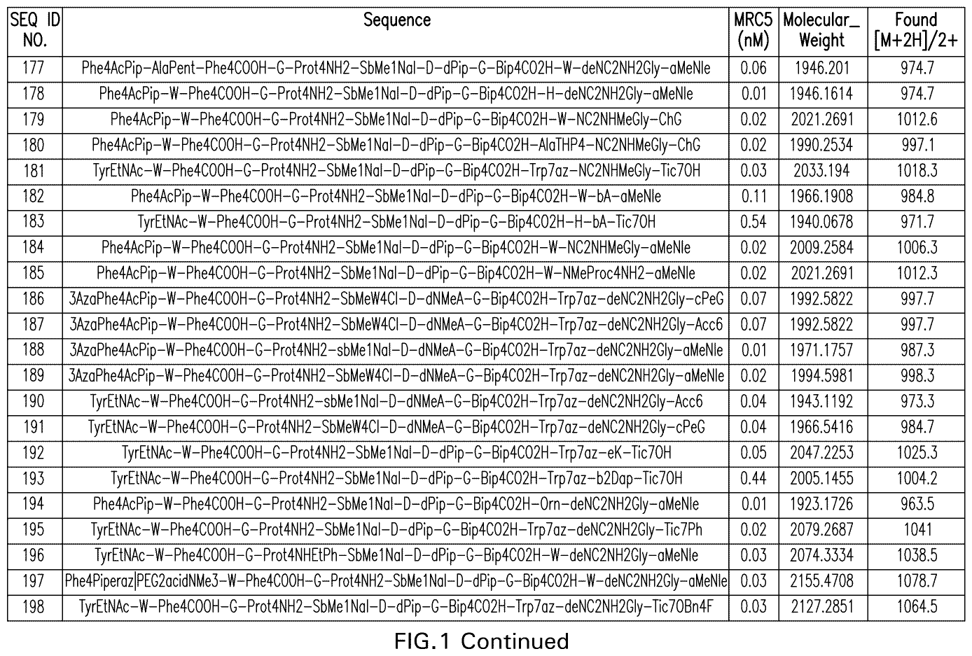

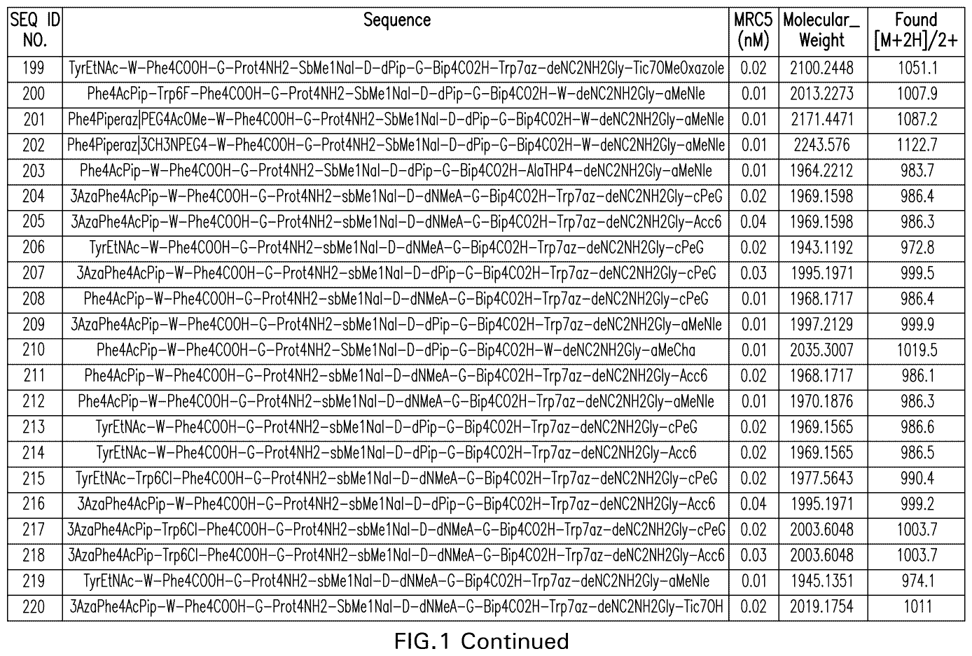

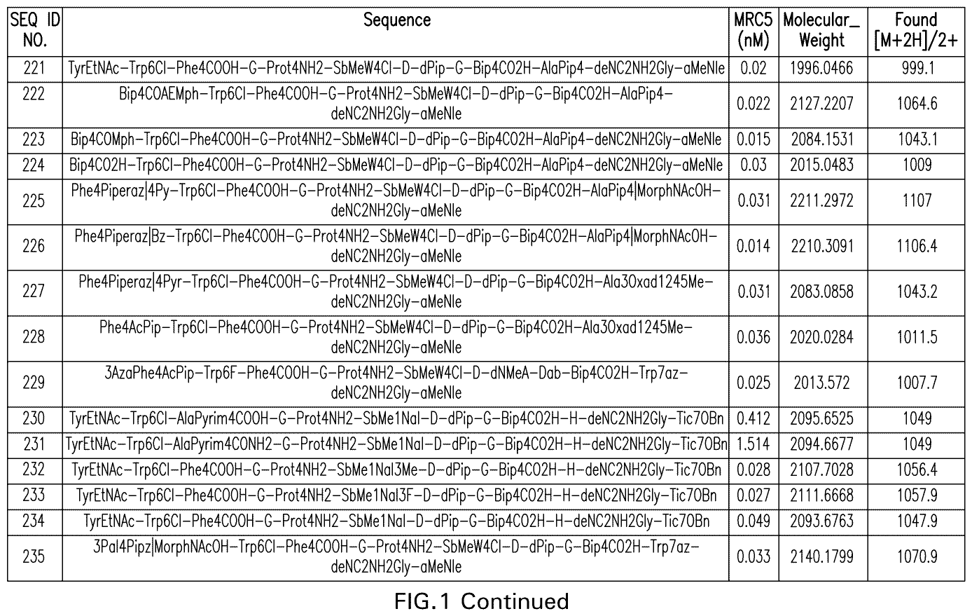

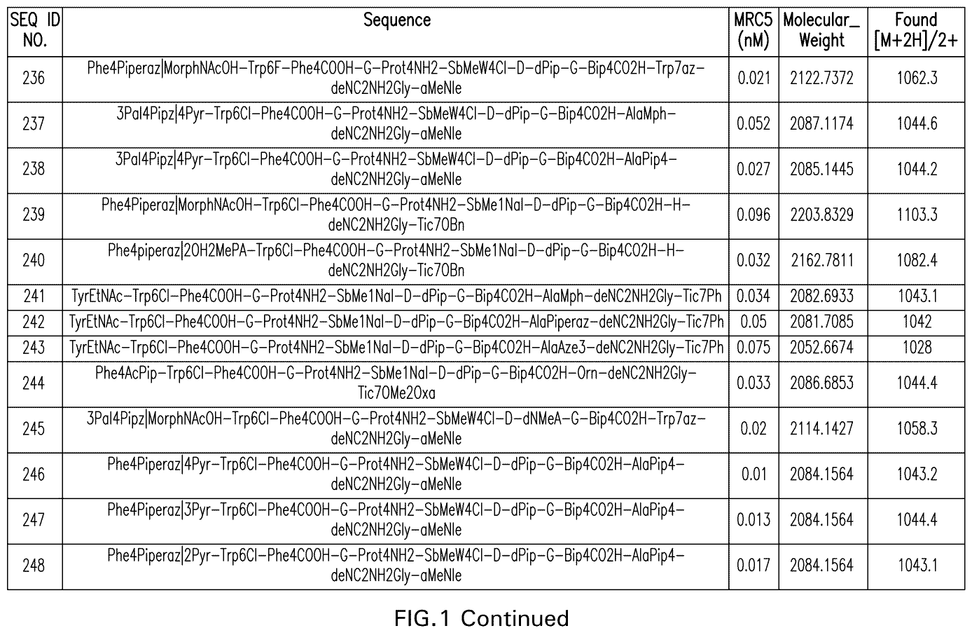

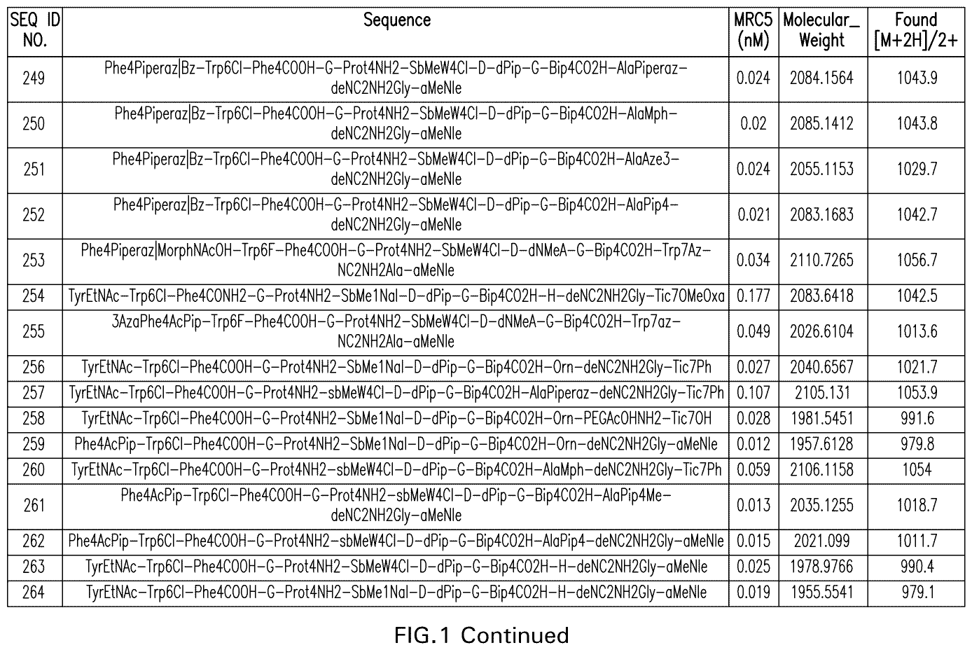

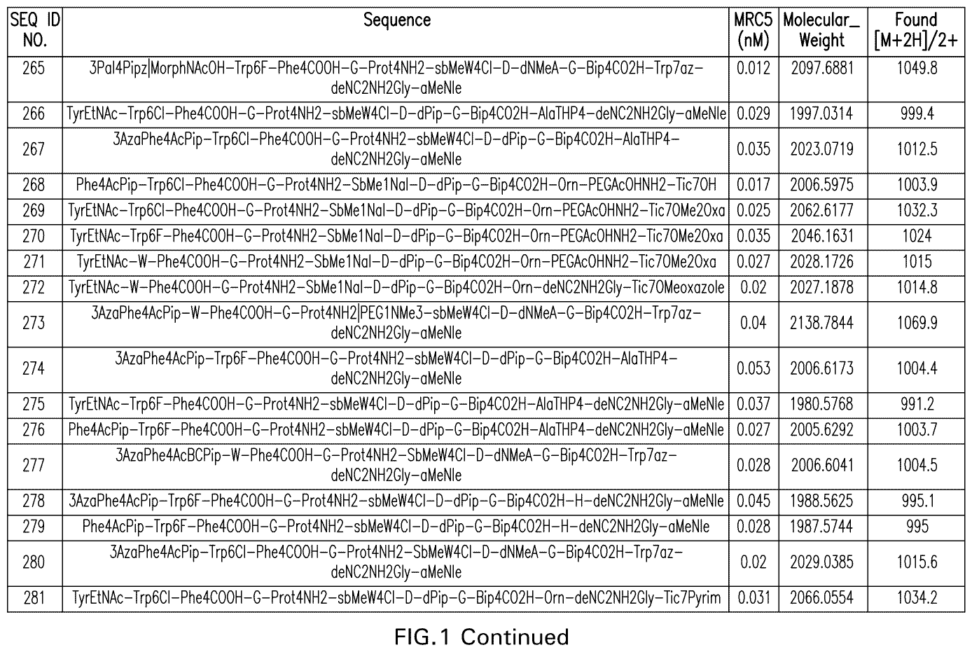

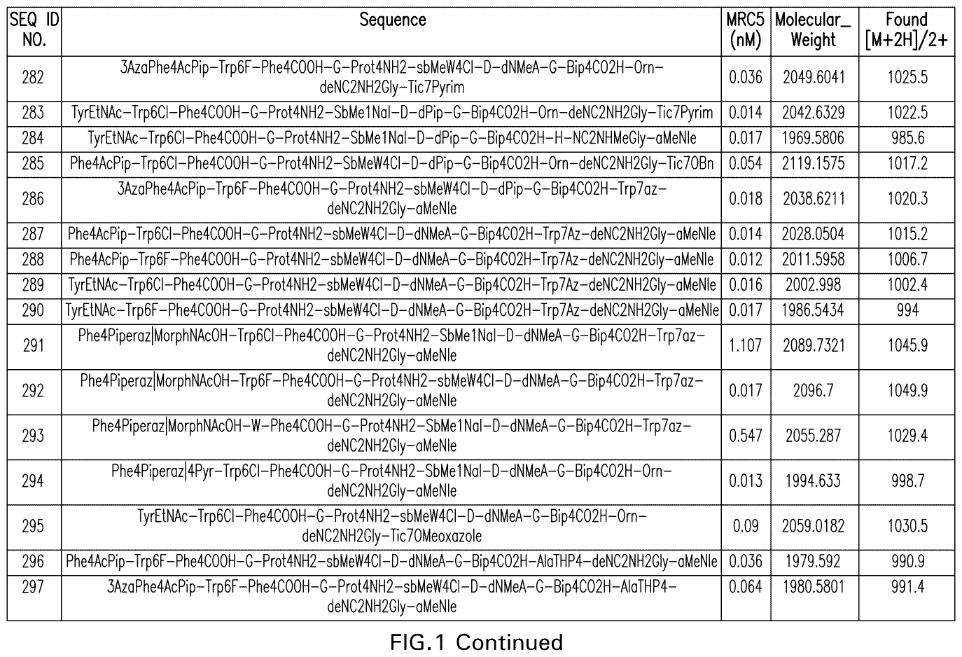

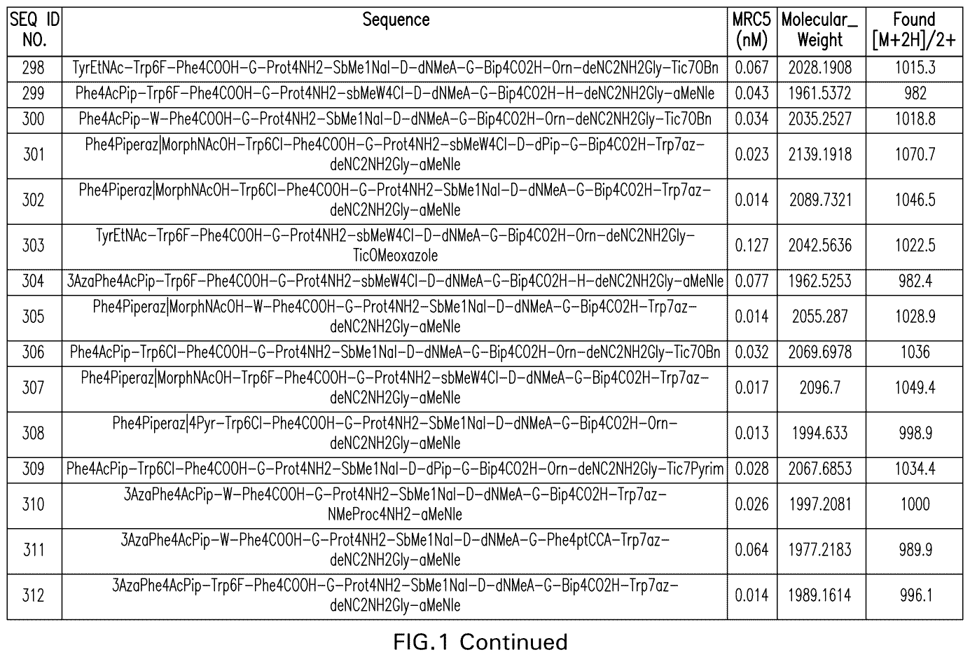

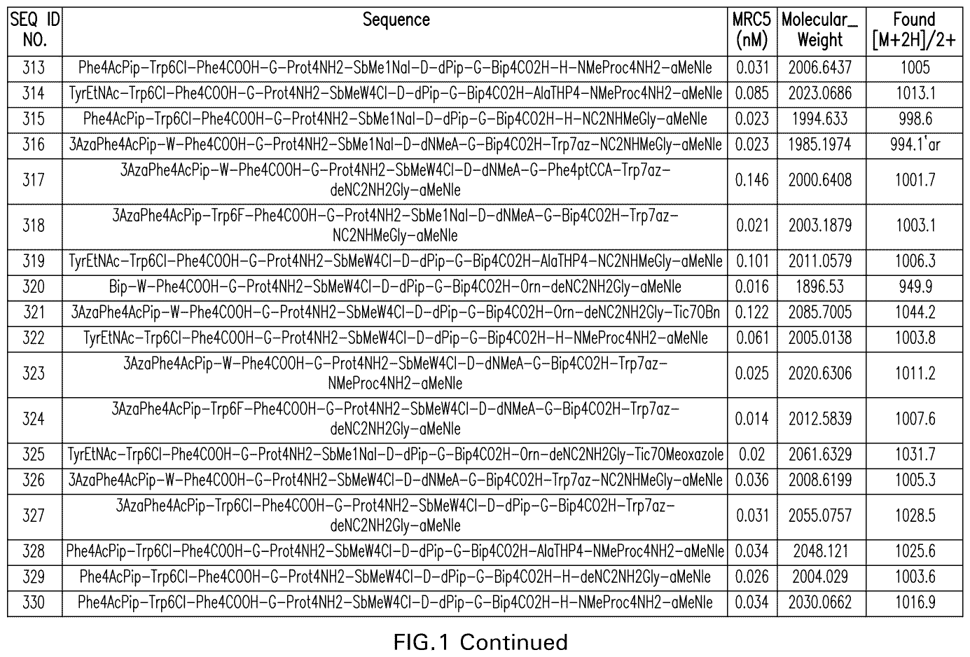

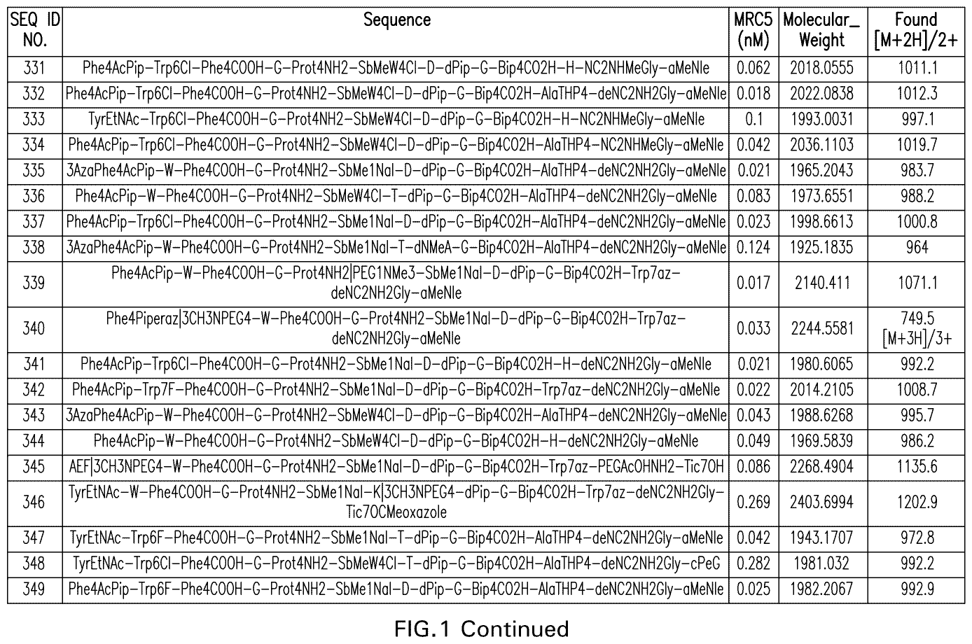

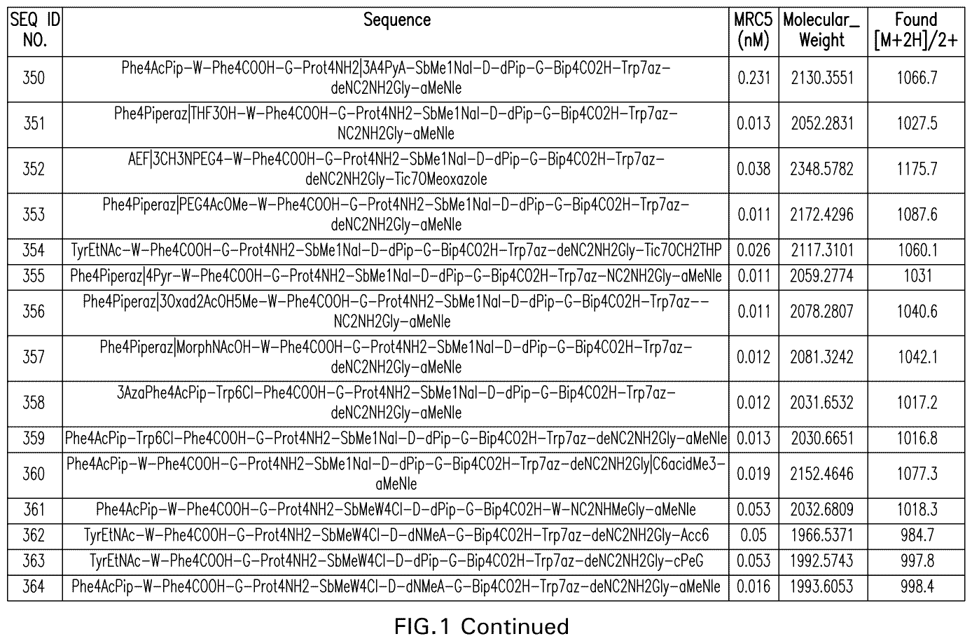

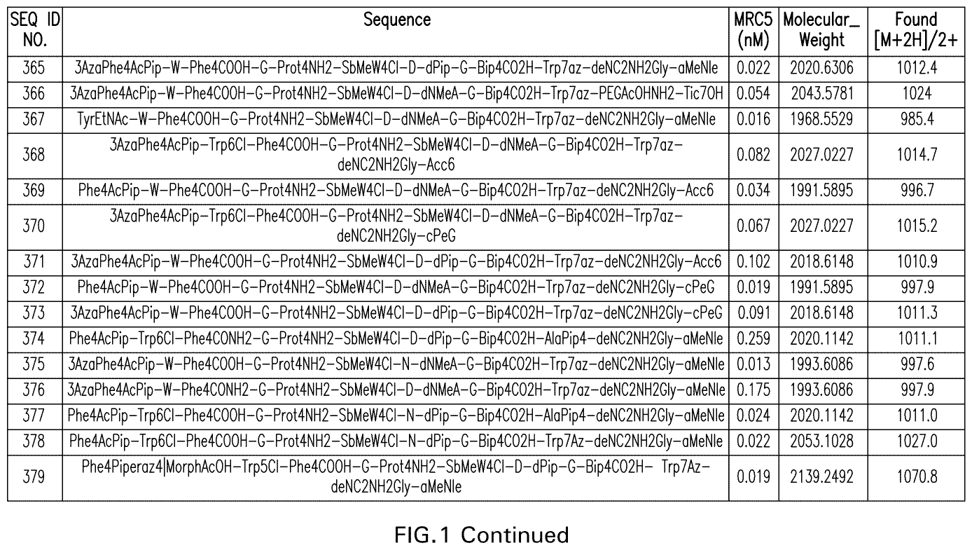

Peptides in FIG. 1 were synthesized using standard solid-phase synthesis using Fmoc/tBu chemistry as exemplified in Chan, W. C.; White, P. D. “Fmoc Solid-Phase Synthesis: a Practical Approach”, Oxford University Press, Oxford, 2000; Steward, J.; Young, J. “Solid Phase Peptide Synthesis”, Pierce Chemical Company, Rockford, 1984; Benoiton, N. L. “Chemistry of Peptide Synthesis”, CRC Press, New York, 2006; and Lloyd-Williams, P.; Albericio, F.; Giralt, E. “Chemical Approaches to the Synthesis of Peptides and Proteins”, CRC Press, New York, 1997.

During peptide chain elongation, the α-amino group of each amino acid was protected with a 9H-fluoren-9-ylmethoxycarbonyl group (Fmoc). To avoid any side reactions during the chain elongation steps, any reactive amino acid side chains also carry acid-labile protecting groups, effectively masking the reactive groups until removal upon treatment with strong acid. After completion of each coupling step, the Fmoc group of the N-terminal amino acid was removed with piperidine or 4-methylpiperidine and the resin was thoroughly washed to prepare for the coupling of the subsequent Fmoc-protected amino acid derivative.

The side chain protecting groups used were: tert-butyl (tBu) for S, hY, Bip4CO2H, Phe4COOH; tert-butoxy-carbonyl (Boc) for Dap, dDab, dK, dOrn, K, Prot4NH₂; and, β-methylpentyl ester (OMpe) for D.

In the description of the synthetic used to prepare cyclic peptides provided below (Protocols A-G), reference is made to specific AA numbers in the peptide. The AA numbering is indicated as shown in the following examples:

(SEQ ID NO: 221)
TyrEtNAc(AA1)-Trp6Cl(AA2)-Phe4COOH(AA3)-G(AA4)-
Prot4NH2(AA5)-SbMeW4Cl(AA6)-D(AA7)-dPip(AA8)-
G(AA9)-Bip4CO2H(AA10)-AlaPip4(AA11)-
deNC2NH2Gly(AA12)-aMeNle(AA13)
(SEQ ID NO: 310)
3AzaPhe4AcPip(AA1)-W(AA2)-Phe4COOH(AA3)-G(AA4)-
Prot4NH2(AA5)-SbMe1Nal(AA6)-D(AA)-dNMeA(AA8)-
G(AA9)-Bip4CO2H(AA10)-Trp7az(AA11)-
NMeProc4NH2(AA12)-aMeNle(AA13);

The solid phase synthesis of cyclic peptides starts from AA9: G(AA9). Glycine was preloaded on the resin.

Fmoc-protected amino acids were typically obtained from vendors such as Sigma-Aldrich, Novabiochem, Chem-Impex, and Combi-Block. Other Fmoc-protected amino acids can be obtained using the synthetic methods protected amino acids described above.

H-Gly-loaded 2-chlorotrityl resin (100-200 mesh, 0.84 mmol/g loading, 1% cross-linked polystyrene, IRIS or Novabiochem) was used for solid phase synthesis.

### B. Synthetic Procedures Used to Prepare Cyclic Peptides

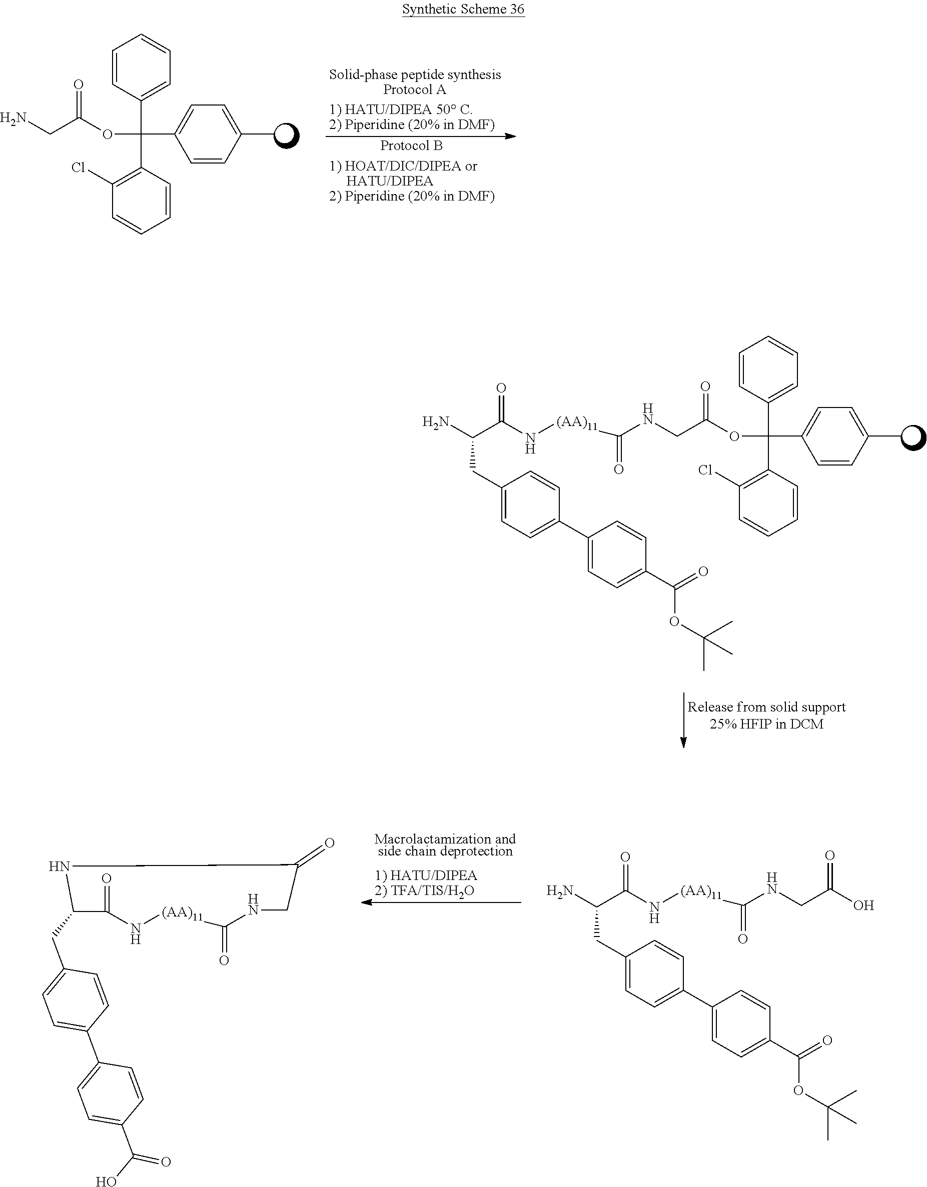

### Solid-Phase Synthesis of Peptides Protocol A

Peptides were synthesized on a Liberty Blue™ synthesizer from CEM Corporation, using standard solid-phase synthesis using Fmoc/tBu or Fmoc/Boc chemistry as summarized above in Scheme 36. HATU with DIPEA were used as coupling agents to create the amide bond between the free amino terminus of the resin-bound protected peptide and the carboxylic acid of the Fmoc-protected amino acid. All the amino acids were dissolved at a 0.2 M concentration in DMF (N,N-dimethylformamide). The amino acids were activated with equimolar amounts of HATU (0.5 M in DMF) and DIPEA solution (2 M in NMP). Reactions were typically performed at the 250 or 100 μmol scale.

Every synthesis cycle included: Fmoc-amino acid deprotection by 20% piperidine in DMF (rt, 2*3 min) and coupling (potentially repeated twice for difficult couplings) with Fmoc-protected amino acid (e.g., PheCOOH, dPip)/HATU/DIPEA (2, 2, and 4 eq respectively; 50° C. microwave assisted heating at 35 W, 240 sec) or Fmoc-protected amino acid (e.g., SbMe1Nal)/HATU/DIPEA (2 or 3, 2 or 3, and 4 or 6 eq respectively; 50° C. microwave assisted heating at 60 W, 480 sec) or with Fmoc-protected amino acid (e.g., G, W, D)/HATU/DIPEA (4, 4, and 8 eq respectively; 50° C. microwave assisted heating at 35 W, 240 sec) or with Fmoc-protected amino acid (e.g., NC2NH₂Gly, aMeNle, TyrEtNAc and Prot4NH(Boc)/(Alloc))/HATU/DIPEA (3, 3, and 6 eq respectively; 50° C. microwave assisted heating at 35 W, 240 sec). Cycles of Fmoc deprotection and Fmoc-protected amino acid coupling were repeated with the desired monomers until the full linear peptide was formed.

Final Fmoc deprotection of resin-bound full linear peptide was frequently performed manually.

### Solid-Phase Synthesis of Peptides Protocol B (Manual Synthesis)

Alternatively, peptides were synthesized totally or partially manually using standard solid-phase synthesis using Fmoc/tBu or Fmoc/Boc chemistry as summarized above in Scheme 36. HOAT with DIC and DIPEA to adjust to pH=7 were used as coupling agents to create the amide bond between the free amino terminus of the resin-bound protected peptide and the carboxylic acid of the Fmoc-protected amino acid. All the amino acids were dissolved at a 0.05-0.2 M concentration in DMF. Reactions were typically performed at the 50-100 μmol scale.

Every synthesis cycle included: Fmoc-amino acid deprotection by 20% piperidine in DMF (rt, 3×3 min or 2×5 min) and coupling (potentially repeated twice for difficult couplings) Fmoc-protected natural amino acid HOAT/DIC (3, 4 or 5 eq, 3, 4 or 5 eq, and 6, 8 or 10 eq respectively; 45 min) or with Fmoc-protected non-natural amino acid HOAT/DIC (1.5 or 2 eq, 1.5 or 2 eq, and 3 or 4 eq respectively; 45 min) with DIPEA 0.5-1 eq typically used (to adjust to pH=7).

Alternatively, peptides were synthesized totally or partially manually using standard solid-phase synthesis using Fmoc/tBu or Fmoc/Boc chemistry as summarized above in Scheme 36. HATU and DIPEA were used as coupling agents to create the amide bond between the free amino terminus of the resin-bound protected peptide and the carboxylic acid of the Fmoc-protected amino acid. All the amino acids were dissolved at a 0.05-0.2 M concentration in DMF.

Reactions were typically performed at the 50-100 μmol scale.

Every synthesis cycle included: Fmoc-amino acid deprotection by 20% piperidine in DMF (rt, 3×3 min or 2×5 min) and coupling (potentially repeated twice for difficult couplings) with Fmoc-protected natural amino acid HATU/DIPEA (3, 4 or 5 eq, 3, 4 or 5 eq, and 6, 8 or 10 eq respectively; 45 min) or with Fmoc-protected non-natural amino acid HATU/DIPEA (1.5 or 2 eq, 1.5 or 2 eq, and 3 or 4 eq respectively; 45 min).

Cycles of Fmoc deprotection and Fmoc-protected amino acid coupling were repeated with the desired monomers until the full linear peptide was formed.

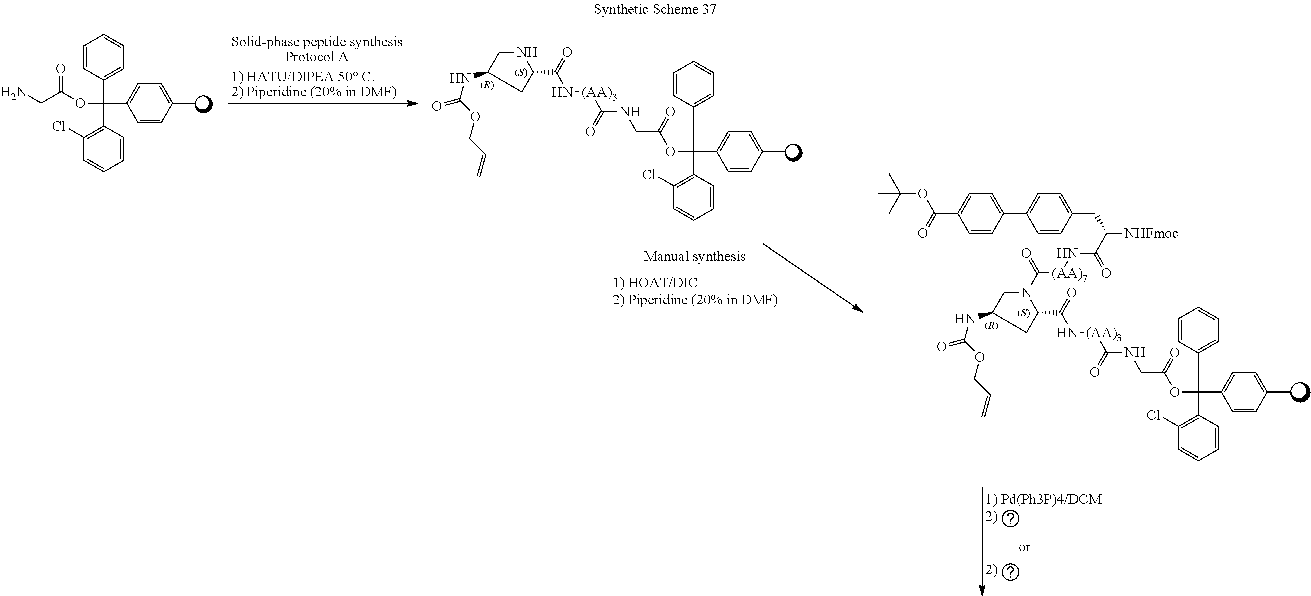

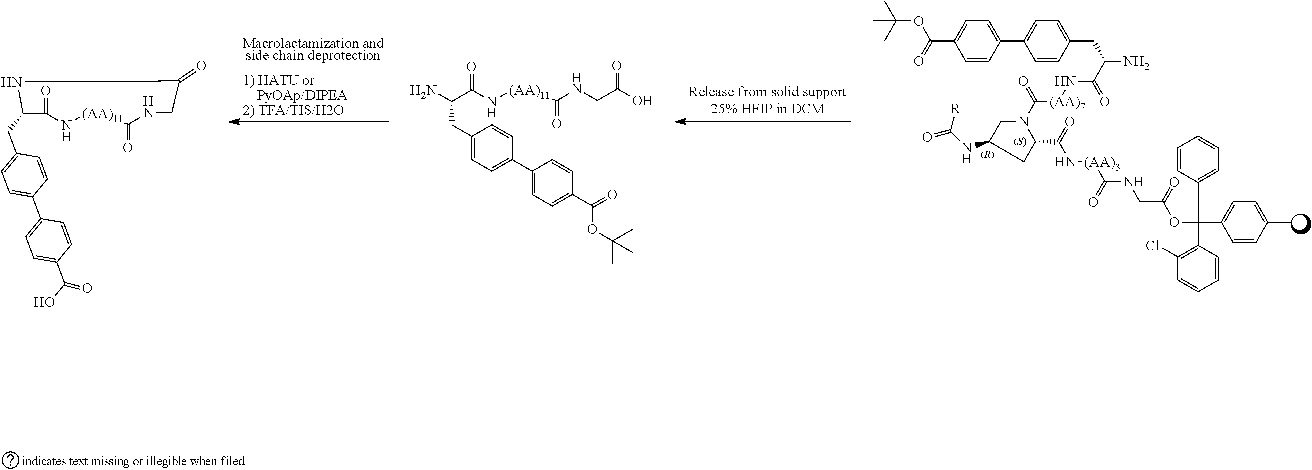

### Solid-Phase Synthesis of Peptides Protocol C

Alternatively, peptides were synthesized according to protocol A up to AA5 and prosecuted manually using standard solid-phase synthesis using Fmoc/tBu, Fmoc/Boc or Alloc/Fmoc chemistry as summarized above in Scheme 37. HOAT with DIC were used as coupling agents to create the amide bond between the free amino terminus of the resin-bound protected peptide and the carboxylic acid of the Fmoc-protected amino acid. For manual couplings all the amino acids were dissolved at a 0.05-0.2 M concentration in DMF or NMP. Before final Fmoc deprotection the Alloc protecting group was removed from AA5, the peptidyl resin was partitioned and coupling with appropriate acid using HOAT with DIC was performed. In the specific case where R=Me (R, in the above scheme), acetic anhydride in NMP was used to acetylate the amino of Prot4NH₂ (AA5). Reactions were typically performed at the 100 μmol scale. Cycles of Fmoc deprotection and Fmoc-protected amino acid coupling were repeated with the desired monomers until the full linear peptide was formed.

### Solid Phase Alloc Deprotection

Pd(Ph₃P)₄ (0.130 g, 0.113 mmol) was dissolved in DCM dry 15 mL and added to the resin-bound protected peptide (˜750 μmol) with phenylsilane (1.948 g, 18.00 mmol) in DCM dry under N₂ bubbling. The reaction was left bubbling under a N₂ atmosphere for 10 min. The resin-bound protected peptide was washed with a solution 0.5% w/v sodium diethylcarbamodithioate (0.017 g, 0.100 mmol), 0.5% V/V DIPEA in DMF (200 mL), then with DMF.

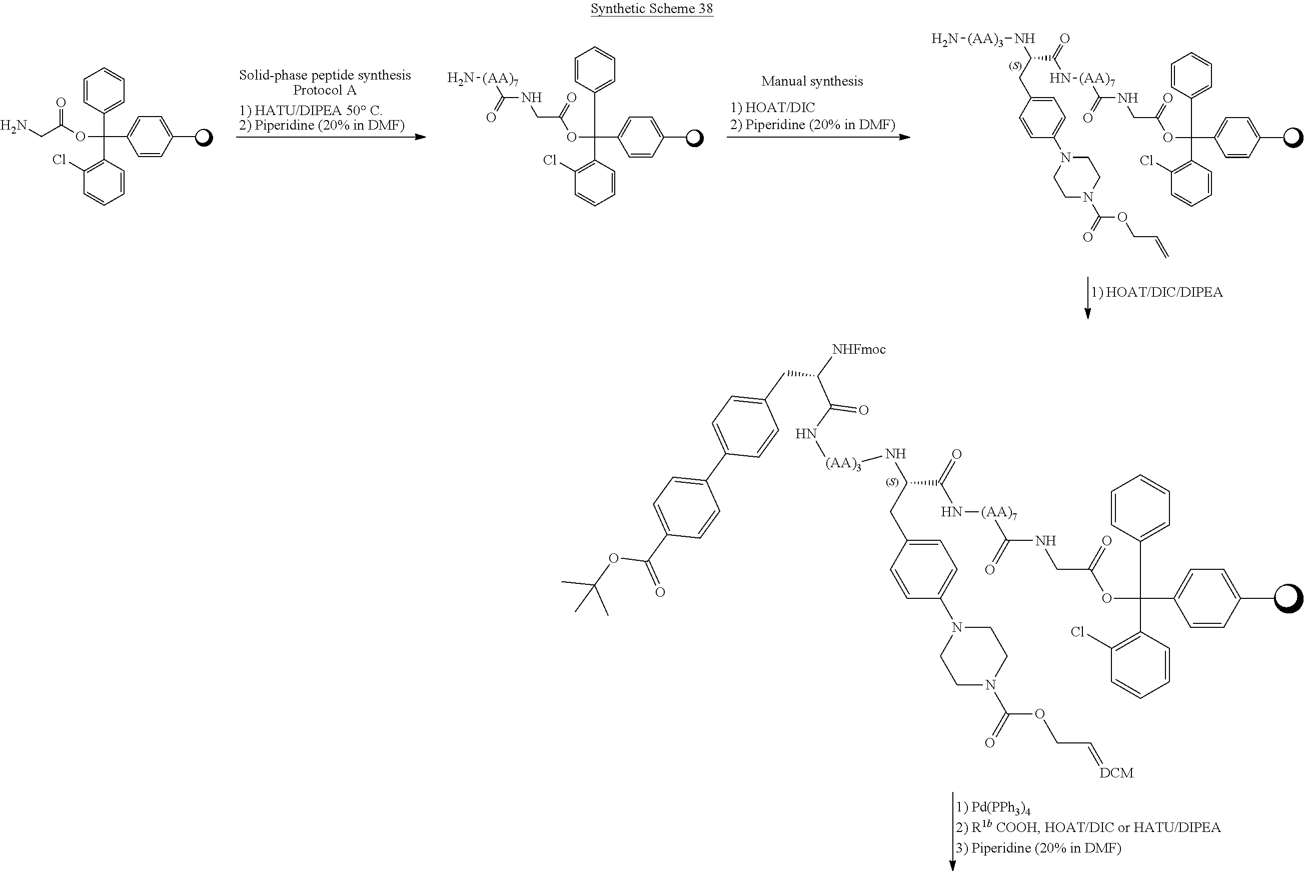

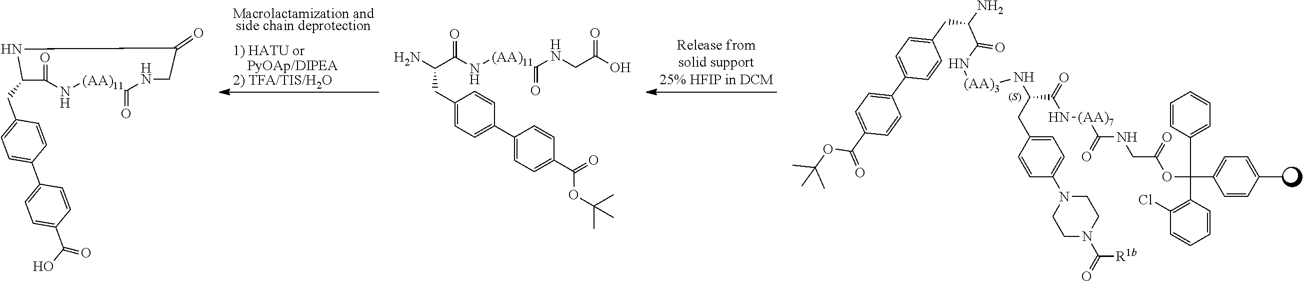

### Solid-Phase Synthesis of Peptides Protocol D

Alternatively, peptides were synthesized according to protocol A up to AA2 (and in some cases up to AA3) and prosecuted manually using standard solid-phase synthesis using Fmoc/tBu, Fmoc/Boc or Fmoc/Alloc chemistry as summarized above in Scheme 38. HOAT with DIC/DIPEA (0.5 eq) or HATU with DIPEA were used as coupling agents to create the amide bond between the free amino terminus of the resin-bound protected peptide and the carboxylic acid of the Fmoc-protected amino acid. H-Gly-loaded 2-chlorotrityl resin. For manual couplings all the amino acids were dissolved at a 0.05-0.2 M concentration in DMF or NMP. Before final Fmoc deprotection, the Alloc protecting group was removed from AA1, the peptidyl resin was partitioned and coupling with appropriate acid using HOAT with DIC was performed. Reactions were typically performed at the 100 μmol scale. Cycles of Fmoc deprotection and Fmoc-protected amino acid coupling were repeated with the desired monomers until the full linear peptide was formed.

### Solid Phase Alloc Deprotection

Pd(Ph₃P)₄ (0.130 g, 0.113 mmol) was dissolved in dry DCM (15 mL) and added to the resin-bound protected peptide (˜750 μmol) with phenylsilane (1.948 g, 18.00 mmol) in DCM dry under N₂ bubbling. The reaction was left bubbling under N₂ atmosphere for 7 min. The resin-bound protected peptide was washed with a solution 0.5% w/v sodium diethylcarbamodithioate (0.017 g, 0.100 mmol), 0.5% V/V DIPEA in DMF (200 mL), then with DMF.

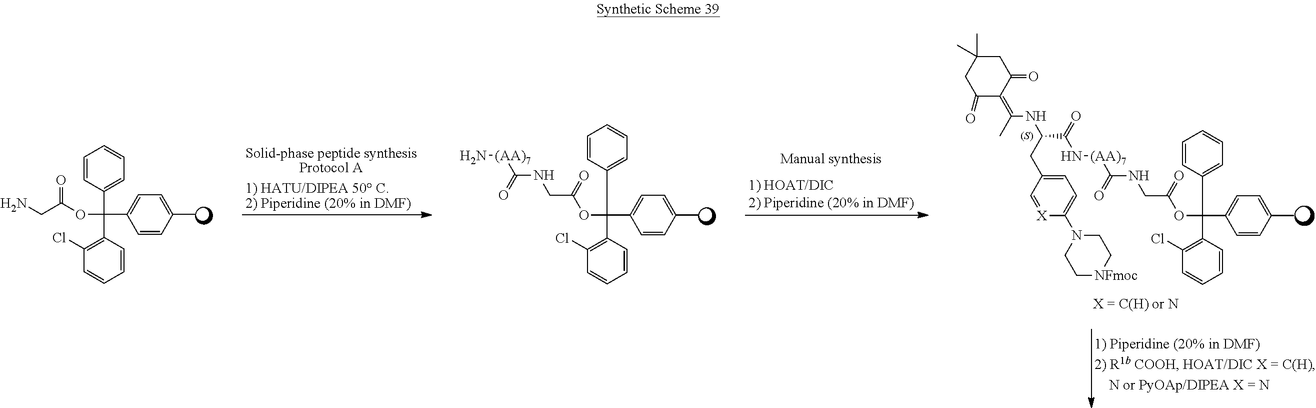

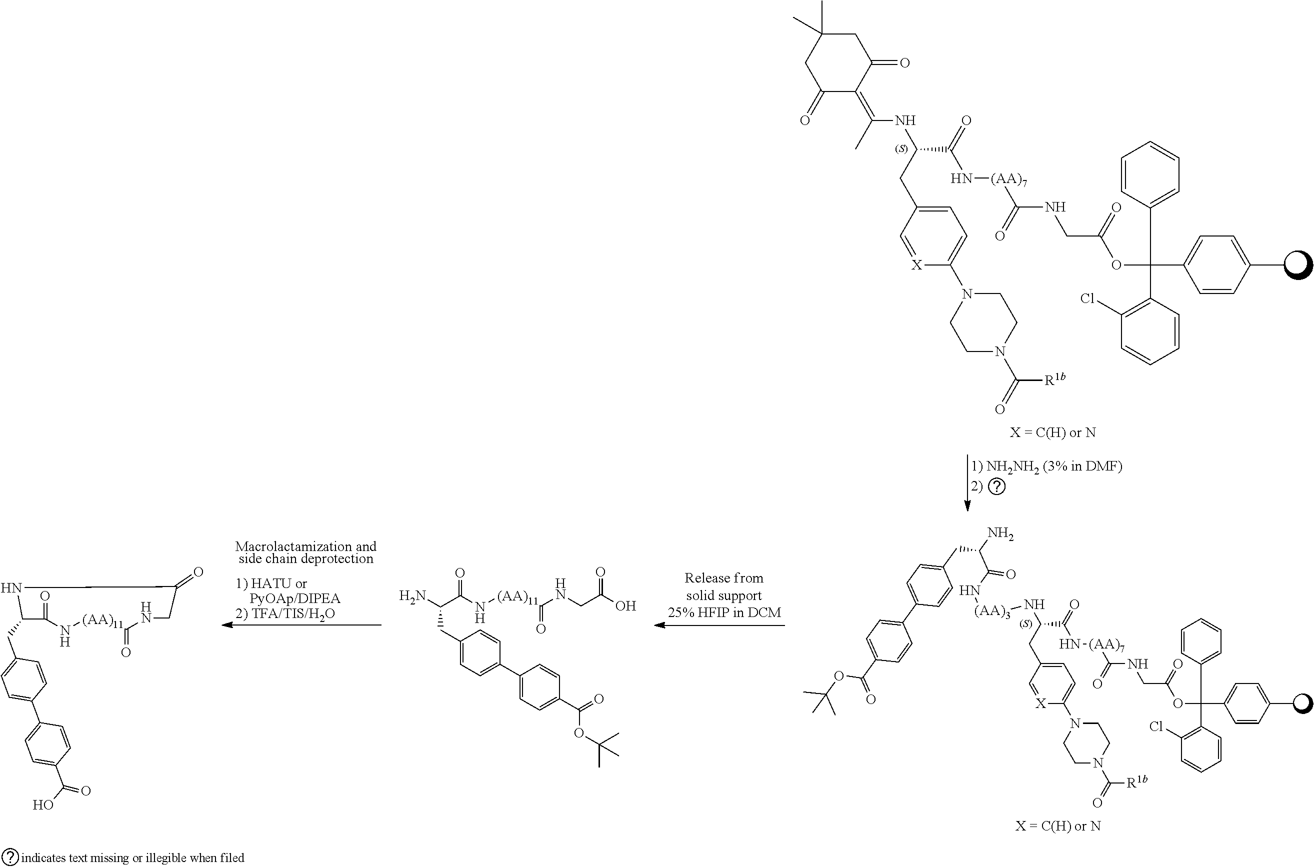

### Solid-Phase Synthesis of Peptides Protocol E

Alternatively, peptides were synthesized according to protocol A up to AA2 (or in some cases only up to AA3) and prosecuted manually using standard solid-phase synthesis using Fmoc/tBu, Fmoc/Boc or Dde/Fmoc chemistry as summarized above in Scheme 39. HOAT with DIC or HATU with DIPEA were used as coupling agents to create the amide bond between the free amino terminus of the resin-bound protected peptide and the carboxylic acid of the Fmoc-protected amino acid. For manual couplings all the amino acids were dissolved at a 0.05-0.2 M concentration in DMF or NMP. When the Fmoc protecting group was removed from AA1, the peptidyl resin was partitioned and coupling with the appropriate acid using HOAT with DIC for X═C(H), N or HOAT with DIC and DIPEA (0.5 eq) or PyOAp and DIPEA for X═N was performed. Then the Dde protecting group ((1-(4,4-dimethyl-2,6-dioxocyclohexylidene)ethyl)) was removed and the synthesis prosecuted to final cyclic peptide as summarized above in Scheme 34. Reactions were typically performed at the 100 μmol scale.

### Solid Phase Dde Deprotection

The resin-bound protected peptide was treated with a solution of hydrazine 3% in DMF v/v (50 mL) by portion wise addition and dropwise draining, then washed with 50 mL of DMF to give resin-bound protected peptide deprotected from Dde.

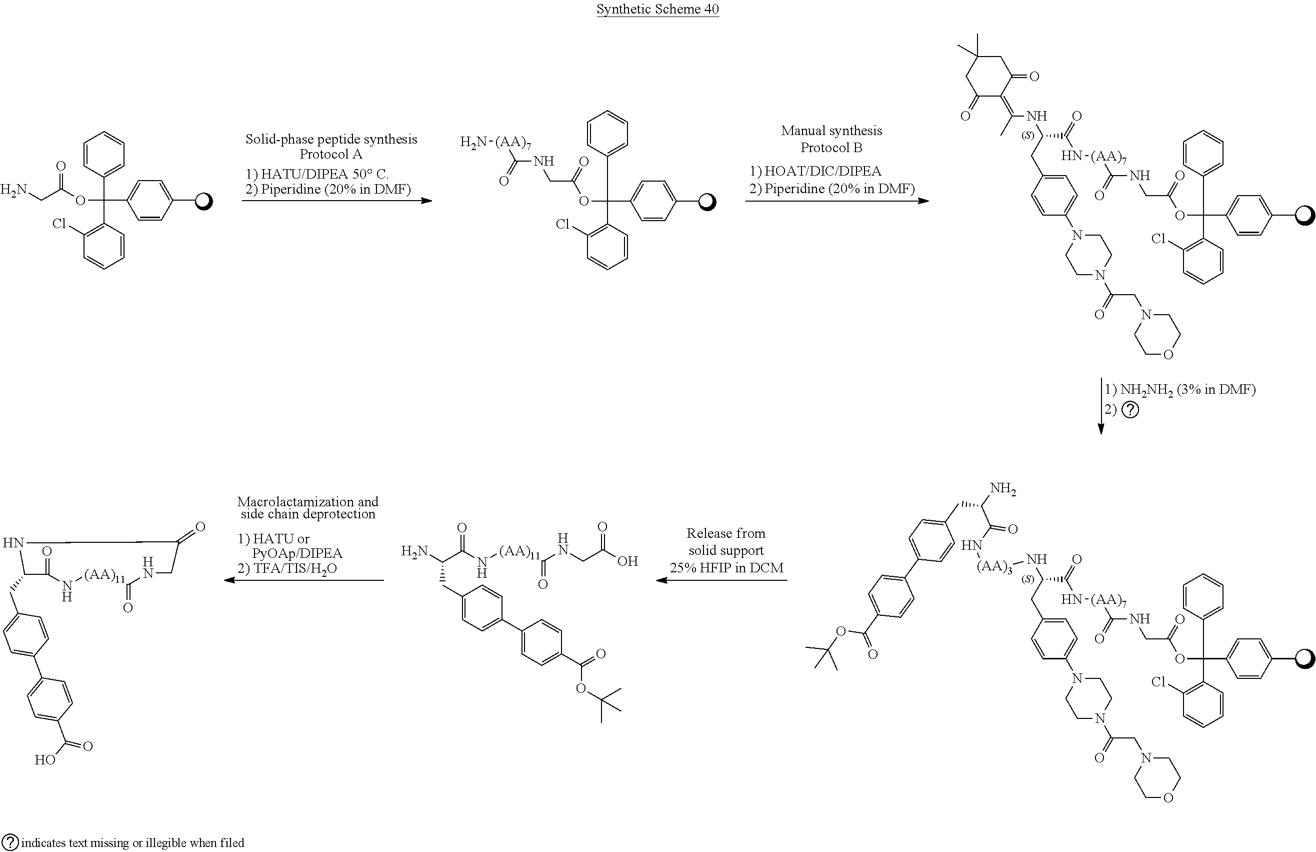

### Solid-Phase Synthesis of Peptides Protocol F

Alternatively, peptides were synthesized according to protocol A up to AA2 (or in some cases only up to AA3) and prosecuted manually using standard solid-phase synthesis using Fmoc/Bu, Fmoc/Boc or Dde/Fmoc chemistry as summarized above in Scheme 40. HOAT with DIC with DIPEA (protocol B) were used as coupling agents to create the amide bond between the free amino terminus of the resin-bound protected peptide and the carboxylic acid of the Fmoc-protected amino acid. For manual couplings all the amino acids were dissolved at a 0.05-0.2 M concentration in DMF or NMP. Then the Dde protecting group ((1-(4,4-dimethyl-2,6-dioxocyclohexylidene)ethyl)) was removed and the synthesis prosecuted to final cyclic peptide as summarized above in Scheme 35. Reactions were typically performed at the 100 μmol scale.

### Solid Phase Dde Deprotection

The resin-bound protected peptide was treated with a solution of hydrazine 3% in DMF v/v (50 mL) by portion wise addition and dropwise draining, then washed with 50 mL of DMF to give resin-bound protected peptide deprotected from Dde.

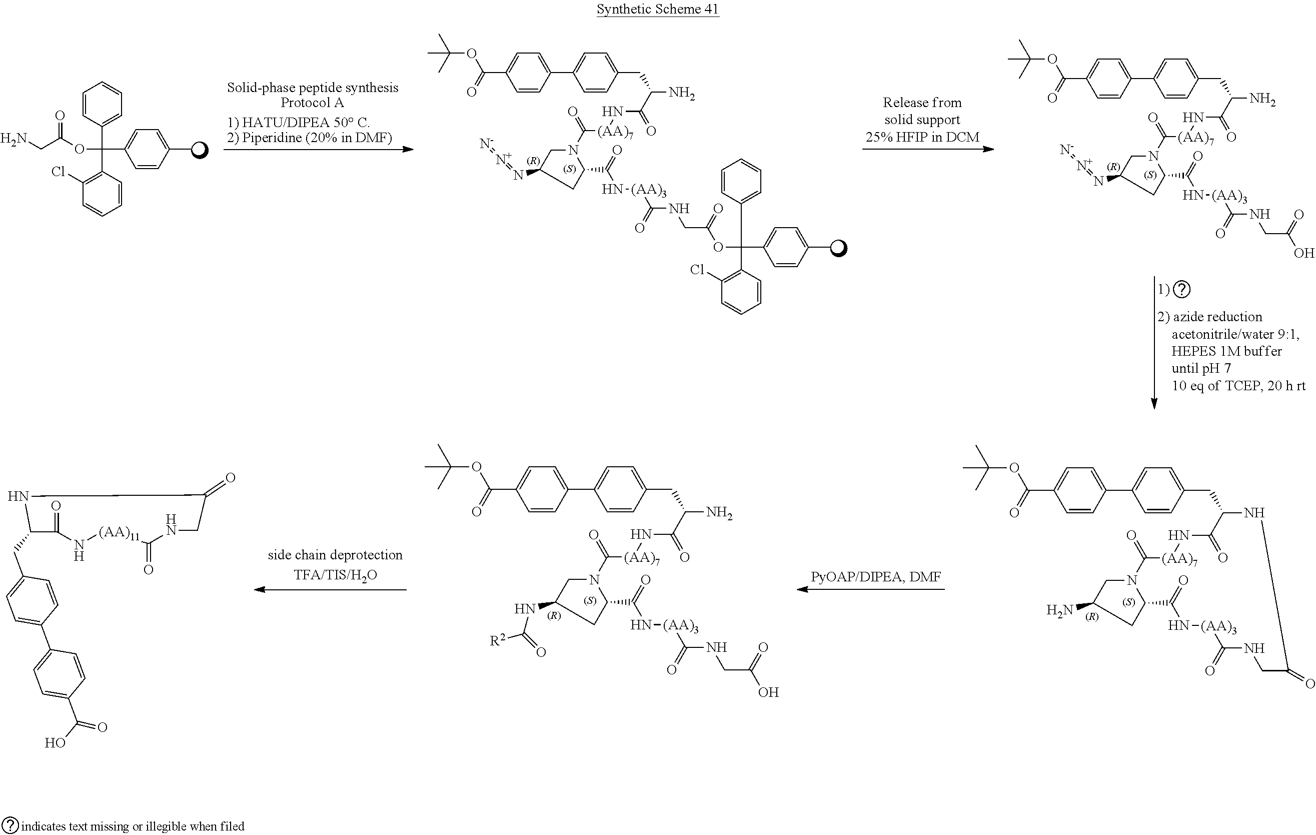

### Solid-Phase Synthesis of Peptides Protocol G

Alternatively, peptides were synthesized according to protocol A and then after selective cleavage of protected peptide from resin and macrolactamization (performed as described below), the azide moiety of Prot4N3 was reduced to Prot4NH2 and manual coupling with PyOAp/DIPEA was performed with the appropriate acid as summarized above in Scheme 41. Then final side chain deprotection was performed. Reactions were typically performed at the 100 μmol scale.

### Solution Phase Azide Reduction

The peptide was dissolved in 50 mL of a mixture acetonitrile/water 9:1 and HEPES (N-2-hydroxyethylpiperazine-N-2-ethane sulfonic acid) 1 M buffer was added until pH 7, 10 eq of TCEP (Tris(2-carboxyethyl)phosphine) were added and the reaction was stirred at room temperature. After 2 h the reaction was almost complete, it was left overnight. The solvent was removed in vacuo with a rotary evaporator.

### Selective Cleavage of Protected Peptide and Macrolactamization

For the cleavage of the protected linear peptide from the solid support, the peptidyl resin (˜16 mg) was treated with 25% hexafluoroisopropanol (HFIP) in DCM for 20 min or 3×3 min at rt, filtered, and the solvent was removed under reduced pressure. The resulting residue was either lyophilized from water/acetonitrile and the resulting powder dissolved in DMF (1 mg/mL) or directly dissolved in DMF (1 mg/mL) and HATU (1 eq) or PyOAp (1 eq) and DIPEA (3 eq) were added. The mixture was stirred for 20 min at rt. Upon completion of the macrolactamization, monitored by UPLC-MS, the solvent was removed under reduced pressure.

### Final Side Chain Deprotection

A solution of TFA/H₂O/TIS (92.5/5/2.5, v/v/v, 25 mL for 100 μmol scale) was added to the crude protected cyclic peptide. The mixture was stirred for 10 min at rt. the volatiles were removed under reduced pressure and the residue dissolved in water/acetonitrile 1:1 and TFA (1 mL) was added, and the mixture stirred for at least 1 h then frozen and lyophilized to give the deprotected cyclic peptide as a solid.

### HPLC Purification

Purification was performed by preparative reversed-phase high performance liquid chromatography (RP-HPLC) on Waters X-bridge C18 50*250 mm, 120 Å, 5 μm, or

Reprosil Gold Longlife DAC C8 250*40 mm 5 μm, 120 or 200 Å; or Reprosil Gold Longlife DAC C8 250*50 mm 5 μm, 200 Å.

Second purification when necessary was performed by preparative reversed-phase high performance liquid chromatography (RP-HPLC): LunaPhenomenex C18 250*30 mm 5 μm, 100 Å or on Waters X-Bridge C8 19*150 mm 5 μm, 300 Å.

Mobile phase: (A) 0.1% TFA in HPLC water and (B) 0.1% TFA in HPLC acetonitrile; UV wavelength λ=215 nm.

UV absorbing fractions containing the target m/z ions were collected and the fractions containing product were confirmed by LC/MS.

Confirmation of identity and purity assessment of final compounds was performed by UPLC-MS, which was measured by a reverse phase Waters Acquity™. UPLC-MS system. ColumnA: Waters BEH C18 Column (130 Å, 1.7 μm, column size 2.1*100 mm). Mobile phase 1: mobile phase 2: (A) 0.1% TFA in HPLC water and (B) 0.1% TFA in HPLC acetonitrile; injection volume: 1 μL; flow rate: 0.4 mL/min; UV wavelength λ=214 nm; or Column B: Waters BEH C4 Column (300 Å, 1.7 μm, column size: 2.1*100 mm, or column size 2:2.1*50 mm, column temperature 45° C.). Mobile phase 1: (A) 0.1% TFA in HPLC water and (B) 0.1% TFA in HPLC acetonitrile; injection volume: 1 μL; flow rate: 0.4 mL/min; UV wavelength λ=214 nm; injection volume: 1 μL; flow rate: 0.4 mL/min; UV wavelength λ=214 nm; Mass range: ES(+): 400-1800 amu. UV detection range: 400-1200 nm. The usage of this methodology is indicated by UPLC-MS in the analytic characterization of the described compounds. Typical examples of gradients are:

Method 1: (% B): 20% in 1 min; 20% to 40% in 4 or 5 min.

Method 2: (% B): 25% in 1 min; 25 to 45% in 4 or 5 min.

Method 3: (% B): 30% in 1 min; 30 to 50% in 4 or 5 min.

Method 4: (% B): 35% in 1 min; 35 to 55% in 4 or 5 min.

Method 5: (% B): 40% in 1 min; 40 to 60% in 4 or 5 min.

Lyophilization of combined fractions containing pure peptide resulted in the final cyclized product as a powder.

### BIOLOGICAL ASSAYS

The inhibition of IL-1β induced IL-6 secretion was evaluated in MRC5 cells. Recombinant human IL-1β (BioLegend 579404) at 2×EC₈₀ concentration was prepared in seeding medium, EMEM (ATCC 30-2003) with 0.025% BSA (Sigma A9576), 1× penicillin/streptomycin (Gibco 15070-063), 1×NEAA (Gibco 11140-050), 1× GlutaMax (Gibco 35050-061), and 1× sodium pyruvate (Gibco 11360-070). 20 μL of IL-1β was added to a 384-well collagen-coated plate (Corning 354664) and pre-incubated at ambient temperature for 1 hour with 200 nL of compound dispensed using an ECHO 555 liquid handler. Human lung fibroblast MRC5 cells (ATCC CCL-171) were added at a density of 3000 cells/20 μL per well. The cells were prepared by passaging three times in growth medium, EMEM (ATCC 30-2003) with 10% fetal bovine serum (Gibco 16140-071), 1× penicillin/streptomycin (Gibco 15070-063), 1×NEAA (Gibco 11140-050), 1× GlutaMax (Gibco 35050-061), and 1× sodium pyruvate (Gibco 11360-070) in collagen-coated T175 flasks (Greiner 661950) and harvested in seeding medium after 5 minutes of 0.25% trypsin-EDTA (Gibco 25200-056) digestion. The 384-well collagen-coated plate, containing a final volume of 40 μL, was incubated at 37° C., 5% CO₂ overnight. 5 μL of the conditioned medium was transferred to a 384-well AlphaLISA plate (PerkinElmer 6005350) for detection of IL-6 using the human AlphaLISA IL-6 kit (PerkinElmer AL223F) as per the manufacturer's protocol. 20 μL of the acceptor bead/biotinylated antibody mix was added to the 384-well AlphaLISA plate and incubated for 1 hour at ambient temperature. The donor bead mix was protected from light and 25 μL was added to the plate and incubated for 30 minutes at ambient temperature. The AlphaLISA plate was read on an En Vision multimode plate reader (Perkin Elmer model 2104) using the AlphaScreen setting (laser exc at 680 nm and emis at 570 nm). Dose response curves and IC₅₀ values were analyzed using a 4-parameter logistic equation in Spotfire software (Tibco, Palo Alto, CA).

The amino acid sequences, biological activities (MRC IC₅₀s), molecular weights and mass spectral data ([M+2H]/2+ or [M+3H]⁺ 3+) of Example Nos. 1-379 (SEQ ID NOS: 1-379) are provided in FIG. 1.

The disclosed subject matter is not to be limited in scope by the specific embodiments and examples described herein. Indeed, various modifications of the disclosure in addition to those described will become apparent to those skilled in the art from the foregoing description and accompanying FIGURES. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A compound of Formula (I) 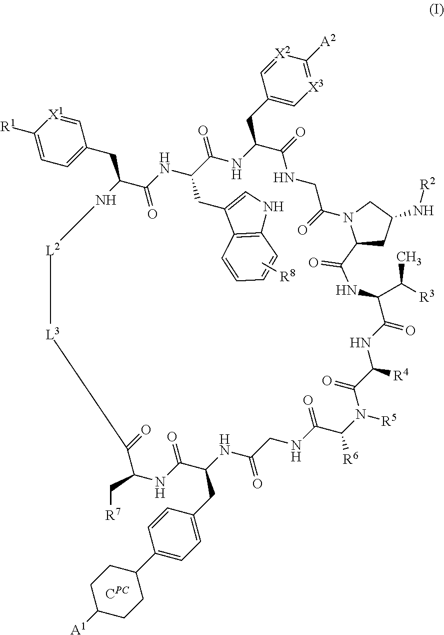
wherein:
L³ is selected from the group consisting of: 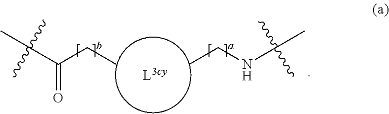
wherein L^{3cy} is C₃-C₆ cycloalkyl, C₅-C₆ cycloalkenyl, or phenyl; 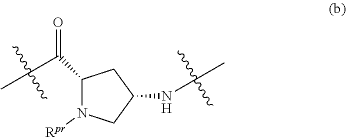
wherein R^{pr} is H or C₁-C₃ alkyl; 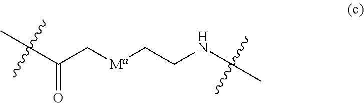
wherein M^{a} is —N(H)—, —N(CH₃)—, —N(CH₃)₂—, —N(H)(C(O)[(CH₂)ᵣN⁺(CH₃)₃]—, or —O—; 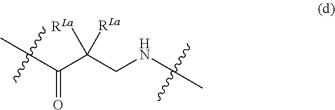
wherein each R^{La} is independently H or C₁-C₃ alkyl, or alternatively the two R^{La}, together with the carbon atom to which they are attached, form a 5- to 6-membered saturated heterocyclic ring containing 1 heteroatom group selected from the group consisting of N(H), N(CH₃), N(CH₃)₂, O and S; and 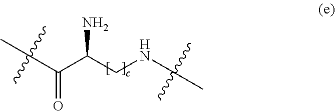
L² is selected from the group consisting of: 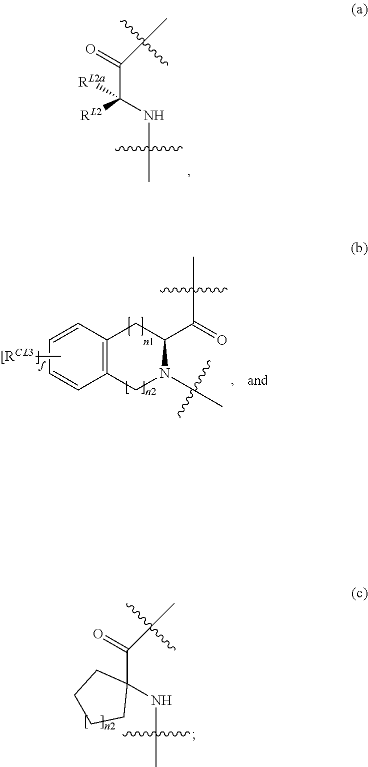
wherein:
R^{L2a} is H or methyl;
R^{L2} is:
(i) C₁-C₅ alkyl; or
(ii) —(CH₂)ₑ—C_{L2}, wherein C_{L2} is:
(i) phenyl,
(ii) C₃-C₆ cycloalkyl,
(iii) a 5- to 6-membered, saturated heterocycle containing one heteroatom selected from the group consisting of N, O, and S; or
(iv) a 5- to 6-membered heteroaryl containing one to two heteroatoms independently selected from the group consisting of N, O, and S
wherein C_{L2} is unsubstituted or substituted by 1 to 3 R^{CL2} substituents independently selected from the group consisting of halo, C₁-C₃ alkyl, phenyl, hydroxy, and C₁-C₃ hydroxyalkyl;
each R^{CL3} is independently selected from the group consisting of H, halo, C₁-C₃ alkyl, hydroxy, phenyl, pyrimidinyl, benzyloxy, and oxazolylmethoxy;
wherein the phenyl, pyrimidinyl, benzyloxy and oxazolylmethoxy of R^{CL3} is unsubstituted or substituted by 1 to 3 substituents independently selected from the group consisting of halo, C₁-C₃ alkyl and C₁-C₃ alkoxy;
each of X¹, X², and X³ are independently C(H) or N;
R¹ is selected from the group consisting of:
(i) R^{1a}—C(O)N(H)—CH₂CH₂—O—, wherein R^{1a} is
(a) C₁-C₃ alkyl; or
(b)(CH₃)₃N⁺CH₂CH₂(OCH₂CH₂)ₘ—;
(ii) a group 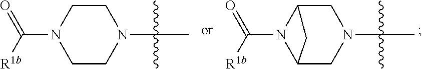
wherein
R^{1b} is:
(a) C₁-C₃ alkyl; or
(b) C_{1b}—(CH₂)ₚ—, wherein C_{1b} is:
(i) a 5- to 6-membered saturated heterocyclyl, wherein the 5- to 6-membered saturated heterocyclyl contains 1 to 2 heteroatoms independently selected from the group consisting of N, O, and S;
(ii) a 5- to 6-membered heteroaryl, wherein the 5- to 6-membered heteroaryl contains 1 to 3 heteroatoms independently selected from the group consisting of N, O, and S; and
(iii) phenyl;
wherein C_{1b} is unsubstituted or substituted by 1 to 2 substituents independently selected from the group consisting of halo, C₁-C₃ alkyl and C₁-C₃ alkoxy;
(c)(CH₃)₃N⁺CH₂CH₂(OCH₂CH₂)_{q}—;
(d)(CH₃)₃N⁺CH₂CH₂OCH₂—; or
(e)CH₃OCH₂CH₂(OCH₂CH₂)_{q}—;
(iii) a group 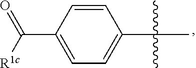
wherein
R^{1c} is hydroxy, amino, morpholinyl, morpholinyl(CH₂CH₂)—N(H)—;
A² is —C(O)OH or —C(O)NH₂;
R² is H, —C(O)—(C₁-C₃ alkyl), —C(O)—(CH₂)g-C₂, —C(O)—C(H)(CH₃)—C₂, or —(CH₂)h-C₂, C₂ is
(i) phenyl or
(ii) a 5- to 6-membered mono- or a 9- to 10-membered bicyclic heteroaryl containing wherein said 5- to 6-membered or 9- to 10-membered bicyclic heteroaryl contains 1 to 2 heteroatoms independently selected from the group consisting of N, O, and S;
wherein C₂ is unsubstituted or substituted by 1 to 3 R^{C2} substituents independently selected from the group consisting of halo, amino, hydroxy, C₁-C₃ alkyl, C₁-C₃ fluoroalkyl, C₁-C₃ alkoxy, and phenyl;
R³ is
(i) naphthyl; or
(ii) a 9- to 10-membered heteroaryl, wherein said heteroaryl contains 1 to 2 heteroatoms independently selected from the group consisting of N, O, and S;
wherein R³ is unsubstituted or substituted by 1 to 2 R^{3a} substituents independently selected from the group consisting of halo, C₁-C₃ alkyl, and C₁-C₃ alkoxy;
R⁴ is —(CH₂)ᵢCO₂H, —(CH₂)ᵢOH, —C(H)(OH)—CH₃, —C^{4a}, —C^{4b}, —CH₂—C^{4a}, or —CH₂—C^{4b};
C^{4a} is tetrahydrofuryl, tetrahydropyranyl, 1,3-dioxolanyl, or 1,3-dioxanyl;
C^{4b} is imidazolyl, wherein said imidazolyl is unsubstituted or substituted by 1 C₁-C₃ alkyl;
R⁵ is C₁-C₃ alkyl;
R⁶ is H or C₁-C₃ alkyl;
or alternatively R⁵ and R⁶ together with the atoms to which they are attached form a pyrrolidinyl, piperidinyl or azepinyl ring;
Ring C^{PC} is phenyl or cyclohexyl;
A¹ is —CO₂H, or —N(H)SO₂CH₃;
R⁷ is —(CH₂)ⱼNH₂; —(CH₂)ⱼN⁺(CH₃)₃;
—(CH₂)ⱼN(H)C(O)CH₂CH₂O(CH₂CH₂—O)ₖ—CH₂CH₂N⁺(CH₃)₃; (CH₂)ⱼC(O)NH₂; —(CH₂)ⱼOH; (CH₂)ⱼOCH₃, or —C^{7a}
C^{7a} is:
(i) a 5- to 6-membered monocyclic aryl or heteroaryl, wherein said 5- to 6-membered heteroaryl contains 1 to 2 heteroatoms independently selected from the group consisting of N, O, and S;
(ii) a 9- to 10-membered bicyclic aryl or heteroaryl, wherein said 9- to 10-membered bicyclic heteroaryl contains 1 to 4 heteroatoms independently selected from the group consisting of N, O, and S; or
(iii) a 4- to 6-membered saturated monocyclic heterocycloalkyl, wherein said 4- to 6-membered saturated monocyclic heterocycloalkyl contains 1 to 2 heteroatoms independently selected from the group consisting of N, O, and S;
wherein C^{7a} is unsubstituted or substituted by 1 to 3 R^{C7a} substituents independently selected from the group consisting of halo, amino, amino (C₁-C₃)alkyl, carbamoyl, hydroxy, carboxy, C₁-C₃ alkyl, C₁-C₃ fluoroalkyl, C₁-C₃ alkoxy, and —C(O)CH₂—C^{7b};
C^{7b} is a 4- to 6-membered saturated monocyclic heterocycloalkyl, wherein said 4- to 6-membered saturated monocyclic heterocycloalkyl contains 1 to 2 heteroatoms independently selected from the group consisting of N and O;
wherein C^{7b} is unsubstituted or substituted by 1 to 2 R^{C7b} substituents independently selected from the group consisting of C₁-C₃ alkyl;
R⁸ is H, halo, or C₁-C₃ alkyl;
subscript a is 0 or 1;
subscript b is 0 or 1;
subscript c is 1, 2, 3, 4, or 5;
subscript d is 1 or 2;
subscript e is 0, 1, or 2;
subscript f is 0, 1, 2, or 3;
subscript g is 0, 1, or 2;
subscript h is 1 or 2;
subscript i is 1, 2, or 3;
subscript j is 1, 2, or 3; and
subscript k is 1, 2, 3, or 4;
subscript m is 0, 1, 2, 3, or 4;
subscript n1 n is 0, 1 or 2;
subscript n2 is 1 or 2;
subscript p is 0, 1, 2, or 3;
subscript q is 0, 1, 2, 3, or 4; and
subscript r is 1, 2, 3, 4, or 5; or
a pharmaceutically acceptable salt thereof.

2. The compound of claim 1 or a pharmaceutically acceptable salt thereof, wherein L³ is

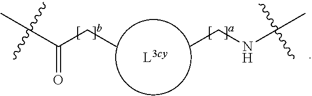

3. The compound of claim 1 or a pharmaceutically acceptable salt thereof, wherein L³ is

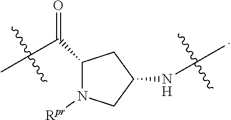

4. The compound of claim 1 or a pharmaceutically acceptable salt thereof, wherein L³ is

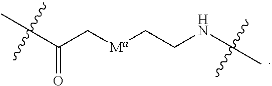

5. The compound of claim 1 or a pharmaceutically acceptable salt thereof, wherein L³ is

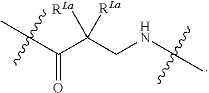

6. The compound of claim 1 or a pharmaceutically acceptable salt thereof, wherein L² is

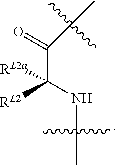

7. The compound of claim 1 or a pharmaceutically acceptable salt thereof, wherein L² is 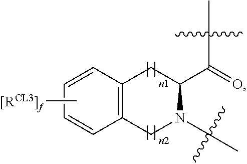
wherein subscript n1 is 0, 1, or 2 and subscript n2 is 1 or 2.

8. The compound of claim 1 or a pharmaceutically acceptable salt thereof wherein R¹ is R^{1a}—C(O)N(H)—CH₂CH₂—O—.

9. The compound of claim 1 or a pharmaceutically acceptable salt thereof wherein R¹ is the group 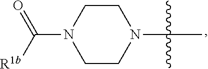
and R^{1b} is methyl or morpholin-4-yl-CH₂—.

10. The compound of claim 1 or a pharmaceutically acceptable salt thereof wherein R³ is substituted or unsubstituted naphthyl or indolyl.

11. The compound of claim 1 or a pharmaceutically acceptable salt thereof wherein R⁴ is —(CH₂)ᵢCO₂H.

12. The compound of claim 1 or a pharmaceutically acceptable salt thereof, wherein R⁵ and R⁶ together with the atoms to which they are attached form a piperidinyl ring.

13. The compound of claim 1 or a pharmaceutically acceptable salt thereof, wherein R⁵ and R⁶ are methyl.

14. The compound of claim 1 or a pharmaceutically acceptable salt thereof, wherein ring C^{PC} is phenyl.

15. The compound of claim 1 or a pharmaceutically acceptable salt thereof, wherein A¹ is —CO₂H.

16. The compound of claim 1 or a pharmaceutically acceptable salt thereof, wherein R⁷ is —C^{7a}.

17. The compound of claim 1 or a pharmaceutically acceptable salt thereof, wherein:
L³ is selected from the group consisting of: 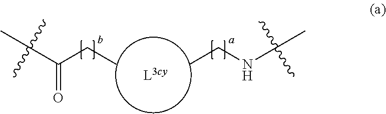
and L^{3cy} is cyclopropyl, cyclopentenyl, or phenyl; 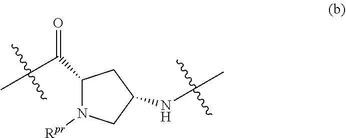
and R^{pr} is H; 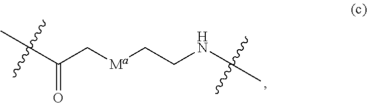
and M^{a} is —N(H)—, N(CH₃)—, or —O—; 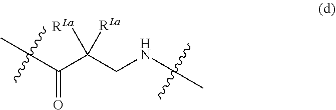
and
each R^{La} is methyl; or
the two R^{La}, together with the carbon atom to which they are attached, form a piperidine ring; and 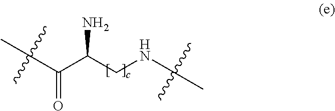
and subscript c is 1, 2, 3, or 4;
L² is selected from the group consisting of: 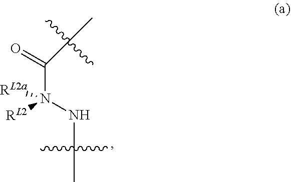
and
R^{L2a} is methyl and R^{L2} is C₁-C₄ alkyl; and 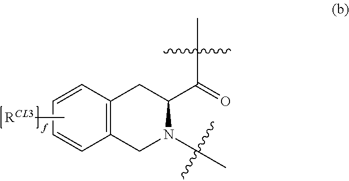
R^{CL3} is independently selected from benzyloxy and 1,3-oxazol-2-yl methoxy; and
subscript f is 0 or 1;
R¹ is:
(i) R^{1a}—C(O)N(H)—CH₂CH₂—O— and R^{1a} is methyl; or
(ii) the group 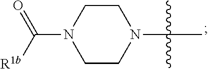
and
R^{1b} is methyl or morpholin-4-yl-CH₂—;
R² is —H;
R³ is substituted or unsubstituted naphthyl or indolyl;
R⁴ is —(CH₂)ᵢCO₂H and subscript i is 1;
R⁵ and R⁶ are methyl, or alternatively, R⁵ and R⁶ together with the atoms to which they are attached form a piperidinyl ring;
Ring C^{PC} is phenyl;
A¹ is —CO₂H;
R⁷ is indolyl or 7-azaindolyl; and
R⁸ is H, F, or Cl.

18. The compound of claim 1 or a pharmaceutically acceptable salt thereof, wherein the compound of Formula (I) has Formula (IA)

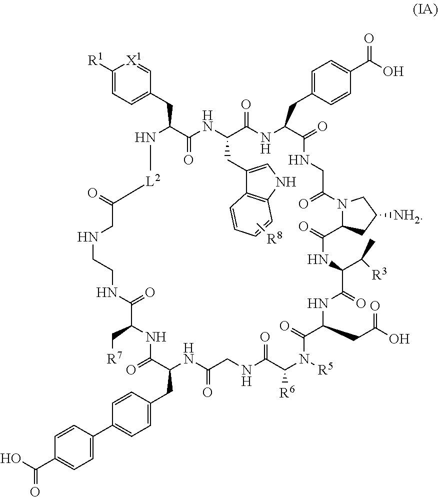

19. The compound of claim 18 or the pharmaceutically acceptable salt thereof, wherein:
L² is selected from the group consisting of: 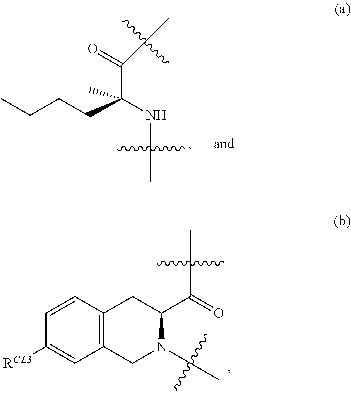
wherein
R^{CL3} is independently selected from benzyloxy and 1,3-oxazol-2-yl methoxy;
R¹ is:
(i) R^{1a} C(O)N(H)—CH₂CH₂—O— and R^{1a} is methyl; or
(ii) the group 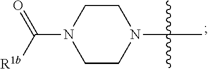
and
R^{1b} is methyl or morpholin-4-yl-CH₂—;
R³ is naphthyl or indolyl, wherein the naphthyl or indolyl is unsubstituted or substituted by 1 halo;
R⁵ and R⁶ are methyl, or alternatively, R⁵ and R⁶ together with the atoms to which they are attached form a piperidinyl ring;
R⁷ is indolyl or 7-azaindolyl; and
R⁸ is H, F, or Cl.

20. The compound of claim 1 selected from the group consisting of SEQ ID NOS: 1-379, or a pharmaceutically acceptable salt thereof.

21. The compound of claim 1 selected from the group consisting of (SEQ ID NOS: 246, 292, 324, 189, 301, 302, 305, 265, 280, 199, 286, 188 and 379, respectively): 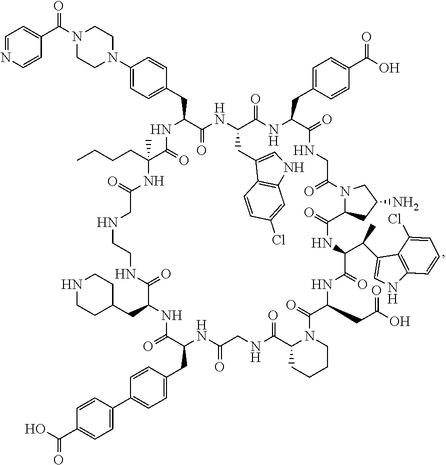 [SMILES: CCCC[C@]1(C)NC(=O)CNCCNC(=O)[C@H](CC2CCNCC2)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)c4ccncc4)CC3)cc2)NC1=O] 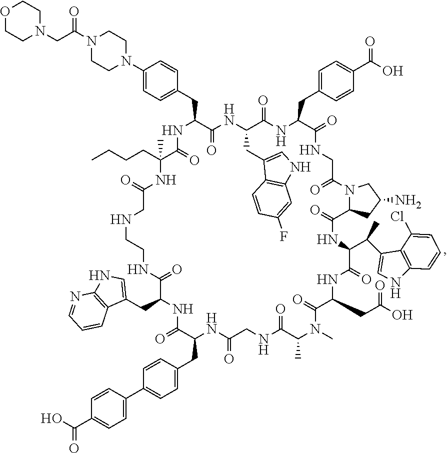 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(=O)CN4CCOCC4)CC3)cc2)NC1=O] 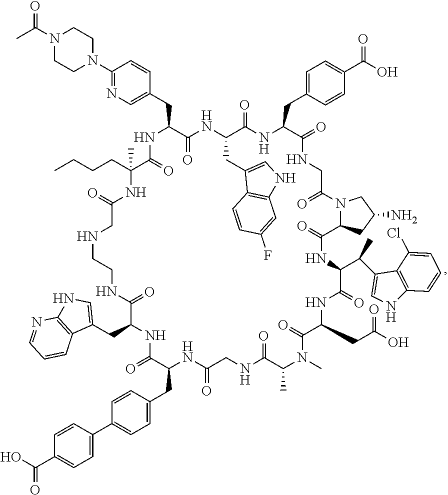 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(C)=O)CC3)nc2)NC1=O] 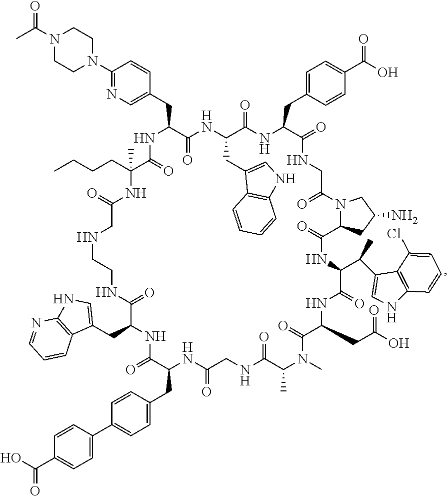 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3ccccc23)NC(=O)[C@H](Cc2ccc(N3CCN(C(C)=O)CC3)nc2)NC1=O] 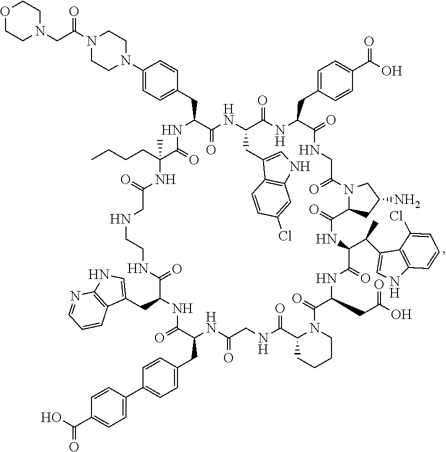 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)CN4CCOCC4)CC3)cc2)NC1=O] 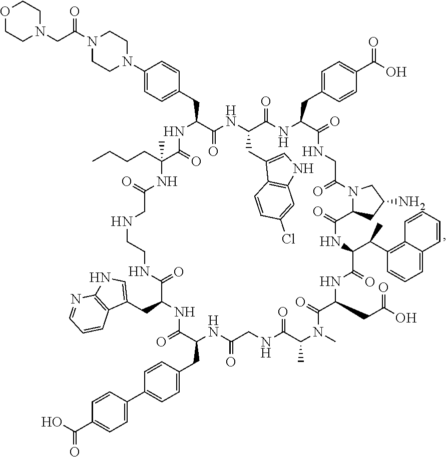 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2cccc3ccccc23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(=O)CN4CCOCC4)CC3)cc2)NC1=O] 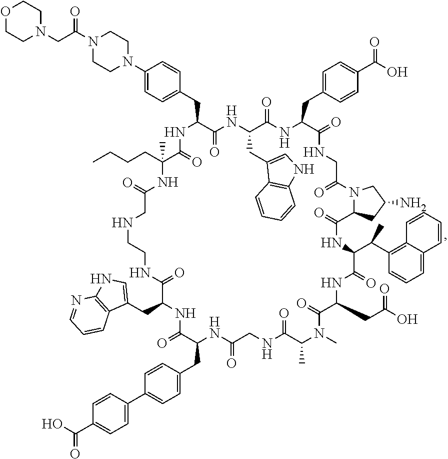 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2cccc3ccccc23)NC(=O)[C@@H]2C[C@@H](N)CN2C(=O)CNC(=O)[C@H](Cc2ccc(C(=O)O)cc2)NC(=O)[C@H](Cc2c[nH]c3ccccc23)NC(=O)[C@H](Cc2ccc(N3CCN(C(=O)CN4CCOCC4)CC3)cc2)NC1=O] 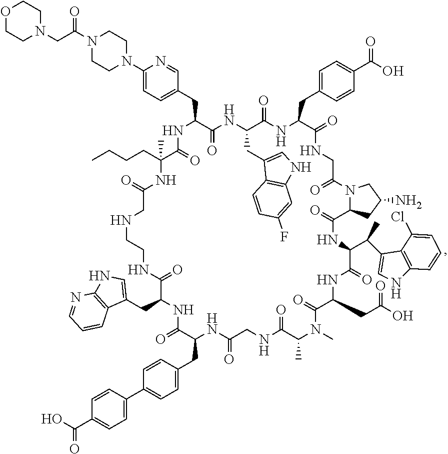 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(=O)CN4CCOCC4)CC3)nc2)NC1=O] 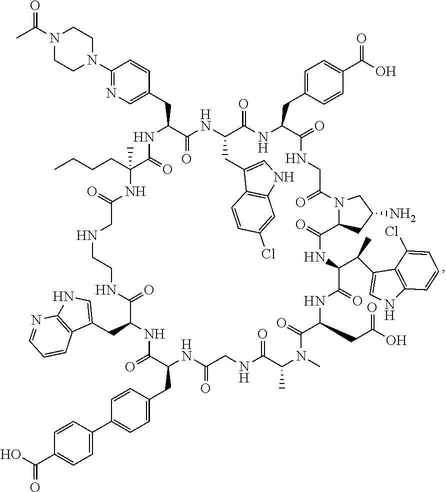 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@@H](C)N(C)C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(Cl)ccc23)NC(=O)[C@H](Cc2ccc(N3CCN(C(C)=O)CC3)nc2)NC1=O] 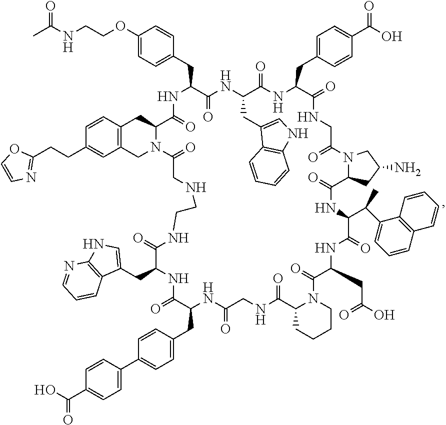 [SMILES: CC(=O)NCCOc1ccc(C[C@@H]2NC(=O)[C@@H]3Cc4ccc(CCc5ncco5)cc4CN3C(=O)CNCCNC(=O)[C@H](Cc3c[nH]c4ncccc34)NC(=O)[C@H](Cc3ccc(-c4ccc(C(=O)O)cc4)cc3)NC(=O)CNC(=O)[C@H]3CCCCN3C(=O)[C@H](CC(=O)O)NC(=O)[C@H]([C@@H](C)c3cccc4ccccc34)NC(=O)[C@@H]3C[C@@H](N)CN3C(=O)CNC(=O)[C@H](Cc3ccc(C(=O)O)cc3)NC(=O)[C@H](Cc3c[nH]c4ccccc34)NC2=O)cc1] 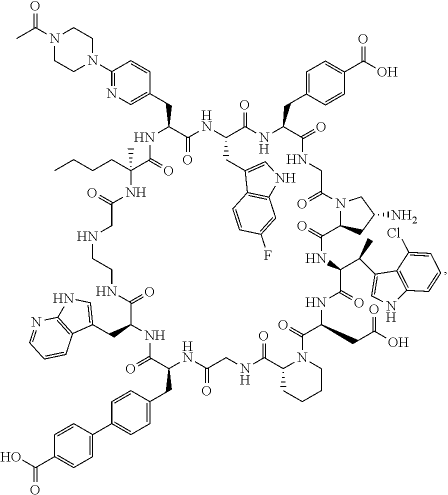 [SMILES: CCCC[C@]1(C)NC(=O)CNCCNC(=O)[C@H](Cc2c[nH]c3ncccc23)NC(=O)[C@H](Cc2ccc(-c3ccc(C(=O)O)cc3)cc2)NC(=O)CNC(=O)[C@H]2CCCCN2C(=O)[C@H](CC(=O)O)NC(=O)[C@H]([C@@H](C)c2c[nH]c3cccc(Cl)c23)NC(=O)[C@@H]2C[C@@H](N)CN2C(=O)CNC(=O)[C@H](Cc2ccc(C(=O)O)cc2)NC(=O)[C@H](Cc2c[nH]c3cc(F)ccc23)NC(=O)[C@H](Cc2ccc(N3CCN(C(C)=O)CC3)nc2)NC1=O] 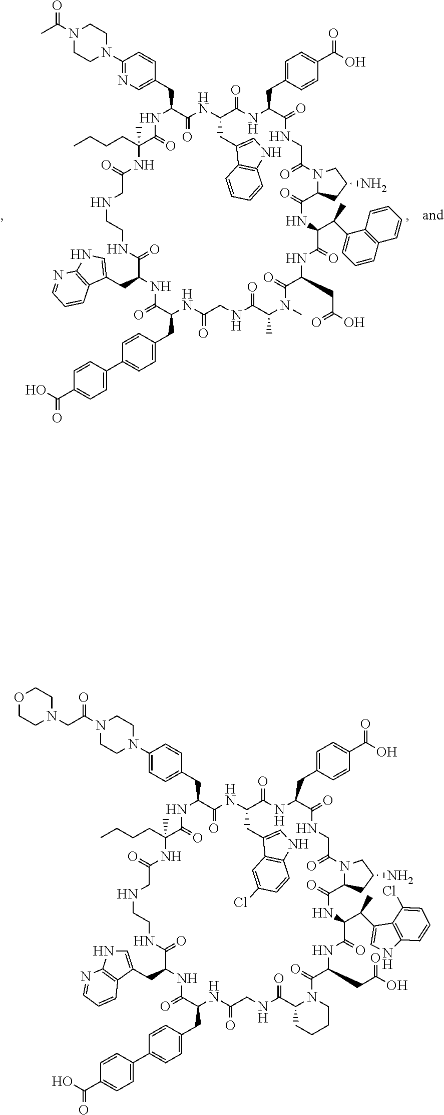
or a pharmaceutically acceptable salt thereof.

22. A pharmaceutical composition comprising the compound of claim 1 or a pharmaceutically acceptable salt thereof, and a pharmaceutically acceptable carrier.

23. A method of treating atherosclerosis, comprising administering a therapeutically effective amount of the compound of claim 1 or a pharmaceutically acceptable salt thereof to a subject in need thereof.

24. A method of treating vascular inflammation, comprising administering a therapeutically effective amount of the compound of claim 1 or a pharmaceutically acceptable salt thereof to a subject in need thereof.

25. A method of treating an inflammatory disorder, comprising administering a therapeutically effective amount of the compound of claim 1 or a pharmaceutically acceptable salt thereof to a subject in need thereof.

26. The method of any one of claim 23, wherein the effective amount of the compound or a pharmaceutically acceptable salt thereof is administered orally to the subject.
